SEQUENCE LISTING (180 sequences)

SEQ ID NO: 1 - protein, 10 aa, synthetic construct
  source 1..10: note = Antibody EXMA-006 VH CDR1 (Exemplary, AbM)
GYTFDVYGIS

SEQ ID NO: 2 - protein, 17 aa, synthetic construct
  source 1..17: note = Antibody EXMA-006, EXMA-007 VH CDR2 (Exemplary, Kabat)
WIAPYSGNTNYAQKLQG

SEQ ID NO: 3 - protein, 14 aa, synthetic construct
  source 1..14: note = Antibody EXMA-006, EXMA-007 VH CDR3 (Exemplary, Kabat, AbM)
DAGTYSPFGYGMDV

SEQ ID NO: 4 - protein, 11 aa, synthetic construct
  source 1..11: note = Antibody EXMA-006 VL CDR1 (Exemplary, Kabat, AbM)
QASQSINNWLA

SEQ ID NO: 5 - protein, 7 aa, synthetic construct
  source 1..7: note = Antibody EXMA-006 VL CDR2 (Exemplary, Kabat, AbM)
KAYNLES

SEQ ID NO: 6 - protein, 10 aa, synthetic construct
  source 1..10: note = Antibody EXMA-006 VL CDR3 (Exemplary, Kabat, AbM, IMGT)
QLFQSLPPFT

SEQ ID NO: 7 - protein, 5 aa, synthetic construct
  source 1..5: note = Antibody EXMA-006 VH CDR1 (Kabat)
VYGIS

SEQ ID NO: 8 - protein, 7 aa, synthetic construct
  source 1..7: note = Antibody EXMA-006 VH CDR1 (Chothia)
GYTFDVY

SEQ ID NO: 9 - protein, 4 aa, synthetic construct
  source 1..4: note = Antibody EXMA-006, EXMA-007 VH CDR2 (Chothia)
PYSG

SEQ ID NO: 10 - protein, 12 aa, synthetic construct
  source 1..12: note = Antibody EXMA-006, EXMA-007 VH CDR3 (Chothia)
AGTYSPFGYGMD

SEQ ID NO: 11 - protein, 7 aa, synthetic construct
  source 1..7: note = Antibody EXMA-006 VL CDR1 (Chothia)
SQSINNW

SEQ ID NO: 12 - skipped

SEQ ID NO: 13 - protein, 7 aa, synthetic construct
  source 1..7: note = Antibody EXMA-006 VL CDR3 (Chothia)
FQSLPPF

SEQ ID NO: 14 - protein, 10 aa, synthetic construct
  source 1..10: note = Antibody EXMA-006, EXMA-007 VH CDR2 (AbM)
WIAPYSGNTN

SEQ ID NO: 15 - protein, 6 aa, synthetic construct
  source 1..6: note = Antibody EXMA-006 VH CDR1 (Contact)
DVYGIS

SEQ ID NO: 16 - protein, 13 aa, synthetic construct
  source 1..13: note = Antibody EXMA-006, EXMA-007 VH CDR2 (Contact)
WMGWIAPYSGNTN

SEQ ID NO: 17 - protein, 15 aa, synthetic construct
  source 1..15: note = Antibody EXMA-006, EXMA-007 VH CDR3 (Contact)
ARDAGTYSPFGYGMD

SEQ ID NO: 18 - protein, 7 aa, synthetic construct
  source 1..7: note = Antibody EXMA-006 VL CDR1 (Contact)
NNWLAWY

SEQ ID NO: 19 - protein, 10 aa, synthetic construct
  source 1..10: note = Antibody EXMA-006 VL CDR2 (Contact)
LLIYKAYNLE

SEQ ID NO: 20 - protein, 9 aa, synthetic construct
  source 1..9: note = Antibody EXMA-006 VL CDR3 (Contact)
QLFQSLPPF

SEQ ID NO: 21 - protein, 8 aa, synthetic construct
  source 1..8: note = Antibody EXMA-006 VH CDR1 (IMGT)
GYTFDVYG

SEQ ID NO: 22 - protein, 8 aa, synthetic construct
  source 1..8: note = Antibody EXMA-006, EXMA-007 VH CDR2 (IMGT)
IAPYSGNT

SEQ ID NO: 23 - protein, 16 aa, synthetic construct
  source 1..16: note = Antibody EXMA-006, EXMA-007 VH CDR3 (IMGT)
ARDAGTYSPFGYGMDV

SEQ ID NO: 24 - protein, 6 aa, synthetic construct
  source 1..6: note = Antibody EXMA-006 VL CDR1 (IMGT)
QSINNW

SEQ ID NO: 25 - protein, 123 aa, synthetic construct
  source 1..123: note = Antibody EXMA-006 VH Sequence
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 26 - protein, 108 aa, synthetic construct
  source 1..108: note = Antibody EXMA-006 VL Sequence
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIK

SEQ ID NO: 27 - protein, 10 aa, synthetic construct
  source 1..10: note = Antibody EXMA-007 VH CDR1 (Exemplary, AbM)
GYTFDAYGIS

SEQ ID NO: 28 - protein, 11 aa, synthetic construct
  source 1..11: note = Antibody EXMA-007 VL CDR1 (Exemplary, Kabat, AbM)
RASESISNWLA

SEQ ID NO: 29 - protein, 7 aa, synthetic construct
  source 1..7: note = Antibody EXMA-007 VL CDR2 (Exemplary, Kabat, AbM)
KAYSLEY

SEQ ID NO: 30 - protein, 10 aa, synthetic construct
  source 1..10: note = Antibody EXMA-007 VL CDR3 (Exemplary, Kabat, AbM, IMGT)
QQFQKLPPFT

SEQ ID NO: 31 - protein, 5 aa, synthetic construct
  source 1..5: note = Antibody EXMA-007 VH CDR1 (Kabat)
AYGIS

SEQ ID NO: 32 - protein, 7 aa, synthetic construct
  source 1..7: note = Antibody EXMA-007 VH CDR1 (Chothia)
GYTFDAY

SEQ ID NO: 33 - protein, 7 aa, synthetic construct
  source 1..7: note = Antibody EXMA-007 VL CDR1 (Chothia)
SESISNW

SEQ ID NO: 34 - protein, 7 aa, synthetic construct
  source 1..7: note = Antibody EXMA-007 VL CDR3 (Chothia)
FQKLPPF

SEQ ID NO: 35 - protein, 6 aa, synthetic construct
  source 1..6: note = Antibody EXMA-007 VH CDR1 (Contact)
DAYGIS

SEQ ID NO: 36 - protein, 7 aa, synthetic construct
  source 1..7: note = Antibody EXMA-007 VL CDR1 (Contact)
SNWLAWY

SEQ ID NO: 37 - protein, 10 aa, synthetic construct
  source 1..10: note = Antibody EXMA-007 VL CDR2 (Contact)
LLIYKAYSLE

SEQ ID NO: 38 - protein, 9 aa, synthetic construct
  source 1..9: note = Antibody EXMA-007 VL CDR3 (Contact)
QQFQKLPPF

SEQ ID NO: 39 - protein, 8 aa, synthetic construct
  source 1..8: note = Antibody EXMA-007 VH CDR1 (IMGT)
GYTFDAYG

SEQ ID NO: 40 - protein, 6 aa, synthetic construct
  source 1..6: note = Antibody EXMA-007 VL CDR1 (IMGT)
ESISNW

SEQ ID NO: 41 - protein, 123 aa, synthetic construct
  source 1..123: note = Antibody EXMA-007 VH Sequence
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSS

SEQ ID NO: 42 - protein, 108 aa, synthetic construct
  source 1..108: note = Antibody EXMA-007 VL Sequence
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIK

SEQ ID NO: 43-75 - skipped (33 entries)

SEQ ID NO: 76 - protein, 222 aa, synthetic construct
  source 1..222: note = Exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 459 aa, synthetic construct
  source 1..459: note = Exemplary antibody that binds to TF
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 459 aa, synthetic construct
  source 1..459: note = Exemplary antibody that binds to TF
  MOD_RES 215: note = FORMYLATION
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSLXTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 459 aa, synthetic construct; identical to SEQ ID NO: 78
  source 1..459: note = Exemplary TF-ADC comprises a heavy chain
  MOD_RES 215: note = the amino acid residue coupled to the drug or active agent through a linker

SEQ ID NO: 80 - protein, 464 aa, synthetic construct
  source 1..464: note = Exemplary antibody that binds to TF
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALCTPSRGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 81 - protein, 464 aa, synthetic construct
  source 1..464: note = Exemplary antibody that binds to TF
  MOD_RES 170: note = FORMYLATION
  MOD_RES 458: note = FORMYLATION
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALXTPSRGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLXTPSRGS

SEQ ID NO: 82 - protein, 464 aa, synthetic construct; identical to SEQ ID NO: 81
  source 1..464: note = Exemplary TF-ADC comprises a heavy chain
  MOD_RES 170: note = the amino acid residue coupled to the drug or active agent through a linker
  MOD_RES 458: note = the amino acid residue coupled to the drug or active agent through a linker

SEQ ID NO: 83 - protein, 459 aa, synthetic construct
  source 1..459: note = Exemplary antibody that binds to TF
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSLCTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 459 aa, synthetic construct
  source 1..459: note = Exemplary antibody that binds to TF
  MOD_RES 215: note = FORMYLATION
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSLXTPSRNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 459 aa, synthetic construct; identical to SEQ ID NO: 84
  source 1..459: note = Exemplary TF-ADC comprises a heavy chain
  MOD_RES 215: note = the amino acid residue coupled to the drug or active agent through a linker

SEQ ID NO: 86 - protein, 464 aa, synthetic construct
  source 1..464: note = Exemplary antibody that binds to TF
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALCTPSRGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLCTPSRGS

SEQ ID NO: 87 - protein, 464 aa, synthetic construct
  source 1..464: note = Exemplary antibody that binds to TF
  MOD_RES 170: note = FORMYLATION
  MOD_RES 458: note = FORMYLATION
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALXTPSRGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSLXTPSRGS

SEQ ID NO: 88 - protein, 464 aa, synthetic construct; identical to SEQ ID NO: 87
  source 1..464: note = Exemplary TF-ADC comprises a heavy chain
  MOD_RES 170: note = the amino acid residue coupled to the drug or active agent through a linker
  MOD_RES 458: note = the amino acid residue coupled to the drug or active agent through a linker

SEQ ID NO: 89 - protein, 329 aa, synthetic construct
  source 1..329: note = Exemplary IgG heavy chain comprising any VH amino acid sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 329 aa, synthetic construct; identical to SEQ ID NO: 89
  source 1..329: note = Exemplary IgG heavy chain comprising any VH amino acid sequence

SEQ ID NO: 91 - protein, 215 aa, synthetic construct
  source 1..215: note = Exemplary antibody that binds to TF comprises a light chain
DIQMTQSPSTLSASVGDRVTITCQASQSINNWLAWYQQKPGKAPKLLIYKAYNLESGVPSRFSGSGSGTEFTLTISSLQPDDFATYYCQLFQSLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 215 aa, synthetic construct
  source 1..215: note = Exemplary antibody that binds to TF comprises a light chain
DIQMTQSPSTLSASVGDRVTITCRASESISNWLAWYQQKPGKAPKLLIYKAYSLEYGVPSRFSGSGSGTEFTLTISSLQPDDFATYYCQQFQKLPPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 452 aa, synthetic construct
  source 1..452: note = Exemplary antibody that binds to TF comprises a heavy chain
QVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 452 aa, synthetic construct
  source 1..452: note = Exemplary antibody that binds to TF comprises a heavy chain
QVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 95 - protein, 107 aa, synthetic construct
  source 1..107: note = Exemplary light chain (e.g., for pairing with an IgG heavy chain) comprising any VL amino acid sequence
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 107 aa, synthetic construct [sequence omitted]

SEQ ID NO: 97 - protein, 19 aa, synthetic construct
  source 1..19: note = Exemplary signal peptide
MMSFVSLLLVGILFHATQA

SEQ ID NO: 98 - protein, 19 aa, synthetic construct
  source 1..19: note = Exemplary signal peptide
MGWSLILLFLVAVATRVHS

SEQ ID NO: 99 - protein, 6 aa, synthetic construct
  source 1..6: note = Exemplary sulfatase motif
  VARIANT 2: note = X is C or S
LXTPSR

SEQ ID NO: 100 - protein, 6 aa, synthetic construct
  source 1..6: note = Exemplary sulfatase motif
LCTPSR

SEQ ID NO: 101 - protein, 6 aa, synthetic construct
  source 1..6: note = Exemplary sulfatase motif
MCTPSR

SEQ ID NO: 102 - protein, 6 aa, synthetic construct
  source 1..6: note = Exemplary sulfatase motif
VCTPSR

SEQ ID NO: 103 - protein, 6 aa, synthetic construct
  source 1..6: note = Exemplary sulfatase motif
LCSPSR

SEQ ID NO: 104 - protein, 6 aa, synthetic construct
  source 1..6: note = Exemplary sulfatase motif
LCAPSR

SEQ ID NO: 105 - protein, 6 aa, synthetic construct
  source 1..6: note = Exemplary sulfatase motif
LCVPSR

SEQ ID NO: 106 - protein, 6 aa, synthetic construct
  source 1..6: note = Exemplary sulfatase motif
LCGPSR

SEQ ID NO: 107 - protein, 6 aa, synthetic construct
  source 1..6: note = Exemplary sulfatase motif
ICTPAR

SEQ ID NO: 108 - protein, 6 aa, synthetic construct
  source 1..6: note = Exemplary sulfatase motif
LCTPSK

SEQ ID NO: 109 - protein, 6 aa, synthetic construct
  source 1..6: note = Exemplary sulfatase motif
MCTPSK

SEQ ID NO: 110 - protein, 6 aa, synthetic construct
  source 1..6: note = Exemplary sulfatase motif
VCTPSK

SEQ ID NO: 111 - protein, 6 aa, synthetic construct
  source 1..6: note = Exemplary sulfatase motif
LCSPSK

SEQ ID NO: 112 - protein, 6 aa, synthetic construct
  source 1..6: note = Exemplary sulfatase motif
LCAPSK

SEQ ID NO: 113 - protein, 6 aa, synthetic construct
  source 1..6: note = Exemplary sulfatase motif
LCVPSK

SEQ ID NO: 114 - protein, 6 aa, synthetic construct
  source 1..6: note = Exemplary sulfatase motif
LCGPSK

SEQ ID NO: 115 - protein, 6 aa, synthetic construct
  source 1..6: note = Exemplary sulfatase motif
LCTPSA

SEQ ID NO: 116 - protein, 6 aa, synthetic construct
  source 1..6: note = Exemplary sulfatase motif
ICTPAA

SEQ ID NO: 117 - protein, 6 aa, synthetic construct
  source 1..6: note = Exemplary sulfatase motif
MCTPSA

SEQ ID NO: 118 - protein, 6 aa, synthetic construct
  source 1..6: note = Exemplary sulfatase motif
VCTPSA

SEQ ID NO: 119 - protein, 6 aa, synthetic construct
  source 1..6: note = Exemplary sulfatase motif
LCSPSA

SEQ ID NO: 120 - protein, 6 aa, synthetic construct
  source 1..6: note = Exemplary sulfatase motif
LCAPSA

SEQ ID NO: 121 - protein, 6 aa, synthetic construct
  source 1..6: note = Exemplary sulfatase motif
LCVPSA

SEQ ID NO: 122 - protein, 6 aa, synthetic construct
  source 1..6: note = Exemplary sulfatase motif
LCGPSA

SEQ ID NO: 123 - protein, 6 aa, synthetic construct
  source 1..6: note = Exemplary fGly-containing sulfatase motif
  MOD_RES 2: note = FORMYLATION
LXTPSR

SEQ ID NO: 124 - protein, 6 aa, synthetic construct
  source 1..6: note = Exemplary fGly-containing sulfatase motif
  MOD_RES 2: note = FORMYLATION
MXTPSR

SEQ ID NO: 125 - protein, 6 aa, synthetic construct
  source 1..6: note = Exemplary fGly-containing sulfatase motif
  MOD_RES 2: note = FORMYLATION
VXTPSR

SEQ ID NO: 126 - protein, 6 aa, synthetic construct
  source 1..6: note = Exemplary fGly-containing sulfatase motif
  MOD_RES 2: note = FORMYLATION
LXSPSR

SEQ ID NO: 127 - protein, 6 aa, synthetic construct
  source 1..6: note = Exemplary fGly-containing sulfatase motif
  MOD_RES 2: note = FORMYLATION
LXAPSR

SEQ ID NO: 128 - protein, 6 aa, synthetic construct
  source 1..6: note = Exemplary fGly-containing sulfatase motif
  MOD_RES 2: note = FORMYLATION
LXVPSR

SEQ ID NO: 129 - protein, 6 aa, synthetic construct
  source 1..6: note = Exemplary fGly-containing sulfatase motif
  MOD_RES 2: note = FORMYLATION
LXGPSR

SEQ ID NO: 130 - protein, 6 aa, synthetic construct
  source 1..6: note = Exemplary fGly-containing sulfatase motif
  MOD_RES 2: note = FORMYLATION
IXTPAR

SEQ ID NO: 131 - protein, 6 aa, synthetic construct
  source 1..6: note = Exemplary fGly-containing sulfatase motif
  MOD_RES 2: note = FORMYLATION
LXTPSK

SEQ ID NO: 132 - protein, 6 aa, synthetic construct
  source 1..6: note = Exemplary fGly-containing sulfatase motif
  MOD_RES 2: note = FORMYLATION
MXTPSK

SEQ ID NO: 133 - protein, 6 aa, synthetic construct
  source 1..6: note = Exemplary fGly-containing sulfatase motif
  MOD_RES 2: note = FORMYLATION
VXTPSK

SEQ ID NO: 134 - protein, 6 aa, synthetic construct
  source 1..6: note = Exemplary fGly-containing sulfatase motif
  MOD_RES 2: note = FORMYLATION
LXSPSK

SEQ ID NO: 135 - protein, 6 aa, synthetic construct
  source 1..6: note = Exemplary fGly-containing sulfatase motif
  MOD_RES 2: note = FORMYLATION
LXAPSK

SEQ ID NO: 136 - protein, 6 aa, synthetic construct
  source 1..6: note = Exemplary fGly-containing sulfatase motif
  MOD_RES 2: note = FORMYLATION
LXVPSK

SEQ ID NO: 137 - protein, 6 aa, synthetic construct
  source 1..6: note = Exemplary fGly-containing sulfatase motif
  MOD_RES 2: note = FORMYLATION
LXGPSK

SEQ ID NO: 138 - protein, 6 aa, synthetic construct
  source 1..6: note = Exemplary fGly-containing sulfatase motif
  MOD_RES 2: note = FORMYLATION
LXTPSA

SEQ ID NO: 139 - protein, 6 aa, synthetic construct
  source 1..6: note = Exemplary fGly-containing sulfatase motif
  MOD_RES 2: note = FORMYLATION
IXTPAA

SEQ ID NO: 140 - protein, 6 aa, synthetic construct
  source 1..6: note = Exemplary fGly-containing sulfatase motif
  MOD_RES 2: note = FORMYLATION
MXTPSA

SEQ ID NO: 141 - protein, 6 aa, synthetic construct
  source 1..6: note = Exemplary fGly-containing sulfatase motif
  MOD_RES 2: note = FORMYLATION
VXTPSA

SEQ ID NO: 142 - protein, 6 aa, synthetic construct
  source 1..6: note = Exemplary fGly-containing sulfatase motif
  MOD_RES 2: note = FORMYLATION
LXSPSA

SEQ ID NO: 143 - protein, 6 aa, synthetic construct
  source 1..6: note = Exemplary fGly-containing sulfatase motif
  MOD_RES 2: note = FORMYLATION
LXAPSA

SEQ ID NO: 144 - protein, 6 aa, synthetic construct
  source 1..6: note = Exemplary fGly-containing sulfatase motif
  MOD_RES 2: note = FORMYLATION
LXVPSA

SEQ ID NO: 145 - protein, 6 aa, synthetic construct
  source 1..6: note = Exemplary fGly-containing sulfatase motif
  MOD_RES 2: note = FORMYLATION
LXGPSA

SEQ ID NO: 146 - protein, 6 aa, synthetic construct
  source 1..6: note = Exemplary fGly'-containing sulfatase motif
  MOD_RES 2: note = the amino acid residue coupled to the drug or active agent through a linker
LXTPSR

SEQ ID NO: 147 - protein, 6 aa, synthetic construct
  source 1..6: note = Exemplary fGly'-containing sulfatase motif
  MOD_RES 2: note = the amino acid residue coupled to the drug or active agent through a linker
MXTPSR

SEQ ID NO: 148 - protein, 6 aa, synthetic construct
  source 1..6: note = Exemplary fGly'-containing sulfatase motif
  MOD_RES 2: note = the amino acid residue coupled to the drug or active agent through a linker
VXTPSR

SEQ ID NO: 149 - protein, 6 aa, synthetic construct
  source 1..6: note = Exemplary fGly'-containing sulfatase motif
  MOD_RES 2: note = the amino acid residue coupled to the drug or active agent through a linker
LXSPSR

SEQ ID NO: 150 - protein, 6 aa, synthetic construct
  source 1..6: note = Exemplary fGly'-containing sulfatase motif
  MOD_RES 2: note = the amino acid residue coupled to the drug or active agent through a linker
LXAPSR

SEQ ID NO: 151 - protein, 6 aa, synthetic construct
  source 1..6: note = Exemplary fGly'-containing sulfatase motif
  MOD_RES 2: note = the amino acid residue coupled to the drug or active agent through a linker
LXVPSR

SEQ ID NO: 152 - protein, 6 aa, synthetic construct
  source 1..6: note = Exemplary fGly'-containing sulfatase motif
  MOD_RES 2: note = the amino acid residue coupled to the drug or active agent through a linker
LXGPSR

SEQ ID NO: 153 - protein, 6 aa, synthetic construct
  source 1..6: note = Exemplary fGly'-containing sulfatase motif
  MOD_RES 2: note = the amino acid residue coupled to the drug or active agent through a linker
IXTPAR

SEQ ID NO: 154 - protein, 6 aa, synthetic construct
  source 1..6: note = Exemplary fGly'-containing sulfatase motif
  MOD_RES 2: note = the amino acid residue coupled to the drug or active agent through a linker
LXTPSK

SEQ ID NO: 155 - protein, 6 aa, synthetic construct
  source 1..6: note = Exemplary fGly'-containing sulfatase motif
  MOD_RES 2: note = the amino acid residue coupled to the drug or active agent through a linker
MXTPSK

SEQ ID NO: 156 - protein, 6 aa, synthetic construct
  source 1..6: note = Exemplary fGly'-containing sulfatase motif
  MOD_RES 2: note = the amino acid residue coupled to the drug or active agent through a linker
VXTPSK

SEQ ID NO: 157 - protein, 6 aa, synthetic construct
  source 1..6: note = Exemplary fGly'-containing sulfatase motif
  MOD_RES 2: note = the amino acid residue coupled to the drug or active agent through a linker
LXSPSK

SEQ ID NO: 158 - protein, 6 aa, synthetic construct
  source 1..6: note = Exemplary fGly'-containing sulfatase motif
  MOD_RES 2: note = the amino acid residue coupled to the drug or active agent through a linker
LXAPSK

SEQ ID NO: 159 - protein, 6 aa, synthetic construct
  source 1..6: note = Exemplary fGly'-containing sulfatase motif
  MOD_RES 2: note = the amino acid residue coupled to the drug or active agent through a linker
LXVPSK

SEQ ID NO: 160 - protein, 6 aa, synthetic construct
  source 1..6: note = Exemplary fGly'-containing sulfatase motif
  MOD_RES 2: note = the amino acid residue coupled to the drug or active agent through a linker
LXGPSK

SEQ ID NO: 161 - protein, 6 aa, synthetic construct
  source 1..6: note = Exemplary fGly'-containing sulfatase motif
  MOD_RES 2: note = the amino acid residue coupled to the drug or active agent through a linker
LXTPSA

SEQ ID NO: 162 - protein, 6 aa, synthetic construct
  source 1..6: note = Exemplary fGly'-containing sulfatase motif
  MOD_RES 2: note = the amino acid residue coupled to the drug or active agent through a linker
IXTPAA

SEQ ID NO: 163 - protein, 6 aa, synthetic construct
  source 1..6: note = Exemplary fGly'-containing sulfatase motif
  MOD_RES 2: note = the amino acid residue coupled to the drug or active agent through a linker
MXTPSA

SEQ ID NO: 164 - protein, 6 aa, synthetic construct
  source 1..6: note = Exemplary fGly'-containing sulfatase motif
  MOD_RES 2: note = the amino acid residue coupled to the drug or active agent through a linker
VXTPSA

SEQ ID NO: 165 - protein, 6 aa, synthetic construct
  source 1..6: note = Exemplary fGly'-containing sulfatase motif
  MOD_RES 2: note = the amino acid residue coupled to the drug or active agent through a linker
LXSPSA

SEQ ID NO: 166 - protein, 6 aa, synthetic construct
  source 1..6: note = Exemplary fGly'-containing sulfatase motif
  MOD_RES 2: note = the amino acid residue coupled to the drug or active agent through a linker
LXAPSA

SEQ ID NO: 167 - protein, 6 aa, synthetic construct
  source 1..6: note = Exemplary fGly'-containing sulfatase motif
  MOD_RES 2: note = the amino acid residue coupled to the drug or active agent through a linker
LXVPSA

SEQ ID NO: 168 - protein, 6 aa, synthetic construct
  source 1..6: note = Exemplary fGly'-containing sulfatase motif
  MOD_RES 2: note = the amino acid residue coupled to the drug or active agent through a linker
LXGPSA

SEQ ID NO: 169-174 - skipped (6 entries)

SEQ ID NO: 175 - protein, 295 aa, Homo sapiens
  source 1..295: note = Human TF protein
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 176 - protein, 264 aa, Macaca fascicularis
  source 1..264: note = Cynomolgus TF protein
SGTTNTVAAYNLTWKSTNFKTILEWEPKPINQVYTVQISTKSGDWKSKCFYTADTECDLTDEIVKDVKQTYLARVFSYPAGHVESTGSTEEPPYENSPEFTPYLETNLGQPTIQSFEQVGTKVNVTVQDEWTLVRRNDTFLSLRDVFGKDLIYTLYYWKSSSSGKKTAKTNTNEFLIDVDKGENYCFSVQAVIPSRRTANRKSTDSPVECMGHEKGESREIFYIIGAVVFVVIILVIILAISLHKCKKARVGRSWKENSPLNVA

SEQ ID NO: 177 - protein, 266 aa, Mus musculus
  source 1..266: note = Mouse TF protein
AGIPEKAFNLTWISTDFKTILEWQPKPTNYTYTVQISDRSRNWKNKCFSTTDTECDLTDEIVKDVTWAYEAKVLSVPRRNSVHGDGDQLVIHGEEPPFTNAPKFLPYRDTNLGQPVIQQFEQDGRKLNVVVKDSLTLVRKNGTFLTLRQVFGKDLGYIITYRKGSSTGKKTNITNTNEFSIDVEEGVSYCFFVQAMIFSRKTNQNSPGSSTVCTEQWKSFLGETLIIVGAVVLLATIFIILLSISLCKRRKNRAGQKGKNTPSRLA

SEQ ID NO: 178 - protein, 260 aa, Sus scrofa
  source 1..260: note = Pig TF protein
TGTTDVIVAYNLTWKSTNFKTILEWEPKPINYVYTVQISPRLGDWKNKCFHTTDTECDVTDEIMRNVKETYVARVLSYPADTVLTAQEPPFTNSPPFTPYLDTNLGQPVIQSFEQVGTKLNVTVEAARTLVRVNGTFLRLRDVFGKDLNYTLYYWRASSTGKKKATTNTNEFLIDVDKGENYCFSVQAVIPSRRVNQKSPESRIECTSQEKAVSRELFLIVGAVVFAVIVFVLVLSVSLYKCRKERAGPSGKENAPLNVA

SEQ ID NO: 179 - protein, 471 aa, synthetic construct
  source 1..471: note = Exemplary TF antibody comprises a heavy chain with a signal peptide
MMSFVSLLLVGILFHATQAQVQLVQSGAEVKKPGASVKVSCKASGYTFDVYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 180 - protein, 471 aa, synthetic construct
  source 1..471: note = Exemplary TF antibody comprises a heavy chain with a signal peptide
MMSFVSLLLVGILFHATQAQVQLVQSGAEVKKPGASVKVSCKASGYTFDAYGISWVRQAPGQGLEWMGWIAPYSGNTNYAQKLQGRVTMTTDTSTSTAYMELRSLRSDDTAVYYCARDAGTYSPFGYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2026/0325667 A1
(43) Pub. Date: Oct. 22, 2026

(54) TISSUE FACTOR ANTIBODY-DRUG CONJUGATES AND USES THEREOF

(71) Applicant: EXELIXIS, INC., Alameda, CA (US)

(72) Inventors: Robyn M. BARFIELD, Berkeley, CA (US); Maxine BAUZON, Hercules, CA (US); Penelope M. DRAKE, Castro Valley, CA (US); Seema KANTAK, Pacifica, CA (US); Brian Alan MENDELSOHN, San Diego, CA (US); Tiffany UNSULANGI, San Ramon, CA (US)

(73) Assignee: EXELIXIS, INC., Alameda, CA (US)

(21) Appl. No.: 19/106,532

(22) PCT Filed: Sep. 6, 2023
(86) PCT No.: PCT/US2023/073516

§ 371 Date: Feb. 25, 2025

Related U.S. Application Data
(60) Provisional application No. 63/404,447, filed on Sep. 7, 2022.
(60) Provisional application No. 63/498,233, filed on Apr. 25, 2023.

(51) Int. Cl. A61K 47/68; C07K 16/36

(52) U.S. Cl. CPC A61K 47/6849; A61K 47/68037; A61K 47/6889; C07K 16/36

(57) ABSTRACT

The present disclosure provides anti-tissue factor antibody-drug conjugate structures. The antibody-drug conjugate structures include a branched linker, where two or more payloads per branched linker are attached to an antibody. In addition, the disclosure also encompasses compounds and methods for production of such conjugates. In addition, the disclosure also encompasses methods of using the conjugates.

### CROSS-REFERENCE TO RELATED APPLICATIONS

This application claims the benefit of U.S. Provisional Patent Application No. 63/404,447, filed Sep. 7, 2022 and of U.S. Provisional Patent Application No. 63/498,233, filed Apr. 25, 2023, the disclosure of each of which is incorporated by reference herein it its entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14529-138-228_SEQ_LISTING.xml”, was created on Sep. 5, 2023, and is 191,857 bytes in size.

### FIELD

The present disclosure relates generally to antibody-drug conjugates (ADCs) that bind to tissue factor (TF, e.g., human TF) and methods of their use.

### INTRODUCTION

Blood coagulation involves a complex set of processes that result in blood clotting. Tissue factor (TF) plays an important role in these coagulation processes. TF is a cell surface receptor for the serine protease factor VIIa (FVIIa). The TF/FVIIa complex catalyzes conversion of the inactive protease factor X (FX) into the active protease factor Xa (FXa). FXa and its co-factor FVa form the prothrombinase complex, which generates thrombin from prothrombin. Thrombin converts soluble fibrinogen into insoluble strands of fibrin and catalyzes many other coagulation-related processes. TF is over-expressed on multiple types of solid tumors. In cancer, TF/FVIIa signaling can support angiogenesis, tumor progression, and metastasis.

Antibody-drug conjugates (ADCs) have emerged over the past two decades as a new class of targeted-delivery therapies. A typical ADC includes an antibody-based targeting element attached to a highly potent pharmaceutical agent (payload) via a chemical linker using an available bioconjugation method. The molar ratio of targeting element (e.g., antibody) to attached payload can vary, and is referred to as the drug-to-antibody ratio (DAR). Commonly used bioconjugation methods either exploit endogenous amino acid residues of a protein (i.e., lysine and cysteine), or rely on selective engagement of a bioorthogonal functional group that has been intentionally introduced into the protein. As an example of the latter approach, the Hydrazino-iso-Pictet-Spengler (HIPS) conjugation method (FIG. 1) takes an advantage of an aldehyde functional group (an “aldehyde tag”), which can be introduced into a protein, such as an antibody, through various means (e.g., by the action of formyl generating enzyme (FGE)), serving as the conjugation handle. The aldehyde group cleanly reacts with the HIPS indole moiety to form a stable carbon-carbon bond that permanently attaches the payload of choice to the protein in a single chemical step.

There remains a need in the art for ADCs that can target TF to treat, prevent, or alleviate TF-mediated diseases, disorders, or conditions, such as those involving tumor cells expressing TF.

### SUMMARY

The present disclosure provides ADCs comprising an antibody that binds to tissue factor (“TF-ADC”). Such TF-ADCs, in some embodiments, bind to the same epitope of human TF as an antibody comprising a heavy chain variable region (VH) and a light chain variable region (VL) described herein.

The present disclosure also provides pharmaceutical compositions comprising a TF-ADC that comprises an antibody or fragment thereof that binds to TF (“TF antibody”) and a drug conjugated (directly or indirectly) thereto. Such pharmaceutical compositions, in some embodiments, include TF-ADCs comprising an antibody or fragment thereof that binds to essentially the same epitope of human TF as an antibody comprising a VH and a VL described herein.

The present disclosure also provides methods of treating, preventing, or alleviating a TF-mediated disease, disorder, or condition, such as alleviating one or more symptoms of the TF-mediated disease, disorder, or condition with a TF-ADC.

More specifically, the present disclosure provides a TF-ADC comprising (a) a TF antibody and (b) one or more pyridazine-pyrrolo coupling moieties comprising a drug conjugated to the pyridazine-pyrrolo coupling moiety through a linker, for example, using the Hydrazino-iso-Pictet-Spengler (HIPS) conjugation method.

Traditionally, the HIPS conjugation method has been used to produce conjugates carrying one payload per HIPS moiety per aldehyde tag, which produces antibody conjugates with DAR values of up to 4. In some embodiments, a TF-ADC as disclosed herein comprises branched HIPS linkers that carry two (or more) molecules of the same or different payload per one HIPS moiety and are therefore capable of conjugating two (or more) small molecule payloads per one aldehyde group in a protein in a single conjugation step (FIG. 2). Consequently, the usage of such branched linkers allows the generation of higher DAR site-specific conjugates (e.g., DAR up to 8) with controlled payload placement, which in the context of therapeutic ADCs would result in larger quantities of pharmaceutical agent delivered to the targeted tissue.

The present disclosure provides TF-ADC structures, which comprises (a) a TF antibody, (b) a branched HIPS linker, and (c) a drug. The disclosure also encompasses compounds and methods for production of such conjugates, as well as methods of using the conjugates.

Aspects of the present disclosure include a TF-ADC comprising (a) a TF antibody; and (b) one or more pyridazine-pyrrolo coupling moieties comprising one or more drugs conjugated to the pyridazine-pyrrolo coupling moiety via one or more linkers.

In some embodiments, a TF-ADC is represented by Formula (I), the TF-ADC comprising:
a. an antibody that binds to tissue factor (TF); and
b. two or more drugs conjugated to a pyridazine-pyrrolo coupling moiety, each via a linker

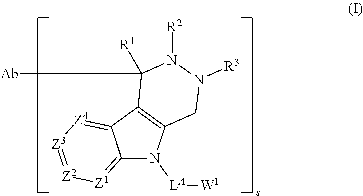

wherein:
Ab represents the antibody that binds to TF;
Z¹, Z², and Z⁴ are each independently CR⁴;
Z³ is C—L^{B}—W²;
R¹, R², R³, and R⁴ are each selected from hydrogen and alkyl;
L^{A} is a first linker comprising:

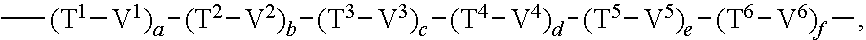

wherein:
a, b, c, d, e and f are each independently 0 or 1, provided at least one of a, b, c, d, e, and f are 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR—, —NR¹⁵(CH₂)_{q}—, —NR¹⁵(C₆H₄)—, —CONR⁵—, —NR¹⁵CO—, —C(O)O—, —OC(O)—, —O—, —S—, —S(O)—, —SO₂—, —SO₂NR⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
L^{B} is a second linker comprising:

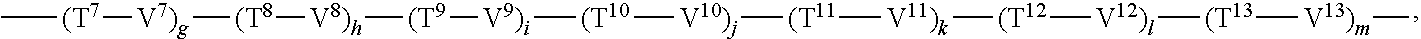

wherein:
g, h, i, j, k, l and m are each independently 0 or 1, provided that at least one of g, h, i, j, k, 1, and m is 1;
T⁷, T⁸, T⁹, T¹⁰, T¹¹, T¹² and T¹³ are each independently selected from a covalent bond, (C₁-C₁₂)alkyl, substituted (C₁-C₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
s is an integer from 1 to 10;
W¹ is a first drug; and
W² is a second drug.

In some embodiments, Z¹ is CR⁴.

In some embodiments, Z³ is C—L^{B}—W².

In some embodiments, one or both of W¹ and W² are camptothecin analogues, for example, belotecan.

In some embodiments, L^{A} comprises:

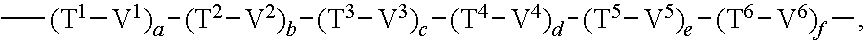

wherein
a, b, c, d, e and f are each independently 0 or 1, provided at least one of a, b, c, d, e, and f is 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments of L^{A}:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V¹, V², V³, V⁴,V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—; wherein:
(PEG)ₙ is

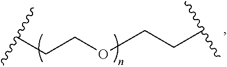

where n is an integer from 1 to 30;
EDA is an ethylene diamine moiety having the following structure:

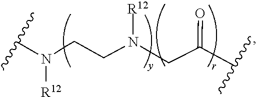

where y is an integer from 1 to 6 and r is 0 or 1;
4-amino-piperidine (4AP) is

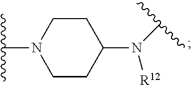

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;

In further embodiments, a, b, c, and d are each 1; and
e and fare 0.

In some embodiments, T¹, T², T³, T⁴, T⁵ and T⁶ are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{A} is a linker wherein:
T¹ is (C₁-C₁₂)alkyl and V¹ is —CONH—;
T² is substituted (C₁-C₁₂)alkyl and V² is —CO—;
T³ is (AA)ₚ and V³ is absent;
T⁴ is PABC and V⁴ is absent;
p is an integer from 1 to 10;
a, b, c, and d are each 1; and
e and f are each 0.

In further embodiments, the PABC is substituted with a glycoside, for example, a hydrogen of PABC is replaced with a glycoside, such as a glucuronide, a galactoside, a glucoside, a mannoside, a fucoside, O-GlcNAc, and O-GalNAc.

In some embodiments, L^{B} comprises:

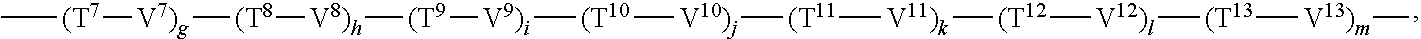

wherein
g, h, i, j, k, l and m are each independently 0 or 1, provided at least one of g, h, i, j, k, l and m is 1;
T⁷, T⁸, T⁹, T¹⁰, T¹¹, T¹² and T¹³ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, T⁷, T⁸, T⁹, T¹⁰, T¹¹, T¹² and T¹³ are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In some embodiments of L^{B}:
T⁷ is a covalent bond;
T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V⁷, V⁸, V⁹, V¹⁰, V¹¹ and V¹² are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;

wherein:
(PEG)ₙ is

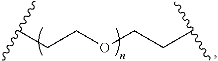

where n is an integer from 1 to 30;
EDA is an ethylene diamine moiety having the following structure:

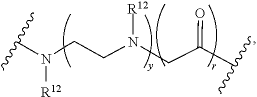

where y is an integer from 1 to 6 and r is 0 or 1;
4-amino-piperidine (4AP) is

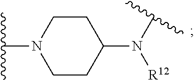

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
g, h, i, j, and k are each 1; and
l and m is 0.

In some embodiments, T¹, T², T³, T⁴, T⁵, T⁶, T⁷, T⁸, T⁹, T¹⁰, T¹¹, and T¹² are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In some embodiments, L^{B} is a linker wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is (AA)ₚ and V¹⁰ is absent;
T¹¹ is PABC and V¹¹ is absent;
p is an integer from 1 to 10;
g, h, i, j, and k are each 1; and
l and m are each 0.

In further embodiments, the PABC is substituted with a glycoside, for example, a hydrogen of PABC is replaced with a glycoside, such as a glucuronide, a galactoside, a glucoside, a mannoside, a fucoside, O-GlcNAc, and O-GalNAc.

In some embodiments, a TF-ADC is represented by Formula (I):

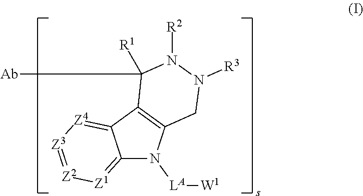

wherein:
Ab represents the antibody that binds to TF;
Z¹, Z², and Z⁴ are each independently CR⁴;
Z³ is C—L^{B}—W²;
R¹, R², R³ and R⁴ are each selected from hydrogen and (C₁-C₁₂)alkyl;
L^{A} is a first linker wherein:
   T¹ is (C₁-C₁₂)alkyl and V¹ is —CONH—;
   T² is substituted (C₁-C₁₂)alkyl and V² is —CO—;
   T³ is (AA)ₚ where p is an integer from 1-20 and V³ is a covalent bond;
   T⁴ is PABC and V⁴ is a covalent bond;
   a, b, c, and d are each 1;
   e and f are each 0; and
L^{B} is a second linker wherein
   T⁷ is a covalent bond and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is (AA)ₚ where p is an integer from 1-20 and V¹⁰ is a covalent bond;
   T¹¹ is PABC and V¹¹ is a covalent bond; and
   g, h, i, j, and k are each 1; and
   l and m are each 0;
s is an integer from 1 to 10;
W¹ is a first drug; and
W² is a second drug.

In some embodiments, one or both of W¹ and W² are camptothecin analogues, for example, belotecan.

In some embodiments, a TF-ADC is represented by Formula (I):

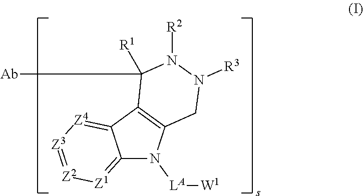

wherein:
Ab represents the antibody that binds to TF;
Z¹, Z², and Z⁴ are each independently CR⁴;
Z³ is C—L^{B}—W²;
R¹, R², R³ and R⁴ are each selected from hydrogen and (C₁-C₁₂)alkyl;
L^{A} is a linker wherein:
   T¹ is (C₁-C₆)alkyl and V¹ is —CONH—;
   T² is (C₁-C₆)alkylene substituted with —NHCO(PEG)ₖ, wherein k is an integer from 2 to 10 and V² is —CO—;
   T³ is (AA)₂ and V³ is a covalent bond;
   T⁴ is PABC substituted with a glycoside and V⁴ is a covalent bond;
   a, b, c, and d are each 1; and
   e and f are each 0; and
L^{B} is a linker wherein
   T⁷ is a covalent bond and V⁷ is —NHCO—;
   T⁸ is (C₁-C₆)alkyl and V⁸ is —CONH—;
   T⁹ is (C₁-C₆)alkylene substituted with —NHCO(PEG)ₖ, wherein k is an integer from 2 to 10 and V⁹ is —CO—;
   T¹⁰ is (AA)₂ and V¹⁰ is a covalent bond;
   T¹¹ is PABC substituted with a glycoside and V¹¹ is a covalent bond;
   g, h, i, j, and k are each 1; and
   l and m are each 0;
s is an integer from 1 to 10;
W¹ is a first drug; and
W² is a second drug.

In some embodiments, the PABC of one or both of T⁴ and T¹¹ is substituted with a glucuronide. In some embodiments, one or both of T¹ and T⁸ is ethyl. In some embodiments, one or both of T² and T⁹ is C₅ alkylene substituted with —NHCO(PEG)ₖ, where k is an integer from 5-10. In some embodiments, one or both of W¹ and W² are camptothecin analogues, for example, belotecan.

In some embodiments, a TF-ADC is represented by Formula (II):

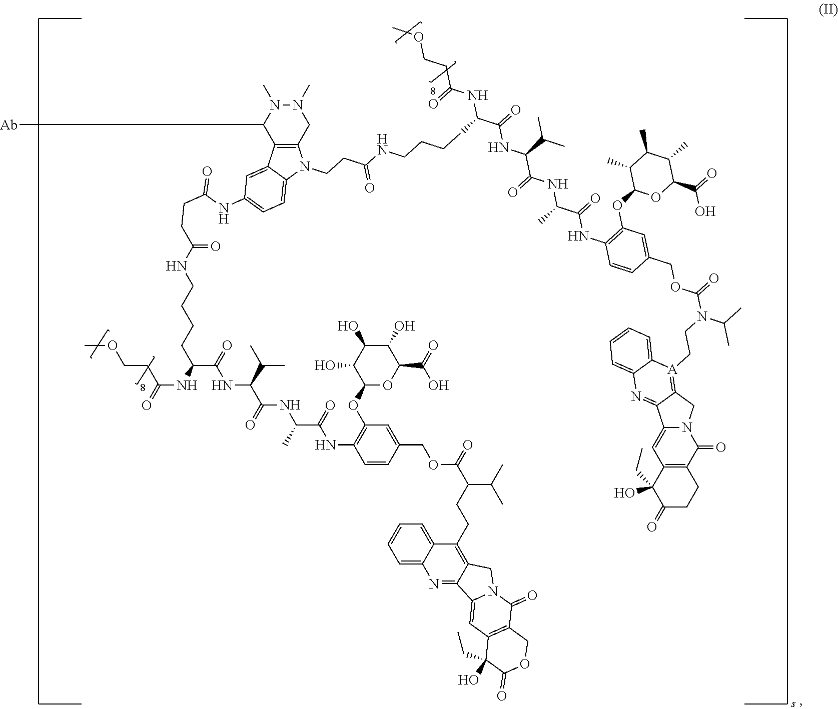

wherein:
Ab represents the antibody that binds to TF; and
s is an integer from 1 to 10.

In some embodiments, s is an integer from 1 to 4.

Formula (II) may be prepared by conjugating one or more linker-payloads of Formula (IIa), shown below, with a TF antibody:

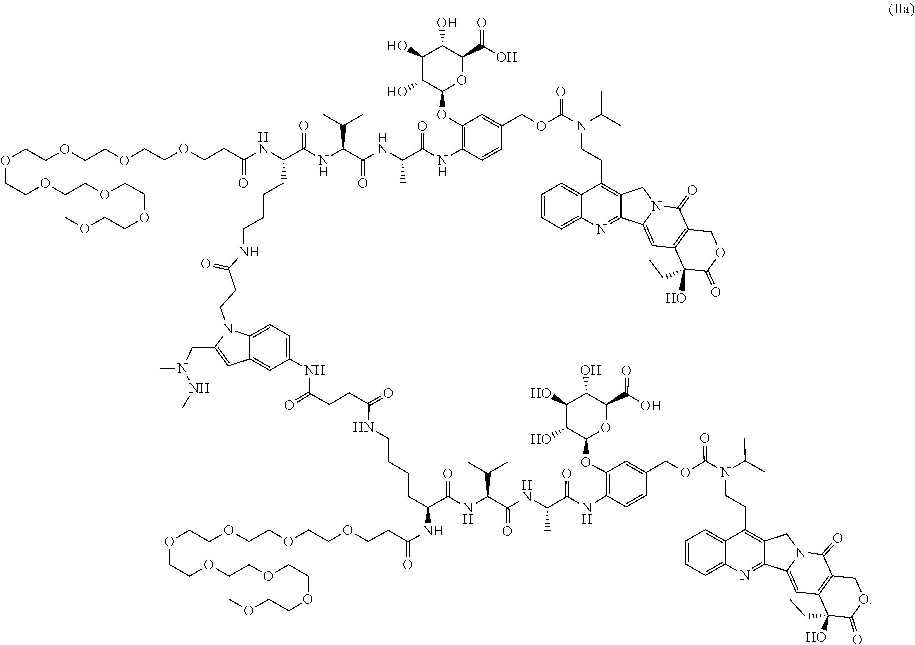 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

In some embodiments, a TF-ADC can be represented by Formula (I) or (II), wherein Ab comprises: a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26.

In some embodiments, a TF-ADC can be represented by Formula (I) or (II), wherein Ab comprises: a VH CDR1, a VH CDR2, and a VH CDR3 as set forth in a VH comprising the amino acid sequence of SEQ TD NO:41 and a VL CDR1, a VL CDR2, and a VL CDR3 as set forth in a VL comprising the amino acid sequence of SEQ ID NO: 42.

In some embodiments, a TF-ADC can be represented by Formula (I) or (II), wherein Ab comprises: (i) a VH region comprising a VH CDR1 comprising the amino acid sequence of SEQ ID NOs:1, 7, 8, 15, 21, 27, 31, 32, 35, or 39, a VH CDR2 comprising the amino acid sequence of SEQ ID NOs:2, 9, 14, 16, or 22, and a VH CDR3 comprising the amino acid sequence of SEQ ID NOs:3, 10, 17, or 23; and (ii) a VL region comprising a VL CDR1 comprising the amino acid sequence of SEQ ID NOs:4, 11, 18, 24, 28, 33, 36, or 40, a VL CDR2 comprising the amino acid sequence of SEQ ID NOs:5, 12, 19, 29, or 37, and a VL CDR3 comprising the amino acid sequence of SEQ ID NOs:6, 13, 20, 30, 34, or 38.

In some embodiments, a TF-ADC can be represented by Formula (I) or (II), wherein the Ab competes with any one of the TF antibodies as disclosed herein in binding to TF.

In some embodiments, a TF-ADC can be represented by Formula (I) or (II) wherein Ab comprises a framework 1 (FR1), a framework 2 (FR2), a framework 3 (FR3) and/or a framework 4 (FR4) sequence as set forth in any one of SEQ ID NOs:25, 26, 41, and 42.

In some embodiments, a TF-ADC can be represented by Formula (I) or (II) wherein Ab comprises human framework sequences.

In some embodiments, a TF-ADC can be represented by Formula (I) or (II) wherein Ab comprises a VH comprising the amino acid sequence of SEQ ID NO:25 and a VL comprising the amino acid sequence of SEQ ID NO:26.

In some embodiments, a TF-ADC can be represented by Formula (I) or (II) wherein Ab comprises a heavy chain comprising the amino acid sequence of SEQ ID NO:82.

Additionally or alternatively, the Ab comprises a VL comprising the amino acid sequence of SEQ ID NO:91. In some embodiments, a TF-ADC can be represented by Formula (I) or (II) wherein Ab comprises a heavy chain comprising the amino acid sequence of SEQ ID NO:82 and a VL comprising the amino acid sequence of SEQ ID NO:91.

In some embodiments, a TF-ADC can be represented by Formula (I) or (II) wherein Ab comprises a heavy chain comprising the amino acid sequence of SEQ ID NO:79. Additionally or alternatively, the Ab comprises a VL comprising the amino acid sequence of SEQ ID NO:91. In some embodiments, a TF-ADC can be represented by Formula (I) or (II) wherein Ab comprises a heavy chain comprising the amino acid sequence of SEQ ID NO:79 and a VL comprising the amino acid sequence of SEQ ID NO:91.

In some embodiments, a TF-ADC can be represented by Formula (I) or (II) wherein Ab comprises (i) a VH comprising the amino acid sequence of SEQ ID NO:41 and a VL comprising the amino acid sequence of SEQ ID NO:42.

In some embodiments, a TF-ADC can be represented by Formula (I) or (II) wherein Ab comprises a heavy chain comprising the amino acid sequence of SEQ ID NO:85.

Additionally or alternatively, the Ab comprises a VL comprising the amino acid sequence of SEQ ID NO:92. In some embodiments, a TF-ADC can be represented by Formula (I) or (II) wherein Ab comprises a heavy chain comprising the amino acid sequence of SEQ ID NO:85 and a VL comprising the amino acid sequence of SEQ ID NO:92.

In some embodiments, a TF-ADC can be represented by Formula (I) or (II) wherein Ab comprises a heavy chain comprising the amino acid sequence of SEQ ID NO:88.

Additionally or alternatively, the Ab comprises a VL comprising the amino acid sequence of SEQ ID NO:92. In some embodiments, a TF-ADC can be represented by Formula (I) or (II) wherein Ab comprises a heavy chain comprising the amino acid sequence of SEQ ID NO:88 and a VL comprising the amino acid sequence of SEQ ID NO:92.

The present disclosure also provides a pharmaceutical composition comprising a TF-ADC, wherein the TF-ADC can be represented by Formula (I) or Formula (II) and a pharmaceutically acceptable excipient, wherein the TF antibody (TF Ab or Ab) is as described in any embodiment described herein. In some embodiments, such a pharmaceutical composition can have a drug-to-antibody ratio (DAR) of the TF-ADC of about 1 to about 20, for example, a DAR of about 2 to about 8, about 1 to about 4, about 2 to about 4, about 3 to about 4, about 4, about 1 to about 8, about 2 to about 8, about 3 to about 8, about 4 to about 8, about 5 to about 8, about 6 to about 8, about 6.5 to about 8, about 6 to about 7, about 6.5 to about 7.5, about 7 to about 8, about 6.5, about 7, about 7.5, or about 8.

The present disclosure also provides a method for treating a cancer or a tumor in a subject comprising administering to the subject the TF-ADC, wherein the TF-ADC can be represented by Formula (I) or (II) or the pharmaceutical composition comprising a TF-ADC of Formula (I) or (II) and a pharmaceutically acceptable excipient, wherein the TF antibody is as described in any embodiment herein.

Provided herein is a kit comprising the antibody-drug conjugate as disclosed herein or the pharmaceutical composition as disclosed herein, and instructions for use.

### BRIEF DESCRIPTION OF THE DRAWINGS

FIG. 1 shows a schematic drawing of the HIPS ligation for the synthesis of ADCs. Antibodies carrying aldehyde moieties are reacted with a Hydrazino-iso-Pictet-Spengler (HIPS) linker and payload to generate a site-specifically conjugated ADC with a stable azacarboline linkage.

FIG. 2 shows a schematic representation of branched HIPS ligation for the synthesis of ADCs. Antibodies carrying four aldehyde moieties are reacted with a branched HIPS linker to generate ADCs with drug-to-antibody (DAR) value of up to 8, according to embodiments of the present disclosure.

FIG. 3A shows a graph of total antibody and ADC concentrations for pharmacokinetic (PK) sample analysis. Square: TF-ADC 6-8—total mAb measurement; triangle: TF-ADC 6-8—total ADC measurement.

FIG. 3B shows a graph of total antibody and ADC concentrations for pharmacokinetic (PK) sample analysis. Diamond: TF-ADC 6-4—total mAb measurement; star: TF-ADC 6-4—total ADC measurement.

FIG. 3C shows a graph of total antibody and ADC concentrations for pharmacokinetic (PK) sample analysis. Inverted triangle: TF-ADC 7-8—total mAb measurement; cross: TF-ADC 7-8—total ADC measurement.

FIG. 3D shows a graph of total antibody and ADC concentrations for pharmacokinetic (PK) sample analysis. Star: TF-ADC 7-4—total mAb measurement; plus: TF-ADC 7-4—total ADC measurement.

FIG. 4 shows graphs of in vitro cytotoxicity assays of free drugs, i.e., Monomethyl auristatin E (MMAE), Belotecan or an Exatecan derivative DxD, in various TF positive cancer cell lines, including RF/6A, A431, HCT-116, BxPC3, AU-565, HPAF-II, MDA-MB-231, AsPC-1, HCC1954, and SKOV-3.

FIG. 5 shows graphs of in vitro cytotoxicity assays of the TF-ADC 6-8 TF antibody conjugated to belotecan (grey circle), the TF-ADC 7-8 TF antibody conjugated to a belotecan (square), a corresponding isotype antibody conjugated to belotecan (triangle), MMAE (diamond), or the free belotecan (black circle), in various TF positive cancer cell lines, including RF/6A, A431, HCT-116, BxPC3, AU-565, HPAF-II, MDA-MB-231, AsPC-1, HCC1954, and SKOV-3.

FIG. 6 shows graphs of in vitro cytotoxicity assays of FITC-ADC 8 (top panel, circle as marked with FITC), TF-ADC 6-8 (top panel, square without any mark), TF-ADC 7-8 (top panel, triangle), Belotecan (top panel, square as marked with B), FITC-ADC 4 (bottom panel, circle as marked with FITC), TF-ADC 6-4 (bottom panel, square), TF-ADC 7-4 (bottom panel, triangle), MMAE (bottom panel, diamond), or Belotecan (bottom panel, circle as marked with B), in three selected TF positive cancer cell lines, including A431, HPAF-II, and BxPC-3.

FIG. 7 shows graphs of in vitro cytotoxicity assays of TF-ADC 6-8 having various DAR levels in three selected TF positive cancer cell lines, including A431, HPAF-II, and BxPC-3.

FIG. 8 shows graphs of in vitro cytotoxicity assays of TF-ADC 6-4 having different DAR levels in three selected TF positive cancer cell lines, including A431, HPAF-II, and BxPC-3.

FIG. 9A shows a graph of mean tumor volume (mm³) vs. days, which indicates in vivo efficacy against a BxPC3 xenograft of TF-targeted ADCs. FIG. 9B shows body weights of the tested mice. FIG. 9C plots the in vivo efficacy data of TF-ADC 6-8 and TF-ADC 7-8.

FIG. 9D plots the in vivo efficacy data of TF-ADC 6-4 and TF-ADC 7-4. FIG. 9E plots the in vivo efficacy data of TF-ADC 6-4 and TF-ADC 6-8. FIG. 9F plots the in vivo efficacy data of TF-ADC 7-4 and TF-ADC 7-8. A single i.v. dose was delivered on Day 0.

FIG. 10A shows a graph of in vivo efficacy against an HPAF-II xenograft of TF-targeted ADCs. FIG. 10B shows body weights of the tested mice. FIG. 10C plots the in vivo efficacy data of TF-ADC 6-8 and TF-ADC 7-8. FIG. 10D plots the in vivo efficacy data of TF-ADC 6-4 and TF-ADC 7-4. FIG. 10E plots the in vivo efficacy data of TF-ADC 6-4 and TF-ADC 6-8. FIG. 10F plots the in vivo efficacy data of TF-ADC 7-4 and TF-ADC 7-8. A single i.v. dose was delivered on Day 0.

FIGS. 11A and 11B show graphs of concentrations of indicated ADC (tADC) and antibody thereof (tAb) in plasma of mice dosed with various ADCs. FIG. 11A plots data from mice dosed with 3 mg/kg or 10 mg/kg of TF-ADC 6-4 or TF-ADC 6-8. FIG. 11B plots data from mice dosed with 1 mg/kg TF-ADC 6-4 or TF-ADC 6-8. EXMA-006 served as a negative control in both graphs, while a benchmark ADC served as a comparator as shown in FIG. 11B.

FIGS. 12A and 12B show graphs of tumor volumes measured in HPAF-II xenograft mice treated with various dosages of TF-ADC 6-4 (FIG. 12A) or TF-ADC 6-8 (FIG. 12B).

FIGS. 13A and 13B provide the concentrations of indicated ADC (ADC) and its antibody (tAb) in plasma of non-human primates (NHP) treated with various dosages of TF-ADC 6-4 (FIG. 13A) or TF-ADC 6-8 (FIG. 13B).

FIGS. 14A and 14B provide the concentrations of payload in plasma of NHP treated with various dosages of TF-ADC 6-4 (FIG. 14A) or TF-ADC 6-8 (FIG. 14B).

FIGS. 15A-15C provides exemplary ATP release results of treated tumor cells (FIGS. 15A-15B, A431; FIG. 15C, SKOV3). FIGS. 15B-15C provide areas under the curve (AUCs) of tumor cells treated at various concentrations of TF-ADC 6-8, isotype control (an isotype IgG1 antibody conjugated to belotecan), or belotecan, while FIG. 15A plots the AUCs in the treatment groups of control (an isotype IgG1 antibody conjugated to belotecan), 33 nM TF-ADC 6-8, 33 nM belotecan, 100 nM MMAE, or 100 nM benchmark ADC.

FIG. 16 plots HMGB1 released by A431 tumor cells treated with control, 33 nM TF-ADC 6-8, 33 nM belotecan, 100 nM MMAE, or 100 nM benchmark ADC.

FIGS. 17A and 17B plot IFNγ released by PBMCs co-cultured for 24 hours (FIG. 17A) or 96 hours (FIG. 17B) with tumor cells pre-treated with isotype ADC, TF-ADC 6-8, free belotecan, or free MMAE plus an anti-PD-1 antibody (referred to herein as MMAE+aPD1). Each bar represents data from one donor.

FIGS. 18A and 18B plot TNFα released by PBMCs co-cultured for 24 hours (FIG. 18A) or 96 hours (FIG. 18B) with tumor cells pre-treated with isotype ADC, TF-ADC 6-8, belotecan, or MMAE plus an anti-PD-1 antibody (referred to herein as MMAE+aPD1). Each bar represents data from one donor.

FIGS. 19A and 19B plot IP-10 released by PBMCs co-cultured for 24 hours (FIG. 19A) or 96 hours (FIG. 19B) with tumor cells pre-treated with isotype ADC, TF-ADC 6-8, belotecan, or MMAE plus an anti-PD-1 antibody (referred to herein as MMAE+aPD1). Each bar represents data from one donor.

FIGS. 20A and 20B plot MIP-1 a released by PBMCs co-cultured for 24 hours (FIG. 20A) or 96 hours (FIG. 20B) with tumor cells pre-treated with isotype ADC, TF-ADC 6-8, belotecan, or MMAE plus an anti-PD-1 antibody (referred to herein as MMAE+aPD1). Each bar represents data from one donor.

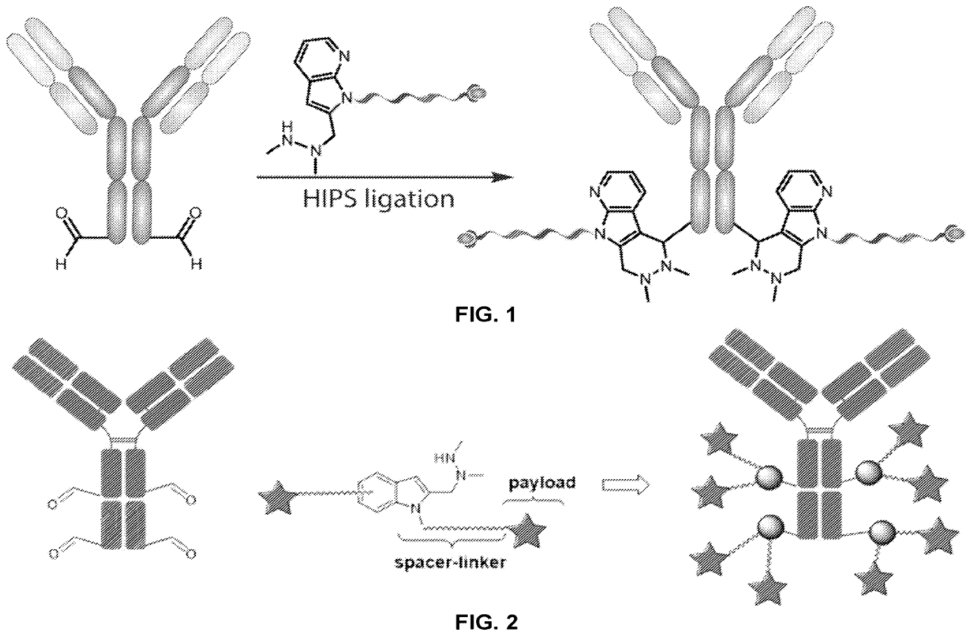

### DETAILED DESCRIPTION

The present disclosure provides antibody-drug conjugates (ADCs) that bind to TF and a drug conjugated (directly or indirectly) thereto. Such TF-ADCs are useful in compositions and in methods of treating, preventing, or alleviating a TF-mediated disease, disorder, or condition, including one or more symptoms of the disease, disorder, or condition. TF-mediated diseases, disorders, and conditions include a variety of cancers, including, but not limited to, any cancer wherein the tumor cells express or overexpress TF. In addition, TF-ADCs are useful for the killing and/or removal of tumor cells. TF-ADCs described herein are useful in compositions and in methods for treating cancer.

### Definitions

The following terms have the following meanings unless otherwise indicated. Any undefined terms have their art-recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms.

This term includes, by way of example, linear and branched hydrocarbyl groups such as methyl(CH₃—), ethyl(CH₃CH₂—), n-propyl(CH₃CH₂CH₂—), isopropyl((CH₃)₂CH—), n-butyl(CH₃CH₂CH₂CH₂—), isobutyl((CH₃)₂CHCH₂—), sec-butyl((CH₃)(CH₃CH₂)CH—), t-butyl((CH₃)₃C—), n-pentyl(CH₃CH₂CH₂CH₂CH₂—), and neopentyl((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂— heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R¹⁵ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group.

Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C—CH), and propargyl (—CH₂C—CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂— aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂— aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO— heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group —SO₂-alkyl, —SO₂-substituted alkyl, —SO₂-alkenyl, —SO₂-substituted alkenyl, —SO₂-cycloalkyl, —SO₂-substituted cycloalkyl, —SO₂-cycloalkenyl, —SO₂-substituted cylcoalkenyl, —SO₂-aryl, —SO₂-substituted aryl, —SO₂-heteroaryl, —SO₂— substituted heteroaryl, —SO₂-heterocyclic, and —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cycloalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, and —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfate” or “sulfate ester” refers the group —O—SO₂—OH, —O—SO₂—O-alkyl, —O—SO₂— O-substituted alkyl, —O—SO₂—O-alkenyl, —O—SO₂—O-substituted alkenyl, —O—SO₂—O-cycloalkyl, —O—SO₂—O-substituted cycloalkyl, —O—SO₂—O-cycloalkenyl, —O—SO₂—O-substituted cylcoalkenyl, —O—SO₂—O-aryl, —O—SO₂—O-substituted aryl, —O—SO₂—O-heteroaryl, —O—SO₂—O-substituted heteroaryl, —O—SO₂—O-heterocyclic, and —O—SO₂—O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O—)₂(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸OR⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C (O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂M⁺, —CO₂R⁷⁰, —C(S)OR⁷⁰, —C(O)NR⁸OR⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷, —N⁷⁰C(S)R⁷⁰, —NR⁷⁰CO₂M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸OR⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O—)₂(M⁺)₂, —P(O)(OR⁷⁰)O⁻M⁺, —P(O)(OR⁷⁰)(OR⁷⁰), —C(O) R⁷⁰, —C(S)R⁷⁰, —C(NR⁷⁰)R⁷⁰, —C(O)OR⁷⁰, —C(S)OR⁷⁰, —C(O)NR⁸OR⁸⁰, —C(NR⁷⁰)NR⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸OR⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)—O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like. In certain embodiments, a polypeptide is an antibody.

“Native amino acid sequence” or “parent amino acid sequence” are used interchangeably herein to refer to the amino acid sequence of a polypeptide prior to modification to include at least one modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” and the like may be used to refer to monomers units and/or polymers of monosaccharides, disaccharides, oligosaccharides, and polysaccharides. The term sugar may be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and may be adapted for use in the subject compounds and conjugates.

The term “glycoside” or “glycosyl” refers to a sugar molecule or group bound to a moiety via a glycosidic bond. For example, the moiety that the glycoside is bound to can be a cleavable linker as described herein. A glycosidic bond can link the glycoside to the other moiety through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some cases, glycosides can be cleaved from the moiety they are attached to, such as by chemically-mediated hydrolysis or enzymatically-mediated hydrolysis.

The term “antibody” is used in the broadest sense and includes monoclonal antibodies (including full length monoclonal antibodies), polyclonal antibodies, and multispecific antibodies (e.g., bispecific antibodies), humanized antibodies, single-chain antibodies, chimeric antibodies, antibody fragments (e.g., Fab fragments), and the like. An antibody is capable of binding a target antigen. (Janeway, C., Travers, P., Walport, M., Shlomchik (2001) Immuno Biology, 5th Ed., Garland Publishing, New York). A target antigen can have one or more binding sites, also called epitopes, recognized by complementarity determining regions (CDRs) formed by one or more variable regions of an antibody.

The term “natural antibody” refers to an antibody in which the heavy and light chains of the antibody have been made and paired by the immune system of a multi-cellular organism. Spleen, lymph nodes, bone marrow and serum are examples of tissues that produce natural antibodies. For example, the antibodies produced by the antibody producing cells isolated from a first animal immunized with an antigen are natural antibodies.

The term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332). In certain embodiments, framework substitutions are identified by modeling of the interactions of the CDR and framework residues to identify framework residues important for antigen binding and sequence comparison to identify unusual framework residues at particular positions (see, e.g., U.S. Pat. No. 5,585,089; Riechmann et al., Nature 332:323 (1988)). Additional methods for humanizing antibodies contemplated for use in the present invention are described in U.S. Pat. Nos. 5,750,078; 5,502,167; 5,705,154; 5,770,403; 5,698,417; 5,693,493; 5,558,864; 4,935,496; and 4,816,567, and PCT publications WO 98/45331 and WO 98/45332. In particular embodiments, a subject rabbit antibody may be humanized according to the methods set forth in US20040086979 and US20050033031. Accordingly, the antibodies described above may be humanized using methods that are well known in the art.

The term “chimeric antibodies” refer to antibodies whose light and heavy chain genes have been constructed, typically by genetic engineering, from antibody variable and constant region genes belonging to different species. For example, the variable segments of the genes from a mouse monoclonal antibody may be joined to human constant segments, such as gamma 1 and gamma 3. An example of a therapeutic chimeric antibody is a hybrid protein composed of the variable or antigen-binding domain from a mouse antibody and the constant or effector domain from a human antibody, although domains from other mammalian species may be used.

An immunoglobulin polypeptide immunoglobulin light or heavy chain variable region is composed of a framework region (FR) interrupted by three hypervariable regions, also called “complementarity determining regions” or “CDRs”. The extent of the framework region and CDRs have been defined (see, “Sequences of Proteins of Immunological Interest,” E. Kabat et al., U.S. Department of Health and Human Services, 1991). The framework region of an antibody, which is the combined framework regions of the constituent light and heavy chains, serves to position and align the CDRs. The CDRs are primarily responsible for binding to an epitope of an antigen.

A “parent Ig polypeptide” is a polypeptide comprising an amino acid sequence which lacks an aldehyde-tagged constant region as described herein. The parent polypeptide may comprise a native sequence constant region, or may comprise a constant region with pre-existing amino acid sequence modifications (such as additions, deletions and/or substitutions).

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

By “reactive partner” is meant a molecule or molecular moiety that specifically reacts with another reactive partner to produce a reaction product. Exemplary reactive partners include a cysteine or serine of a sulfatase motif and Formylglycine Generating Enzyme (FGE), which react to form a reaction product of a converted aldehyde tag containing a formylglycine (fGly) in lieu of cysteine or serine in the motif. Other exemplary reactive partners include an aldehyde of an fGly residue of a converted aldehyde tag (e.g., a reactive aldehyde group) and an “aldehyde-reactive reactive partner”, which comprises an aldehyde-reactive group and a moiety of interest, and which reacts to form a reaction product of a polypeptide having the moiety of interest conjugated to the polypeptide through the fGly residue.

“N-terminus” refers to the terminal amino acid residue of a polypeptide having a free amine group, which amine group in non-N-terminus amino acid residues normally forms part of the covalent backbone of the polypeptide.

“C-terminus” refers to the terminal amino acid residue of a polypeptide having a free carboxyl group, which carboxyl group in non-C-terminus amino acid residues normally forms part of the covalent backbone of the polypeptide.

By “internal site” as used in referenced to a polypeptide or an amino acid sequence of a polypeptide means a region of the polypeptide that is not at the N-terminus or at the C-terminus.

The term “subject” refers to human and non-human subjects, especially mammalian subjects.

The term “treating” or “treatment” as used herein means the treating or treatment of a disease or medical condition in a subject, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a subject; (c) suppressing the disease or medical condition, for example by, slowing or arresting the development of the disease or medical condition in a subject; or (d) alleviating a symptom of the disease or medical condition in a subject. In some embodiments, the term “treating,” or “treatment” excludes a prophylactic treatment.

The terms “native amino acid sequence” as used herein refers to the amino acid sequence of a polypeptide prior to modification to include a modified amino acid residue.

The terms “amino acid analog,” “unnatural amino acid,” and the like is used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some embodiments, the amino acid analogs share backbone structures, and/or the side chain structures of one or more natural amino acids, with difference(s) being one or more modified groups in the molecule. Such modification can include, but is not limited to, substitution of an atom (such as N) for a related atom (such as S), addition of a group (such as methyl, or hydroxyl, and the like) or an atom (such as Cl or Br, and the like), deletion of a group, substitution of a covalent bond (single bond for double bond, and the like), or combinations thereof. For example, amino acid analogs can include α-hydroxy acids, and α-amino acids, and the like.

The term “amino acid side chain” is used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

The term “carbohydrate” is used to refer to monomer units and/or polymers of monosaccharides, disaccharides, oligosaccharides, and polysaccharides. The term sugar is be used to refer to the smaller carbohydrates, such as monosaccharides, disaccharides. The term “carbohydrate derivative” includes compounds where one or more functional groups of a carbohydrate of interest are substituted (replaced by any convenient substituent), modified (converted to another group using any convenient chemistry) or absent (e.g., eliminated or replaced by H). A variety of carbohydrates and carbohydrate derivatives are available and can be adapted for use in the subject compounds and conjugates.

The term “glycoside” or “glycosyl” refers to a sugar molecule or group bound to a moiety via a glycosidic bond. For example, the moiety that the glycoside is bound to can be a cleavable linker as described herein. A glycosidic bond can link the glycoside to the other moiety through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl). In some cases, glycosides can be cleaved from the moiety they are attached to, such as by chemically-mediated hydrolysis or enzymatically-mediated hydrolysis.

The terms “Tissue Factor,” “TF,” “platelet tissue factor,” “factor III,” “thromboplastin,” and “CD142” are used interchangeably herein to refer to TF, or any variants (e.g., splice variants and allelic variants), isoforms, and species homologs of TF that are naturally expressed by cells, or that are expressed by cells transfected with a TF gene. In some aspects, the TF protein is a TF protein naturally expressed by a primate (e.g., a monkey or a human), a rodent (e.g., a mouse or a rat), a dog, a camel, a cat, a cow, a goat, a horse, a pig or a sheep. The term TF encompasses “full-length” TF, as well as any form of TF or any fragment thereof, for example those resulted from processing in a cell. In some embodiments, the TF comprises a signal sequence. In some embodiments, the TF does not include a signal sequence. In some embodiments, the term TF refers to a fragment of the full-length TF, which comprises an TF extracellular domain (ECD). The term TF also encompasses naturally occurring variants of TF, such as SNP variants, splice variants and allelic variants. In some aspects, the TF protein is human TF (hTF;

(SEQ ID NO: 175)
METPAWPRVPRPETAVARTLLLGWVFAQVAGASGTTNTVAAYNLTWKSTN
FKTILEWEPKPVNQVYTVQISTKSGDWKSKCFYTTDTECDLTDEIVKDVK
QTYLARVFSYPAGNVESTGSAGEPLYENSPEFTPYLETNLGQPTIQSFEQ
VGTKVNVTVEDERTLVRRNNTFLSLRDVFGKDLIYTLYYWKSSSSGKKTA
KTNTNEFLIDVDKGENYCFSVQAVIPSRTVNRKSTDSPVECMGQEKGEFR
EIFYIIGAVVFVVIILVIILAISLHKCRKAGVGQSWKENSPLNVS

wherein the underline denotes signal peptide). In further embodiments, the human TF does not comprise a signal peptide, for example amino acid (aa) 33 to aa 295 of SEQ ID NO:175. In yet further embodiments, the human TF as used herein refers to the extracellular domain (ECD) of the human TF, for example, aa 33 to aa 251 of SEQ ID NO:175. In some aspects, the TF protein is cynomolgus TF (cTF; SGTTNTVAAYNLTWKSTNFKTILEWEPKPINQVYTVQISTKSGDWKSKCFYTADTEC DLTDEIVKDVKQTYLARVF SYPAGHVESTGSTEEPPYENSPEFTPYLETNLGQPTIQSF EQVGTKVNVTVQDEWTLVRRNDTFL SLRDVFGKDLIYTLYYWKSSSSGKKTAKTNT NEFLIDVDKGENYCFSVQAVIPSRRTANRKSTDSPVECMGHEKGESREIFYIIGAVVF VVIILVIILAISLHKCKKARVGRSWKENSPLNVA (SEQ ID NO:176)). In further embodiments, the cynomolgus TF as used herein refers to its ECD, for example aa 1 to aa 220 of SEQ ID NO:176. In some aspects, the TF protein is mouse TF (mTF; AGIPEKAFNLTWISTDFKTILEWQPKPTNYTYTVQISDRSRNWKNKCFSTTDTECDLT DEIVKDVTWAYEAKVL SVPRRNSVHGDGDQLVIHGEEPPF TNAPKFLPYRDTNLGQP VIQQFEQDGRKLNVVVKDSLTLVRKNGTFLTLRQVFGKDLGYIITYRKGSSTGKKTN ITNTNEFSIDVEEGVSYCFFVQAMIFSRKTNQNSPGSSTVCTEQWKSFLGETLIIVGAV VLLATIFIILLSISLCKRRKNRAGQKGKNTPSRLA (SEQ ID NO:177)). In further embodiments, the mouse TF as used herein refers to its ECD, for example aa 1 to aa 223 of SEQ ID NO:177. In some aspects, the TF protein is pig TF (pTF; TGTTDVIVAYNLTWKSTNFKTILEWEPKPINYVYTVQISPRLGDWKNKCFHTTDTEC DVTDEIMRNVKETYVARVLSYPADTVLTAQEPPFTNSPPFTPYLDTNLGQPVIQSFEQ VGTKLNVTVEAARTLVRVNGTFLRLRDVFGKDLNYTLYYWRASSTGKKKATTNTN EFLIDVDKGENYCFSVQAVIPSRRVNQKSPESRIECTSQEKAVSRELFLIVGAVVFAVI VFVLVLSVSLYKCRKERAGPSGKENAPLNVA (SEQ ID NO:178)). In some embodiments, the pig TF as used herein refers to its ECD, for example aa to aa 216 of SEQ ID NO:178. TF is a cell surface receptor for the serine protease factor VIIa. It is often times constitutively expressed by certain cells surrounding blood vessels and in some disease settings.

In some embodiments, the term TF as used herein refers to a TF epitope.

The terms “antibody,” “immunoglobulin,” or “Ig” are used interchangeably herein and are used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, single domain antibodies, monospecific antibodies, multispecific antibodies (including bispecific antibodies), synthetic antibodies, chimeric antibodies, humanized antibodies, or human versions of antibodies having full length heavy and/or light chains. Antibodies also include single antibody domains as well as antibody fragments (and/or polypeptides that comprise antibody fragments) that retain TF binding characteristics. Non-limiting examples of antibody fragments include antigen-binding regions and/or effector regions of the antibody, e.g., Fab, Fab′, F(ab′)₂, Fv, scFv, (scFv)₂, single-chain antibody molecule, dual variable domain antibody, single variable domain antibody, linear antibody, V region, a multispecific antibody formed from antibody fragments, F(ab)₂, Fd, Fc, diabody, di-diabody, disulfide-linked Fvs (dsFv), single-domain antibody (e.g., nanobody) or other fragments (e.g., fragments consisting of the variable regions of the heavy and light chains that are non-covalently coupled). In general terms, a variable (V) region can be any suitable arrangement of immunoglobulin heavy (VH) and/or light (VL) variable regions. For example, antibodies also include tetrameric antibodies comprising two heavy chain and two light chain molecules, an antibody light chain monomer, and an antibody heavy chain monomer. Thus, for example, the V region can be dimeric and contain VH-VH, VH-VL, or VL-VL dimers that bind TF. In any embodiment, a VH region and a VL region can be covalently coupled either directly or through a linker to form a single-chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable regions”) can be obtained by constructing polynucleotides that encode one or more CDRs of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments can be incorporated, for example, into single domain antibodies, maxibodies, minibodies, intrabodies, diabodies, triabodies, tetrabodies, variable regions of new antigen receptors (v-NAR), and bis-single-chain Fv regions (see, e.g., Hollinger and Hudson, Nature Biotechnology, 23(9):1126-1136, 2005). In some embodiments, antibodies comprising a VH and/or VL contain a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgAQ1, and IgA2) of immunoglobulin molecule.

The term “monospecific,” as used herein denotes an antibody that has one or more binding sites each of which binds to the same epitope of the same antigen.

The term “bispecific” means that the antibody can specifically bind to at least two distinct antigenic determinants, for example two binding sites each formed by a pair of an antibody heavy chain variable region (VH) and an antibody light chain variable region (VL) binding to different antigens or to different epitopes on the same antigen. Such a bispecific antibody can have a 1+1 format (comprising one binding site for a first antigen or epitope and one binding site for a second antigen or epitope). Other bispecific antibody formats can be 2+1 or 1+2 formats (comprising two binding sites for a first antigen or epitope and one binding site for a second antigen or epitope) or 2+2 format (comprising two binding sites for a first antigen or epitope and two binding sites for a second antigen or epitope). When a bispecific antibody comprises two antigen binding sites, each can bind to a different antigenic determinant. Such a bispecific antibody can bind to two different epitopes on the same antigen (e.g., epitopes on TF).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well known in the art. These include, but are not limited to, basic local alignment search tool (BLAST), ALIGN, MegAlign, BestFit, GCG Wisconsin Package, and variants thereof. In some embodiments, two nucleic acids or polypeptides are substantially identical, meaning they have at least 70%, at least 75%, at least 80%, at least 85%, or at least 90%, and in some embodiments, at least 95%, 96%, 97%, 98%, or 99% nucleotide or amino acid residue identity, when compared and aligned for maximum correspondence, as measured using a sequence comparison algorithm or by visual inspection.

In some embodiments, identity exists over a region of the amino acid sequences that is at least about ten residues, at least about 20 residues, at least about 40-60 residues, at least about 60-80 residues in length or any integral value there between. In some embodiments, identity exists over a longer region than 60-80 residues, such as at least about 80-100 residues, and in some embodiments, the sequences are substantially identical over the full-length of the sequences being compared, such as the coding region of a target protein or an antibody. In some embodiments, identity exists over a region of the nucleotide sequences that is at least about ten bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments, the sequences are substantially identical over the full length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions that do not eliminate binding are well known in the art.

The term “polypeptide” refers to a polymer of amino acids of any length. The polymer can be linear or branched, it can comprise modified amino acids, and it can include (e.g., be substituted with) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component or a drug (e.g., toxin). Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains or dimers of single chains.

As used herein, an “antigen” is a moiety or molecule that contains an epitope to which an antibody can bind. As such, an antigen can be bound by an antibody. In some embodiments, the antigen to which an antibody described herein binds is TF (e.g., human TF), or a fragment thereof, such as a fragment that comprises one or more regions of TF.

As used herein, an “epitope” is a term in the art and refers to a localized region of an antigen to which an antibody can bind. An epitope can be a linear epitope or a conformational, non-linear, or discontinuous epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human TF. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three-dimensional protein structure. In other embodiments, an antibody requires amino acid residues making up the epitope to exhibit a particular conformation (e.g., bend, twist, turn or fold) in order to recognize and bind the epitope.

An antibody binds “an epitope,” “essentially the same epitope,” or “the same epitope” as a reference antibody. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

Additional details regarding methods for epitope binning and determining epitope binding of antibodies are described herein, as shown in Example 5.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as understood by one skilled in the art.

In some embodiments, “specifically binds” means, for instance, that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen can bind to other peptides or polypeptides, generally with lower affinity as determined by, e.g., immunoassays, BIACORE™, KinExA 3000 instrument (Sapidyne Instruments, Boise, ID), the OctetQK384 system (ForteBio, Menlo Park, CA), or other assays known in the art. In some embodiments, an antibody or antigen binding region binds to or specifically binds to an antigen when it binds to the antigen with higher affinity than to any cross-reactive antigen as determined using experimental techniques, such as radioimmunoassays (RIAs) and enzyme linked immunosorbent assays (ELISAs). Typically, a specific or selective reaction will be at least twice background signal or noise and can be more than ten times background. See, e.g., Fundamental Immunology 332-36 (Paul ed., 2d ed. 1989) for a discussion regarding binding specificity. In some embodiments, the extent of binding of an antibody or antigen-binding region to a “non-target” protein is less than about 10% of the binding of the antibody or antigen-binding region to its target antigen, for example, as determined by fluorescence activated cell sorting (FACS) analysis or RIAs. In some embodiments, molecules that specifically bind to an antigen bind to the antigen with a KA that is at least 2 logs, 2.5 logs, 3 logs, 4 logs or greater than the KA when the molecules bind to another antigen. In some embodiments, molecules that specifically bind to an antigen do not cross react with other proteins. In another specific embodiment, molecules that specifically bind to an antigen do not cross react with other non-TF proteins. In some embodiments, “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, in some embodiments, a polypeptide or molecule can specifically bind more than one target. In some embodiments, multiple targets can be bound by the same antigen-binding site on the polypeptide or molecule. For example, in some embodiments, an antibody can comprise two identical antigen-binding sites, each of which specifically binds the same epitope on two or more proteins. In alternative embodiments, an antibody can be bispecific and comprise at least two antigen-binding sites with differing specificities. Generally, but not necessarily, reference to “binding” means “specific binding.”

The term “binding affinity” generally refers to the strength of the sum of noncovalent interactions between a single binding site of a molecule (e.g., antibody) and its binding partner (e.g., an antigen such as TF). Unless indicated otherwise, as used herein, “binding affinity” refers to intrinsic binding affinity that reflects a 1:1 interaction between members of a binding pair (e.g., antibody and antigen). The affinity of a binding molecule X for its binding partner Y can generally be represented by the dissociation constant (K_{D}).

Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity is known in the art, any of which can be used for purposes of the present disclosure. In one embodiment, the “K_{D}” or “K_{D} value” can be measured by biolayer interferometry (BLI) using, for example, the OctetQK384 system (ForteBio, Menlo Park, CA). Alternatively, the K_{D} may also be measured in a radiolabeled antigen binding assay (RIA), for example, performed with the Fab version of an antibody of interest and its antigen (Chen et al., (1999) J. Mol Biol 293:865-881) or using surface plasmon resonance (SPR) assays by BIACORE™, using, for example, a BIACORE™-2000 or a BIACORE™-3000 (BIAcore, Inc., Piscataway, NJ). An “on-rate” or “rate of association” or “association rate” or “kₒₙ,” as well as an “off-rate” or “rate of dissociation” or “dissociation rate” or “k_{off},” can also be determined with the same SPR or BLI techniques described herein using, for example, the OctetQK384 system (ForteBio, Menlo Park, CA) or a BIACORE™-2000 or a BIACORE™-3000 (BIACORE™, Inc., Piscataway, NJ), respectively.

The term “compete,” when used in the context of a TF antibody, describes a binding agent that, in the presence of another binding agent, is at least partially inhibited from binding to an epitope or binding site due to binding of the other binding agent. Competition can be determined by an assay in which the binding agent under study prevents or inhibits the specific binding of a reference molecule (e.g., a reference ligand, or reference antigen binding protein, such as a reference antibody) to a common antigen (e.g., TF). Numerous types of competitive binding assays can be used to determine if a test binding agent competes with a reference molecule for binding to TF (e.g., human TF). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619 or Cheung et al., (1990) Virology 176:546-552); solid phase direct labeled assay, solid phase direct labeled sandwich assay (see, e.g., Harlow and Lane, (1988) Antibodies, A Laboratory Manual, Cold Spring Harbor Press); solid phase direct label RIA using I-125 label (see, e.g., Morel et al., (1988) Molec. Immunol. 25:7-15); and direct labeled RIA (Moldenhauer et al., (1990) Scand. J. Immunol. 32:77-82). Typically, such an assay involves the use of a purified antigen (e.g., TF, such as human TF) bound to a solid surface or cells bearing either of an unlabeled test antigen binding protein (e.g., test TF antibody or ADC) or a labeled reference antigen binding protein (e.g., reference TF antibody or ADC). Competitive inhibition can be measured by determining the amount of label bound to the solid surface or cells in the presence of the test antigen binding protein. Usually, the test antigen binding protein is present in excess.

Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70%, or 75%. In some embodiments, binding is inhibited by at least 80%, 85%, 90%, 95%, 96%, 97%, 98%, 99% or more.

The terms “constant region” and “constant domain” are used interchangeably herein, are well-known antibody terms of art, and refer to an antibody portion, for example, a carboxyl terminal portion of a light and/or heavy chain which is not directly involved in binding of an antibody to an antigen, but which can exhibit various effector functions, such as interaction with an Fc receptor. The term includes the portion of an immunoglobulin molecule having a generally more conserved amino acid sequence relative to an immunoglobulin variable region.

Antibody “effector functions” refer to those biological activities attributable to the Fc region (e.g., a native sequence Fc region or amino acid sequence variant Fc region) of an antibody and which vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (of the EU numbering system) or from Pro230 (of the EU numbering system) to the carboxyl-terminus thereof. The C-terminal lysine (residue 447 of the EU numbering system) of the Fc region can be removed, for example, during production or purification of the antibody, or by recombinantly engineering the nucleic acid encoding a heavy chain of the antibody. An exemplary Fc region sequence is provided below (CH₂ domain=bold text; CH₃ domain=underline text):

(SEQ ID NO: 76)
CPPCPAPELLGGPSVFLFPPKPKDTLMISRTPEVTCVVVDVSHEDPEVKE
NWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSC
SVMHEALHNHYTQKSLSLSPGK.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), and the like. Such effector functions generally require the Fc region to be combined with a binding region or binding domain (e.g., an antibody variable region or domain) and can be assessed using various assays as disclosed.

A “native sequence Fc region” comprises an amino acid sequence identical to the amino acid sequence of an Fc region found in nature, and not manipulated, modified, and/or changed (e.g., isolated, purified, selected, including, or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region; as well as naturally occurring variants thereof.

A “variant Fc region” comprises an amino acid sequence that differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein can have a loss of effector function (e.g., silent Fc).

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant region, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) or lambda (λ) based on the amino acid sequence of the constant regions. Light chain amino acid sequences are well known in the art.

The terms “antigen binding fragment,” “antigen binding domain,” “antigen binding region,” and similar terms refer to a portion of an antibody that comprises amino acid residues that interact with an antigen and confer on the binding fragment or region its specificity and affinity for the antigen (e.g., the CDRs). “Antigen binding fragment” as used herein includes “antibody fragment,” which comprises a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, and the like), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, such as molecules that contain one or more antigen binding sites that bind to TF.

A TF antibody, as described herein, can be of any type (e.g., IgG, IgE, IgM, IgD, IgA or IgY), any class (e.g., IgG1, IgG2, IgG3, IgG4, IgAQ1 or IgA2), or any subclass (e.g., IgG2a or IgG2b) of immunoglobulin molecule. In some embodiments, a TF antibody, as described herein, is an IgG antibody (e.g., human IgG), or a class (e.g., human IgG1, IgG2, IgG3, or IgG4) or a subclass thereof.

In some embodiments, a TF antibody is a 4-chain antibody unit comprising two heavy (H) chain/light (L) chain pairs, wherein the amino acid sequences of the H chains are identical and the amino acid sequences of the L chains are identical. In some embodiments, the H and/or L chains comprise constant regions, for example, human constant regions. In some embodiments, the L chain constant region of a TF antibody is a kappa or lambda light chain constant region, for example, a human kappa or lambda light chain constant region. In some embodiments, the H chain constant region of a TF antibody comprises a gamma heavy chain constant region, for example, a human gamma heavy chain constant region. In some embodiments, a TF antibody comprise an IgG constant region, for example, human IgG constant regions (e.g., IgG1, IgG2, IgG3, and/or IgG4 constant regions).

As used herein, “TF antibody” and “antibody that binds to TF” are used interchangeably and refer to an antibody that preferentially binds to TF. An antibody or fragment thereof can preferentially bind to TF, such as human TF, which means that the antibody or fragment thereof binds to TF, such as human TF, with greater affinity than it binds to an unrelated control protein. For example, the antibody or fragment thereof can specifically recognize and bind to TF or a portion thereof “Specific binding” means that the TF antibody or fragment thereof binds to TF with an affinity that is at least 5, 10, 15, 20, 25, 50, 100, 250, 500, 1000, or 10,000 times greater than the affinity for an unrelated control protein (e.g., hen egg white lysozyme). In some embodiments, the TF antibody or fragment thereof can bind TF substantially exclusively (e.g., is able to distinguish TF from other known polypeptides, for example, by virtue of measurable differences in binding affinity). In some embodiments, a TF antibody can react with TF sequences other than human TF sequences (e.g., cynomolgus TF sequences).

The terms “variable region” and “variable domain” are used interchangeably to refer to a portion of the light and heavy chains of an antibody that are generally located at the amino-terminal of the light and heavy chain, has a length of about 120 to 130 amino acids in the heavy chain, about 100 to 110 amino acids in the light chain, and is used in the binding and specificity of each antibody for its antigen. The variable region of the heavy chain is referred to herein as “VH.” The variable region of the light chain is referred to herein as “VL.” The term “variable” refers to the fact that certain segments of the variable regions differ extensively in sequence among antibodies. The V region mediates antigen binding and defines specificity of a particular antibody for its antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four frameworks (FR1, FR2, FR3 and FR4), largely adopting a R sheet configuration, connected by three hypervariable regions, which form loops connecting, and in some cases forming part of, the R sheet structure. The hypervariable regions in each chain are held together in proximity by the frameworks and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, (1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region, “and “CDR” when used herein refers to the regions of an antibody variable region that are hypervariable in sequence and/or form structurally defined loops. Generally, antibodies comprise six hypervariable regions: three in the VH (H1 or VH CDR1, H2 or VH CDR2, and H3 or VH CDR3), and three in the VL (L1 or VL CDR1, L2 or VL CDR2, and L3 or VL CDR3). Several hypervariable region delineations are in use and are encompassed herein. The Kabat CDRs are based on sequence variability and are the most used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT®) Information System (Lefranc et al., Dev. Comp. Immunol. 27(1):55-77 (2003)). IMGT® is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable region is conserved between species and present in structures called loops, by using numbering systems that align variable region sequences of structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Pluckthun, J. Mol. Biol. 309: 657-670 (2001). Correspondence between the numbering system, including, for example, the Kabat numbering and the IMGT® unique numbering system, is well known to one skilled in the art (see, e.g., Kabat, supra; Chothia and Lesk, supra; Martin, supra; Lefranc et al., supra) and is also illustrated below. Various systems known in the art or described herein represent different ways of delineating CDRs, and when they are used to define the same antibody, they are often considered equivalent. An exemplary system, shown herein combines Kabat and Chothia.

| | Exemplary | IMGT ® | Kabat | AbM | Chothia | Contact |
|---|---|---|---|---|---|---|
| V_{H} CDR1 | 26-35 | 27-38 | 31-35 | 26-35 | 26-32 | 30-35 |
| V_{H} CDR2 | 50-65 | 56-65 | 50-65 | 50-58 | 53-55 | 47-58 |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions can comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR″ are used interchangeably.

The term “vector” refers to a substance that is used to carry or include a nucleic acid sequence, for example, to introduce a nucleic acid sequence into a host cell. Examples of vectors include expression vectors, plasmids, phage vectors, viral vectors, episomes and artificial chromosomes, which can include selection sequences or markers operable for stable integration into a host cell's chromosome. Additionally, a vector can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g., both an antibody heavy and light chain or an antibody VH and VL region) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., a TF antibody), and it is further understood that expression levels can be optimized to obtain sufficient expression using methods well known in the art.

The terms “TF-mediated disease,” “TF-mediated disorder”, and “TF-mediated condition” are used interchangeably and refer to any disease, disorder or condition associated with or characterized by TF-expressing cells, such as TF-expressing tumor cells. A TF-mediated disease includes a cancer including, but not limited to, cancers that express or overexpress TF.

The term “tumor,” in any embodiment herein, refers to any neoplastic cell growth or proliferation, whether malignant or benign, and to all pre-cancerous and cancerous cells and tissues.

The terms “cancer” and “cancerous” refer to or describe the physiological condition in mammals that is typically characterized by unregulated cell growth.

The term “ADC” refers to an antibody-drug conjugate, which in the context of the present invention refers to a TF antibody, which is coupled to another moiety which includes a drug, as described herein.

As used herein, “drug” refers to a compound that has biological activity, such as a cytotoxic compound (e.g., a cytotoxic small molecule, a cytotoxic synthetic peptide, and the like).

Examples of drugs include small molecule drugs, such as a cancer chemotherapeutic agent. For example, where the polypeptide is an antibody (or fragment thereof) that has specificity for a tumor cell, the antibody can be modified as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca)alkaloids, and steroid hormones. Peptidic compounds can also be used.

Suitable cancer chemotherapeutic agents include dolastatin and active analogs and derivatives thereof, and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in a TF-ADC of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD)).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (CYNOTAN™), melphalan (L-sarcolysin), carmustine (BCNU), lomustine (CCNU), semustine(methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U®), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatin, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (TAXOL®), docetaxel (TAXOTERE®), deoxycoformycin, mitomycin-C, L-asparaginase, azathioprine; brequinar; alkaloids, e.g. vincristine, vinblastine, vinorelbine, vindesine, and the like; podophyllotoxins, e.g. etoposide, teniposide, and the like; antibiotics, e.g. anthracycline, daunorubicin hydrochloride (daunomycin, rubidomycin, cerubidine), idarubicin, doxorubicin, epirubicin and morpholino derivatives, and the like; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, and the like; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (TAXOL®), TAXOL® derivatives, docetaxel (TAXOTERE®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, and the like; estrogens and pregestins, e.g. hydroxyprogesterone caproate, medroxyprogesterone acetate, megestrol acetate, estradiol, clomiphene, tamoxifen; and the like; adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, flutamide (DROGENIL®), toremifene (FARESTON®), and goserelin (ZOLADEX®), and the like. Estrogens stimulate proliferation and differentiation; therefore, compounds that bind to the estrogen receptor are used to block this activity. Corticosteroids can inhibit T cell proliferation.

Other suitable chemotherapeutic agents include metal complexes, e.g. cisplatin (cis-DDP), carboplatin, and the like; ureas, e.g. hydroxyurea; hydrazines, e.g. N-methylhydrazine; epidophyllotoxin; a topoisomerase inhibitor; procarbazine; mitoxantrone; leucovorin; tegafur; and the like Other anti-proliferative agents of interest include immunosuppressants, e.g. mycophenolic acid, thalidomide, desoxyspergualin, azasporine, leflunomide, mizoribine, azaspirane (SKF 105685); gefitinib (IRESSA®, ZD 1839, 4-(3-chloro-4-fluorophenylamino)-7-methoxy-6-(3-(4-morpholinyl)propoxy)quinazoline); and the like.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL®, TAXOTERE® (a formulation of docetaxel), 10-desacetyl analogs of paclitaxel and 3′N-desbenzoyl-3′N-t-butoxycarbonyl analogs of paclitaxel) can be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis). Paclitaxel should be understood to refer to not only the common chemically available form of paclitaxel, but analogs and derivatives (e.g., TAXOTERE® docetaxel, as noted herein) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but are not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

An “effective amount” is generally an amount sufficient to reduce the severity and/or frequency of symptoms, eliminate the symptoms and/or underlying cause, prevent the occurrence of symptoms and/or their underlying cause, and/or improve or remediate the damage that results from or is associated with a disease, disorder, or condition. In some embodiments, the effective amount is a therapeutically effective amount.

The term “therapeutically effective amount” as used herein refers to the amount of an antibody or ADC described herein that is sufficient to reduce and/or ameliorate the severity and/or duration of a given disease, disorder, or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the therapeutic effect of another therapy (e.g., a therapy other than the administration of an antibody or ADC described herein). A “therapeutically effective amount” of a substance/molecule/agent of the present disclosure (e.g., a TF antibody or ADC) can vary based on a number of factors such as the disease state, age, sex, and weight of the individual, and the ability of the substance/molecule/agent, to elicit a desired response in the individual.

A therapeutically effective amount encompasses an amount in which any toxic or detrimental effects of the substance/molecule/agent are outweighed by the therapeutically beneficial effects. In some embodiments, the term “therapeutically effective amount” refers to an amount of an antibody or other agent (e.g., or drug) effective to “treat” a disease, disorder, or condition, in a subject or mammal.

In some embodiments, the drug is a microtubule affecting agent that has anti-proliferative activity, such as a maytansinoid. In some embodiments, the drug is an antimitotic agent, such as an auristatin or an active auristatin analog or derivative thereof. In some embodiments, the drug is a DNA alkylating agent.

The term “pharmaceutically acceptable” as used herein means being approved by a regulatory agency of the federal or a state government, or listed in the U.S. Pharmacopeia, European Pharmacopeia, or other generally recognized Pharmacopeia for use in animals, and more particularly in humans.

“Excipients” include carriers, excipients, preservatives, or stabilizers that are nontoxic to the cell or mammal being exposed thereto at the dosages and concentrations employed and can be included, for example, to affect stability, bulk up formulations, or to confer a therapeutic enhancement on the active ingredient in the final dosage form (e.g., facilitating absorption, reducing viscosity, enhancing solubility). An “excipient” can be an organic or inorganic ingredient, natural or synthetic with which the active ingredient is combined to facilitate the use of the active ingredient, e.g., the administration of the active ingredient to a subject. Examples of excipients include buffers such as phosphate, citrate, and other organic acids; antioxidants such as ascorbic acid; low molecular weight (e.g., less than about ten amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates such as glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™ The term “excipient” can also refer to a diluent, adjuvant (e.g., Freund's adjuvant (complete or incomplete)), excipient, or vehicle with which the therapeutic is administered. Such excipients can be sterile liquids, such as water and oils, such as those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, in any embodiment, can also contain minor amounts of wetting or emulsifying agents, or pH buffering agents. Compositions can take the form of solutions, suspensions, emulsion, tablets, pills, capsules, powders, sustained-release formulations, and the like. Oral compositions, such as formulations, can include standard excipients such as pharmaceutical grades of mannitol, lactose, starch, magnesium stearate, sodium saccharine, cellulose, magnesium carbonate, and the like. Examples of suitable excipients are described in Remington: The Science and Practice of Pharmacy (2020) (Elsevier Science, Amsterdam, Netherlands), including pharmaceutical compounds, can contain an effective amount or therapeutically effective amount of a TF-ADC, for example, in isolated or purified form, together with a suitable amount of excipient to provide the form for proper administration to the subject. The formulation should suit the mode of administration.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### TF-ADCS

An antibody that binds to TF (also referred to herein as “TF antibody,” “anti-TF antibody,” “TF Ab,” “Ab” or “antibody”) and a drug can be linked directly or indirectly to each other via a pyridazine-pyrrolo coupling moiety to form a TF-ADC as described herein.

In certain embodiments, the TF antibody and the two or more drugs or active agents are bound to each other through one or more functional groups and covalent bonds. For example, the one or more functional groups and covalent bonds can include a branched linker as described herein.

Moieties of interest (e.g., drugs or active agents) can be conjugated to the TF antibody at any desired site of the antibody. Thus, the present disclosure provides, for example, a TF antibody having moieties conjugated at two or more sites on the antibody, such as a site at or near the C-terminus of the antibody, a position at or near the N-terminus of the antibody, and a position between the C-terminus and the N-terminus of the antibody (e.g., at an internal site of the antibody). Combinations of the above conjugation sites are also possible.

In certain embodiments, a conjugate of the present disclosure includes two (or more) drugs or active agents conjugated to an amino acid residue of a TF antibody at the α-carbon of an amino acid residue. Stated another way, a conjugate includes a TF antibody where the side chain of an amino acid residue in the antibody has been modified and attached to two (or more) drugs or active agents (e.g., attached to two drugs or active agents through a branched linker as described herein). For example, a conjugate includes a TF antibody where the α-carbon of an amino acid residue in the antibody has been modified and attached to two drugs or active agents (e.g., attached to two drugs or active agents through a branched linker as described herein).

Embodiments of the present disclosure include conjugates where a TF antibody is conjugated to two or more moieties, such as 3 moieties, 4 moieties, 5 moieties, 6 moieties, 7 moieties, 8 moieties, 9 moieties, 10 moieties, 11 moieties, 12 moieties, 13 moieties, 14 moieties, 15 moieties, 16 moieties, 17 moieties, 18 moieties, 19 moieties, or 20 or more moieties. The moieties may be conjugated to the TF antibody at multiple sites in the antibody. In some embodiments, two moieties may be conjugated to a single amino acid residue of the TF antibody. For instance, two moieties may be conjugated to the same amino acid residue of the TF antibody. In other embodiments, two moieties are conjugated to a first amino acid residue of the TF antibody and two other moieties are conjugated to a second amino acid residue of the TF antibody. For example, a TF antibody can be conjugated to first and second moieties at a first amino acid residue and conjugated to third and fourth moieties at a second amino acid residue, etc. In some cases, two or more amino acid residues in the TF antibody are each conjugated to a pair of moieties (i.e., two moieties), where each pair of moieties is conjugated to the TF antibody through a branched linker as described herein. In some cases, 1 amino acid residue in the TF antibody is conjugated to a pair of moieties through a branched linker as described herein. In other instances, 2 or more amino acid residues, such as 3, 4, 5, 6, 7, 8, 9, or 10 or more amino acid residues in the TF antibody are each conjugated to a pair of moieties through a branched linker as described herein.

The one or more amino acid residues of the TF antibody that are conjugated to the moieties of interest may be naturally occurring amino acids, unnatural amino acids, or combinations thereof. For instance, the conjugate may include moieties of interest (e.g., drugs or active agents) conjugated to a naturally occurring amino acid residue of the TF antibody. In other instances, the conjugate may include moieties of interest conjugated to an unnatural amino acid residue of the TF antibody. The moieties of interest may be conjugated to the TF antibody at a single natural or unnatural amino acid residue as described above. One or more natural or unnatural amino acid residues in the TF antibody may be conjugated to the moieties of interest as described herein. For example, two (or more) amino acid residues (e.g., natural or unnatural amino acid residues) in the TF antibody may each be conjugated to two moieties through a branched linker, such that multiple sites in the TF antibody are conjugated to the moieties of interest.

As described herein, a TF antibody may be conjugated to two or more moieties of interest. In certain embodiments, the moiety of interest is a payload, for instance, a chemical entity, such as a drug, an active agent, or a detectable label. For example, drugs (or active agents, such as cytokines) may be conjugated to the TF antibody, or in other embodiments, detectable labels may be conjugated to the TF antibody. In other embodiments, combinations of different payloads may be conjugated to the TF antibody. Thus, for instance, embodiments of the present disclosure include, but are not limited to, the following: a conjugate of a TF antibody and two or more drugs; a conjugate of a TF antibody and two or more active agents, such as cytokines; a conjugate of a TF antibody and two or more detectable labels; and combinations thereof.

In certain embodiments, the TF antibody and the moieties of interest (e.g., drugs or active agents) are conjugated through a conjugation moiety. For example, the TF antibody and the moieties of interest may each be bound (e.g., covalently bonded) to the conjugation moiety, thus indirectly binding the TF antibody and the moieties of interest together through the conjugation moiety. In some cases, the conjugation moiety includes a hydrazinyl-indolyl or a hydrazinyl-pyrrolo-pyridinyl compound, or a derivative of a hydrazinyl-indolyl or a hydrazinyl-pyrrolo-pyridinyl compound. For instance, a general scheme for coupling moieties of interest to a TF antibody through a hydrazinyl-indolyl or a hydrazinyl-pyrrolo-pyridinyl conjugation moiety is shown in the general reaction scheme below. Hydrazinyl-indolyl and hydrazinyl-pyrrolo-pyridinyl conjugation moieties are also referred to herein as a hydrazino-iso-Pictet-Spengler (HIPS) conjugation moiety and an aza-hydrazino-iso-Pictet-Spengler (azaHIPS) conjugation moiety, respectively. Accordingly, in some embodiments, it would be understood by one of skill in the art that the carbon which is denoted in a formula as disclosed herein, such as Formula (I) or (II), as being immediately adjacent to Ab was originally a part of the antibody prior to the conjugation. In some embodiments, this carbon is conjugated to a (fGly′) residue, thus conjugating the antibody and the linker-payload. In other embodiments, this carbon is interpreted as a part of a (fGly′) residue conjugating the antibody and the linker-payload.

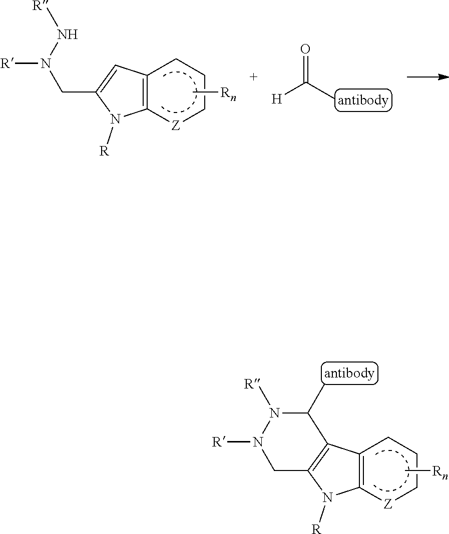

In the reaction scheme above, each R independently includes a moiety of interest (e.g., drug or active agent) that is conjugated to the TF antibody (e.g., conjugated to the TF antibody through a linker as described herein), where n is an integer from 1 to 4. As shown in the reaction scheme above, a conjugation moiety (e.g., a hydrazinyl-indolyl or a hydrazinyl-pyrrolo-pyridinyl conjugation moiety) is attached to two or more drugs or active agents, R. A TF antibody that includes a 2-formylglycine residue (fGly) is reacted with the conjugation moiety to produce a TF antibody conjugate, thus attaching the two or more drugs or active agents to the TF antibody through the conjugation moiety.

As described herein, the moieties can be any of a variety of moieties such as, but not limited to, chemical entities, such as detectable labels, or a drugs or active agents. R′ and R″ may each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. Z may be CR²¹, NR²², N, O or S, where R²¹ and R²² are each independently selected from any of the substituents described for R′ and R″ above.

Other hydrazinyl-indolyl or hydrazinyl-pyrrolo-pyridinyl conjugation moieties are also possible, as shown in the conjugates and compounds described herein. For example, the hydrazinyl-indolyl or hydrazinyl-pyrrolo-pyridinyl conjugation moieties may be attached (e.g., covalently attached) to two or more linkers. As such, embodiments of the present disclosure include a hydrazinyl-indolyl or hydrazinyl-pyrrolo-pyridinyl conjugation moiety attached to two or more drugs or active agents each through a corresponding linker. Thus, conjugates of the present disclosure may include two or more linkers, where each linker attaches a corresponding drug or active agent to the hydrazinyl-indolyl or hydrazinyl-pyrrolo-pyridinyl conjugation moiety. Accordingly, the hydrazinyl-indolyl or hydrazinyl-pyrrolo-pyridinyl conjugation moiety and two or more linkers may be viewed overall as a “branched linker”, where the hydrazinyl-indolyl or hydrazinyl-pyrrolo-pyridinyl conjugation moiety is attached to two of more “branches”, where each branch includes a linker attached to a drug or active agent.

Combinations of the same or different payloads may be conjugated to the TF antibody through the branched linker. In certain embodiments, the two payloads (e.g., drugs, active agents or detectable labels) attached to the branched linker are the same payload (e.g., drug, active agent or detectable label). For example, a first branch of a branched linker may be attached to a payload (e.g., drug, active agent or detectable label) and a second branch of the branched linker may be attached to the same payload (e.g., drug, active agent or detectable label) as the first branch.

In other embodiments, the two payloads (e.g., drugs, active agents or detectable labels) attached to the branched linker are different payloads (e.g., drugs, active agents or detectable labels). For example, a first branch of a branched linker may be attached to a first payload (e.g., a first drug, active agent or detectable label) and a second branch of the branched linker may be attached to a second payload (e.g., a second drug, active agent or detectable label) different from the first payload (e.g., the first drug, active agent or detectable label) attached to the first branch.

In some embodiments, where two different drugs or active agents are attached to the branched linker, the drugs or active agents may be selected from drugs and active agents that have a synergistic therapeutic effect. By “synergistic”, “synergism” or “synergy” is meant a therapeutic effect that is greater than the sum of the effects of the drugs or active agents taken separately. For example, in some instances, the use of two different drugs or active agents attached to the branched linker may provide a lower therapeutically effective concentration at which both payloads act, thereby increasing overall potency of the ADC.

In some embodiments, where two different drugs or active agents are attached to the branched linker, the drugs or active agents may be selected from drugs and active agents that provide an enhanced therapeutic benefit as compared to the use of the drugs or active agents separately. For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the branched linker, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different payloads are attached to the branched linker, the payloads may be selected from combinations of drugs or active agents and detectable labels. For example, a first payload may be a detectable label that is used as an imaging agent or tracer to detect the location of the ADC in vivo, while a second payload may be a drug or active agent that provides a therapeutic activity.

Various embodiments of the linkers that may couple the hydrazinyl-indolyl or hydrazinyl-pyrrolo-pyridinyl conjugation moiety to the drugs or active agents are described in detail herein. For example, in some instances, the linker is a cleavable linker, such as a cleavable linker as described herein.

In certain embodiments, the TF antibody may be conjugated to two or more moieties of interest, where one or more amino acids of the TF antibody are modified before conjugation to the moieties of interest. Modification of one or more amino acids of the TF antibody may produce a TF antibody that contains one or more reactive groups suitable for conjugation to the moieties of interest. In some cases, the TF antibody may include one or more modified amino acid residues to provide one or more reactive groups suitable for conjugation to the moieties of interest (e.g., where two or more moieties are attached to a conjugation moiety, such as a hydrazinyl-indolyl or a hydrazinyl-pyrrolo-pyridinyl conjugation moiety as described above). For example, an amino acid of the TF antibody may be modified to include a reactive aldehyde group (e.g., a reactive aldehyde). A reactive aldehyde may be included in an “aldehyde tag” or “ald-tag”, which, as used herein, refers to an amino acid sequence derived from a sulfatase motif (e.g., L(C/S)TPSR, SEQ ID NO:99) that has been converted by action of a formylglycine generating enzyme (FGE) to contain a 2-formylglycine residue (referred to herein as “fGly”). The fGly residue generated by an FGE may also be referred to as a “formylglycine”. Stated differently, the term “aldehyde tag” is used herein to refer to an amino acid sequence that includes a “converted” sulfatase motif (i.e., a sulfatase motif in which a cysteine or serine residue has been converted to fGly by action of an FGE, e.g., L(fGly)TPSR, SEQ ID NO:123). A converted sulfatase motif may be produced from an amino acid sequence that includes an “unconverted” sulfatase motif (i.e., a sulfatase motif in which the cysteine or serine residue has not been converted to fGly by an FGE, but is capable of being converted, e.g., an unconverted sulfatase motif with the sequence: LCTPSR, SEQ ID NO:100). By “conversion” as used in the context of action of a formylglycine generating enzyme (FGE) on a sulfatase motif refers to biochemical modification of a cysteine or serine residue in a sulfatase motif to a formylglycine (fGly) residue (e.g., Cys to fGly, or Ser to fGly). Additional aspects of aldehyde tags and uses thereof in site-specific protein modification are described in U.S. Pat. Nos. 7,985,783 and 8,729,232, the disclosures of each of which are incorporated herein by reference.

In some cases, to produce the conjugate, the TF antibody containing the fGly residue may be conjugated to the moieties of interest by reaction of the fGly with a compound (e.g., a compound containing a hydrazinyl-indolyl or a hydrazinyl-pyrrolo-pyridinyl conjugation moiety, as described above). For example, an fGly-containing TF antibody may be contacted with a reactive partner under conditions suitable to provide for conjugation of two or more drugs to the TF antibody. In some instances, the reactive partner may include a hydrazinyl-indolyl or a hydrazinyl-pyrrolo-pyridinyl conjugation moiety as described above. For example, two or more drugs or active agents may be attached to a hydrazinyl-indolyl or a hydrazinyl-pyrrolo-pyridinyl conjugation moiety. In some cases, the drugs or active agents are attached to a hydrazinyl-indolyl or a hydrazinyl-pyrrolo-pyridinyl conjugation moiety, such as covalently attached to a hydrazinyl-indolyl or a hydrazinyl-pyrrolo-pyridinyl, where each drug or active agent is attached through a corresponding linker to the hydrazinyl-indolyl or a hydrazinyl-pyrrolo-pyridinyl conjugation moiety.

In certain embodiments, a conjugate of the present disclosure includes a TF antibody having at least one amino acid residue that has been attached to two or more moieties of interest (e.g., drugs or active agents). In order to make the conjugate, an amino acid residue of the TF antibody may be modified and then coupled to two or more drugs or active agents attached to a hydrazinyl-indolyl or a hydrazinyl-pyrrolo-pyridinyl conjugation moiety as described above. In certain embodiments, an amino acid residue of the TF antibody is a cysteine or serine residue that is modified to an fGly residue, as described above. In certain embodiments, the modified amino acid residue (e.g., fGly residue) is conjugated to two or more drugs or active agents containing a hydrazinyl-indolyl or a hydrazinyl-pyrrolo-pyridinyl conjugation moiety as described above to provide a conjugate of the present disclosure where the two or more drugs or active agents are conjugated to the TF antibody through the hydrazinyl-indolyl or hydrazinyl-pyrrolo-pyridinyl conjugation moiety. As used herein, the term fGly′ refers to the amino acid residue of the TF antibody that is coupled to the moieties of interest (e.g., drugs or active agents).

In certain embodiments, the conjugate includes a TF antibody having at least one amino acid residue attached to a branched linker as described herein, which in turn is attached to two or more drugs or active agents. For instance, the conjugate may include a TF antibody having at least one amino acid residue (fGly′) that is conjugated to the moieties of interest (e.g., drugs or active agents) as described above.

Aspects of the present disclosure include a conjugate of Formula (I):

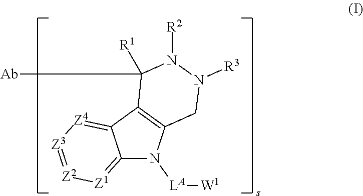

wherein:
Ab represents the antibody that binds to TF;
Z¹, Z², Z³ and Z⁴ are each independently selected from CR⁴, N and C—L^{B}—W², wherein at least one Z¹, Z², Z³ and Z⁴ is C—L^{B}—W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker;
L^{B} is a second linker;
s is an integer from 1 to 10;
W¹ is a first drug; and
W² is a second drug.

The substituents related to conjugates of Formula (I) are described in more detail below.

In certain embodiments, Z¹, Z², Z³ and Z⁴ are each independently selected from CR⁴, N and C—L^{B}—W², wherein at least one Z¹, Z², Z³ and Z⁴ is C—L^{B}—W². In certain embodiments, Z¹ is CR⁴. In certain embodiments, Z¹ is N. In certain embodiments, Z¹ is C—L^{B}—W². In certain embodiments, Z² is CR⁴. In certain embodiments, Z² is N. In certain embodiments, Z² is C—L^{B}—W². In certain embodiments, Z³ is CR⁴. In certain embodiments, Z³ is N. In certain embodiments, Z³ is C—L^{B}—W². In certain embodiments, Z⁴ is CR⁴. In certain embodiments, Z⁴ is N. In certain embodiments, Z⁴ is C—L^{B}—W². In some embodiments, each of Z¹, Z³, and Z⁴ is CR⁴. In some embodiments, Z³ is C—L^{B}—W².

Combinations of various Z¹, Z², Z³ and Z⁴ are possible. For example, in some instances, Z^{L} is C—L^{B}—W², Z² is CR⁴, Z³ is CR⁴, and Z⁴ is CR⁴. In some instances, Z^{L} is CR⁴, Z² is C—L^{B}—W², Z³ is CR⁴, and Z⁴ is CR⁴. In some instances, Z¹ is CR⁴, Z² is CR⁴, Z³ is C—L^{B}—W², and Z⁴ is CR⁴. In some instances, Z^{L} is CR⁴, Z² is CR⁴, Z³ is CR⁴, and Z⁴ is C—L^{B}—W².

In certain embodiments, R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, substituted heterocyclyl. In certain embodiments, R¹ is hydrogen. In certain embodiments, R¹ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R¹ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is alkynyl or substituted alkynyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₅ cycloalkyl or C₃₋₅ substituted cycloalkyl. In certain embodiments, R¹ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₅ heterocyclyl or C₃₋₅ substituted heterocyclyl.

In certain embodiments, R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl.

In certain embodiments, R² is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R² is hydrogen. In certain embodiments, R² is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R² is methyl. In certain embodiments, R² is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R² is alkynyl or substituted alkynyl. In certain embodiments, R² is alkoxy or substituted alkoxy. In certain embodiments, R² is amino or substituted amino. In certain embodiments, R² is carboxyl or carboxyl ester. In certain embodiments, R² is acyl or acyloxy. In certain embodiments, R² is acyl amino or amino acyl. In certain embodiments, R² is alkylamide or substituted alkylamide. In certain embodiments, R² is sulfonyl. In certain embodiments, R² is thioalkoxy or substituted thioalkoxy. In certain embodiments, R² is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R² is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R² is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R² is heterocyclyl or substituted heterocyclyl, such as a C₃₋₆ heterocyclyl or C₃₋₆ substituted heterocyclyl, or a C₃₋₈ heterocyclyl or C₃₋₈ substituted heterocyclyl.

In certain embodiments, R³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R³ is hydrogen. In certain embodiments, R³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R³ is methyl. In certain embodiments, R³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R³ is alkynyl or substituted alkynyl. In certain embodiments, R³ is alkoxy or substituted alkoxy. In certain embodiments, R³ is amino or substituted amino. In certain embodiments, R³ is carboxyl or carboxyl ester. In certain embodiments, R³ is acyl or acyloxy. In certain embodiments, R³ is acyl amino or amino acyl. In certain embodiments, R³ is alkylamide or substituted alkylamide. In certain embodiments, R³ is sulfonyl. In certain embodiments, R³ is thioalkoxy or substituted thioalkoxy. In certain embodiments, R³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₈ substituted heterocyclyl.

In certain embodiment, both R² and R³ are methyl.

In certain embodiments, R² and R³ are optionally cyclically linked to form a 5 or 6-membered heterocyclyl. In certain embodiments, R² and R³ are cyclically linked to form a 5 or 6-membered heterocyclyl. In certain embodiments, R² and R³ are cyclically linked to form a 5-membered heterocyclyl. In certain embodiments, R² and R³ are cyclically linked to form a 6-membered heterocyclyl.

In certain embodiments, each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

The various possibilities for each R⁴ are described in more detail as follows. In certain embodiments, R⁴ is hydrogen. In certain embodiments, each R⁴ is hydrogen. In certain embodiments, R⁴ is halogen, such as F, Cl, Br or I. In certain embodiments, R⁴ is F. In certain embodiments, R⁴ is C₁. In certain embodiments, R⁴ is Br. In certain embodiments, R⁴ is I. In certain embodiments, R⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl. In certain embodiments, R⁴ is methyl. In certain embodiments, R⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R⁴ is alkynyl or substituted alkynyl. In certain embodiments, R⁴ is alkoxy or substituted alkoxy. In certain embodiments, R⁴ is amino or substituted amino. In certain embodiments, R⁴ is carboxyl or carboxyl ester. In certain embodiments, R⁴ is acyl or acyloxy. In certain embodiments, R⁴ is acyl amino or amino acyl. In certain embodiments, R⁴ is alkylamide or substituted alkylamide. In certain embodiments, R⁴ is sulfonyl. In certain embodiments, R⁴ is thioalkoxy or substituted thioalkoxy. In certain embodiments, R⁴ is aryl or substituted aryl, such as C₅₋₈ aryl or C₅₋₈ substituted aryl, such as a C₅ aryl or C₅ substituted aryl, or a C₆ aryl or C₆ substituted aryl (e.g., phenyl or substituted phenyl). In certain embodiments, R⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₈ substituted heterocyclyl.

In certain embodiments, L^{A} is a first linker. Examples of linkers that can be used in the conjugates of the present disclosure are described in more detail below.

In certain embodiments, L^{B} is a second linker. Examples of linkers that can be used in the conjugates of the present disclosure are described in more detail below.

In certain embodiments, W¹ is a first drug (or a first active agent). Examples of drugs and active agents that can be used in the conjugates of the present disclosure are described in more detail below.

In certain embodiments, W² is a second drug (or a second active agent). Examples of drugs and active agents that can be used in the conjugates of the present disclosure are described in more detail below.

In certain embodiments, Ab represents an antibody that binds to tissue factor (“TF antibody”). In certain embodiments, Ab comprises one or more fGly′ residues as described herein. In certain embodiments, the TF antibody is attached to the rest of the conjugate through an fGly′ residue as described herein. Examples of TF antibodies that can be used in the conjugates of the present disclosure are described in more detail below.

In certain embodiments, the conjugate of Formula (I) includes a first linker, L^{A}. The first linker, L^{A}, may be utilized to bind a first moiety of interest (e.g., a first drug or active agent) to a TF antibody through a conjugation moiety. The first linker, L^{A}, may be bound (e.g., covalently bonded) to the conjugation moiety (e.g., as described herein). For example, the first linker, L^{A}, may attach a hydrazinyl-indolyl or a hydrazinyl-pyrrolo-pyridinyl conjugation moiety to a first drug. The hydrazinyl-indolyl or hydrazinyl-pyrrolo-pyridinyl conjugation moiety may be used to conjugate the first linker, L^{A}, (and thus the first drug) to a TF antibody.

For example, as shown in Formula (I) above, L^{A} is attached to Ab through a conjugation moiety, and thus Ab is indirectly bonded to the linker L^{A} through the hydrazinyl-indolyl or a hydrazinyl-pyrrolo-pyridinyl conjugation moiety. As described above, Ab is a TF antibody, and thus L^{A} is attached through the hydrazinyl-indolyl or a hydrazinyl-pyrrolo-pyridinyl conjugation moiety to the TF antibody, e.g., the linker L^{A} is indirectly bonded to the TF antibody through the hydrazinyl-indolyl or a hydrazinyl-pyrrolo-pyridinyl conjugation moiety.

Any convenient linker may be utilized for the first linker L^{A} in the subject conjugates and compounds. In certain embodiments, the first linker L^{A} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the first linker L^{A} may include an alkyl or substituted alkyl group. In certain embodiments, the first linker L^{A} may include an alkenyl or substituted alkenyl group.

In certain embodiments, the first linker L^{A} may include an alkynyl or substituted alkynyl group. In certain embodiments, the first linker L^{A} may include an alkoxy or substituted alkoxy group. In certain embodiments, the first linker L^{A} may include an amino or substituted amino group. In certain embodiments, the first linker L^{A} may include a carboxyl or carboxyl ester group. In certain embodiments, the first linker L^{A} may include an acyl amino group. In certain embodiments, the first linker L^{A} may include an alkylamide or substituted alkylamide group. In certain embodiments, the first linker L^{A} may include an aryl or substituted aryl group. In certain embodiments, the first linker L^{A} may include a heteroaryl or substituted heteroaryl group. In certain embodiments, the first linker L^{A} may include a cycloalkyl or substituted cycloalkyl group. In certain embodiments, the first linker L^{A} may include a heterocyclyl or substituted heterocyclyl group.

In certain embodiments, the first linker L^{A} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{A} is a first linker described by the formula:

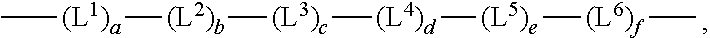

wherein L¹, L², L³, L⁴, L⁵ and L⁶ are each independently a linker subunit, and a, b, c, d, e and f are each independently 0 or 1, provided that at least one of a, b, c, d, e and f is 1.

In certain embodiments, the sum of a, b, c, d, e and f is 1 to 6. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

In certain embodiments, the linker subunit L¹ is attached to the hydrazinyl-indolyl or a hydrazinyl-pyrrolo-pyridinyl conjugation moiety (e.g., as shown in Formula (I) above).

In certain embodiments, the linker subunit L², if present, is attached to the first drug or active agent W¹. In certain embodiments, the linker subunit L³, if present, is attached to the first drug or active agent W¹. In certain embodiments, the linker subunit L⁴, if present, is attached to the first drug or active agent W¹. In certain embodiments, the linker subunit L⁵, if present, is attached to the first drug or active agent W¹. In certain embodiments, the linker subunit L⁶, if present, is attached to the first drug or active agent W¹.

Any convenient linker subunits may be utilized in the first linker L^{A}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L¹, L², L³, L⁴, L⁵ and L⁶ (if present) comprises one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L¹(if present) comprises a polyethylene glycol, a modified polyethylene glycol, an amino acid residue, an alkyl group, a substituted alkyl, an aryl group, a substituted aryl group, or a diamine. In some embodiments, L¹ comprises a polyethylene glycol. In some embodiments, L¹ comprises a modified polyethylene glycol. In some embodiments, L¹ comprises an amino acid residue. In some embodiments, L¹ comprises an alkyl group or a substituted alkyl. In some embodiments, L¹ comprises an aryl group or a substituted aryl group. In some embodiments, L¹ comprises a diamine (e.g., a linking group comprising an alkylene diamine).

In some embodiments, L² (if present) comprises a polyethylene glycol, a modified polyethylene glycol, an amino acid residue, an alkyl group, a substituted alkyl, an aryl group, a substituted aryl group, or a diamine. In some embodiments, L² comprises a polyethylene glycol. In some embodiments, L² comprises a modified polyethylene glycol. In some embodiments, L² comprises an amino acid residue. In some embodiments, L² comprises an alkyl group or a substituted alkyl. In some embodiments, L² comprises an aryl group or a substituted aryl group. In some embodiments, L² comprises a diamine (e.g., a linking group comprising an alkylene diamine).

In some embodiments, L³ (if present) comprises a polyethylene glycol, a modified polyethylene glycol, an amino acid residue, an alkyl group, a substituted alkyl, an aryl group, a substituted aryl group, or a diamine. In some embodiments, L³ comprises a polyethylene glycol. In some embodiments, L³ comprises a modified polyethylene glycol. In some embodiments, L³ comprises an amino acid residue. In some embodiments, L³ comprises an alkyl group or a substituted alkyl. In some embodiments, L³ comprises an aryl group or a substituted aryl group. In some embodiments, L³ comprises a diamine (e.g., a linking group comprising an alkylene diamine).

In some embodiments, L⁴ (if present) comprises a polyethylene glycol, a modified polyethylene glycol, an amino acid residue, an alkyl group, a substituted alkyl, an aryl group, a substituted aryl group, or a diamine. In some embodiments, L⁴ comprises a polyethylene glycol. In some embodiments, L⁴ comprises a modified polyethylene glycol. In some embodiments, L⁴ comprises an amino acid residue. In some embodiments, L⁴ comprises an alkyl group or a substituted alkyl. In some embodiments, L⁴ comprises an aryl group or a substituted aryl group. In some embodiments, L⁴ comprises a diamine (e.g., a linking group comprising an alkylene diamine).

In some embodiments, L⁵(if present) comprises a polyethylene glycol, a modified polyethylene glycol, an amino acid residue, an alkyl group, a substituted alkyl, an aryl group, a substituted aryl group, or a diamine. In some embodiments, L⁵ comprises a polyethylene glycol. In some embodiments, L⁵ comprises a modified polyethylene glycol. In some embodiments, L⁵ comprises an amino acid residue. In some embodiments, L⁵ comprises an alkyl group or a substituted alkyl. In some embodiments, L⁵ comprises an aryl group or a substituted aryl group. In some embodiments, L⁵ comprises a diamine (e.g., a linking group comprising an alkylene diamine).

In some embodiments, L⁶ (if present) comprises a polyethylene glycol, a modified polyethylene glycol, an amino acid residue, an alkyl group, a substituted alkyl, an aryl group, a substituted aryl group, or a diamine. In some embodiments, L⁶ comprises a polyethylene glycol. In some embodiments, L⁶ comprises a modified polyethylene glycol. In some embodiments, L⁶ comprises an amino acid residue. In some embodiments, L⁶ comprises an alkyl group or a substituted alkyl. In some embodiments, L⁶ comprises an aryl group or a substituted aryl group. In some embodiments, L⁶ comprises a diamine (e.g., a linking group comprising an alkylene diamine).

In some embodiments, L^{A} is a first linker comprising

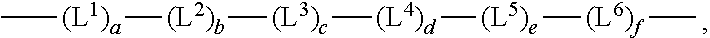

—(L¹)ₐ— is —(T¹—V¹)ₐ—;
—(L²)_{b}— is —(T²—V²)_{b}—;
—(L³)ₑ— is —(T³—V³)_{c}—;
—(L⁴)_{d}— is —(T⁴—V⁴)_{d}—;
—(L⁵)ₑ— is —(T¹—V⁵)ₑ—; and
—(L⁶)_{f}— is —(T⁶—V⁶)_{f}—,
wherein T¹, T², T³, T⁴, T⁵ and T⁶, if present, are tether groups;
V¹, V², V³, V⁴, V⁵ and V⁶, if present, are covalent bonds or linking functional groups; and
a, b, c, d, e and f are each independently 0 or 1, provided that at least one of a, b, c, d, e and f is 1.

In certain embodiments, the sum of a, b, c, d, e and f is 1 to 6. In certain embodiments, the sum of a, b, c, d, e and f is 1. In certain embodiments, the sum of a, b, c, d, e and f is 2. In certain embodiments, the sum of a, b, c, d, e and f is 3. In certain embodiments, the sum of a, b, c, d, e and f is 4. In certain embodiments, the sum of a, b, c, d, e and f is 5. In certain embodiments, the sum of a, b, c, d, e and f is 6. In certain embodiments, a, b, c, d, e and f are each 1. In certain embodiments, a, b, c, d and e are each 1 and f is 0. In certain embodiments, a, b, c and d are each 1 and e and f are each 0. In certain embodiments, a, b, and c are each 1 and d, e and f are each 0. In certain embodiments, a and b are each 1 and c, d, e and f are each 0. In certain embodiments, a is 1 and b, c, d, e and f are each 0.

As described above, in certain embodiments, L¹ is attached to the hydrazinyl-indolyl or a hydrazinyl-pyrrolo-pyridinyl conjugation moiety (e.g., as shown in Formula (I) above). As such, in certain embodiments, T¹ is attached to the hydrazinyl-indolyl or a hydrazinyl-pyrrolo-pyridinyl conjugation moiety (e.g., as shown in Formula (I) above). In certain embodiments, V¹ is attached to the first drug or active agent. In certain embodiments, L², if present, is attached to the first drug or active agent. As such, in certain embodiments, T², if present, is attached to the first drug or active agent, or V², if present, is attached to the first drug or active agent. In certain embodiments, L³, if present, is attached to the first drug or active agent. As such, in certain embodiments, T³, if present, is attached to the first drug or active agent, or V³, if present, is attached to the first drug or active agent. In certain embodiments, L⁴, if present, is attached to the first drug or active agent. As such, in certain embodiments, T⁴, if present, is attached to the first drug or active agent, or V⁴, if present, is attached to the first drug or active agent. In certain embodiments, L⁵, if present, is attached to the first drug or active agent. As such, in certain embodiments, T⁵, if present, is attached to the first drug or active agent, or V⁵, if present, is attached to the first drug or active agent. In certain embodiments, L⁶, if present, is attached to the first drug or active agent. As such, in certain embodiments, T⁶, if present, is attached to the first drug or active agent, or V⁶, if present, is attached to the first drug or active agent.

In certain embodiments, the conjugate of Formula (I) includes a second linker, L^{B}. The second linker, L^{B}, may be utilized to bind a second moiety of interest (e.g., a second drug or active agent) to a TF antibody through a conjugation moiety. The second linker, L^{B}, may be bound (e.g., covalently bonded) to the conjugation moiety (e.g., as described herein). For example, the second linker, L^{B}, may attach a hydrazinyl-indolyl or a hydrazinyl-pyrrolo-pyridinyl conjugation moiety to a second drug. The hydrazinyl-indolyl or hydrazinyl-pyrrolo-pyridinyl conjugation moiety may be used to conjugate the second linker, L^{B}, (and thus the second drug) to a TF antibody.

For example, as shown in Formula (I) above, L^{B} is attached to Ab through a conjugation moiety, and thus Ab is indirectly bonded to the second linker L^{B} through the hydrazinyl-indolyl or a hydrazinyl-pyrrolo-pyridinyl conjugation moiety. As described above, Ab is a TF antibody, and thus L^{B} is attached through the hydrazinyl-indolyl or a hydrazinyl-pyrrolo-pyridinyl conjugation moiety to the TF antibody, e.g., the linker L^{B} is indirectly bonded to the TF antibody through the hydrazinyl-indolyl or a hydrazinyl-pyrrolo-pyridinyl conjugation moiety.

Any convenient linker may be utilized for the second linker L^{B} in the subject conjugates and compounds. In certain embodiments, the second linker L^{B} may include a group selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In certain embodiments, the second linker L^{B} may include an alkyl or substituted alkyl group. In certain embodiments, the second linker L^{B} may include an alkenyl or substituted alkenyl group. In certain embodiments, the second linker L^{B} may include an alkynyl or substituted alkynyl group. In certain embodiments, the second linker L^{B} may include an alkoxy or substituted alkoxy group. In certain embodiments, the second linker L^{B} may include an amino or substituted amino group. In certain embodiments, the second linker L^{B} may include a carboxyl or carboxyl ester group. In certain embodiments, the second linker L^{B} may include an acyl amino group. In certain embodiments, the second linker L^{B} may include an alkylamide or substituted alkylamide group. In certain embodiments, the second linker L^{B}may include an aryl or substituted aryl group. In certain embodiments, the second linker L^{B}may include a heteroaryl or substituted heteroaryl group. In certain embodiments, the second linker L^{B} may include a cycloalkyl or substituted cycloalkyl group. In certain embodiments, the second linker L^{B} may include a heterocyclyl or substituted heterocyclyl group.

In certain embodiments, the second linker L^{B} may include a polymer. For example, the polymer may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In certain embodiments, the polymer is a polyalkylene glycol. In certain embodiments, the polymer is a polyethylene glycol. Other linkers are also possible, as shown in the conjugates and compounds described in more detail below.

In some embodiments, L^{B} is a second linker described by the formula:

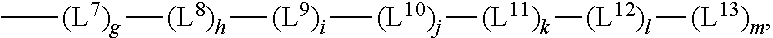

wherein L⁷, L⁸, L⁹, L¹⁰, Li¹, L¹² and L¹³ are each independently a linker subunit, and g, h, i, j, k, l and m are each independently 0 or 1, provided at least one of g, h, i, j, k, l and m is 1.

In certain embodiments, the sum of g, h, i, j, k, l and m is 1 to 7. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each l and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

In certain embodiments, the linker subunit L⁷ is attached to the hydrazinyl-indolyl or a hydrazinyl-pyrrolo-pyridinyl conjugation moiety (e.g., as shown in Formula (I) above). In certain embodiments, the linker subunit L⁸, if present, is attached to the second drug or active agent W². In certain embodiments, the linker subunit L⁹, if present, is attached to the second drug or active agent W². In certain embodiments, the linker subunit L¹⁰, if present, is attached to the second drug or active agent W². In certain embodiments, the linker subunit L¹¹, if present, is attached to the second drug or active agent W². In certain embodiments, the linker subunit L¹², if present, is attached to the second drug or active agent W². In certain embodiments, the linker subunit L¹³, if present, is attached to the second drug or active agent W².

Any convenient linker subunits may be utilized in the second linker L^{B}. Linker subunits of interest include, but are not limited to, units of polymers such as polyethylene glycols, polyethylenes and polyacrylates, amino acid residue(s), carbohydrate-based polymers or carbohydrate residues and derivatives thereof, polynucleotides, alkyl groups, aryl groups, heterocyclic groups, combinations thereof, and substituted versions thereof. In some embodiments, each of L⁷, L⁸, L⁹, L¹⁰, Li¹¹, L¹² and L¹³ (if present) comprise one or more groups independently selected from a polyethylene glycol, a modified polyethylene glycol, an amino acid residue, an alkyl group, a substituted alkyl, an aryl group, a substituted aryl group, and a diamine (e.g., a linking group that includes an alkylene diamine).

In some embodiments, L⁷ (if present) comprises a polyethylene glycol, a modified polyethylene glycol, an amino acid residue, an alkyl group, a substituted alkyl, an aryl group, a substituted aryl group, or a diamine. In some embodiments, L⁷ comprises a polyethylene glycol. In some embodiments, L⁷ comprises a modified polyethylene glycol. In some embodiments, L⁷ comprises an amino acid residue. In some embodiments, L⁷ comprises an alkyl group or a substituted alkyl. In some embodiments, L⁷ comprises an aryl group or a substituted aryl group. In some embodiments, L⁷ comprises a diamine (e.g., a linking group comprising an alkylene diamine).

In some embodiments, L⁸ (if present) comprises a polyethylene glycol, a modified polyethylene glycol, an amino acid residue, an alkyl group, a substituted alkyl, an aryl group, a substituted aryl group, or a diamine. In some embodiments, L⁸ comprises a polyethylene glycol. In some embodiments, L⁸ comprises a modified polyethylene glycol. In some embodiments, L⁸ comprises an amino acid residue. In some embodiments, L⁸ comprises an alkyl group or a substituted alkyl. In some embodiments, L⁸ comprises an aryl group or a substituted aryl group. In some embodiments, L⁸ comprises a diamine (e.g., a linking group comprising an alkylene diamine).

In some embodiments, L⁹ (if present) comprises a polyethylene glycol, a modified polyethylene glycol, an amino acid residue, an alkyl group, a substituted alkyl, an aryl group, a substituted aryl group, or a diamine. In some embodiments, L⁹ comprises a polyethylene glycol. In some embodiments, L⁹ comprises a modified polyethylene glycol. In some embodiments, L⁹ comprises an amino acid residue. In some embodiments, L⁹ comprises an alkyl group or a substituted alkyl. In some embodiments, L⁹ comprises an aryl group or a substituted aryl group. In some embodiments, L⁹ comprises a diamine (e.g., a linking group comprising an alkylene diamine).

In some embodiments, L¹⁰ (if present) comprises a polyethylene glycol, a modified polyethylene glycol, an amino acid residue, an alkyl group, a substituted alkyl, an aryl group, a substituted aryl group, or a diamine. In some embodiments, L¹⁰ comprises a polyethylene glycol. In some embodiments, L¹⁰ comprises a modified polyethylene glycol. In some embodiments, L¹⁰ comprises an amino acid residue. In some embodiments, L¹⁰ comprises an alkyl group or a substituted alkyl. In some embodiments, L¹⁰ comprises an aryl group or a substituted aryl group. In some embodiments, L¹⁰ comprises a diamine (e.g., a linking group comprising an alkylene diamine).

In some embodiments, L¹¹ (if present) comprises a polyethylene glycol, a modified polyethylene glycol, an amino acid residue, an alkyl group, a substituted alkyl, an aryl group, a substituted aryl group, or a diamine. In some embodiments, L¹¹ comprises a polyethylene glycol. In some embodiments, L¹¹ comprises a modified polyethylene glycol. In some embodiments, L¹¹ comprises an amino acid residue. In some embodiments, L¹¹ comprises an alkyl group or a substituted alkyl. In some embodiments, L¹¹ comprises an aryl group or a substituted aryl group. In some embodiments, L¹¹ comprises a diamine (e.g., a linking group comprising an alkylene diamine).

In some embodiments, L¹² (if present) comprises a polyethylene glycol, a modified polyethylene glycol, an amino acid residue, an alkyl group, a substituted alkyl, an aryl group, a substituted aryl group, or a diamine. In some embodiments, L¹² comprises a polyethylene glycol. In some embodiments, L¹² comprises a modified polyethylene glycol. In some embodiments, L¹² comprises an amino acid residue. In some embodiments, L¹² comprises an alkyl group or a substituted alkyl. In some embodiments, L¹² comprises an aryl group or a substituted aryl group. In some embodiments, L¹² comprises a diamine (e.g., a linking group comprising an alkylene diamine).

In some embodiments, L¹³ (if present) comprises a polyethylene glycol, a modified polyethylene glycol, an amino acid residue, an alkyl group, a substituted alkyl, an aryl group, a substituted aryl group, or a diamine. In some embodiments, L¹³ comprises a polyethylene glycol. In some embodiments, L¹³ comprises a modified polyethylene glycol. In some embodiments, L¹³ comprises an amino acid residue. In some embodiments, L¹³ comprises an alkyl group or a substituted alkyl. In some embodiments, L¹³ comprises an aryl group or a substituted aryl group. In some embodiments, L¹³ comprises a diamine (e.g., a linking group comprising an alkylene diamine).

In some embodiments, L^{B} is a second linker comprising

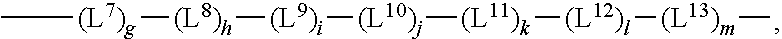

(L⁷)_{g}— is —(T⁷—V⁷)_{g}—;
(L⁸)ₕ— is —(T⁸—V⁸)ₕ—;
(L⁹)ᵢ— is —(T⁹—V⁹)ᵢ—;
(L¹⁰)ⱼ— is —(T¹⁰—V¹⁰)ⱼ—;
(L¹¹)ₖ— is —(T¹¹—V¹¹)ₖ—;
(L¹²)ₗ— is —(T¹²—V¹²)ᵢ—; and
(L¹³)ₘ— is —(T¹³—V¹³)ₘ—,
wherein T⁷, T⁸, T⁹, T¹⁰, T¹¹, T¹ and T¹³, if present, are tether groups;
V⁷, V⁸, V⁹, V¹⁰, V¹¹, V¹² and V¹³, if present, are covalent bonds or linking functional groups; and
g, h, i, j, k, l and m are each independently 0 or 1, provided at least one of g, h, i, j, k, 1, and m is 1.

In certain embodiments, the sum of g, h, i, j, k, l and m is 1 to 7. In certain embodiments, the sum of g, h, i, j, k, l and m is 1. In certain embodiments, the sum of g, h, i, j, k, l and m is 2. In certain embodiments, the sum of g, h, i, j, k, l and m is 3. In certain embodiments, the sum of g, h, i, j, k, l and m is 4. In certain embodiments, the sum of g, h, i, j, k, l and m is 5. In certain embodiments, the sum of g, h, i, j, k, l and m is 6. In certain embodiments, the sum of g, h, i, j, k, l and m is 7. In certain embodiments, g, h, i, j, k, l and m are each 1. In certain embodiments, g, h, i, j, k and 1 are each l and m is 0. In certain embodiments, g, h, i, j and k are each 1 and l and m are each 0. In certain embodiments, g, h, i and j are each 1 and k, l and m are each 0. In certain embodiments, g, h, and i are each 1 and j, k, l and m are each 0. In certain embodiments, g and h are each 1 and i, j, k, l and m are each 0. In certain embodiments, g is 1 and h, i, j, k, l and m are each 0. In certain embodiments, g, h, i, j, k, l and m are each 0.

As described above, in certain embodiments, L⁷ is attached to the hydrazinyl-indolyl or a hydrazinyl-pyrrolo-pyridinyl conjugation moiety (e.g., as shown in Formula (I) above).

As such, in certain embodiments, T⁷ is attached to the hydrazinyl-indolyl or a hydrazinyl-pyrrolo-pyridinyl conjugation moiety (e.g., as shown in Formula (I) above). In certain embodiments, V⁷ is attached to the second drug or active agent. In certain embodiments, L⁸, if present, is attached to the second drug or active agent. As such, in certain embodiments, T⁸, if present, is attached to the second drug or active agent, or V⁸, if present, is attached to the second drug or active agent. In certain embodiments, L⁹, if present, is attached to the second drug or active agent. As such, in certain embodiments, T⁹, if present, is attached to the second drug or active agent, or V⁹, if present, is attached to the second drug or active agent. In certain embodiments, L¹⁰, if present, is attached to the second drug or active agent. As such, in certain embodiments, T¹⁰, if present, is attached to the second drug or active agent, or V10⁴, if present, is attached to the second drug or active agent. In certain embodiments, L¹¹, if present, is attached to the second drug or active agent. As such, in certain embodiments, T¹¹, if present, is attached to the second drug or active agent, or V¹¹, if present, is attached to the second drug or active agent. In certain embodiments, L¹², if present, is attached to the second drug or active agent. As such, in certain embodiments, T¹², if present, is attached to the second drug or active agent, or V¹², if present, is attached to the second drug or active agent. In certain embodiments, L¹³, if present, is attached to the second drug or active agent. As such, in certain embodiments, T¹³, if present, is attached to the second drug or active agent, or V¹³, if present, is attached to the second drug or active agent.

Regarding the tether groups, T¹, T², T³, T⁴, T⁵, T⁶, T⁷, T⁸, T⁹, T¹⁰, T¹¹, T¹² and T¹³, any convenient tether groups may be utilized in the subject linkers. In some embodiments, T¹, T², T³, T⁴, T, T⁶, T⁷, T⁸, T⁹, T¹⁰, T¹¹, T¹² and T¹³ each comprise one or more groups independently selected from a covalent bond, a (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, where each w is an integer from 1 to 20, each n is an integer from 1 to 30, each p is an integer from 1 to 20, and each x is an integer from 1 to 12.

In certain embodiments, the tether group (e.g., T¹, T², T³, T⁴, T⁵, T⁶, T⁷, T⁸, T⁹, T¹⁰, T¹¹, T¹² and/or T¹³) includes a (C₁-C₁₂)alkyl or a substituted (C₁-C₁₂)alkyl. In certain embodiments, (C₁-C₁₂)alkyl is a straight chain or branched alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, (C₁-C₁₂)alkyl may be an alkyl or substituted alkyl, such as C₁-C₁₂ alkyl, or C₁-C₁₀ alkyl, or C₁-C₆ alkyl, or C₁-C₃ alkyl. In some instances, (C₁-C₁₂)alkyl is a C₂-alkyl. For example, (C₁-C₁₂)alkyl may be an alkylene or substituted alkylene, such as C₁-C₁₂ alkylene, or C₁-C₁₀ alkylene, or C₁-C₆ alkylene, or C₁-C₃ alkylene. In some instances, (C₁-C₁₂)alkyl is a C₁-alkylene (e.g., CH₂). In some instances, (C₁-C₁₂)alkyl is a C₂-alkylene (e.g., CH₂CH₂). In some instances, (C₁-C₁₂)alkyl is a C₃-alkylene (e.g., CH₂CH₂CH₂).

In certain embodiments, substituted (C₁-C₁₂)alkyl is a straight chain or branched substituted alkyl group that includes from 1 to 12 carbon atoms, such as 1 to 10 carbon atoms, or 1 to 8 carbon atoms, or 1 to 6 carbon atoms, or 1 to 5 carbon atoms, or 1 to 4 carbon atoms, or 1 to 3 carbon atoms. In some instances, substituted (C₁-C₁₂)alkyl may be a substituted alkyl, such as substituted C₁-C₁₂ alkyl, or substituted C₁-C₁₀ alkyl, or substituted C₁-C₆ alkyl, or substituted C₁-C₃ alkyl. In some instances, substituted (C₁-C₁₂)alkyl is a substituted C₂-alkyl. For example, substituted (C₁-C₁₂)alkyl may be a substituted alkylene, such as substituted C₁-C₁₂ alkylene, or substituted C₁-C₁₀ alkylene, or substituted C₁-C₆ alkylene, or substituted C₁-C₃ alkylene. In some instances, substituted (C₁-C₁₂)alkyl is a substituted C₁-alkylene (e.g., C₁-alkylene substituted with —SO₃H). In some instances, substituted (C₁-C₁₂)alkyl is a substituted C₂-alkylene. In some instances, substituted (C₁-C₁₂)alkyl is a substituted C₃-alkylene. For example, substituted (C₁-C₁₂)alkyl may include C₁-C₁₂ alkylene (e.g., C₃-alkylene or C₅-alkylene) substituted with a (PEG)ₖ group as described herein (e.g., —CONH(PEG)ₖ, such as —CONH(PEG)₃ or —CONH(PEG)₅; or —NHCO(PEG)ₖ, such as —NHCO(PEG)₇), or may include C₁-C₁₂ alkylene (e.g., C₃-alkylene) substituted with a —CONHCH₂CH₂SO₃H group, or may include C₁-C₁₂ alkylene (e.g., C₅-alkylene) substituted with a —NHCOCH₂SO₃H group.

In certain embodiments, the tether group (e.g., T¹, T², T³, T⁴, T⁵, T⁶, T⁷, T⁸, T⁹, T¹⁰, T¹¹, T¹² and/or T¹³) includes an aryl, substituted aryl, heteroaryl, substituted heteroaryl, cycloalkyl, substituted cycloalkyl, heterocyclyl, or substituted heterocyclyl. In some instances, the tether group (e.g., T¹, T², T³, T⁴, T⁵, T⁶, T⁷, T⁸, T⁹, T¹, T¹¹, T¹² and/or T¹³) includes an aryl or substituted aryl. For example, the aryl can be phenyl. In some cases, the substituted aryl is a substituted phenyl. The substituted phenyl can be substituted with one or more substituents selected from (C₁-C₁₂)alkyl, a substituted (C₁-C₁₂)alkyl, aryl, substituted aryl, heteroaryl, substituted heteroaryl, cycloalkyl, substituted cycloalkyl, heterocyclyl, and substituted heterocyclyl. In some instances, the substituted aryl is a substituted phenyl, where the substituent includes a cleavable moiety as described herein (e.g., an enzymatically cleavable moiety, such as a glycoside or glycoside derivative).

In some instances, the tether group (e.g., T¹, T², T³, T⁴, T⁵, T⁶, T⁷, T⁸, T⁹, T¹, T¹¹, T¹² and/or T¹³) includes a heteroaryl or substituted heteroaryl, such triazolyl (e.g., 1,2,3-triazolyl). In some instances, the tether group (e.g., T¹, T², T³, T⁴, T⁵, T⁶, T⁷, T⁸, T⁹, T¹, T¹¹, T¹² and/or T¹³) includes a cycloalkyl or substituted cycloalkyl. In some instances, the tether group (e.g., T¹, T², T³, T⁴, T⁵, T⁶, T⁷, T⁸, T⁹, T¹⁰, T¹¹, T¹² and/or T¹³) includes a heterocyclyl or substituted heterocyclyl. In some instances, the substituent on the substituted heteroaryl, substituted cycloalkyl or substituted heterocyclyl includes a cleavable moiety as described herein (e.g., an enzymatically cleavable moiety, such as a glycoside or glycoside derivative).

In certain embodiments, the tether group (e.g., T¹, T², T³, T⁴, T⁵, T⁶, T⁷, T⁸, T⁹, T¹⁰, T¹¹, T¹² and/or T¹³) includes an ethylene diamine (EDA) moiety, e.g., an EDA containing tether group. In certain embodiments, (EDA)_{w} includes one or more EDA moieties, such as where w is an integer from 1 to 50, such as from 1 to 40, from 1 to 30, from 1 to 20, from 1 to 12 or from 1 to 6, such as 1, 2, 3, 4, 5 or 6). The linked ethylene diamine (EDA) moieties may optionally be substituted at one or more convenient positions with any convenient substituents, e.g., with an alkyl, a substituted alkyl, an acyl, a substituted acyl, an aryl or a substituted aryl. In certain embodiments, the EDA moiety is described by the structure:

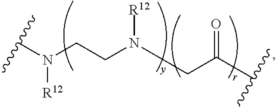

where y is an integer from 1 to 6, r is 0 or 1, and each R¹² is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, y is 1, 2, 3, 4, 5 or 6. In certain embodiments, y is 1 and r is 0. In certain embodiments, y is 1 and r is 1. In certain embodiments, y is 2 and r is 0. In certain embodiments, y is 2 and r is 1. In certain embodiments, each R¹² is independently selected from hydrogen, an alkyl, a substituted alkyl, an aryl and a substituted aryl. In certain embodiments, any two adjacent R¹² groups of the EDA may be cyclically linked, e.g., to form a piperazinyl ring. In certain embodiments, y is 1 and the two adjacent R¹² groups are an alkyl group, cyclically linked to form a piperazinyl ring. In certain embodiments, y is 1 and the adjacent R¹² groups are selected from hydrogen, an alkyl (e.g., methyl) and a substituted alkyl (e.g., lower alkyl-OH, such as ethyl-OH or propyl-OH).

In certain embodiments, the tether group (e.g., T¹, T², T³, T⁴, T⁵, T⁶, T⁷, T⁸, T⁹, T¹⁰, T¹¹, T¹² and/or T¹³) includes a 4-amino-piperidine (4AP) moiety (also referred to herein as piperidin-4-amino, P4A). The 4AP moiety may optionally be substituted at one or more convenient positions with any convenient substituents, e.g., with an alkyl, a substituted alkyl, a polyethylene glycol moiety, an acyl, a substituted acyl, an aryl or a substituted aryl. In certain embodiments, the 4AP moiety is described by the structure:

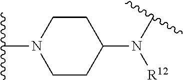

where R¹² is selected from hydrogen, alkyl, substituted alkyl, a polyethylene glycol moiety (e.g., a polyethylene glycol or a modified polyethylene glycol), alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹² is a polyethylene glycol moiety. In certain embodiments, R¹² is a carboxy modified polyethylene glycol.

In certain embodiments, R¹² includes a polyethylene glycol moiety described by the formula: (PEG)ₖ, which may be represented by the structure:

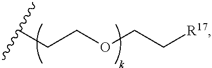

where k is an integer from 1 to 20, such as from 1 to 18, or from 1 to 16, or from 1 to 14, or from 1 to 12, or from 1 to 10, or from 1 to 8, or from 1 to 6, or from 1 to 4, or 1 or 2, such as 1, 2, 3, 4, 5, 6, 7, 8, 9, 10, 11, 12, 13, 14, 15, 16, 17, 18, 19 or 20. In some instances, k is 2. In certain embodiments, R¹⁷ is selected from OH, COOH, OR, or COOR, where R is selected from alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl. In certain embodiments, R¹⁷ is COOH. In certain embodiments, R¹⁷ is OH. In certain embodiments, R¹⁷ is OCH₃.

In certain embodiments, a tether group (e.g., T¹, T², T³, T⁴, T⁵, T⁶, T⁷, T⁸, T⁹, T¹⁰, T¹¹, T¹² and/or T¹³) includes (PEG)ₙ, where (PEG)ₙ is a polyethylene glycol or a modified polyethylene glycol linking unit. In certain embodiments, (PEG)ₙ is described by the structure:

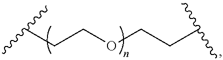

where n is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In some instances, n is 2. In some instances, n is 3. In some instances, n is 6. In some instances, n is 12.

In certain embodiments, a tether group (e.g., T¹, T², T³, T⁴, T⁵, T⁶, T⁷, T⁸, T⁹, T¹⁰, T¹¹, T¹² and/or T¹³) includes (AA)ₚ, where AA is an amino acid residue. Any convenient amino acids may be utilized. Amino acids of interest include but are not limited to, L- and D-amino acids, naturally occurring amino acids such as any of the 20 primary alpha-amino acids and beta-alanine, non-naturally occurring amino acids (e.g., amino acid analogs), such as a non-naturally occurring alpha-amino acid or a non-naturally occurring beta-amino acid, etc. In certain embodiments, p is an integer from 1 to 50, such as from 1 to 40, from 1 to 30, from 1 to 20, from 1 to 12 or from 1 to 6, such as 1, 2, 3, 4, 5, 6, 7, 8, 9, 10, 11, 12, 13, 14, 15, 16, 17, 18, 19 or 20. In certain embodiments, p is 1. In certain embodiments, p is 2.

In certain embodiments, a tether group (e.g., T¹, T², T³, T⁴, T⁵, T⁶, T⁷, T⁸, T⁹, T¹⁰, T¹¹, T¹² and/or T¹³) includes an amino acid analog. Amino acid analogs include compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like. Examples of amino acid analogs include, but are not limited to, sulfoalanine, and the like.

In certain embodiments, a tether group (e.g., T¹, T², T³, T⁴, T⁵, T⁶, T⁷, T⁸, T⁹, T¹⁰, T¹¹, T¹² and/or T¹³) includes a moiety described by the formula —(CR¹³OH)ₓ—, where x is 0 or x is an integer from 1 to 50, such as from 1 to 40, from 1 to 30, from 1 to 20, from 1 to 12 or from 1 to 6, such as 1, 2, 3, 4, 5, 6, 7, 8, 9, 10, 11 or 12. In certain embodiments, x is 1. In certain embodiments, x is 2. In certain embodiments, R¹³ is selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl. In certain embodiments, R¹³ is hydrogen. In certain embodiments, R¹³ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl.

In certain embodiments, R¹³ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹³ is alkynyl or substituted alkynyl. In certain embodiments, R¹³ is alkoxy or substituted alkoxy. In certain embodiments, R¹³ is amino or substituted amino. In certain embodiments, R¹³ is carboxyl or carboxyl ester. In certain embodiments, R¹³ is acyl or acyloxy. In certain embodiments, R¹³ is acyl amino or amino acyl. In certain embodiments, R¹³ is alkylamide or substituted alkylamide. In certain embodiments, R¹³ is sulfonyl. In certain embodiments, R¹³ is thioalkoxy or substituted thioalkoxy. In certain embodiments, R¹³ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹³ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹³ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R¹³ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₈ substituted heterocyclyl.

In certain embodiments, R¹³ is selected from hydrogen, alkyl, substituted alkyl, aryl, and substituted aryl. In these embodiments, alkyl, substituted alkyl, aryl, and substituted aryl are as described above for R¹³.

In certain embodiments, the tether group (e.g., T¹, T², T³, T⁴, T⁵, T⁶, T⁷, T⁸, T⁹, T¹⁰, T¹¹, T¹² and/or T¹³) includes an acetal group, a disulfide, a hydrazine, or an ester. In some embodiments, the tether group includes an acetal group. In some embodiments, the tether group includes a hydrazine. In some embodiments, the tether group includes a disulfide. In some embodiments, the tether group includes an ester.

In certain embodiments, a tether group (e.g., T¹, T², T³, T⁴, T⁵, T⁶, T⁷, T⁸, T⁹, T¹⁰, T¹¹, T¹² and/or T¹³) includes a meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), or para-hydroxy-phenyl (PUP).

In some embodiments, a tether group includes a MABO group described by the following structure:

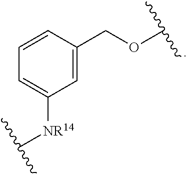

In some embodiments, a tether group includes a MABC group described by the following structure:

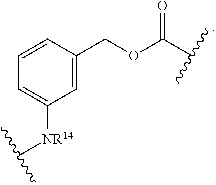

In some embodiments, a tether group includes a PABO group described by the following structure:

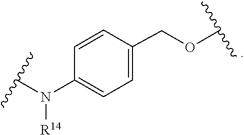

In some embodiments, a tether group includes a PABC group described by the following structure:

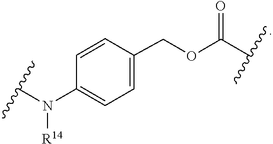

In some embodiments, a tether group includes a PAB group described by the following structure:

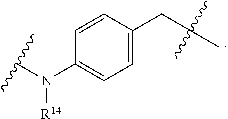

In some embodiments, a tether group includes a PABA group described by the following structure:

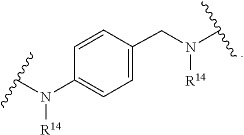

In some embodiments, a tether group includes a PAP group described by the following structure:

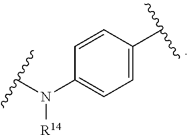

In some embodiments, a tether group includes a PUP group described by the following structure:

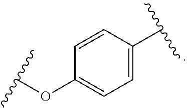 [SMILES: *Oc1ccc(*)cc1]

In certain embodiments, each R¹⁴ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁴ is hydrogen. In certain embodiments, each R¹⁴ is hydrogen. In certain embodiments, R¹⁴ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl.

In certain embodiments, R¹⁴ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁴ is alkynyl or substituted alkynyl. In certain embodiments, R¹⁴ is alkoxy or substituted alkoxy. In certain embodiments, R¹⁴ is amino or substituted amino. In certain embodiments, R¹⁴ is carboxyl or carboxyl ester. In certain embodiments, R¹⁴ is acyl or acyloxy. In certain embodiments, R¹⁴ is acyl amino or amino acyl. In certain embodiments, R¹⁴ is alkylamide or substituted alkylamide. In certain embodiments, R¹⁴ is sulfonyl. In certain embodiments, R¹⁴ is thioalkoxy or substituted thioalkoxy. In certain embodiments, R¹⁴ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁴ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁴ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R¹⁴ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₈ substituted heterocyclyl.

In some embodiments of the MABO, MABC, PABO, PABC, PAB, PABA, PAP, and PHP tether structures shown above, the phenyl ring may be substituted with one or more additional groups selected from halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, one or more of the tether groups T¹, T², T³, T⁴, T⁵, T⁶, T⁷, T⁸, T⁹, T¹⁰, T¹¹, T¹² and/or T¹³ is each optionally substituted with a glycoside or glycoside derivative. For example, in some instances, T¹, T², T³, T⁴, T⁵ and T⁶ are each optionally substituted with a glycoside. In some instances, T⁷, T⁸, T⁹, T¹⁰, T¹¹, T¹² and T¹³ are each optionally substituted with a glycoside. In certain embodiments, the glycoside or glycoside derivative is selected from a glucuronide, a galactoside, a glucoside, a mannoside, a fucoside, O-GlcNAc, and O-GalNAc.

In certain embodiments, the MABO, MABC, PABO, PABC, PAB, PABA, PAP, and PUP tether structures shown above may be substituted with one or more additional groups selected from a glycoside and a glycoside derivative. For example, in some embodiments of the MABO, MABC, PABO, PABC, PAB, PABA, PAP, and PUP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc. In some embodiments, the PABC is substituted with a glycoside, for example, a hydrogen of PABC is replaced with a glycoside, such as a glucuronide, a galactoside, a glucoside, a mannoside, a fucoside, O-GlcNAc, and O-GalNAc.

For example, in some embodiments, the glycoside or glycoside derivative can be selected from the following structures:

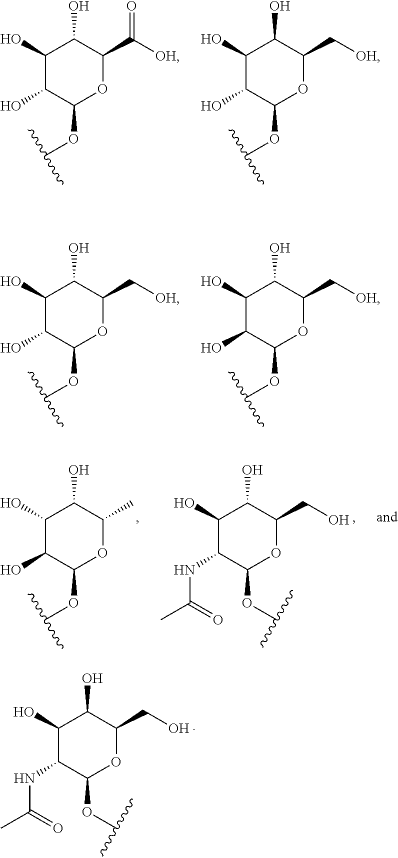

Regarding the linking functional groups, V¹, V², V³, V⁴, V⁵, V⁶, V⁷, V⁸, V⁹, V¹⁰, V¹¹, V¹² and V¹³ any convenient linking functional groups may be utilized in the subject linkers. Linking functional groups of interest include, but are not limited to, amino, carbonyl, amido, oxycarbonyl, carboxy, sulfonyl, sulfoxide, sulfonylamino, aminosulfonyl, thio, oxy, phospho, phosphoramidate, thiophosphoraidate, and the like. In some embodiments, V¹, V², V³, V⁴, V⁵, V⁶, V⁷, V⁸, V⁹, V¹⁰, V¹¹, V¹² and V¹³ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, where q is an integer from 1 to 6. In certain embodiments, q is an integer from 1 to 6 (e.g., 1, 2, 3, 4, 5 or 6). In certain embodiments, q is 1. In certain embodiments, q is 2. In certain embodiments, q is 3. In certain embodiments, q is 4. In certain embodiments, q is 5. In certain embodiments, q is 6.

In some embodiments, each R¹⁵ is independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl.

In certain embodiments, R¹⁵ is hydrogen. In certain embodiments, each R¹⁵ is hydrogen. In certain embodiments, R¹⁵ is alkyl or substituted alkyl, such as C₁₋₆ alkyl or C₁₋₆ substituted alkyl, or C₁₋₄ alkyl or C₁₋₄ substituted alkyl, or C₁₋₃ alkyl or C₁₋₃ substituted alkyl.

In certain embodiments, R¹⁵ is alkenyl or substituted alkenyl, such as C₂₋₆ alkenyl or C₂₋₆ substituted alkenyl, or C₂₋₄ alkenyl or C₂₋₄ substituted alkenyl, or C₂₋₃ alkenyl or C₂₋₃ substituted alkenyl. In certain embodiments, R¹⁵ is alkynyl or substituted alkynyl. In certain embodiments, R¹⁵ is alkoxy or substituted alkoxy. In certain embodiments, R¹⁵ is amino or substituted amino. In certain embodiments, R¹⁵ is carboxyl or carboxyl ester. In certain embodiments, R¹⁵ is acyl or acyloxy. In certain embodiments, R¹⁵ is acyl amino or amino acyl. In certain embodiments, R¹⁵ is alkylamide or substituted alkylamide. In certain embodiments, R¹⁵ is sulfonyl. In certain embodiments, R¹⁵ is thioalkoxy or substituted thioalkoxy. In certain embodiments, R¹⁵ is aryl or substituted aryl, such as C₅₋₈ aryl or C₅₋₈ substituted aryl, such as a C₅ aryl or C₅ substituted aryl, or a C₆ aryl or C₆ substituted aryl. In certain embodiments, R¹⁵ is heteroaryl or substituted heteroaryl, such as C₅₋₈ heteroaryl or C₅₋₈ substituted heteroaryl, such as a C₅ heteroaryl or C₅ substituted heteroaryl, or a C₆ heteroaryl or C₆ substituted heteroaryl. In certain embodiments, R¹⁵ is cycloalkyl or substituted cycloalkyl, such as C₃₋₈ cycloalkyl or C₃₋₈ substituted cycloalkyl, such as a C₃₋₆ cycloalkyl or C₃₋₆ substituted cycloalkyl, or a C₃₋₈ cycloalkyl or C₃₋₈ substituted cycloalkyl. In certain embodiments, R¹⁵ is heterocyclyl or substituted heterocyclyl, such as C₃₋₈ heterocyclyl or C₃₋₈ substituted heterocyclyl, such as a C₃₋₆ heterocyclyl or C₃₋₆ substituted heterocyclyl, or a C₃₋₈ heterocyclyl or C₃₋₈ substituted heterocyclyl.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵.

As described above, in some embodiments, L^{A} is a first linker comprising —(T¹—V¹)ₐ—(T²—V²)_{b}—(T³—V³)_{c}—(T⁴—V⁴)ₐ—(T⁵—V⁵)ₑ—(T⁶—V⁶)_{f}—, where a, b, c, d, e and f are each independently 0 or 1, provided at least one of a, b, c, d, e, and f is 1.

In some embodiments, in the first linker L^{A}:
T¹ is selected from a (C₁-C₁₂)alkyl and a substituted (C₁-C₁₂)alkyl;
T², T³, T⁴, T⁵ and T⁶ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a disulfide, a hydrazine, and an ester; and
V¹, V², V³, V⁴,V⁵ and V⁶ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6; wherein:
(PEG)ₙ is

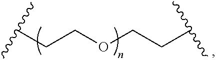

where n is an integer from 1 to 30;
EDA is an ethylene diamine moiety having the following structure:

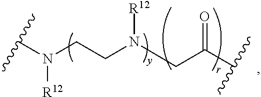

where y is an integer from 1 to 6 and r is 0 or 1;
4-amino-piperidine (4AP) is

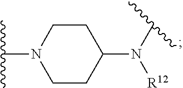

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments, L^{A} comprises:

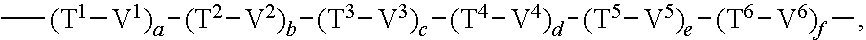

wherein
a, b, c, d, e and f are each independently 0 or 1, provided at least one of a, b, c, d, e, and f is 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴, V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

In some embodiments of L^{A}:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V¹, V², V³, V⁴,V⁵ and V⁶ are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—; wherein:
(PEG)ₙ is

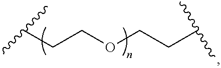

where n is an integer from 1 to 30;
EDA is an ethylene diamine moiety having the following structure:

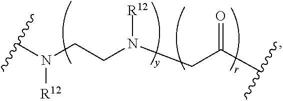

where y is an integer from 1 to 6 and r is 0 or 1;
4-amino-piperidine (4AP) is

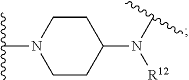

each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
a, b, c, and d are each 1; and
e and fare 0.

In some embodiments, T, T², T³, T⁴, T⁵ and T⁶ are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In certain embodiments, T, T², T³, T⁴, T⁵ and T⁶ and V¹, V², V³, V⁴,V⁵ and V⁶ are selected from the following:

wherein:
T¹ is (C₁-C₁₂)alkyl and V¹ is —CO—;
T² is (AA)ₚ and V² is absent;
T³ is PABC and V³ is absent;
p is an integer from 1 to 10; and
d, e and f are each 0; or
   wherein:
T¹ is (C₁-C₁₂)alkyl and V¹ is —CONH—;
T² is (PEG)ₙ and V² is —CO—;
T³ is (AA)ₚ and V³ is absent;
T⁴ is PABC and V⁴ is absent;
p is an integer from 1 to 10; and
e and f are each 0; or
   wherein:
T¹ is (C₁-C₁₂)alkyl and V¹ is —CO—;
T² is an amino acid analog and V² is —NH—;
T³ is (PEG)ₙ and V³ is —CO—;
T⁴ is (AA)ₚ and V⁴ is absent;
T⁵ is PABC and V⁵ is absent;
p is an integer from 1 to 10; and
f is 0; or
   wherein:
T¹ is (C₁-C₁₂)alkyl and V¹ is —CONH—;
T² is (PEG)ₙ and V² is —CO—;
T³ is (AA)ₚ and V³ is absent;
T⁴ is PABC and V⁴ is absent;
p is an integer from 1 to 10; and
e and f are each 0; or
   wherein:
T¹ is (C₁-C₁₂)alkyl and V¹ is —CONH—;
T² is substituted (C₁-C₁₂)alkyl and V² is —CO—;
T³ is (AA)ₚ and V³ is absent;
T⁴ is PABC and V⁴ is absent;
p is an integer from 1 to 10; and
e and f are each 0; or
   wherein:
T¹ is (C₁-C₁₂)alkyl and V¹ is —CONH—;
T² is (PEG)ₙ and V² is —CO—;
T³ is (AA)ₚ and V³ is absent;
T⁴ is PABA and V⁴ is —CO—;
T⁵ is (C₁-C₁₂)alkyl and V⁵ is absent;
p is an integer from 1 to 10; and
f is 0; or
   wherein:
T¹ is (C₁-C₁₂)alkyl and V¹ is —CO—;
T² is 4AP and V² is —CO—;
T³ is (C₁-C₁₂)alkyl and V³ is —CO—;
T⁴ is (AA)ₚ and V⁴ is absent;
T⁵ is PABC and V⁵ is absent;
p is an integer from 1 to 10; and
f is 0; or
   wherein:
T¹ is (C₁-C₁₂)alkyl and V¹ is —CO—;
T² is 4AP and V² is —CO—;
T³ is (C₁-C₁₂)alkyl and V³ is —O—;
T⁴ is (C₁-C₁₂)alkyl and V⁴ is —CO—;
T⁵ is (AA)ₚ and V⁵ is absent;
p is an integer from 1 to 10; and
T⁶ is PABC and V⁶ is absent; or
   wherein:
T¹ is (C₁-C₁₂)alkyl and V¹ is —CO—;
T² is an amino acid analog and V² is absent;
T³ is (AA)ₚ and V³ is absent;
T⁴ is PABC and V⁴ is absent;
p is an integer from 1 to 10; and
e and f are each 0; or
   wherein:
T¹ is (C₁-C₁₂)alkyl and V¹ is —CONH—;
T² is (PEG)ₙ and V² is —CONH—;
T³ is substituted (C₁-C₁₂)alkyl and V³ is —CO—;
T⁴ is (AA)ₚ and V⁴ is absent;
T⁵ is PABC and V⁵ is absent;
p is an integer from 1 to 10; and
f is 0; or
   wherein:
T¹ is (C₁-C₁₂)alkyl and V¹ is —CO—;
T² is an (AA)ₚ and V² is —NH—;
T³ is (PEG)ₙ and V³ is —CO—;
T⁴ is (AA)ₚ and V⁴ is absent;
T⁵ is PABC and V⁵ is absent;
p is an integer from 1 to 10; and
f is 0; or
   wherein:
T¹ is (C₁-C₁₂)alkyl and V¹ is —CONH—;
T² is (PEG)ₙ and V² is —CO—;
T³ is (AA)ₚ and V³ is absent;
T⁴ is PAP and V⁴ is —C(O)O—;
p is an integer from 1 to 10; and
e and f are each 0; or
   wherein:
T¹ is (C₁-C₁₂)alkyl and V¹ is —CONH—;
T² is substituted (C₁-C₁₂)alkyl and V² is —CO—;
T³ is (AA)ₚ and V³ is absent;
T⁴ is PABC and V⁴ is absent;
p is an integer from 1 to 10; and
e and f are each 0; or
   wherein:
T¹ is (C₁-C₁₂)alkyl and V¹ is —CONH—;
T² is substituted (C₁-C₁₂)alkyl and V² is —CO—;
T³ is PABC and V³ is absent; and
d, e and f are each 0.

In certain embodiments, the left-hand side of the above linker structure for the first linker L^{A} is attached to the hydrazinyl-indolyl or a hydrazinyl-pyrrolo-pyridinyl conjugation moiety, and the right-hand side of the above linker structure for the first linker L^{A} is attached to the first drug or active agent.

As described above, in some embodiments, L^{B} is a second linker comprising —(T⁷—V⁷)_{g}—(T⁸—V⁸)ₕ—(T⁹—V⁹)ᵢ—(T¹⁰—V¹⁰)ⱼ—(T¹¹—V¹¹)ₖ—(T¹²—V¹²)ᵢ—(T¹³—V¹³)ₘ—, where g, h, i, j, k, l and m are each independently 0 or 1, provided that at least one of g, h, i, j, k, l and m is 1.

In some embodiments, in the second linker L^{B}:
T⁷ is selected from a (C₁-C₁₂)alkyl and a substituted (C₁-C₁₂)alkyl;
T⁸, T⁹, T¹⁰, T¹¹, T¹² and T¹³ are each independently selected from (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a disulfide, a hydrazine, and an ester; and
V⁷, V⁸, V⁹, V¹⁰, V¹¹, V¹² and V¹³ are each independently selected from a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂— and —P(O)OH—, wherein q is an integer from 1 to 6;
   wherein:
(PEG)ₙ is

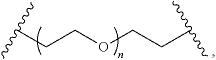

where n is an integer from 1 to 30;
EDA is an ethylene diamine moiety having the following structure:

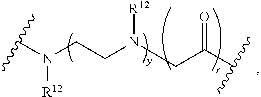

here y is an integer from 1 to 6 and r is 0 or 1;
4-amino-piperidine (4AP) is

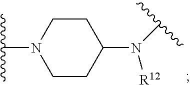

AA is an amino acid residue, where p is an integer from 1 to 20; and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
each R¹³ is independently selected from hydrogen, alkyl, substituted alkyl, aryl, and substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl.

Any convenient tether groups may be utilized for T⁷, T⁸, T⁹, T¹⁰, T¹¹, T¹² and T¹³. For example, any of the tether groups described above in relation to T¹, T², T³, T⁴, T⁵ and T⁶ may be used for the tether groups T⁷, T⁸, T⁹, T¹⁰, T¹¹, T¹ and T¹³.

Any convenient linking functional groups may be utilized for V⁷, V⁸, V⁹, V¹⁰, V¹¹, V¹² and V¹³. For example, any of the linking functional groups described above in relation to V¹, V², V³, V⁴, V⁵ and V⁶ may be used for the linking functional groups V⁷, V⁸, V⁹, V¹⁰, V¹¹, V¹² and V¹³.

In certain embodiments, each R¹³ is independently selected from hydrogen, alkyl, substituted alkyl, aryl, and substituted aryl. In these embodiments, alkyl, substituted alkyl, aryl, and substituted aryl are as described above for R¹³.

In certain embodiments, each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl. In these embodiments, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl are as described above for R¹⁵. In these embodiments, various possible substituents are as described above for R¹⁵.

In certain embodiments of the second linker L^{B}, one or more of the tether groups T⁷, T⁸, T⁹, T¹⁰, T¹¹, T¹² and T¹³ is each optionally substituted with a glycoside or glycoside derivative. In certain embodiments, the glycoside or glycoside derivative is selected from a glucuronide, a galactoside, a glucoside, a mannoside, a fucoside, O-GlcNAc, and O-GalNAc.

In certain embodiments of the second linker L^{B}, the MABO, MABC, PABO, PABC, PAB, PABA, PAP, and PUP tether structures shown above may be substituted with one or more additional groups selected from a glycoside and a glycoside derivative. For example, in some embodiments of the MABO, MABC, PABO, PABC, PAB, PABA, PAP, and PUP tether structures shown above, the phenyl ring may be substituted with one or more additional groups selected from a glycoside and a glycoside derivative. In certain embodiments, the glycoside or glycoside derivative is selected from a glucuronide, a galactoside, a glucoside, a mannoside, a fucoside, O-GlcNAc, and O-GalNAc.

In some embodiments, T⁷, T, T⁹, T¹⁰, T¹¹, T¹² and T¹³ are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In some embodiments of L^{B}:
g, h, i, j, and k are each 1;
l and m is 0;
T⁷ is a covalent bond;
T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V⁷, V⁸, V⁹, V¹⁰, V¹¹ and V¹² are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
   wherein:
(PEG)ₙ is

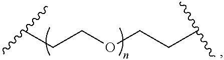

where n is an integer from 1 to 30;
EDA is an ethylene diamine moiety having the following structure:

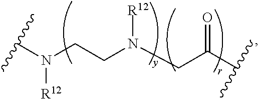

where y is an integer from 1 to 6 and r is 0 or 1;
4-amino-piperidine (4AP) is

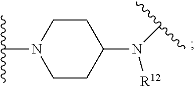

and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring.

In some embodiments, T¹, T², T³, T⁴, T⁵, T⁶, T⁷, T⁸, T⁹, T¹⁰, T¹¹, and T¹² are each optionally substituted with a glycoside.

In some embodiments, MABO, MABC, PABO, PABC, PAB, PABA, PAP and PUP are each optionally substituted with a glycoside.

In some embodiments, the glycoside is selected from a glucuronide, a galactoside, a glucoside, a mannoside, a fucoside, O-GlcNAc, and O-GalNAc.

In certain embodiments, T⁷, T⁸, T⁹, T¹⁰, T¹¹, T¹² and T¹³ and V⁷, V⁸, V⁹, V¹⁰V¹¹, V¹² and V¹³ are selected from the following:

wherein:
T⁷ is absent and V⁷ is —NHCO—;
T⁸ is (C₁-C₁₂)alkyl and V⁸ is —CO—;
T⁹ is (AA)ₚ and V⁹ is absent;
T¹⁰ is PABC and V¹⁰ is absent; and
k, l and m are each 0; or
   wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is (AA)ₚ and V¹⁰ is absent; and
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CO—;
T⁹ is an amino acid analog and V⁹ is —NH—;
T¹⁰ is (PEG)ₙ and V¹⁰ is —CO—;
T¹¹ is (AA)ₚ and V¹¹ is absent;
T¹² is PABC and V¹² is absent; and
m is 0; or
   wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is (AA)ₚ and V¹⁰ is absent;
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is (AA)ₚ and V¹⁰ is absent;
T¹¹ is PABC and V¹¹ is absent; and
l and m are each 0; or
   wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is (AA)ₚ and V¹⁰ is absent;
T¹¹ is PABA and V¹¹ is —CO—;
T¹² is (C₁-C₁₂)alkyl and V¹² is absent; and
m is 0; or
   wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —CO—;
T¹¹ is (AA)ₚ and V¹¹ is absent;
T¹² is PABC and V¹² is absent; and
m is 0; or
   wherein:
T⁷ is absent and V⁷ is —NHCO—;
T⁸ is (C₁-C₁₂)alkyl and V⁸ is —CO—;
T⁹ is 4AP and V⁹ is —CO—;
T¹⁰ is (C₁-C₁₂)alkyl and V¹⁰ is —O—;
T¹¹ is (C₁-C₁₂)alkyl and V¹¹ is —CO—;
T¹² is (AA)ₚ and V¹² is absent; and
T¹³ PABC and V¹³ is absent; or
   wherein:
T⁷ is absent and V⁷ is —NHCO—;
T⁸ is (C₁-C₁₂)alkyl and V⁸ is —CO—;
T⁹ is an amino acid analog and V⁹ is absent;
T¹⁰ is (AA)ₚ and V¹⁰ is absent;
T¹¹ is PABC and V¹¹ is absent; and
l and m are each 0; or
   wherein:
T⁷ is absent and V⁷ is —NHCO—;
TV is (C₁-C₁₂)alkyl and V⁸ is —CONH—;
T⁹ is (PEG)ₙ and V⁹ is —CONH—;
T¹⁰ is substituted (C₁-C₁₂)alkyl and V¹⁰ is —CO—;
T¹¹ is (AA)ₚ and V¹¹ is absent;
T¹² is PABC and V¹² is absent; and
m is 0; or
   wherein:
T⁷ is absent and V⁷ is —NHCO—;
T⁸ is (C₁-C₁₂)alkyl and V⁸ is —CO—;
T⁹ is (AA)ₚ and V⁹ is —NH—;
T¹⁰ is (PEG)ₙ and V¹⁰ is —CO—;
T¹¹ is (AA)ₚ and V¹¹ is absent;
T¹² is PABC and V¹² is absent; and
m is 0; or
   wherein:
T⁷ is absent and V⁷ is —NHCO—;
T⁸ is (C₁-C₁₂)alkyl and V⁸ is —CONH—;
T⁹ is (PEG)ₙ and V⁹ is —CO—;
T¹⁰ is (AA)ₚ and V¹⁰ is absent;
T¹¹ is PAP and V¹¹ is —C(O)O—; and
l and m are each 0; or
   wherein:
T⁷ is absent and V⁷ is —NHCO—;
T⁸ is (C₁-C₁₂)alkyl and V⁸ is —CO—;
T⁹ is (AA)ₚ and V⁹ is absent;
T¹⁰ is PABC and V¹⁰ is absent;
T¹¹ is PAP and V¹¹ is —C(O)O—; and
l and m are each 0; or
   wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is PABC and V¹⁰ is absent; and
k, l and m are each 0; or
   wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is (PEG)ₙ and V¹¹ is —CO—; and
l and m are each 0; or
   wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is (AA)ₚ and V¹² is absent; and
T¹³ PAB and V¹³ is absent; or
   wherein:
T⁷ is absent and V⁷ is —NHCO—;
T⁸ is (C₁-C₁₂)alkyl and V⁸ is absent;
T⁹ is heteroaryl and V⁹ is absent;
T¹⁰ is (C₁-C₁₂)alkyl and V¹⁰ is —CONH—;
T¹¹ is substituted (C₁-C₁₂)alkyl and V¹¹ is —CO—;
T¹² is (AA)ₚ and V¹² is absent; and
T¹³ PABC and V¹³ is absent.

In certain embodiments, the left-hand side of the above linker structure for the second linker L^{B} is attached to the hydrazinyl-indolyl or a hydrazinyl-pyrrolo-pyridinyl conjugation moiety, and the right-hand side of the above linker structure for the second linker L^{B} is attached to the second drug or active agent.

In certain embodiments, the conjugate is an antibody-drug conjugate where the TF antibody and the drugs are linked together by linkers as described above. In some instances, the linker m(e.g., L^{A} and/or L^{B}) is a cleavable linker. A cleavable linker is a linker that includes one or more cleavable moieties, where the cleavable moiety includes one or more bonds that can dissociate under certain conditions, thus separating the cleavable linker into two or more separable portions. For example, the cleavable moiety may include one or more covalent bonds, which under certain conditions, can dissociate or break apart to separate the cleavable linker into two or more portions. As such the linkers that are included in an antibody-drug conjugate can be cleavable linkers, such that under appropriate conditions, the cleavable linker is cleaved to separate or release the drug from the antibody at a desired target site of action for the drug.

In some instances, a cleavable linker includes two cleavable moieties, such as a first cleavable moiety and a second cleavable moiety. The cleavable moieties can be configured such that cleavage of both cleavable moieties is needed in order to separate or release the drug from the TF antibody at a desired target site of action for the drug. For example, cleavage of a cleavable linker can be achieved by initially cleaving one of the two cleavable moieties and then cleaving the other of the two cleavable moieties. In certain embodiments, a cleavable linker includes a first cleavable moiety and a second cleavable moiety that hinders cleavage of the first cleavable moiety. By “hinders cleavage” is meant that the presence of an uncleaved second cleavable moiety reduces the likelihood or substantially inhibits the cleavage of the first cleavable moiety, thus substantially reducing the amount or preventing the cleavage of the cleavable linker. For instance, the presence of uncleaved second cleavable moiety can hinder cleavage of the first cleavable moiety. The hinderance of cleavage of the first cleavable moiety by the presence of the second cleavable moiety, in turn, substantially reduces the amount or prevents the release of the drug from the antibody. For example, the premature release of the drug from the antibody can be substantially reduced or prevented until the antibody-drug conjugate is at or near the desired target site of action for the drug.

In some cases, since the second cleavable moiety hinders cleavage of the first cleavable moiety, cleavage of the cleavable linker can be achieved by initially cleaving the second cleavable moiety and then cleaving the first cleavable moiety. Cleavage of the second cleavable moiety can reduce or eliminate the hinderance on the cleavage of the first cleavable moiety, thus allowing the first cleavable moiety to be cleaved. Cleavage of the first cleavable moiety can result in the cleavable linker dissociating or separating into two or more portions as described above to release the drug from the antibody-drug conjugate. In some instances, cleavage of the first cleavable moiety does not substantially occur in the presence of an uncleaved second cleavable moiety. By substantially is meant that about 10% or less cleavage of the first cleavable moiety occurs in the presence of an uncleaved second cleavable moiety, such as about 9% or less, or about 8% or less, or about 7% or less, or about 6% or less, or about 5% or less, or about 4% or less, or about 3% or less, or about 2% or less, or about 1% or less, or about 0.5% or less, or about 0.1% or less cleavage of the first cleavable moiety occurs in the presence of an uncleaved second cleavable moiety.

Stated another way, the second cleavable moiety can protect the first cleavable moiety from cleavage. For instance, the presence of uncleaved second cleavable moiety can protect the first cleavable moiety from cleavage, and thus substantially reduce or prevent premature release of the drug from the antibody until the antibody-drug conjugate is at or near the desired target site of action for the drug. As such, cleavage of the second cleavable moiety exposes the first cleavable moiety (e.g., deprotects the first cleavable moiety), thus allowing the first cleavable moiety to be cleaved, which results in cleavage of the cleavable linker, which, in turn, separates or releases the drug from the antibody at a desired target site of action for the drug as described above. In certain instances, cleavage of the second cleavable moiety exposes the first cleavable moiety to subsequent cleavage, but cleavage of the second cleavable moiety does not in and of itself result in cleavage of the cleavable linker (i.e., cleavage of the first cleavable moiety is still needed in order to cleave the cleavable linker).

The cleavable moieties included in the cleavable linker may each be an enzymatically cleavable moiety. For example, the first cleavable moiety can be a first enzymatically cleavable moiety and the second cleavable moiety can be a second enzymatically cleavable moiety. An enzymatically cleavable moiety is a cleavable moiety that can be separated into two or more portions as described above through the enzymatic action of an enzyme. The enzymatically cleavable moiety can be any cleavable moiety that can be cleaved through the enzymatic action of an enzyme, such as, but not limited to, an ester, a peptide, a glycoside, and the like. In some instances, the enzyme that cleaves the enzymatically cleavable moiety is present at a desired target site of action, such as the desired target site of action of the drug that is to be released from the antibody-drug conjugate. In some cases, the enzyme that cleaves the enzymatically cleavable moiety is not present in a significant amount in other areas, such as in whole blood, plasma or serum. As such, the cleavage of an enzymatically cleavable moiety can be controlled such that substantial cleavage occurs at the desired site of action, whereas cleavage does not significantly occur in other areas or before the antibody-drug conjugate reaches the desired site of action.

For example, as described herein, antibody-drug conjugates of the present disclosure can be used for the treatment of cancer, such as for the delivery of a cancer therapeutic drug to a desired site of action where the cancer cells are present. In some cases, enzymes, such as an esterase that cleaves ester bonds or a glycosidase that cleaves glycosidic bonds, can be a biomarker for cancer that is overexpressed in cancer cells. The overexpression, and thus localization, of certain enzymes in cancer can be used in the context of the enzymatically cleavable moieties included in the cleavable linkers of the antibody-drug conjugates of the present disclosure to specifically release the drug at the desired site of action (i.e., the site of the cancer (and overexpressed enzyme)). Thus, in some embodiments, the enzymatically cleavable moiety is a cleavable moiety (e.g., an ester or a glycoside) that can be cleaved by an enzyme that is overexpressed in cancer cells. For instance, the enzyme can be an esterase. As such, in some instances, the enzymatically cleavable moiety is a cleavable moiety (e.g., an ester) that can be cleaved by an esterase enzyme. In some instances, the enzyme can be a glycosidase. As such, in some instances, the enzymatically cleavable moiety is a cleavable moiety (e.g., a glycoside or glycoside derivative) that can be cleaved by a glycosidase enzyme.

In certain embodiments, the enzymatically cleavable moiety is an ester bond. For example, the first cleavable moiety described above (i.e., the cleavable moiety protected from premature cleavage by the second cleavable moiety) can include an ester. The presence of uncleaved second cleavable moiety can protect the first cleavable moiety (ester) from cleavage by an esterase enzyme, and thus substantially reduce or prevent premature release of the drug from the antibody until the antibody-drug conjugate is at or near the desired target site of action for the drug. In some instances, a portion of the linker adjacent to the first cleavable moiety is linked to or includes a substituent, where the substituent comprises the second cleavable moiety. In some instances, the second cleavable moiety includes a glycoside or glycoside derivative.

In some embodiments, the enzymatically cleavable moiety is sugar moiety, such as a glycoside (or glyosyl) or glycoside derivative. In some cases, the glycoside or glycoside derivative can facilitate an increase in the hydrophilicity of the cleavable linker as compared to a cleavable linker that does not include the glycoside or glycoside derivative. The glycoside or glycoside derivative can be any glycoside or glycoside derivative suitable for use in the cleavable linker and that can be cleaved through the enzymatic action of an enzyme. For example, the second cleavable moiety (i.e., the cleavable moiety that protects the first cleavable moiety from premature cleavage) can be a glycoside or glycoside derivative. For instance, in some embodiments, the first cleavable moiety includes an ester and the second cleavable moiety includes a glycoside or glycoside derivative. In certain embodiments, the second cleavable moiety is a glycoside or glycoside derivative selected from a glucuronide, a galactoside, a glucoside, a mannoside, a fucoside, O-GlcNAc, and O-GalNAc. In some instances, the second cleavable moiety is a glucuronide. In some instances, the second cleavable moiety is a galactoside. In some instances, the second cleavable moiety is a glucoside. In some instances, the second cleavable moiety is a mannoside. In some instances, the second cleavable moiety is a fucoside. In some instances, the second cleavable moiety is O-GlcNAc. In some instances, the second cleavable moiety is O-GalNAc.

The glycoside or glycoside derivative can be attached (covalently bonded) to the cleavable linker through a glycosidic bond. The glycosidic bond can link the glycoside or glycoside derivative to the cleavable linker through various types of bonds, such as, but not limited to, an O-glycosidic bond (an O-glycoside), an N-glycosidic bond (a glycosylamine), an S-glycosidic bond (a thioglycoside), or C-glycosidic bond (a C-glycoside or C-glycosyl).

In some instances, the glycosidic bond is an O-glycosidic bond (an O-glycoside). In some cases, the glycoside or glycoside derivative can be cleaved from the cleavable linker it is attached to by an enzyme (e.g., through enzymatically-mediated hydrolysis of the glycosidic bond). A glycoside or glycoside derivative can be removed or cleaved from the cleavable linker by any convenient enzyme that is able to carry out the cleavage (hydrolysis) of the glycosidic bond that attaches the glycoside or glycoside derivative to the cleavable linker. An example of an enzyme that can be used to mediate the cleavage (hydrolysis) of the glycosidic bond that attaches the glycoside or glycoside derivative to the cleavable linker is a glycosidase, such as a glucuronidase, a galactosidase, a glucosidase, a mannosidase, a fucosidase, and the like. Other suitable enzymes may also be used to mediate the cleavage (hydrolysis) of the glycosidic bond that attaches the glycoside or glycoside derivative to the cleavable linker. In some cases, the enzyme used to mediate the cleavage (hydrolysis) of the glycosidic bond that attaches the glycoside or glycoside derivative to the cleavable linker is found at or near the desired site of action for the drug of the antibody-drug conjugate. For instance, the enzyme can be a lysosomal enzyme, such as a lysosomal glycosidase, found in cells at or near the desired site of action for the drug of the antibody-drug conjugate. In some cases, the enzyme is an enzyme found at or near the target site where the enzyme that mediates cleavage of the first cleavable moiety is found.

In some embodiments, a TF-ADC is represented by Formula (I):

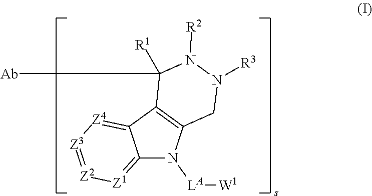

wherein:
Ab represents the antibody that binds to TF;
Z¹, Z², and Z⁴ are each independently CR⁴;
Z³ is C—L^{B}—W²;
R¹, R², R³ and R⁴ are each selected from hydrogen and (C₁-C₁₂)alkyl;
L^{A} is a first linker wherein:
   T¹ is (C₁-C₁₂)alkyl and V¹ is —CONH—;
   T² is substituted (C₁-C₁₂)alkyl and V² is —CO—;
   T³ is (AA)ₚ where p is an integer from 1-20 and V³ is a covalent bond;
   T⁴ is PABC and V⁴ is a covalent bond;
   a, b, c, and d are each 1;
   e and f are each 0; and
L^{B} is a second linker wherein
   T⁷ is a covalent bond and V⁷ is —NHCO—;
   T⁸ is (C₁-C₁₂)alkyl and V⁸ is —CONH—;
   T⁹ is substituted (C₁-C₁₂)alkyl and V⁹ is —CO—;
   T¹⁰ is (AA)ₚ where p is an integer from 1-20 and V¹⁰ is a covalent bond;
   T¹¹ is PABC and V¹¹ is a covalent bond; and
   h, i, j, and k are each 1; and
   l and m are each 0;
s is an integer from 1 to 10;
W¹ is a first drug; and
W² is a second drug.

In some embodiments, one or both of W¹ and W² are camptothecin analogues, for example, belotecan.

In some embodiments, a TF-ADC is represented by Formula (I):

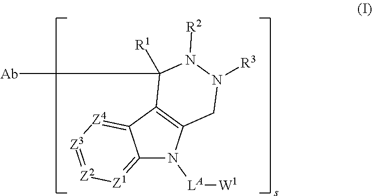

wherein:
Ab represents the antibody that binds to TF;
Z¹, Z², and Z⁴ are each independently CR⁴;
Z³ is C—L^{B}—W²;
R¹, R², R³ and R⁴ are each selected from hydrogen and (C₁-C₁₂)alkyl;
L^{A} is a linker wherein:
   T¹ is (C₁-C₆)alkyl and V¹ is —CONH—;
   T² is (C₁-C₆)alkylene substituted with —NHCO(PEG)ₖ, wherein k is an integer from 2 to 10 and V² is —CO—;
   T³ is (AA)₂ and V³ is a covalent bond;
   T⁴ is PABC substituted with a glycoside and V⁴ is a covalent bond;
   a, b, c, and d are each 1; and
   e and f are each 0; and
L^{B} is a linker wherein
   T⁷ is a covalent bond and V⁷ is —NHCO—;
   T⁸ is (C₁-C₆)alkyl and V⁸ is —CONH—;
   T⁹ is (C₁-C₆)alkylene substituted with —NHCO(PEG)ₖ, wherein k is an integer from 2 to 10 and V⁹ is —CO—;
   T¹⁰ is (AA)₂ and V¹⁰ is a covalent bond;
   T¹¹ is PABC substituted with a glycoside and V¹¹ is a covalent bond;
   h, i, j, and k are each 1; and
   l and m are each 0;
s is an integer from 1 to 10;
W¹ is a first drug; and
W² is a second drug.

In some embodiments, the PABC of one or both of T⁴ and T¹¹ is substituted with a glucuronide. In some embodiments, one or both of T¹ and T⁸ is ethyl. In some embodiments, one or both of T² and T⁹ is C₅ alkylene substituted with —NHCO(PEG)ₖ, where k is an integer from 5 to 10. In some embodiments, one or both of W¹ and W² are camptothecin analogues, for example, belotecan.

In some embodiments, a TF-ADC as disclosed herein comprises any payload, any linker, or any linker-payload as disclosed in US Patent Application No. 2022-0241423 and International Publication No. WO 2022187370, each of which is incorporated herein by reference in its entirety.

In some embodiments, the TF-ADC is represented by Formula (II).

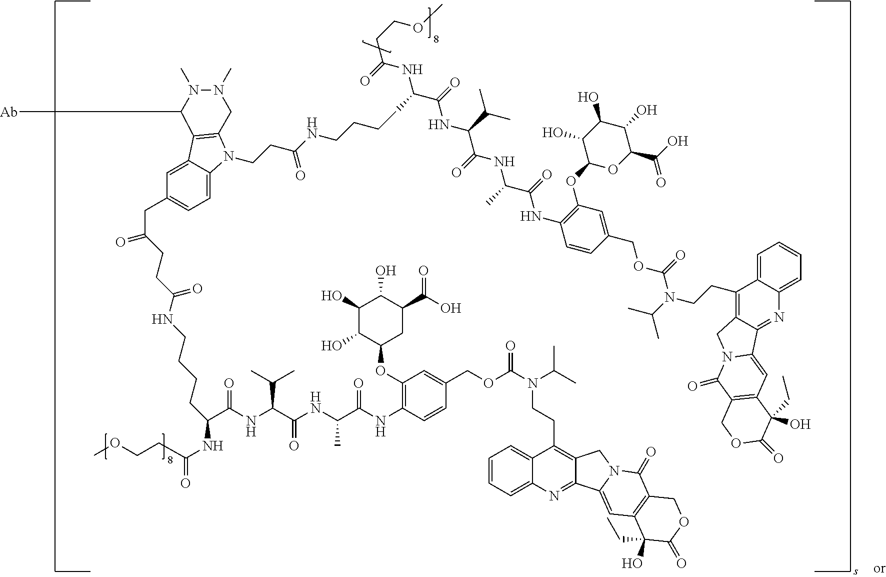

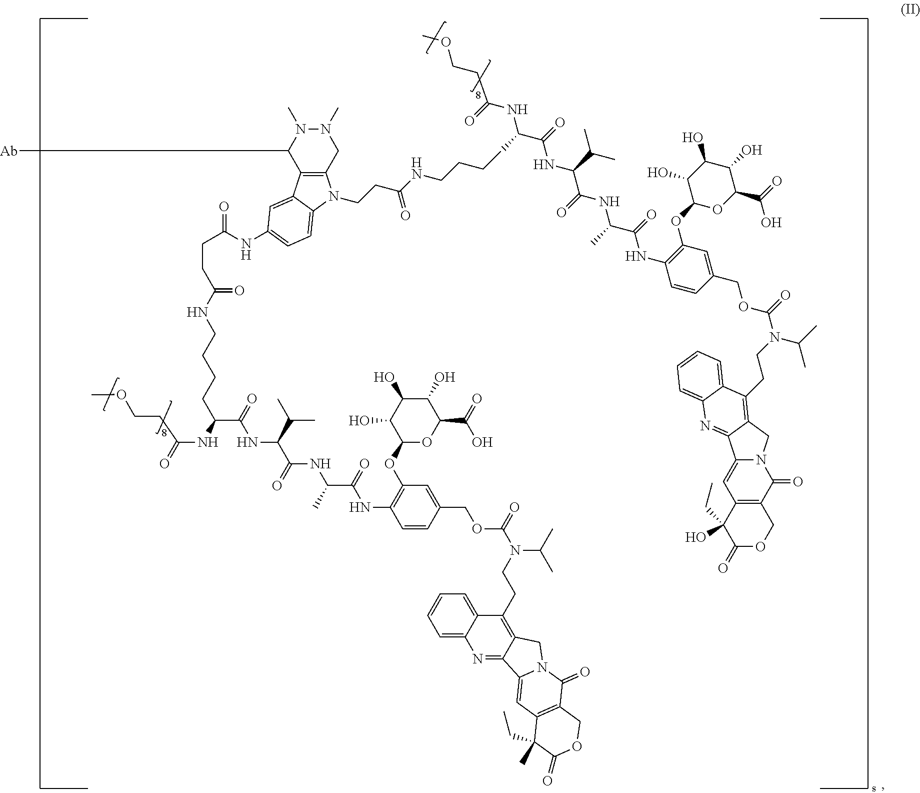

wherein:
Ab represents the antibody that binds to TF; and
s is an integer from 1 to 10.

In some embodiments, s is an integer from 1 to 4.

Any of the chemical entities, linkers and conjugation moieties set forth in the structures above may be adapted for use in the subject compounds and conjugates.

Additional disclosure related to hydrazinyl-indolyl and hydrazinyl-pyrrolo-pyridinyl compounds and methods for producing a conjugate is found in U.S. Pat. Nos. 9,310,374 and 9,493,413, the disclosures of each of which are incorporated herein by reference.

### Tissue Factor (TF) Antibodies

As noted above, a subject conjugate comprises an antibody (Ab) that binds to TF. The amino acid sequence of the antibody can be modified to include a 2-formylglycine (fGly) residue. As used herein, amino acids may be referred to by their standard name, their standard three letter abbreviation and/or their standard one letter abbreviation, such as: Alanine or Ala or A; Cysteine or Cys or C; Aspartic acid or Asp or D; Glutamic acid or Glu or E; Phenylalanine or Phe or F; Glycine or Gly or G; Histidine or His or H; Isoleucine or IIe or I; Lysine or Lys or K; Leucine or Leu or L; Methionine or Met or M; Asparagine or Asn or N; Proline or Pro or P; Glutamine or Gln or Q; Arginine or Arg or R; Serine or Ser or S; Threonine or Thr or T; Valine or Val or V; Tryptophan or Trp or W; and Tyrosine or Tyr or Y.

TF-ADCs described herein comprise a drug and TF antibody conjugated thereto. In some embodiments, a TF antibody refers to an antibody, specifically binding to TF, such as a TF protein, a TF polypeptide, a TF polypeptide fragment, a TF peptide, or a TF epitope. In some embodiments, the TF antibody is a human or humanized antibody (e.g., comprising human constant regions) that binds to TF. In some embodiments, a TF antibody can bind to TF which is expressed on the surface of a mammalian (e.g., human) cell, including a TF-expressing tumor cell. In some embodiments, a TF antibody binds a TF extracellular epitope expressed on a cell such as a tumor cell (e.g., an extracellular TF epitope). In some embodiments, TF is a human TF. An exemplary amino acid sequence of human TF is described herein (SEQ ID NO:175).

In some embodiments, the TF antibody competes for binding to TF with a reference TF antibody that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as described in any one of Tables 1-2. Accordingly, in some embodiments, the TF antibody competes for binding to TF with a reference TF antibody that comprises one, two, and/or three VH CDRs and/or one, two, and/or three VL CDRs from: (a) the antibody designated as EXMA-006; or (b) the antibody designated as EXMA-007. In some embodiments, a TF-ADC comprises a drug conjugated (directly or indirectly) to a TF antibody that competes for binding to TF with a reference TF antibody that comprises one, two, and/or three VH CDRs and one, two, and/or three VL CDRs from: (a) the antibody designated as EXMA-006; or (b) the antibody designated as EXMA-007. In some embodiments, the TF antibody competes for binding to TF with a reference TF antibody that comprises a VH region and VL region from: (a) the antibody designated as EXMA-006; or (b) the antibody designated as EXMA-007. In some embodiments, the TF antibody competes for binding to TF with a reference TF antibody that comprises: (a) a VH region comprising the amino acid sequence of SEQ ID NO:25 and a VL region comprising the amino acid sequence of SEQ ID NO:26; or (b) a VH region comprising the amino acid sequence of SEQ ID NO:41 and a VL region comprising the amino acid sequence of SEQ ID NO:42. In some embodiments, other suitable TF antibodies can be used, see, for example, International Publication Nos. WO2019136309 and WO2021003399, each of which in incorporated herein by reference in its entirety.

In some embodiments, the TF antibody comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as described in any one of Tables 1-2. Accordingly, in some embodiments, the TF antibody comprises one, two, and/or three heavy chain CDRs and/or one, two, and/or three light chain CDRs from: (a) the antibody designated as EXMA-006; or (b) the antibody designated as EXMA-007. In some embodiments, the TF antibody comprises one, two, and/or three heavy chain CDRs and one, two, and/or three light chain CDRs from: (a) the antibody designated as EXMA-006; or (b) the antibody designated as EXMA-007.

In some embodiments, the TF antibody comprises a VH region, which comprises one or more (such as one, two, or three) of VH CDR1, VH CDR2, and VH CDR3 as described herein, such as in any one of Tables 1-2; and/or a VL region, which comprises one or more (such as one, two, or three) of VL CDR1, VL CDR2, and VL CDR3 as described herein, such as in any one of Tables 1-2. In some embodiments, the TF antibody is bispecific and comprises a first binding region that comprises one, two, and/or three heavy chain CDRs and/or one, two, and/or three light chain CDRs as described in any one of Tables 1-2 and a second region that comprises one, two, and/or three heavy chain CDRs and/or one, two, and/or three light chain CDRs from a binding agent that binds to a second target antigen that is not TF. In some embodiments, the TF antibody is bispecific and comprises a first binding domain that comprises one, two, and/or three heavy chain CDRs and/or one, two, and/or three light chain CDRs as described in any one of Tables 1-2 and a second binding domain that comprises one, two, and/or three heavy chain CDRs and/or one, two, and/or three light chain CDRs from a binding agent that binds to a second TF epitope.

The antibody designated as EXMA-006 comprises a VH amino acid sequence of SEQ ID NO:25 and a VL amino acid sequence of SEQ ID NO:26.

The antibody designated as EXMA-007 comprises a VH amino acid sequence of SEQ ID NO:41 and a VL amino acid sequence of SEQ ID NO:42.

TABLE 1
Antibody EXMA-006-CDR Sequences

| | | Exemplary* | Kabat | Chothia | AbM | Contact | IMGT |
|---|---|---|---|---|---|---|---|
| VH CDR | VH | GYTFD | VYGIS | GYTFD | GYTFD | DVYGI | GYTFD |
| Seq. | CDR1 | VYGIS | (SEQ ID | VY | VYGIS | S | VYG |
| | | (SEQ ID | NO: 7) | (SEQ ID | (SEQ ID | (SEQ ID | (SEQ ID |
| | | NO: 1) | | NO: 8) | NO: 1) | NO: 15) | NO: 21) |
| | VH | WIAPY | WIAPY | PYSG | WIAPY | WMGWI | IAPYS |
| | CDR2 | SGNTN | SGNTN | (SEQ ID | SGNTN | APYSG | GNT |
| | | YAQKL | YAQKL | NO: 9) | (SEQ ID | NTN | (SEQ ID |
| | | QG | QG | | NO: 14) | (SEQ ID | NO: 22) |
| | | (SEQ ID | (SEQ ID | | | NO: 16) | |
| | | NO: 2) | NO: 2) | | | | |
| | VH | DAGTY | DAGTY | AGTYS | DAGTY | ARDAG | ARDAG |
| | CDR3 | SPFGY | SPFGY | PFGYG | SPFGY | TYSPF | TYSPF |
| | | GM | GMDV | MD | GMDV | GYGMD | GY |
| | | DV | (SEQ ID | (SEQ ID | (SEQ ID | (SEQ ID | GMDV |
| | | (SEQ ID | NO: 3) | NO: 10) | NO: 3) | NO: 17) | (SEQ ID |
| | | NO: 3) | | | | | NO: 23) |
| VL CDR | VL | QASQS | QASQS | SQSIN | QASQS | NNWLA | QSINN |
| Seq. | CDR1 | INNWL | INNWL | NW | INNWL | WY | W |
| | | A | A | (SEQ ID | A | (SEQ ID | (SEQ ID |
| | | (SEQ ID | (SEQ ID | NO: 11) | (SEQ ID | NO: 18) | NO: 24) |
| | | NO: 4) | NO: 4) | | NO: 4) | | |
| | VL | KAYNL | KAYNL | KAY | KAYNL | LLIYK | KAY |
| | CDR2 | ES | ES | (SEQ ID | ES | AYNLE | (SEQ ID |
| | | (SEQ ID | (SEQ ID | NO: 12) | (SEQ ID | (SEQ ID | NO: 12) |
| | | NO: 5) | NO: 5) | | NO: 5) | NO: 19) | |
| | VL | QLFQS | QLFQS | FQSLP | QLFQS | QLFQS | QLFQS |
| | CDR3 | LPPFT | LPPFT | PF | LPPFT | LPPF | LPPFT |
| | | (SEQ ID | (SEQ ID | (SEQ ID | (SEQ ID | (SEQ ID | (SEQ ID |
| | | NO: 6) | NO: 6) | NO: 13) | NO: 6) | NO: 20) | NO: 6) |

VH Sequence*:
QVQLVQSGAEVKKPGASVKVSCKASGYTFDVYGISWVRQAPGQGLEWMGWIAPYSGNTNYAQKLQGRVTMTTDTSTS
TAYMELRSLRSDDTAVYYCARDAGTYSPFGYGMDVWGQGTTVTVSS
(SEQ ID NO: 25)
VL Sequence*:
DIQMTQSPSTLSASVGDRVTITCQASQSINNWLAWYQQKPGKAPKLLIYKAYNLESGVPSRFSGSGSGTEFTLTISSL
QPDDFATYYCQLFQSLPPFTFGGGTKVEIK
(SEQ ID NO: 26)
*Exemplary CDR sequences encompass amino acids as determined by Kabat plus Chothia

TABLE 2
Antibody EXMA-007-CDR Sequences

| | | Exemplary* | Kabat | Chothia | AbM | Contact | IMGT |
|---|---|---|---|---|---|---|---|
| VH CDR | VH | GYTFD | AYGIS | GYTFD | GYTFD | DAYGI | GYTFD |
| Seq. | CDR1 | AYGIS | (SEQ ID | AY | AYGIS | S | AYG |
| | | (SEQ ID | NO: 31) | (SEQ ID | (SEQ ID | (SEQ ID | (SEQ ID |
| | | NO: 27) | | NO: 32) | NO: 27) | NO: 35) | NO: 39) |
| | VH | WIAPY | WIAPY | PYSG | WIAPY | WMGWI | IAPYS |
| | CDR2 | SGNTN | SGNTN | (SEQ ID | SGNTN | APYSG | GNT |
| | | YA | YA | NO: 9) | (SEQ ID | NTN | (SEQ ID |
| | | QKLQG | QKLQG | | NO: 14) | (SEQ ID | NO: 22) |
| | | (SEQ ID | (SEQ ID | | | NO: 16) | |
| | | NO: 2) | NO: 2) | | | | |
| | VH | DAGTY | DAGTY | AGTYS | DAGTY | ARDAG | ARDAG |
| | CDR3 | SPFGY | SPFGY | PFGYG | SPFGY | TYSPF | TYSPF |
| | | GMDV | GMDV | MD | GMDV | GYGMD | GYGMD |
| | | (SEQ ID | (SEQ ID | (SEQ ID | (SEQ ID | (SEQ ID | V |
| | | NO: 3) | NO: 3) | NO: 10) | NO: 3) | NO: 17) | (SEQ ID |
| | | | | | | | NO: 23) |
| VL CDR | VL | RASES | RASES | SESIS | RASES | SNWLA | ESISN |
| Seq. | CDR1 | ISNWL | ISNWL | NW | ISNWL | WY | W |
| | | A | A | (SEQ ID | A | (SEQ ID | (SEQ ID |
| | | (SEQ ID | (SEQ ID | NO: 33) | (SEQ ID | NO: 36) | NO: 40) |
| | | NO: 28) | NO: 28) | | NO: 28) | | |
| | VL | KAYSL | KAYSL | KAY | KAYSL | LLIYK | KAY |
| | CDR2 | EY | EY | (SEQ ID | EY | AYSLE | (SEQ ID |
| | | (SEQ ID | (SEQ ID | NO: 12) | (SEQ ID | (SEQ ID | NO: 12) |
| | | NO: 29) | NO: 29) | | NO: 29) | NO: 37) | |
| | VL | QQFQK | QQFQK | FQKLP | QQFQK | QQFQK | QQFQK |
| | CDR3 | LPPFT | LPPFT | PF | LPPFT | LPPF | LPPFT |
| | | (SEQ ID | (SEQ ID | (SEQ ID | (SEQ ID | (SEQ ID | (SEQ ID |
| | | NO: 30) | NO: 30) | NO: 34) | NO: 30) | NO: 38) | NO: 30) |

VH Sequence*:
QVQLVQSGAEVKKPGASVKVSCKASGYTFDAYGISWVRQAPGQGLEWMGWIAPYSGNTNYAQKLQGRVTMTTDTSTSTA
YMELRSLRSDDTAVYYCARDAGTYSPFGYGMDVWGQGTTVTVSS
(SEQ ID NO: 41)
VL Sequence*:
DIQMTQSPSTLSASVGDRVTITCRASESISNWLAWYQQKPGKAPKLLIYKAYSLEYGVPSRFSGSGSGTEFTLTISSLQ
PDDFATYYCQQFQKLPPFTFGGGTKVEIK
(SEQ ID NO: 42)
*Exemplary CDR sequences encompass amino acids as determined by Kabat plus Chothia

In some embodiments, the TF antibody comprises a VH region. In some embodiments, the TF antibody comprises a VL region. In some embodiments, the TF antibody has a combination of (i) a VH region; and (ii) a VL region.

In some embodiments, the TF antibody comprises a heavy chain having a combination of (i) a VH as described herein, such as in any one of Tables 1-2, and (ii) one or more heavy chain constant regions (e.g., CH1, hinge, CH2, and CH3). An exemplary IgG heavy chain can comprise any VH amino acid sequence as described herein and the following CH1, hinge, CH2, and CH3 amino acid sequence:

(SEQ ID NO: 89)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG.

In some embodiments, the TF antibody comprises a heavy chain having a combination of (i) a VH as described herein, such as in any one of Tables 1-2, and (ii) one or more heavy chain constant regions (e.g., CH1, hinge, CH2, and CH3). An exemplary IgG heavy chain can comprise any VH amino acid sequence as described herein and the following CH1, hinge, CH2, and CH3 amino acid sequence:

(SEQ ID NO: 90)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKRVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPG.

In some embodiments, a TF antibody comprises a light chain having a combination of (i) a VL region as described herein, such as in any one of Tables 1-2; and (ii) a light chain constant region (CL). An exemplary light chain (e.g., for pairing with an IgG heavy chain) can comprise any VL amino acid sequence described herein and the following CL amino acid sequence:

(SEQ ID NO: 95)
RTVAAPSVFIFPPSDSQLKSGTASVVCLLNNFYPREAKVQWKVDNALQSG
NSQESVTEQDSKDSTYSLSSTLTLSKADYEKHKVYACEVTHQGLSSPVTK
SFNRGEC.

In some embodiments, a TF antibody comprises a light chain having a combination of (i) a VL region as described herein, such as in any one of Tables 1-2; and (ii) a light chain constant region (CL). An exemplary light chain (e.g., for pairing with an IgG heavy chain) can comprise any VL amino acid sequence described herein and the following CL amino acid sequence:

(SEQ ID NO: 96)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC

In some embodiments, the TF antibody comprises (a) a heavy chain having a combination of (i) a VH as described herein, such as in any one of Tables 1-2, and (ii) one or more heavy chain constant regions (e.g., CH1, hinge, CH2, and CH3); and (b) a light chain having a combination of (i) a VL as described herein, such as in any one of Tables 1-2, and (ii) a light chain constant region in an IgG format (CL1).

In some embodiments, the TF antibody comprises a VH having an amino acid sequence of:

(SEQ ID NO: 26)
DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIK.

In some embodiments, the TF antibody comprises a VH having an amino acid sequence of:

(SEQ ID NO: 42)
DIQMTQSPSTLSASVGDRVTITCRASESISNWLAWYQQKPGKAPKLLIYK
AYSLEYGVPSRFSGSGSGTEFTLTISSLQPDDFATYYCQQFQKLPPFTFG
GGTKVEIK

In some embodiments, the antibody that binds to TF comprises a heavy chain having an amino acid sequence of: QVQLVQSGAEVKKPGASVKVSCKASGYTFDVYGISWVRQAPGQGLEWMGWIAPYS GNTNYAQKLQGRVTMTTDTSTSTAYMELRSLRSDDTAVYYCARDAGTYSPFGYGM DVWGQGTTVTVSSASTKGPSVFPLAPSSKSTSGGTAALGCLVKDYFPEPVTVSWNSG ALTSGVHTFPAVLQSSGLYSL SSVVTVPSSSLGTQTYICNVNHKPSNTKVDKRVEPKS CDKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSREEMTKNQVSLT CLVKGFYPSDIAVEWESNGQPEN NYKTTPPVLDSDGSFFLYSKLTVDKSRWQQGNVF SCSVMHEALHNHYTQKSLSLSP G (SEQ ID NO:93); and a light chain having an amino acid sequence of:

(SEQ ID NO: 91)
DIQMTQSPSTLSASVGDRVTITCQASQSINNWLAWYQQKPGKAPKLLIYK
AYNLESGVPSRFSGSGSGTEFTLTISSLQPDDFATYYCQLFQSLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, the antibody that binds to TF comprises a heavy chain having an amino acid sequence of: QVQLVQSGAEVKKPGASVKVSCKASGYTFDAYGISWVRQAPGQGLEWMGWIAPYS GNTNYAQKLQGRVTMTTDTSTSTAYMELRSLRSDDTAVYYCARDAGTYSPFGYGM DVWGQGTTVTVSSASTKGPSVFPLAPSSKSTSGGTAALGCLVKDYFPEPVTVSWNSG ALTSGVHTFPAVLQSSGLYSL SSVVTVPSSSLGTQTYICNVNHKPSNTKVDKRVEPKS CDKTHTCPPCPAPELLGGPSVFLFPPKPKDTLMISRTPEVTCVVVDVSHEDPEVKFNW YVDGVEVHNAKTKPREEQYNSTYRVVSVLTVLHQDWLNGKEYKCKVSNKALPAPI EKTISKAKGQPREPQVYTLPPSREEMTKNQVSLTCLVKGFYPSDIAVEWESNGQPEN NYKTTPPVLDSDGSFFLYSKLTVDKSRWQQGNVFSCSVMHEALHNHYTQKSLSLSP G (SEQ ID NO:94); and a light chain having an amino acid sequence of:

(SEQ ID NO: 92)
DIQMTQSPSTLSASVGDRVTITCRASESISNWLAWYQQKPGKAPKLLIYK
AYSLEYGVPSRFSGSGSGTEFTLTISSLQPDDFATYYCQQFQKLPPFTFG
GGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC.

In some embodiments, one or both of the heavy chains of the TF antibody further comprises a signal peptide, for example at the N terminus of the chain. Additionally, or alternatively, one or both of the light chains of the TF antibody further comprises a signal peptide, for example at the N terminus of the chain. In some embodiments, the signal peptide comprises an amino acid sequence of MMSFVSLLLVGILFHATQA (SEQ ID NO:97). In some embodiments, the signal peptide comprises an amino acid sequence of

| (SEQ ID NO: 98) | |
|---|---|
| | MGWSLILLFLVAVATRVHS. |

In some embodiments, the TF antibody comprises a heavy chain with a signal peptide and therefore have an amino acid sequence of:

(SEQ ID NO: 179)
MMSFVSLLLVGILFHATQAQVQLVQSGAEVKKPGASVKVSCKASGYTFDV
YGISWVRQAPGQGLEWMGWIAPYSGNTNYAQKLQGRVTMTTDTSTSTAYM
ELRSLRSDDTAVYYCARDAGTYSPFGYGMDVWGQGTTVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPG,

wherein the underlined amino acids represent the signal peptide sequence.

In some embodiments, the TF antibody comprises a heavy chain with a signal peptide and therefore have an amino acid sequence of:

(SEQ ID NO: 180)
MMSFVSLLLVGILFHATQAQVQLVQSGAEVKKPGASVKVSCKASGYTFDA
YGISWVRQAPGQGLEWMGWIAPYSGNTNYAQKLQGRVTMTTDTSTSTAYM
ELRSLRSDDTAVYYCARDAGTYSPFGYGMDVWGQGTTVTVSSASTKGPSV
FPLAPSSKSTSGGTAALGCLVKDYFPEPVTVSWNSGALTSGVHTFPAVLQ
SSGLYSLSSVVTVPSSSLGTQTYICNVNHKPSNTKVDKRVEPKSCDKTHT
CPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSN
KALPAPIEKTISKAKGQPREPQVYTLPPSREEMTKNQVSLTCLVKGFYPS
DIAVEWESNGQPENNYKTTPPVLDSDGSFFLYSKLTVDKSRWQQGNVFSC
SVMHEALHNHYTQKSLSLSPG,

wherein the underlined amino acids represent the signal peptide sequence.

In some embodiments, a TF antibody comprises one or more CDRs (e.g., one, two, three, four, five, or six CDRs), for example, a VH CDR1, VH CDR2, VH CDR3, VL CDR1, VL CDR2, and/or VL CDR3 as described in Table 1. In some embodiments, a TF antibody comprises one or more CDRs, (e.g., one, two, three, four, five, or six CDRs), for example, a VH CDR1, VH CDR2, VH CDR3, VL CDR1, VL CDR2, and/or VL CDR3 as described in Table 2.

In some embodiments, a TF antibody comprises one or more CDRs, (e.g., one, two, or three VH CDRs), for example, a VH CDR1, VH CDR2, VH CDR3, as described in Table 1. In some embodiments, a TF antibody comprises one or more CDRs, (e.g., one, two, or three VL CDRs), for example, a VL CDR1, VL CDR2, and/or VL CDR3, as described in Table 1. In some embodiments, a TF antibody comprises one or more CDRs, (e.g., one, two, or three VH CDRs), for example, a VH CDR1, VH CDR2, VH CDR3, as described in Table 1 and one or more CDRs, (e.g., one, two, or three VL CDRs), for example, a VL CDR1, VL CDR2, and/or VL CDR3, as described in Table 1.

In some embodiments, a TF antibody comprises one or more CDRs, (e.g., one, two, or three VH CDRs), for example, a VH CDR1, VH CDR2, VH CDR3, as described in Table 2. In other embodiments, a TF antibody comprises one or more CDRs, (e.g., one, two, or three VL CDRs), for example, a VL CDR1, VL CDR2, and/or VL CDR3, as described in Table 2. In some embodiments, a TF antibody comprises one or more CDRs, (e.g., one, two, or three VH CDRs), for example, a VH CDR1, VH CDR2, VH CDR3, as described in Table 2 and one or more CDRs, (e.g., one, two, or three VL CDRs), for example, a VL CDR1, VL CDR2, and/or VL CDR3, as described in Table 2.

In some embodiments, a TF antibody comprises one or more (e.g., one, two, or three) VH CDRs as described herein, such as in Tables 1-2. In other embodiments, a TF antibody comprises one or more (e.g., one, two, or three) VL CDRs as described herein, such as in Tables 1-2. In some embodiments, a TF antibody comprises one or more (e.g., one, two, or three) VH CDRs as described herein, such as in Tables 1-2 and one or more (e.g., one, two, or three) VL CDRs as described herein, such as in Tables 1-2. Accordingly, in some embodiments, a TF antibody comprises a VH CDR1 comprising an amino acid sequence comprising any one of SEQ ID NOs:1, 7, 8, 15, 21, 27, 31, 32, 35, and 39. In some embodiments, a TF antibody comprises a VH CDR2 comprising an amino acid sequence comprising any one of SEQ ID NOs:2, 9, 14, 16, and 22. In some embodiments, a TF antibody comprises a VH CDR3 comprising an amino acid sequence of any one of SEQ ID NOs:3, 10, 17, and 23. In some embodiments, a TF antibody comprises a VH CDR1 and/or a VH CDR2 and/or a VH CDR3 independently selected from a VH CDR1, VH CDR2, VH CDR3 as described in any one of Tables 1-2. In some embodiments, a TF antibody comprises a VL CDR1 comprising an amino acid sequence of any one of SEQ ID NOs:4, 11, 18, 24, 28, 33, 36, and 40. In some embodiments, a TF antibody comprises a VL CDR2 comprising an amino acid sequence of any one of SEQ ID NOs:5, 12, 19, 29, and 37. In some embodiments, a TF antibody comprises a VL CDR3 comprising an amino acid sequence of any one of SEQ ID NOs:6, 13, 20, 30, 34, and 38. In some embodiments, a TF antibody comprises a VL CDR1 and/or a VL CDR2 and/or a VL CDR3 independently selected from a VL CDR1, VL CDR2, VL CDR3 as described herein, such as in any one of Tables 1-2.

In some embodiments, a TF-ADC comprises a TF antibody, wherein the antibody comprises all three heavy chain CDRs and/or all three light chain CDRs from: (a) the antibody designated as EXMA-006 that comprises a VH amino acid sequence of SEQ ID NO:25 and a VL amino acid sequence of SEQ ID NO:26; or (b) the antibody designated as EXMA-007 that comprises a VH amino acid sequence of SEQ ID NO:41 and a VL amino acid sequence of SEQ ID NO:42. In some embodiments, a TF-ADC comprises a TF antibody, wherein the TF antibody comprises all three heavy chain CDRs and/or all three light chain CDRs from the antibody designated as EXMA-006. In some embodiments, a TF-ADC comprises a TF antibody, wherein the TF antibody comprises all three heavy chain CDRs and/or all three light chain CDRs from the antibody designated as EXMA-006.

In some embodiments, a TF-ADC comprises a TF antibody, wherein the antibody comprises: (a) a VH region comprising a VH CDR1, a VH CDR2, and a VH CDR3 amino acid sequence as described herein, such as in Tables 1-2; and/or (b) a VL region comprising a VL CDR1, a VL CDR2, and a VL CDR3 amino acid sequence as described herein, such as in Tables 1-2. In some embodiments, a TF-ADC comprises a TF antibody wherein the antibody comprises: a VH region comprising a VH CDR1, a VH CDR2, and a VH CDR3 amino acid sequence as described herein, such as in Tables 1-2. In some embodiments, a TF-ADC comprises a TF antibody, wherein the antibody comprises a VL region comprising a VL CDR1, a VL CDR2, and a VL CDR3 amino acid sequence as described herein, such as in Tables 1-2.

In some embodiments, the TF antibody comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the TF antibody comprises a VH CDR1 comprising the amino acid sequence of SEQ ID NO:7, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the TF antibody comprises a VH CDR1 comprising the amino acid sequence of SEQ ID NO:8, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:11, a VL CDR2 comprising the amino acid sequence of SEQ ID NO:12, and a VL CDR3 comprising the amino acid sequence of SEQ ID NO:13.

In some embodiments, the TF antibody comprises a VH CDR1 comprising the amino acid sequence of SEQ ID NO:1, a VH CDR2 comprising the amino acid sequence of SEQ ID NO:14, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

In some embodiments, the TF antibody comprises a VH CDR1 comprising the amino acid sequence of SEQ ID NO:15, a VH CDR2 comprising the amino acid sequence of SEQ ID NO:16, a VH CDR3 comprising the amino acid sequence of SEQ ID NO:17, a VL CDR1 comprising the amino acid sequence of SEQ ID NO:18, a VL CDR2 comprising the amino acid sequence of SEQ ID NO:19, and a VL CDR3 comprising the amino acid sequence of SEQ ID NO:20.

In some embodiments, the TF antibody comprises a VH CDR1 comprising the amino acid sequence of SEQ ID NO:21, a VH CDR2 comprising the amino acid sequence of SEQ ID NO:22, a VH CDR3 comprising the amino acid sequence of SEQ ID NO:23, a VL CDR1 comprising the amino acid sequence of SEQ ID NO:24, a VL CDR2 comprising the amino acid sequence of SEQ ID NO:12, and a VL CDR3 comprising the amino acid sequence of SEQ ID NO:6.

In some embodiments, the TF antibody comprises a VH CDR1 comprising the amino acid sequence of SEQ ID NO:27, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:28, a VL CDR2 comprising the amino acid sequence of SEQ ID NO:29, and a VL CDR3 comprising the amino acid sequence of SEQ ID NO:30.

In some embodiments, the TF antibody comprises a VH CDR1 comprising the amino acid sequence of SEQ ID NO:31, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:28, a VL CDR2 comprising the amino acid sequence of SEQ ID NO:29, and a VL CDR3 comprising the amino acid sequence of SEQ ID NO:30.

In some embodiments, the TF antibody comprises a VH CDR1 comprising the amino acid sequence of SEQ ID NO:32, a VH CDR2 comprising the amino acid sequence of SEQ ID NO:9, a VH CDR3 comprising the amino acid sequence of SEQ ID NO:10, a VL CDR1 comprising the amino acid sequence of SEQ ID NO:33, a VL CDR2 comprising the amino acid sequence of SEQ ID NO:12, and a VL CDR3 comprising the amino acid sequence of SEQ ID NO:34.

In some embodiments, the TF antibody comprises a VH CDR1 comprising the amino acid sequence of SEQ ID NO:27, a VH CDR2 comprising the amino acid sequence of SEQ ID NO:14, a VH CDR3 comprising the amino acid sequence of SEQ ID NO:3, a VL CDR1 comprising the amino acid sequence of SEQ ID NO:28, a VL CDR2 comprising the amino acid sequence of SEQ ID NO:29, and a VL CDR3 comprising the amino acid sequence of SEQ ID NO:30.

In some embodiments, the TF antibody comprises a VH CDR1 comprising the amino acid sequence of SEQ ID NO:35, a VH CDR2 comprising the amino acid sequence of SEQ ID NO:16, a VH CDR3 comprising the amino acid sequence of SEQ ID NO:17, a VL CDR1 comprising the amino acid sequence of SEQ ID NO:36, a VL CDR2 comprising the amino acid sequence of SEQ ID NO:37, and a VL CDR3 comprising the amino acid sequence of SEQ ID NO:38.

In some embodiments, the TF antibody comprises a VH CDR1 comprising the amino acid sequence of SEQ ID NO:39, a VH CDR2 comprising the amino acid sequence of SEQ ID NO:22, a VH CDR3 comprising the amino acid sequence of SEQ ID NO:23, a VL CDR1 comprising the amino acid sequence of SEQ ID NO:40, a VL CDR2 comprising the amino acid sequence of SEQ ID NO:12, and a VL CDR3 comprising the amino acid sequence of SEQ ID NO:30.

In some embodiments, a TF-ADC comprises a TF antibody, wherein the antibody comprises a VH region and/or VL region described herein, wherein an VH and/or VL comprises human framework sequences. In some embodiments, an VH region and/or VL region comprises a framework 1 (FR1), a framework 2 (FR2), a framework 3 (FR3) and/or a framework 4 (FR4) sequence, such as a human FR1, a human FR2, a human FR3 and/or a human FR4.

In some embodiments, an CDRs of a TF antibody can be determined by the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In some embodiments, CDRs of a TF antibody can be determined by the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A. et al., 1990, J. Mol. Biol. 215(1):175-82; and U.S. Pat. No. 7,709,226).

In some embodiments, CDRs of a TF antibody can be determined by the ImMunoGeneTics (IMGT®) system, for example, as described in Lefranc, M.-P., 1999, The Immunologist, 7:132-136 and Lefranc, M.-P. et al., 1999, Nucleic Acids Res., 27:209-212 (“IMGT® CDRs”).

In some embodiments, CDRs of a TF antibody can be determined by the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001).

In some embodiments, CDRs of a TF antibody can be determined by the Contact system, which will be referred to herein as the “Contact CDRs” (see, e.g., MacCallum R M et al., 1996, J Mol Biol 5:732-745). The Contact CDRs are based on an analysis of the available complex crystal structures.

In some embodiments, a TF antibody comprises a heavy chain variable region (VH) comprising: (1) a VH CDR1 comprising an amino acid sequence of any one of: (i) SEQ ID NO:1, (ii) SEQ ID NO:7, (iii) SEQ ID NO:8, (iv) SEQ ID NO:15, and (v) SEQ ID NO:21; (2) a VH CDR2 comprising an amino acid sequence of any one of: (i) SEQ ID NO:2, (ii) SEQ ID NO:9, (iii) SEQ ID NO:14, (iv) SEQ ID NO:16, and (v) SEQ ID NO:22; and (3) a VH CDR3 comprising an amino acid sequence of any one of: (i) SEQ ID NO:3, (ii) SEQ ID NO:10, (iii) SEQ ID NO:17, and (iv) SEQ ID NO:23; and/or a VL region comprising: (1) a VL CDR1 comprising an amino acid sequence of any one of: (i) SEQ ID NO:4, (ii) SEQ ID NO:11, (iii) SEQ ID NO:18, and (iv) SEQ ID NO:24; (2) a VL CDR2 comprising an amino acid sequence of any one of: (i) SEQ ID NO:5, (ii) SEQ ID NO:12, and (iii) SEQ ID NO:19; and (3) a VL CDR3 comprising an amino acid sequence of any one of: (i) SEQ ID NO:6, (ii) SEQ ID NO:13, and (iii) SEQ ID NO:20.

In some embodiments, a TF antibody comprises a VH region comprising: (1) a VH CDR1 comprising an amino acid sequence of any one of: (i) SEQ ID NO:1, (ii) SEQ ID NO:7, (iii) SEQ ID NO:8, (iv) SEQ ID NO:15, and (v) SEQ ID NO:21; (2) a VH CDR2 comprising an amino acid sequence of any one of: (i) SEQ ID NO:2, (ii) SEQ ID NO:9, (iii) SEQ ID NO:14, (iv) SEQ ID NO:16, and (v) SEQ ID NO:22; and (3) a VH CDR3 comprising an amino acid sequence of any one of: (i) SEQ ID NO:3, (ii) SEQ ID NO:10, (iii) SEQ ID NO:17, and (iv) SEQ ID NO:23.

In some embodiments, a TF antibody comprises a VL region comprising: (1) a VL CDR1 comprising an amino acid sequence of any one of: (i) SEQ ID NO:4, (ii) SEQ ID NO:11, (iii) SEQ ID NO:18, and (iv) SEQ ID NO:24; (2) a VL CDR2 comprising an amino acid sequence of any one of: (i) SEQ ID NO:5, (ii) SEQ ID NO:12, and (iii) SEQ ID NO:19; and (3) a VL CDR3 comprising an amino acid sequence of any one of: (i) SEQ ID NO:6, (ii) SEQ ID NO:13, and (iii) SEQ ID NO:20.

In some embodiments, a TF-ADC comprises a TF antibody, wherein the antibody comprises: (1) a VL CDR1 comprising an amino acid sequence of any one of: SEQ ID NOs:4, 11, 18, and 24; (2) a VL CDR2 comprising an amino acid sequence of any one of: SEQ ID NOs:5, 12, and 19; and (3) a VL CDR3 comprising an amino acid sequence of any one of: SEQ ID NOs:6, 13, and 20.

In some embodiments, a TF-ADC comprises a TF antibody, wherein the antibody comprises a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:6.

In some embodiments, a TF-ADC comprises a TF antibody, wherein the antibody comprises: (a) a VH region comprising: (1) a VH CDR1 comprising an amino acid sequence of SEQ ID NO:1; and (b) a VL region comprising: (1) a VL CDR1 comprising an amino acid sequence of any one of SEQ ID NOs:4, 11, 18, and 24; (2) a VL CDR2 comprising an amino acid sequence of any one of SEQ ID NOs:5, 12, and 19; and (3) a VL CDR3 comprising an amino acid sequence of any one of SEQ ID NOs:6, 13, and 20.

In some embodiments, a TF-ADC comprises a TF antibody, wherein the antibody comprises: (a) a VH region comprising: (1) a VH CDR1 comprising an amino acid sequence of SEQ ID NO:1; and (b) a VL region comprising: (1) a VL CDR1 comprising an amino acid sequence of SEQ ID NO:4; (2) a VL CDR2 comprising an amino acid sequence of SEQ ID NO:5; and (3) a VL CDR3 comprising an amino acid sequence of SEQ ID NO:6.

In some embodiments, a TF-ADC is prepared from a TF antibody, wherein the antibody comprises a VH comprising an amino acid sequence of SEQ ID NO:25, and/or a VL comprising an amino acid sequence of SEQ ID NO:26.

In some embodiments, a TF-ADC is prepared from a TF antibody, wherein the antibody comprises a heavy chain that is inserted with one or more fGly site(s) and comprises an amino acid sequence of SEQ ID NO:78 or 81 and/or a light chain comprising an amino acid sequence of SEQ ID NO:26. In some embodiments, a TF-ADC is prepared from a TF antibody, wherein the antibody comprises a heavy chain that is inserted with one or more fGly site(s) and comprises an amino acid sequence of SEQ ID NO:78 or 81 and/or a light chain comprising an amino acid sequence of SEQ ID NO:91.

In some embodiments, a TF-ADC comprises a TF antibody, wherein the antibody comprises a heavy chain that is inserted with one or more fGly site(s) conjugated to a linker-drug construct as disclosed herein and comprises an amino acid sequence of SEQ ID NO:84 or 87, and/or a light chain comprising an amino acid sequence of SEQ ID NO:26. In some embodiments, a TF-ADC comprises a TF antibody, wherein the antibody comprises a heavy chain that is inserted with one or more fGly site(s) conjugated to a linker-drug construct as disclosed herein and comprises an amino acid sequence of SEQ ID NO:84 or 87, and/or a light chain comprising an amino acid sequence of SEQ ID NO:91.

In some embodiments, a TF antibody comprises a heavy chain variable region (VH) comprising: (1) a VH CDR1 comprising an amino acid sequence of any one of: (i) SEQ ID NO:27, (ii) SEQ ID NO:31, (iii) SEQ ID NO:32, (iv) SEQ ID NO:35, and (v) SEQ ID NO:39; (2) a VH CDR2 comprising an amino acid sequence of any one of: (i) SEQ ID NO:2, (ii) SEQ ID NO:9, (iii) SEQ ID NO:14, (iv) SEQ ID NO:16, and (v) SEQ ID NO:22; and (3) a VH CDR3 comprising an amino acid sequence of any one of: (i) SEQ ID NO:3, (ii) SEQ ID NO:10, (iii) SEQ ID NO:17, and (iv) SEQ ID NO:23; and/or a VL region comprising: (1) a VL CDR1 comprising an amino acid sequence of any one of: (i) SEQ ID NO:28, (ii) SEQ ID NO:33, (iii) SEQ ID NO:36, and (iv) SEQ ID NO:40; (2) a VL CDR2 comprising an amino acid sequence of any one of: (i) SEQ ID NO:12, (ii) SEQ ID NO:29, and (iii) SEQ ID NO:37; and (3) a VL CDR3 comprising an amino acid sequence of any one of: (i) SEQ ID NO:30, (ii) SEQ ID NO:34, and (iii) SEQ ID NO:38.

In some embodiments, a TF antibody comprises a VH region comprising: (1) a VH CDR1 comprising an amino acid sequence of any one of: (i) SEQ ID NO:27, (ii) SEQ ID NO:31, (iii) SEQ ID NO:32, (iv) SEQ ID NO:35, and (v) SEQ ID NO:39; (2) a VH CDR2 comprising an amino acid sequence of any one of: (i) SEQ ID NO:2, (ii) SEQ ID NO:9, (iii) SEQ ID NO:14, (iv) SEQ ID NO:16, and (v) SEQ ID NO:22; and (3) a VH CDR3 comprising an amino acid sequence of any one of: (i) SEQ ID NO:3, (ii) SEQ ID NO:10, (iii) SEQ ID NO:17, and (iv) SEQ ID NO:23.

In some embodiments, a TF antibody comprises a VL region comprising: (1) a VL CDR1 comprising an amino acid sequence of any one of: (i) SEQ ID NO:28, (ii) SEQ ID NO:33, (iii) SEQ ID NO:36, and (iv) SEQ ID NO:40; (2) a VL CDR2 comprising an amino acid sequence of any one of: (i) SEQ ID NO:12, (ii) SEQ ID NO:29, and (iii) SEQ ID NO:37; and (3) a VL CDR3 comprising an amino acid sequence of any one of: (i) SEQ ID NO:30, (ii) SEQ ID NO:34, and (iii) SEQ ID NO:38.

In some embodiments, a TF-ADC comprises a TF antibody, wherein the antibody comprises: a VL CDR1 comprising an amino acid sequence of any one of SEQ ID NOs:28, 33, 36, and 40; (2) a VL CDR2 comprising an amino acid sequence of any one of SEQ ID SEQ ID NOs:12, 29, and 37; and (3) a VL CDR3 comprising an amino acid sequence of any one of SEQ ID SEQ ID NOs:30, 34, and 38.

In some embodiments, a TF-ADC comprises a TF antibody, wherein the antibody comprises a VL region comprising: (1) a VL CDR1 comprising an amino acid sequence of SEQ ID NO:28; (2) a VL CDR2 comprising an amino acid sequence of SEQ ID NO:12; and (3) a VL CDR3 comprising an amino acid sequence of SEQ ID NO:13.

In some embodiments, a TF-ADC comprises a TF antibody, wherein the antibody comprises: (a) a VH region comprising: (1) a VH CDR1 comprising an amino acid sequence of SEQ ID NO:27; and (b) a VL region comprising: (1) a VL CDR1 comprising an amino acid sequence of any one of SEQ ID NOs:28, 33, 36, and 40; (2) a VL CDR2 comprising an amino acid sequence of any one of SEQ ID NOs:12, 29, and 37; and (3) a VL CDR3 comprising an amino acid sequence of any one of SEQ ID SEQ ID NOs:30, 34, and 38.

In some embodiments, a TF-ADC comprises a TF antibody, wherein the antibody comprises: (a) a VH region comprising: (1) a VH CDR1 comprising an amino acid sequence of SEQ ID NO:27; and (b) a VL region comprising: (1) a VL CDR1 comprising an amino acid sequence of SEQ ID NO:28; and (2) a VL CDR2 comprising an amino acid sequence of SEQ ID NO:29; and (3) a VL CDR3 comprising an amino acid sequence of SEQ ID SEQ ID NO:30.

In some embodiments, a TF-ADC is prepared from a TF antibody, wherein the antibody comprises a heavy chain comprising an amino acid sequence of SEQ ID NO:41, and/or a light chain comprising an amino acid sequence of SEQ ID NO:42.

In some embodiments, a TF-ADC is prepared from a TF antibody, wherein the antibody comprises a heavy chain that is inserted with one or more fGly site(s) and comprises an amino acid sequence of SEQ ID NO:78 or 81 and/or a light chain comprising an amino acid sequence of SEQ ID NO:42. In some embodiments, a TF-ADC is prepared from a TF antibody, wherein the antibody comprises a heavy chain that is inserted with one or more fGly site(s) and comprises an amino acid sequence of SEQ ID NO:78 or 81 and/or a light chain comprising an amino acid sequence of SEQ ID NO:92.

In some embodiments, a TF-ADC comprises a TF antibody, wherein the antibody comprises a heavy chain that is conjugated to one or more fGly site(s) conjugated to a linker-drug construct as disclosed herein and comprises an amino acid sequence of SEQ ID NO:84 or 87, and/or a light chain comprising an amino acid sequence of SEQ ID NO:42. In some embodiments, a TF-ADC comprises a TF antibody, wherein the antibody comprises a heavy chain that is conjugated to one or more fGly site(s) conjugated to a linker-drug construct as disclosed herein and comprises an amino acid sequence of SEQ ID NO:84 or 87, and/or a light chain comprising an amino acid sequence of SEQ ID NO:92.

In some embodiments, the position of one or more (e.g., one, two, three) CDRs along the VH (e.g., CDR1, CDR2, or CDR3) and/or VL (e.g., CDR1, CDR2, or CDR3) region of a TF antibody can vary by one, two, three, four, five, or six amino acid positions provided that binding to TF (e.g., human TF) is maintained (e.g., substantially maintained, for example, at least 50%, at least 60%, at least 70%, at least 80%, at least 90%, or at least 95%).

For example, in some embodiments, the position defining a CDR of any of Table 1 or Table 2 can vary by shifting the N-terminal and/or C-terminal boundary of the CDR by one, two, three, four, five, or six amino acids, relative to the current CDR position, provided that binding to TF (e.g., human TF) is maintained (e.g., substantially maintained, for example, at least 50%, at least 60%, at least 70%, at least 80%, at least 90%, or at least 95%).

Additionally or alternatively, in some embodiments, the length of one or more (e.g., one, two, three) CDRs along the VH (e.g., CDR1, CDR2, or CDR3) and/or VL (e.g., CDR1, CDR2, or CDR3) region of a TF antibody (e.g., a human TF antibody) described herein can vary (e.g., be shorter or longer) by one, two, three, four, five, or more amino acids, provided that binding to TF (e.g., human TF) is maintained (e.g., substantially maintained, for example, at least 50%, at least 60%, at least 70%, at least 80%, at least 90%, or at least 95%). For example, in some embodiments, a VH and/or VL CDR1, CDR2, and/or CDR3 can be one, two, three, four, five or more amino acids shorter than one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). In other embodiments, a VH and/or VL CDR1, CDR2, and/or CDR3 can be one, two, three, four, five or more amino acids longer than one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). Additionally or alternatively, in some embodiments, the amino terminus of a VH and/or VL CDR1, CDR2, and/or CDR3 can be extended by one, two, three, four, five or more amino acids compared to one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). Additionally or alternatively, in some embodiments, the carboxy terminus of a VH and/or VL CDR1, CDR2, and/or CDR3 can be extended by one, two, three, four, five or more amino acids compared to one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). Additionally or alternatively, in some embodiments, the amino terminus of a VH and/or VL CDR1, CDR2, and/or CDR3 can be shortened by one, two, three, four, five or more amino acids compared to one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). In some embodiments, the carboxy terminus of a VH and/or VL CDR1, CDR2, and/or CDR3 can be shortened by one, two, three, four, five or more amino acids compared to one or more of the CDRs described by SEQ ID NOs:1-24 and 27-40, provided that binding to TF (e.g., human TF) is maintained (e.g., substantially maintained, for example, at least 50%, at least 60%, at least 70%, at least 80%, at least 90%, or at least 95%). Any method known in the art can be used to ascertain whether binding to TF (e.g., human TF) is maintained, for example, the binding assays and conditions described in the “Examples” section described herein.

In some embodiments, a TF-ADC comprises a TF antibody that comprises one or more (e.g., one, two, three, four, or more) conservative sequence modifications. With respect to polypeptides that are TF antibodies, such as human TF antibodies, conservative sequence modifications include conservative amino acid substitutions in which an amino acid residue is replaced with an amino acid residue having a similar side chain. Families of amino acid residues having similar side chains have been defined in the art. These families are disclosed herein. Thus, in some embodiments, a predicted nonessential amino acid residue in a TF antibody can be replaced with another amino acid residue from the same side chain family.

Methods of identifying amino acid conservative substitutions which do not eliminate antigen binding and nucleotides encoding thereof are well known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the conservative sequence modifications described herein modify the amino acid sequences of a TF antibody (e.g., a human TF antibody) by 50%, 55%, 60%, 65%, 70%, 75%, 80%, 85%, 90%, 95%, 98%, or 99%. In some embodiments, the amino acid sequence modifications refer to at most one, two, three, four, five, or six amino acid substitutions to the CDRs, such as those described in any one of Tables 1-2. Thus, for example, each such CDR can contain up to five conservative amino acid substitutions, for example up to (not more than) four conservative amino acid substitutions, for example up to (not more than) three conservative amino acid substitutions, for example up to (not more than) two conservative amino acid substitutions, or no more than one conservative amino acid substitution. In some embodiments, a TF-ADC comprises a TF antibody that contains one or more, (e.g., one, two, three, four, five, or six CDRs) having at least 75%, 80%, 85%, 90%, 95%, 96%, 97%, 98%, or 99% identity to the CDRs of EXMA006 or EXMA007 (see, e.g., Tables 1-2). In some embodiments, a TF-ADC comprises a TF antibody that contains a VH and a VL comprising CDRs identical to those of EXMA006 or EXMA007 (see, e.g., Tables 1-2). In some embodiments, the amino acid sequence modifications do not include any modification within a specificity determining residue (SDR). In some embodiments, the amino acid sequence modifications do not include any modification within a CDR (such as CDR1, CDR2, CDR3, or any combination thereof). In further embodiments, the amino acid sequence modifications are in the framework or constant region.

In some embodiments, the antibody in a TF-ADC comprises a VH comprising an amino acid sequence having at least 75%, 80%, 85%, 90%, 95%, 96%, 97%, 98%, or 99% identity to SEQ ID NO:25 and a VL comprising an amino acid sequence having at least 75%, 80%, 85%, 90%, 95%, 96%, 97%, 98%, or 99% identity to SEQ ID NO:26, and the binding of the antibody to TF (e.g., human TF) is maintained (e.g., substantially maintained, for example, at least 50%, at least 60%, at least 70%, at least 80%, at least 90%, or at least 95%).

In some embodiments, the antibody in a TF-ADC comprises a VH comprising an amino acid sequence having at least 75%, 80%, 85%, 90%, 95%, 96%, 97%, 98%, or 99% identity to SEQ ID NO:41 and a VL comprising an amino acid sequence having at least 75%, 80%, 85%, 90%, 95%, 96%, 97%, 98%, or 99% identity to SEQ ID NO:42, and the binding of the antibody to TF (e.g., human TF) is maintained (e.g., substantially maintained, for example, at least 50%, at least 60%, at least 70%, at least 80%, at least 90%, or at least 95%).

In some embodiments, the antibody in a TF-ADC comprises a heavy chain comprising an amino acid sequence having at least 75%, 80%, 85%, 90%, 95%, 96%, 97%, 98%, or 99% identity to any one of SEQ ID NOs:77-90 and a light chain comprising an amino acid sequence having at least 75%, 80%, 85%, 90%, 95%, 96%, 97%, 98%, or 99% identity to SEQ ID NO:91 or 92, and the binding of the antibody to TF (e.g., human TF) is maintained (e.g., substantially maintained, for example, at least 50%, at least 60%, at least 70%, at least 80%, at least 90%, or at least 95%). In some embodiments, the antibody in a TF-ADC comprises a heavy chain comprising an amino acid sequence having at least 75%, 80%, 85%, 90%, 95%, 96%, 97%, 98%, or 99% identity to SEQ ID NO:93 and a light chain comprising an amino acid sequence having at least 75%, 80%, 85%, 90%, 95%, 96%, 97%, 98%, or 99% identity to SEQ ID NO:91, and the binding of the antibody to TF (e.g., human TF) is maintained (e.g., substantially maintained, for example, at least 50%, at least 60%, at least 70%, at least 80%, at least 90%, or at least 95%). In some embodiments, the antibody in a TF-ADC comprises a heavy chain comprising an amino acid sequence having at least 75%, 80%, 85%, 90%, 95%, 96%, 97%, 98%, or 99% identity to SEQ ID NO:94 and a light chain comprising an amino acid sequence having at least 75%, 80%, 85%, 90%, 95%, 96%, 97%, 98%, or 99% identity to SEQ ID NO:92, and the binding of the antibody to TF (e.g., human TF) is maintained (e.g., substantially maintained, for example, at least 50%, at least 60%, at least 70%, at least 80%, at least 90%, or at least 95%).

In some embodiments, the antibody in the TF-ADC competes with the antibody designated as EXMA-006. Additionally or alternatively, the antibody in the TF-ADC competes with the antibody designated EXMA-007. In some embodiments, the antibody in the TF-ADC competes with any TF antibody as disclosed herein.

Additionally provided is a nucleic acid encoding a TF binding agent (e.g., antibody or antibody fragment), a nucleic acid complementary thereto, a vector comprising a nucleic acid as disclosed herein, a cell comprising a nucleic acid or a vector as disclosed herein. In some embodiments, the cell expresses the TF binding agent. In some embodiments, the cell replicates the nucleic acid or the vector. In some embodiments, further provided are materials for generating TF antibodies, including human TF antibodies, useful for the preparation of ADCs. In this regard, a cell (e.g., an isolated cell) can produce an antibody comprising a VH and a VL as described herein. In some embodiments, a polynucleotide can comprise one or more nucleic acid sequences encoding a TF antibody or antibody fragment. In some embodiments, the polynucleotide is an isolated and/or recombinant polynucleotide. In some embodiments, the isolated polynucleotide comprises a nucleotide sequence that encodes an antibody heavy chain variable region (VH) and/or an antibody light chain variable region (VL), wherein the VH and the VL comprise CDRs identical to CDRs as described herein.

In some embodiments, one or more vectors (e.g., expression vectors) can comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides are operatively linked to one or more polynucleotides comprising expression control sequences. Autonomously replicating recombinant expression constructs such as plasmid and viral DNA vectors incorporating one or more polynucleotides encoding antibody sequences that bind TF are specifically contemplated. Expression control DNA sequences include promoters, enhancers, and operators, and are generally selected based on the expression systems in which the expression construct is to be utilized. Promoter and enhancer sequences are generally selected for the ability to increase gene expression, while operator sequences are generally selected for the ability to regulate gene expression.

Expression constructs can also include sequences encoding one or more selectable markers that permit identification of host cells bearing the construct. Expression constructs can also include sequences that facilitate, e.g., promote, homologous recombination in a host cell. In some embodiments, expression constructs also include sequences necessary for replication in a host cell.

In some embodiments, an expression control sequence may include a promoter/enhancer sequence, e.g., cytomegalovirus promoter/enhancer (Lehner et al., J. Clin. Microbiol., 29:2494-2502, 1991; Boshart et al., Cell, 41:521-530, 1985); Rous sarcoma virus promoter (Davis et al., Hum. Gene Ther., 4:151, 1993); Tie promoter (Korhonen et al., Blood, 86(5):1828-1835, 1995); simian virus 40 promoter; DRA (downregulated in adenoma; Alrefai et al., Am. J. Physiol. Gastrointest. Liver Physiol., 293:G923-G934, 2007); MCT1 (monocarboxylate transporter 1; Cuff et al., Am. J Physiol. Gastrointest. Liver Physiol., G977-G979. 2005); and Math1 (mouse atonal homolog 1; Shroyer et al., Gastroenterology, 132:2477-2478, 2007), for expression in mammalian cells, the promoter being operatively linked upstream (e.g., 5′) of a polypeptide coding sequence. In some embodiments, a promoter can be an epithelial-specific promoter or endothelial-specific promoter. A polynucleotide can also optionally include a suitable polyadenylation sequence (e.g., the SV40 or human growth hormone gene polyadenylation sequence) operably linked downstream (e.g., 3′) of the polypeptide coding sequence.

In any embodiment, one or more polynucleotides can optionally additionally comprise one or more nucleotide sequences encoding one or more secretory signal peptides fused in frame with the polypeptide sequences. The one or more secretory signal peptides can direct secretion of the antibody polypeptides by the cells that express the one or more polynucleotides and can be cleaved by the cell from the secreted polypeptide. In any embodiment, one or more polynucleotides can further optionally comprise one or more sequences whose only intended function is to facilitate large scale production of the vector.

Expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, e.g., deleting or disrupting select genes required for viral replication.

A cell can comprise one or more polynucleotides and/or one or more vectors. For example, in any embodiment a cell can be transformed or transfected with one or more polynucleotides encoding a TF antibody (e.g., a human TF antibody) or one or more vectors comprising the one or more polynucleotides encoding a TF antibody (e.g., a human TF antibody). In some embodiments, a cell can express a TF antibody (e.g., a human TF antibody) containing one or more, (e.g., one, two, three, four, five, or six) CDRs having at least 75%, 80%, 85%, 90%, 95%, 96%, 97%, 98%, or 99% identity to CDRs as described herein, such as EXMA006 or EXMA007 (see, e.g., Tables 1-2). In some embodiments, a cell can express a TF antibody (e.g., a human TF antibody) containing VH regions and VL regions comprising CDRs identical to those as described herein, such as of EXMA006 or EXMA007 (see, e.g., Tables 1-2). Suitable cells for generating a TF antibody as described herein include prokaryotic cells, such as Escherichia coli (see, e.g., Plückthun et al., Methods Enzymol., 178:497-515, 1989), and eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells can provide for translational modifications (e.g., glycosylation, truncation, lipidation, and phosphorylation) that can be desirable to confer optimal biological activity on recombinant expression products. Similarly, in any embodiment, an antibody (e.g., TF antibody, including a human TF antibody) can be glycosylated or non-glycosylated and/or have been covalently modified to include one or more water-soluble polymer attachments such as polyethylene glycol, polyoxyethylene glycol, or polypropylene glycol.

Methods for introducing DNA or RNA into a host cell are well known and include, but are not limited to, transformation, transfection, electroporation, nuclear injection, and fusion with carriers such as liposomes, micelles, ghost cells, and protoplasts. Such host cells are useful for amplifying polynucleotides and for expressing polypeptides (e.g., antibodies) encoded by the polynucleotides. In this regard, a process to produce a TF antibody can comprise introducing RNA or DNA that encodes for a TF antibody, as described herein, into a host cell, culturing the host cell, and isolating the TF antibody thus produced.

A variety of methods for producing an antibody from a polynucleotide is generally well known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, U K, 1992); and Current Protocols in Molecular Biology, Ausubel ed., Wiley Interscience, New York, 1999).

In certain embodiments, the amino acid sequence of a TF antibody is modified to include a sulfatase motif that contains a serine or cysteine residue that is capable of being converted (oxidized) to a 2-formylglycine (fGly) residue by action of a formylglycine generating enzyme (FGE) either in vivo (e.g., at the time of translation of an aldehyde tag-containing protein in a cell) or in vitro (e.g., by contacting an aldehyde tag-containing protein with an FGE in a cell-free system). Such sulfatase motifs may also be referred to herein as an FGE-modification site.

### Sulfatase Motifs

A minimal sulfatase motif of an aldehyde tag is usually 5 or 6 amino acid residues in length, usually no more than 6 amino acid residues in length. Sulfatase motifs provided in an Ig polypeptide are at least 5 or 6 amino acid residues, and can be, for example, from 5 to 16, 6-16, 5-15, 6-15, 5-14, 6-14, 5-13, 6-13, 5-12, 6-12, 5-11, 6-11, 5-10, 6-10, 5-9, 6-9, 5-8, or 6-8 amino acid residues in length, so as to define a sulfatase motif of less than 16, 15, 14, 13, 12, 11, 10, 9, 8, 7 or 6 amino acid residues in length.

In certain embodiments, TF antibodies of interest include those where one or more amino acid residues, such as 2 or more, or 3 or more, or 4 or more, or 5 or more, or 6 or more, or 7 or more, or 8 or more, or 9 or more, or 10 or more, or 11 or more, or 12 or more, or 13 or more, or 14 or more, or 15 or more, or 16 or more, or 17 or more, or 18 or more, or 19 or more, or 20 or more amino acid residues have been inserted, deleted, substituted (replaced) relative to the native amino acid sequence to provide for a sequence of a sulfatase motif in the TF antibody. In certain embodiments, the TF antibody includes a modification (insertion, addition, deletion, and/or substitution/replacement) of less than 20, 19, 18, 17, 16, 15, 14, 13, 12, 11, 10, 9, 8, 7, 6, 5, 4, 3 or 2 amino acid residues of the amino acid sequence relative to the native amino acid sequence of the TF antibody. Where an amino acid sequence native to the TF antibody contains one or more residues of the desired sulfatase motif, the total number of modifications of residues can be reduced, e.g., by site-specification modification (insertion, addition, deletion, substitution/replacement) of amino acid residues flanking the native amino acid residues to provide a sequence of the desired sulfatase motif.

In certain embodiments, the extent of modification of the native amino acid sequence of the target antibody is minimized, so as to minimize the number of amino acid residues that are inserted, deleted, substituted (replaced), or added (e.g., to the N- or C-terminus). Minimizing the extent of amino acid sequence modification of the target antibody may minimize the impact such modifications may have upon antibody function and/or structure.

It should be noted that while aldehyde tags of particular interest are those comprising at least a minimal sulfatase motif (also referred to a “consensus sulfatase motif”), it will be readily appreciated that longer aldehyde tags are both contemplated and encompassed by the present disclosure and can find use in the compositions and methods of the present disclosure. Aldehyde tags can thus comprise a minimal sulfatase motif of 5 or 6 residues, or can be longer and comprise a minimal sulfatase motif which can be flanked at the N- and/or C-terminal sides of the motif by additional amino acid residues. Aldehyde tags of, for example, 5 or 6 amino acid residues are contemplated, as well as longer amino acid sequences of more than 5, 6, 7, 8, 9, 10, 11, 12, 13, 14, 15, 16, 17, 18, 19, 20 or more amino acid residues.

An aldehyde tag can be present at or near the C-terminus of an Ig heavy chain; e.g., an aldehyde tag can be present within 1, 2, 3, 4, 5, 6, 7, 8, 9, or 10 amino acids of the C-terminus of a native, wild-type Ig heavy chain. An aldehyde tag can be present within a CH1 domain of an Ig heavy chain. An aldehyde tag can be present within a CH2 domain of an Ig heavy chain. An aldehyde tag can be present within a CH3 domain of an Ig heavy chain. An aldehyde tag can be present in an Ig light chain constant region, e.g., in a kappa light chain constant region or a lambda light chain constant region.

In certain embodiments, the sulfatase motif used may be described by the formula:

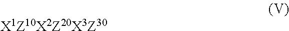

where
Z¹⁰ is cysteine or serine (which can also be represented by (C/S));
Z²⁰ is either a proline or alanine residue (which can also be represented by (P/A));
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I;
X¹ is present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M, S or V, with the proviso that when the sulfatase motif is at the N-terminus of the target TF antibody, X¹ is present; and
X² and X³ independently can be any amino acid, though usually an aliphatic amino acid, a polar, uncharged amino acid, or a sulfur containing amino acid (i.e., other than an aromatic amino acid or a charged amino acid), e.g., S, T, A, V, G or C, e.g., S, T, A, V or G.

The amino acid sequence of an antibody heavy and/or light chain can be modified to provide a sequence of at least 5 amino acids of the formula X¹Z¹⁰X²Z²⁰X³Z³⁰, where
Z¹⁰ is cysteine or serine;
Z²⁰ is a proline or alanine residue;
Z³⁰ is an aliphatic amino acid or a basic amino acid;
X¹ is present or absent and, when present, is any amino acid, with the proviso that when the heterologous sulfatase motif is at an N-terminus of the TF antibody, X¹ is present;
X² and X³ are each independently any amino acid.

The sulfatase motif is generally selected so as to be capable of conversion by a selected FGE, e.g., an FGE present in a host cell in which the aldehyde-tagged antibody is expressed or an FGE which is to be contacted with the aldehyde-tagged antibody in a cell-free in vitro method.

For example, where the FGE is a eukaryotic FGE (e.g., a mammalian FGE, including a human FGE), the sulfatase motif can be of the formula:

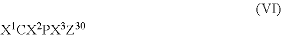

where
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, S or V, with the proviso that when the sulfatase motif is at the N-terminus of the target TF antibody, X¹ is present;
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G, or C, e.g., S, T, A, V or G; and
Z³⁰ is a basic amino acid (e.g., arginine (R), and may be lysine (K) or histidine (H), e.g., lysine), or an aliphatic amino acid (alanine (A), glycine (G), leucine (L), valine (V), isoleucine (I), or proline (P), e.g., A, G, L, V, or I.

Specific examples of sulfatase motifs include LCTPSR (SEQ ID NO:100), MCTPSR (SEQ ID NO:101), VCTPSR (SEQ ID NO:102), LCSPSR (SEQ ID NO:103), LCAPSR (SEQ ID NO:104), LCVPSR (SEQ ID NO:105), LCGPSR (SEQ ID NO:106), ICTPAR (SEQ ID NO:107), LCTPSK (SEQ ID NO:108), MCTPSK (SEQ ID NO:109), VCTPSK (SEQ ID NO:110), LCSPSK (SEQ ID NO:111), LCAPSK (SEQ ID NO:112), LCVPSK (SEQ ID NO:113), LCGPSK (SEQ ID NO:114), LCTPSA (SEQ ID NO:115), ICTPAA (SEQ ID NO:116), MCTPSA (SEQ ID NO:117), VCTPSA (SEQ ID NO:118), LCSPSA (SEQ ID NO:119), LCAPSA (SEQ ID NO:120), LCVPSA (SEQ ID NO:121), and LCGPSA (SEQ ID NO:122).

fGly-Containing Sequences

Upon action of FGE on the antibody heavy and/or light chain, the serine or the cysteine in the sulfatase motif is modified to fGly. Thus, the fGly-containing sulfatase motif can be of the formula:

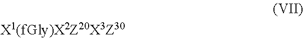

where
fGly is the formylglycine residue;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target TF antibody, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

Specific examples of fGly-containing sulfatase motifs include L(fGly)TPSR (SEQ ID NO:123), M(fGly)TPSR (SEQ ID NO:124), V(fGly)TPSR (SEQ ID NO:125), L(fGly)SPSR (SEQ ID NO:126), L(fGly)APSR (SEQ ID NO:127), L(fGly)VPSR (SEQ ID NO:128), L(fGly)GPSR (SEQ ID NO:129), I(fGly)TPAR (SEQ ID NO:130), L(fGly)TPSK (SEQ ID NO:131), M(fGly)TPSK (SEQ ID NO:132), V(fGly)TPSK (SEQ ID NO:133), L(fGly)SPSK (SEQ ID NO:134), L(fGly)APSK (SEQ ID NO:135), L(fGly)VPSK (SEQ ID NO:136), L(fGly)GPSK (SEQ ID NO:137), L(fGly)TPSA (SEQ ID NO:138), I(fGly)TPAA (SEQ ID NO:139), M(fGly)TPSA (SEQ ID NO:140), V(fGly)TPSA (SEQ ID NO:141), L(fGly)SPSA (SEQ ID NO:142), L(fGly)APSA (SEQ ID NO:143), L(fGly)VPSA (SEQ ID NO:144), and L(fGly)GPSA (SEQ ID NO:145).

As described above, to produce the conjugate, the TF antibody containing the fGly residue may be conjugated to a drug or active agent by reaction of the fGly with a reactive moiety (e.g., a hydrazinyl-indolyl or a hydrazinyl-pyrrolo-pyridinyl conjugation moiety, as described above) of a linker attached to the drug or active agent to produce an fGly′-containing sulfatase motif. As used herein, the term fGly′ refers to the amino acid residue of the sulfatase motif that is coupled to the drug or active agent through a linker (e.g., a branched linker) as described herein. Thus, the fGly′-containing sulfatase motif can be of the formula:

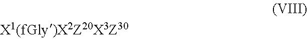

where
fGly′ is the amino acid residue coupled to the drug or active agent through a linker (e.g., a branched linker) as described herein;
Z²⁰ is either a proline or alanine residue (which can also be represented by (P/A));
Z³⁰ is a basic amino acid (e.g., arginine (R), and may be lysine (K) or histidine (H), usually lysine), or an aliphatic amino acid (alanine (A), glycine (G), leucine (L), valine (V), isoleucine (I), or proline (P), e.g., A, G, L, V, or I;
X¹ may be present or absent and, when present, can be any amino acid, e.g., an aliphatic amino acid, a sulfur-containing amino acid, or a polar, uncharged amino acid, (i.e., other than an aromatic amino acid or a charged amino acid), e.g., L, M, V, S or T, e.g., L, M or V, with the proviso that when the sulfatase motif is at the N-terminus of the target TF antibody, X¹ is present; and
X² and X³ independently can be any amino acid, e.g., an aliphatic amino acid, a sulfur-containing amino acid, or a polar, uncharged amino acid, (i.e., other than an aromatic amino acid or a charged amino acid), e.g., S, T, A, V, G or C, e.g., S, T, A, V or G.

Specific examples of fGly′-containing sulfatase motifs include L(fGly′)TPSR (SEQ ID NO:146), M(fGly′)TPSR (SEQ ID NO:147), V(fGly′)TPSR (SEQ ID NO:148), L(fGly′)SPSR (SEQ ID NO:149), L(fGly′)APSR (SEQ ID NO:150), L(fGly′)VPSR (SEQ ID NO:151), L(fGly′)GPSR (SEQ ID NO: 152), I(fGly′)TPAR (SEQ ID NO:153), L(fGly′)TPSK (SEQ ID NO:154), M(fGly′)TPSK (SEQ ID NO:155), V(fGly′)TPSK (SEQ ID NO:156), L(fGly′)SPSK (SEQ ID NO:157), L(fGly′)APSK (SEQ ID NO:158), L(fGly′)VPSK (SEQ ID NO:159), L(fGly′)GPSK (SEQ ID NO:160), L(fGly′)TPSA (SEQ ID NO:161), I(fGly′)TPAA (SEQ ID NO:162), M(fGly′)TPSA (SEQ ID NO:163), V(fGly′)TPSA (SEQ ID NO:164), L(fGly′)SPSA (SEQ ID NO:165), L(fGly′)APSA (SEQ ID NO:166), L(fGly′)VPSA (SEQ ID NO:167), and L(fGly′)GPSA (SEQ ID NO:168).

As noted above, the amino acid sequence of an antibody is modified to include a sulfatase motif that contains a serine or cysteine residue that is capable of being converted (oxidized) to an fGly residue by action of an FGE either in vivo (e.g., at the time of translation of an aldehyde tag-containing protein in a cell) or in vitro (e.g., by contacting an aldehyde tag-containing protein with an FGE in a cell-free system). The antibody used to generate a conjugate of the present disclosure include at least an Ig constant region, e.g., an Ig heavy chain constant region (e.g., at least a CH1 domain; at least a CH1 and a CH2 domain; a CH1, a CH2, and a CH3 domain; or a CH1, a CH2, a CH3, and a CH4 domain), or an Ig light chain constant region. Such Ig antibodies are referred to herein as “target Ig polypeptides” or “target antibodies”.

The site in an antibody into which a sulfatase motif is introduced can be any convenient site. As noted above, in some instances, the extent of modification of the native amino acid sequence of the target polypeptide is minimized, so as to minimize the number of amino acid residues that are inserted, deleted, substituted (replaced), and/or added (e.g., to the N- or C-terminus). Minimizing the extent of amino acid sequence modification of the target antibody may minimize the impact such modifications may have upon antibody function and/or structure.

An antibody heavy chain constant region can include Ig constant regions of any heavy chain isotype, non-naturally occurring Ig heavy chain constant regions (including consensus Ig heavy chain constant regions). An Ig constant region amino acid sequence can be modified to include an aldehyde tag, where the aldehyde tag is present in or adjacent a solvent-accessible loop region of the Ig constant region. An Ig constant region amino acid sequence can be modified by insertion and/or substitution of 1, 2, 3, 4, 5, 6, 7, 8, 9, 10, 11, 12, 13, 14, 15, or 16 amino acids, or more than 16 amino acids, to provide an amino acid sequence of a sulfatase motif as described above.

In some cases, an aldehyde-tagged antibody comprises an aldehyde-tagged Ig heavy chain constant region (e.g., at least a CH1 domain; at least a CH1 and a CH2 domain; a CH1, a CH2, and a CH3 domain; or a CH1, a CH2, a CH3, and a CH4 domain). The aldehyde-tagged Ig heavy chain constant region can include heavy chain constant region sequences of an IgA, IgM, IgD, IgE, IgG1, IgG2, IgG3, or IgG4 isotype heavy chain or any allotypic variant of same, e.g., human heavy chain constant region sequences or mouse heavy chain constant region sequences, a hybrid heavy chain constant region, a synthetic heavy chain constant region, or a consensus heavy chain constant region sequence, etc., modified to include at least one sulfatase motif that can be modified by an FGE to generate an fGly-modified Ig polypeptide. Allotypic variants of Ig heavy chains are known in the art. See, e.g., Jefferis and Lefranc (2009)MAbs 1:4.

In some cases, an aldehyde-tagged antibody comprises an aldehyde-tagged Ig light chain constant region. The aldehyde-tagged Ig light chain constant region can include constant region sequences of a kappa light chain, a lambda light chain, e.g., human kappa or lambda light chain constant regions, a hybrid light chain constant region, a synthetic light chain constant region, or a consensus light chain constant region sequence, etc., modified to include at least one sulfatase motif that can be modified by an FGE to generate an fGly-modified antibody. Exemplary constant regions include human gamma 1 and gamma 3 regions. With the exception of the sulfatase motif, a constant region may have a wild-type amino acid sequence, or it may have an amino acid sequence that is at least 70% identical (e.g., at least 80%, at least 90% or at least 95% identical) to a wild type amino acid sequence.

In some embodiments the sulfatase motif is at a position other than, or in addition to, the C-terminus of the Ig polypeptide heavy chain. As noted above, an isolated aldehyde-tagged antibody can comprise a heavy chain constant region amino acid sequence modified to include a sulfatase motif as described above, where the sulfatase motif is in or adjacent a surface-accessible loop region of the antibody heavy chain constant region.

A sulfatase motif can be provided within or adjacent one or more of these amino acid sequences of such modification sites of an Ig heavy chain. For example, an Ig heavy chain polypeptide amino acid sequence can be modified (e.g., where the modification includes one or more amino acid residue insertions, deletions, and/or substitutions) at one or more of these amino acid sequences to provide a sulfatase motif adjacent and N-terminal and/or adjacent and C-terminal to these modification sites. Alternatively or in addition, an Ig heavy chain polypeptide amino acid sequence can be modified (e.g., where the modification includes one or more amino acid residue insertions, deletions, and/or substitutions) at one or more of these amino acid sequences to provide a sulfatase motif between any two residues of the Ig heavy chain modifications sites. In some embodiments, an Ig heavy chain polypeptide amino acid sequence may be modified to include two motifs, which may be adjacent to one another, or which may be separated by one, two, three, four or more (e.g., from about 1 to about 25, from about 25 to about 50, or from about 50 to about 100, or more), amino acids.

Alternatively or in addition, where a native amino acid sequence provides for one or more amino acid residues of a sulfatase motif sequence, selected amino acid residues of the modification sites of an Ig heavy chain polypeptide amino acid sequence can be modified (e.g., where the modification includes one or more amino acid residue insertions, deletions, and/or substitutions) so as to provide a sulfatase motif at the modification site.

Other suitable sulfatase motif and their location in an antibody can also be used, such as International Publication Nos. WO2012097333, WO2017189432, and WO2018169953, each of which is incorporated herein by reference in its entirety.

An antibody used in an antibody-drug conjugate of the present disclosure can have any of a variety of antigen-binding specificities, including but not limited to, e.g., an antigen present on a cancer cell; an antigen present on an autoimmune cell; an antigen present on a pathogenic microorganism; an antigen present on a virus-infected cell (e.g., a human immunodeficiency virus-infected cell); an antigen present on a diseased cell; and the like. For example, an antibody conjugate can bind an antigen, where the antigen is present on the surface of the cell. An antibody conjugate of the present disclosure can bind antigen with a suitable binding affinity, e.g., from 5×10⁻⁶ M to 10⁻⁷ M, from 10⁻⁷ M to 5×10⁻⁷ M, from 5×10⁻⁷ M to 10⁻⁸ M, from 10⁻⁸ M to 5×10⁻⁸ M, from 5×10⁻⁸ M to 10⁻⁹ M, or a binding affinity greater than 10⁻⁹ M.

As non-limiting examples, a subject antibody conjugate can bind an antigen present on a cancer cell (e.g., a tumor-specific antigen; an antigen that is over-expressed on a cancer cell; etc.), and the conjugated moiety can be a drug, such as a cytotoxic compound (e.g., a cytotoxic small molecule, a cytotoxic synthetic peptide, etc.). For example, a subject antibody conjugate can be specific for an antigen on a cancer cell, where the conjugated moiety is a drug, such as a cytotoxic compound (e.g., a cytotoxic small molecule, a cytotoxic synthetic peptide, etc.).

As further non-limiting examples, a subject antibody conjugate can bind an antigen present on a cell infected with a virus (e.g., where the antigen is encoded by the virus; where the antigen is expressed on a cell type that is infected by a virus; etc.), and the conjugated moiety can be a drug, such as a viral fusion inhibitor. For example, a subject antibody conjugate can bind an antigen present on a cell infected with a virus, and the conjugated moiety can be a drug, such as a viral fusion inhibitor.

In some embodiments, the antibody that binds to TF can have a heavy chain which has been modified to include an unconverted sulfatase motif (e.g., any one of SEQ ID Nos:100-122) that may be and therefore have the amino acid sequence of: QVQLVQSGAEVKKPGASVKVSCKASGYTFDVYGI SWVRQAPGQGLEWMGWIAPYSGNTNYAQ KLQGRVTMTTDTSTSTAYMELRSLRSDDTAVYYCARDAGTYSPFGYGMDVWGQGTTVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSLCTPSRNTKVDKRVEPKSCDKTHTCPPCPAPELLG GPSVFLFPPKPKDTLMI SRTPEVTCVVVDVSHEDPEVKFNWYVDGVEVHNAKTKPREEQYNS TYRVVSVLTVLHQDWLNGKEYKCKVSNKALPAPIEKTI SKAKGQPREPQVYTLPPSREEMTK NQVSLTCLVKGFYPSDIAVEWESNGQPENNYKTTPPVLDSDGSFFLYSKLTVDKSRWQQGNV FSCSVMHEALHNHYTQKSLSLSPGK (SEQ ID NO:77), where the underline portion comprises the unconverted sulfatase motif of SEQ ID NO:100 but, in any embodiment, may be replaced with any one of SEQ ID NOs:101-122. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody. Additionally or alternatively, the sulfatase motif is converted and conjugated to a linker-payload in an TF-ADC as disclosed herein.

In some embodiments, the antibody that binds to TF can have a heavy chain which has been modified to include a formyl glycine residue and therefore have the amino acid sequence of:

(SEQ ID NO: 78)
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSL(fGly)TPSRNTKVDKRVEPKSCDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALH
NHYTQKSLSLSPGK,

wherein f(Gly) is the formyl glycine residue, where the underline portion comprises the fGly-containing sulfatase motif of SEQ ID NO:123 but, in any embodiment, may be replaced with any one of SEQ ID NOs:124-145. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus.

In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody. Additionally or alternatively, the sulfatase motif is conjugated to a linker-payload in an TF-ADC as disclosed herein.

In some embodiments, a TF-ADC comprises a heavy chain having the amino acid sequence of:

(SEQ ID NO: 79)
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSL(fGly′)TPSRNTKVDKRVEPKSCDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK,

wherein (fGly′) is an amino acid having its side chain replaced by a linker-drug as disclosed wherein, for example, the ADC of Formula (I) or Formula (II). In SEQ ID NO:79, the underline portion comprises the fGly′-containing sulfatase motif of SEQ ID NO:146 but, in any embodiment, may be replaced with any one of SEQ ID NOs:147-168. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody.

In some embodiments, the antibody that binds to TF can have a heavy chain which has been modified to include two unconverted sulfatase motifs (e.g., L(C/S)TPSR, SEQ ID NO:99) and therefore have the amino acid sequence of: QVQLVQSGAEVKKPGASVKVSCKASGYTFDVYGISWVRQAPGQGLEWMGWIAPYSGNTNYAQ KLQGRVTMTTDTSTSTAYMELRSLRSDDTAVYYCARDAGTYSPFGYGMDVWGQGTTVTVSSA STKGPSVFPLAPSSKSTSGGTAALGCLVKDYFPEPVTVSWNSGALCTPSRGVHTFPAVLQSS GLYSLSSVVTVPSSSLGTQTYICNVNHKPSNTKVDKRVEPKSCDKTHTCPPCPAPELLGGPS VFLFPPKPKDTLMISRTPEVTCVVVDVSHEDPEVKFNWYVDGVEVHNAKTKPREEQYNSTYR VVSVLTVLHQDWLNGKEYKCKVSNKALPAPIEKTISKAKGQPREPQVYTLPPSREEMTKNQV SLTCLVKGFYPSDIAVEWESNGQPENNYKTTPPVLDSDGSFFLYSKLTVDKSRWQQGNVFSC SVMHEALHNHYTQKSLSLSPGSLCTPSRGS (SEQ ID NO:80), where underline portions comprise an unconverted sulfatase motif of SEQ ID NO:100 but, in any embodiment, may independently be replaced with any one of SEQ ID NOs:101-122. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, either or both of the sulfatase motifs is/are converted and conjugated to a linker-payload in an TF-ADC as disclosed herein.

In some embodiments, the antibody that binds to TF can have a heavy chain which has been modified to include two formyl glycine residues and therefore have the amino acid sequence of:

(SEQ ID NO: 81)
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGAL(fGly)TPSRGVHTFPAVLQSSGLYSLSSVV
TVPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGSL(fGly)TPSRGS,

wherein each f(Gly) is a formyl glycine residue, where each underline portion comprises the fGly-containing sulfatase motif of SEQ ID NO:123 but, in any embodiment, one or both may be independently replaced with any one of SEQ ID NOs:124-145. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, either or both of the sulfatase motifs is/are conjugated to a linker-payload in an TF-ADC as disclosed herein.

In some embodiments, a TF-ADC comprises a heavy chain having the amino acid sequence of:

(SEQ ID NO: 82)
QVQLVQSGAEVKKPGASVKVSCKASGYTFDVYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGAL(fGly′)TPSRGVHTFPAVLQSSGLYSLSSV
VTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGSL(fGly′)TPSRGS,

wherein each (fGly′) is an amino acid having its side chain replaced by a linker-drug as disclosed herein, for example, the ADC of Formula (I) or Formula (II). In SEQ ID NO:82, each underline portion comprises the fGly′-containing sulfatase motif of SEQ ID NO:146 but, in any embodiment, one or both may be independently replaced with any one of SEQ ID NOs:147-168. In further embodiments, the antibody further comprises a light chain comprising an amino acid sequence of SEQ ID NO:91. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus.

In some embodiments, the antibody that binds to TF can have a heavy chain which has been modified to include an unconverted sulfatase motif (e.g., L(C/S)TPSR, SEQ ID NO:99) and have the amino acid sequence of: QVQLVQSGAEVKKPGASVKVSCKASGYTFDAYGISWVRQAPGQGLEWMGWIAPYSGNTNYAQ KLQGRVTMTTDTSTSTAYMELRSLRSDDTAVYYCARDAGTYSPFGYGMDVWGQGTTVTVSSA STKGPSVFPLAPSSKSTSGGTAALGCLVKDYFPEPVTVSWNSGALTSGVHTFPAVLQSSGLY SLSSVVTVPSSSLGTQTYICNVNHKPSLCTPSRNTKVDKRVEPKSCDKTHTCPPCPAPELLG GPSVFLFPPKPKDTLMISRTPEVTCVVVDVSHEDPEVKFNWYVDGVEVHNAKTKPREEQYNS TYRVVSVLTVLHQDWLNGKEYKCKVSNKALPAPIEKTISKAKGQPREPQVYTLPPSREEMTK NQVSLTCLVKGFYPSDIAVEWESNGQPENNYKTTPPVLDSDGSFFLYSKLTVDKSRWQQGNV FSCSVMHEALHNHYTQKSLSLSPGK (SEQ ID NO:83), where the underline portion comprises the unconverted sulfatase motif of SEQ ID NO:100 but, in any embodiment, may be replaced with any one of SEQ ID NOs:101-122. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody. Additionally or alternatively the sulfatase motif is converted and conjugated to a linker-payload in an TF-ADC as disclosed herein.

In some embodiments, the antibody that binds to TF can have a heavy chain which has been modified to include a formyl glycine residue and have the amino acid sequence of

(SEQ ID NO: 84)
QVQLVQSGAEVKKPGASVKVSCKASGYTFDAYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSL(fGly)TPSRNTKVDKRVEPKSCDKTHTCPPCPAPE
LLGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALH
NHYTQKSLSLSPGK,

wherein f(Gly) is the formyl glycine residue, where the underline portion comprises the fGly-containing sulfatase motif of SEQ ID NO:123 but, in any embodiment, may be replaced with any one of SEQ ID NOs:124-145. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus.

In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody. Additionally or alternatively, the sulfatase motif is conjugated to a linker-payload in an TF-ADC as disclosed herein.

In some embodiments, a TF-ADC comprises a heavy chain having the amino acid sequence of:

(SEQ ID NO: 85)
QVQLVQSGAEVKKPGASVKVSCKASGYTFDAYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGALTSGVHTFPAVLQSSGLYSLSSVVTVPSSSLG
TQTYICNVNHKPSL(fGly′)TPSRNTKVDKRVEPKSCDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK,

wherein each (fGly′) is an amino acid having its side chain replaced by a linker-drug as disclosed herein, for example, the ADC of Formula (I) or Formula (II). In SEQ ID NO:85, the underline portion comprises the fGly′-containing sulfatase motif of SEQ ID NO:146 but, in any embodiment, may be replaced with any one of SEQ ID NOs:147-168. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, the C terminal K residue of the heavy chain is removed, for example, during production of the antibody.

In some embodiments, the antibody that binds to TF can have a heavy chain which has been modified to include two unconverted sulfatase motifs (e.g., LCTPSR, SEQ ID NO:100) and have the amino acid sequence of: QVQLVQSGAEVKKPGASVKVSCKASGYTFDAYGI SWVRQAPGQGLEWMGWIAPYSGNTNYAQ KLQGRVTMTTDTSTSTAYMELRSLRSDDTAVYYCARDAGTYSPFGYGMDVWGQGTTVTVSSA STKGPSVFPLAPSSKSTSGGTAALGCLVKDYFPEPVTVSWNSGALCTPSRGVHTFPAVLQSS GLYSLSSVVTVPSSSLGTQTYICNVNHKPSNTKVDKRVEPKSCDKTHTCPPCPAPELLGGPS VFLFPPKPKDTLMI SRTPEVTCVVVDVSHEDPEVKFNWYVDGVEVHNAKTKPREEQYNSTYR VVSVLTVLHQDWLNGKEYKCKVSNKALPAPIEKTI SKAKGQPREPQVYTLPPSREEMTKNQV SLTCLVKGFYPSDIAVEWESNGQPENNYKTTPPVLDSDGSFFLYSKLTVDKSRWQQGNVFSC SVMHEALHNHYTQKSLSLSPGSLCTPSRGS (SEQ ID NO:86), where each underline portion comprises the unconverted sulfatase motif of SEQ ID NO:100 but, in any embodiment, one or both may independently be replaced with any one of SEQ ID NOs:101-122. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, either or both of the sulfatase motifs is/are converted and conjugated to a linker-payload in an TF-ADC as disclosed herein.

In some embodiments, the antibody that binds to TF can have a heavy chain which has been modified to include two formyl glycine residues and have the amino acid sequence of:

(SEQ ID NO: 87)
QVQLVQSGAEVKKPGASVKVSCKASGYTFDAYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGAL(fGly)TPSRGVHTFPAVLQSSGLYSLSSVV
TVPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHY
TQKSLSLSPGSL(fGly)TPSRGS,

wherein f(Gly) is the formyl glycine residue. In SEQ ID NO:87, each underline portion comprises an fGly-containing sulfatase motif of SEQ ID NO:123 but, in any embodiment, one or both may be replaced with any one of SEQ ID NOs:124-145. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus. Additionally or alternatively, either or both of the sulfatase motifs is/are conjugated to a linker-payload in an TF-ADC as disclosed herein.

In some embodiments, a TF-ADC comprises a heavy chain having the amino acid sequence of:

(SEQ ID NO: 88)
QVQLVQSGAEVKKPGASVKVSCKASGYTFDAYGISWVRQAPGQGLEWMGW
IAPYSGNTNYAQKLQGRVTMTTDTSTSTAYMELRSLRSDDTAVYYCARDA
GTYSPFGYGMDVWGQGTTVTVSSASTKGPSVFPLAPSSKSTSGGTAALGC
LVKDYFPEPVTVSWNSGAL(fGly′)TPSRGVHTFPAVLQSSGLYSLSSV
VTVPSSSLGTQTYICNVNHKPSNTKVDKRVEPKSCDKTHTCPPCPAPELL
GGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKT
ISKAKGQPREPQVYTLPPSREEMTKNQVSLTCLVKGFYPSDIAVEWESNG
QPENNYKTTPPVLDSDGSFFLYSKLTVDKSRWQQGNVFSCSVMHEALHNH
YTQKSLSLSPGSL(fGly′)TPSRGS,

wherein each (fGly′) is an amino acid having its side chain replaced by a linker-drug as disclosed herein, for example, the ADC of Formula (I) or Formula (II). In SEQ ID NO:88, the underline portions comprise the fGly′-containing sulfatase motif of SEQ ID NO:146 but, in any embodiment, one or both may be independently replaced with any one of SEQ ID NOs:147-168. In some embodiments, the heavy chain further comprises a signal peptide (such as SEQ ID NO:97) at its N terminus. In other embodiments, the heavy chain does not further comprise a signal peptide (such as SEQ ID NO:97) at its N terminus. In further embodiments, the antibody further comprises a light chain comprising an amino acid sequence of SEQ ID NO:92. In some embodiments, either or both of the heavy and light chains further comprise a signal peptide (such as SEQ ID NO:97) at its N terminus. In other embodiments, either or both of the heavy and light chains do not further comprise a signal peptide (such as SEQ ID NO:97) at its N terminus.

A general scheme for coupling an antibody to a pyridazine-pyrrolo coupling moiety is shown below.

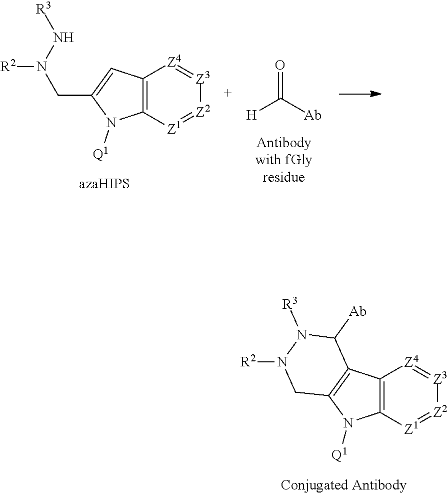

A hydrazinyl-pyrrolo coupling moiety, which can interchangeably be referred to herein as an aza-hydrazino-iso-Pictet-Spengler (azaHIPS) coupling moiety, upon conjugation to a formyl-glycine, forms a pyridazine-pyrrolo coupling moiety as shown above. A TF antibody can include a 2-formylglycine residue (fGly) that is reacted with azaHIPS coupling moiety, thus conjugating the two together. To generate a TF-ADC, a drug can be coupled directly or indirectly (e.g., through a linker moiety) to the azaHIPS moiety at any location of the azaHIPs moiety (e.g., Z¹, Z², Z³, Z⁴, or Q¹). R² and R³ can each independently be any desired substituent, such as, but not limited to, hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, or substituted heterocyclyl. Z¹, Z², Z³, and Z⁴ can be as described herein, such as for Formula (I). Q¹ may be —L^{A}—W¹, as described herein, such as for Formula (I).

Formula (X) below represents a hydrazinyl-pyrrolo coupling moiety that can be used to link a TF antibody and a drug in any TF-ADC described herein.

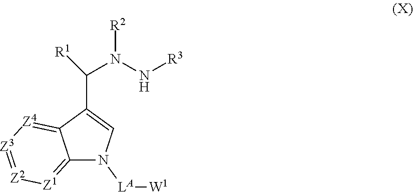

wherein:
Z¹, Z², Z³ and Z⁴ are each independently selected from CR⁴, N and C—L^{B}—W², wherein at least one Z¹, Z², Z³ and Z⁴ is C—L^{B}—W²;
R¹ is selected from hydrogen, alkyl, substituted alkyl, alkenyl, substituted alkenyl, alkynyl, substituted alkynyl, aryl, substituted aryl, heteroaryl, substituted heteroaryl, cycloalkyl, substituted cycloalkyl, heterocyclyl, and substituted heterocyclyl;
R² and R³ are each independently selected from hydrogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl, or R² and R³ are optionally cyclically linked to form a 5 or 6-membered heterocyclyl;
each R⁴ is independently selected from hydrogen, halogen, alkyl, substituted alkyl, alkenyl, substituted alkenyl, alkynyl, substituted alkynyl, alkoxy, substituted alkoxy, amino, substituted amino, carboxyl, carboxyl ester, acyl, acyloxy, acyl amino, amino acyl, alkylamide, substituted alkylamide, sulfonyl, thioalkoxy, substituted thioalkoxy, aryl, substituted aryl, heteroaryl, substituted heteroaryl, cycloalkyl, substituted cycloalkyl, heterocyclyl, and substituted heterocyclyl;
L^{A} is a first linker as described herein;
L^{B} is a second linker as described herein;
W¹ is a first drug as described herein; and
W² is a second drug as described herein.

For example, in a TF-ADC of Formula (II) may be prepared by conjugating one or more linker-payloads of Formula (IIa), shown below, to a TF antibody:

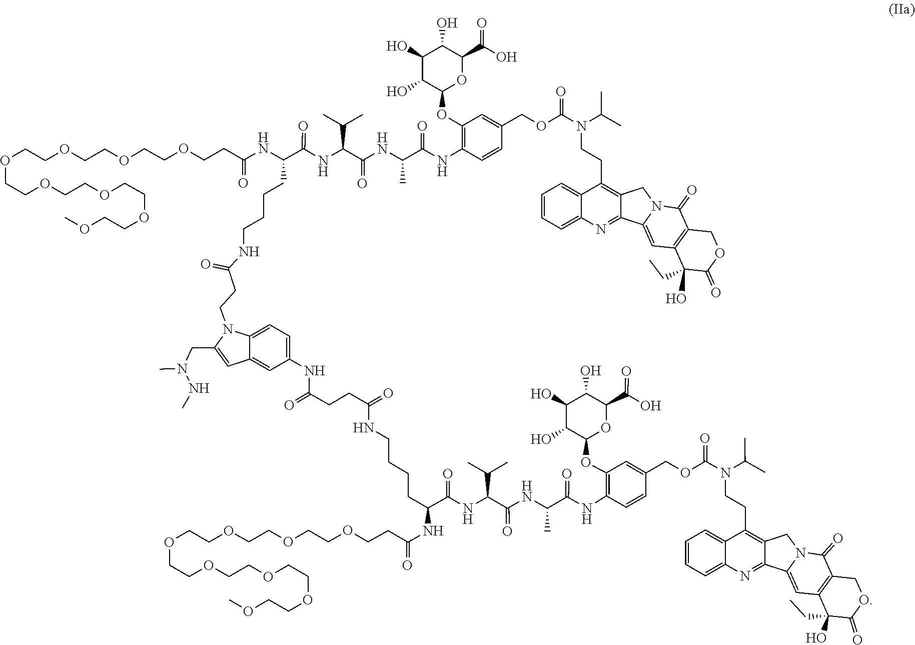 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

An exemplary scheme for making a TF-ADC is shown in Scheme A:

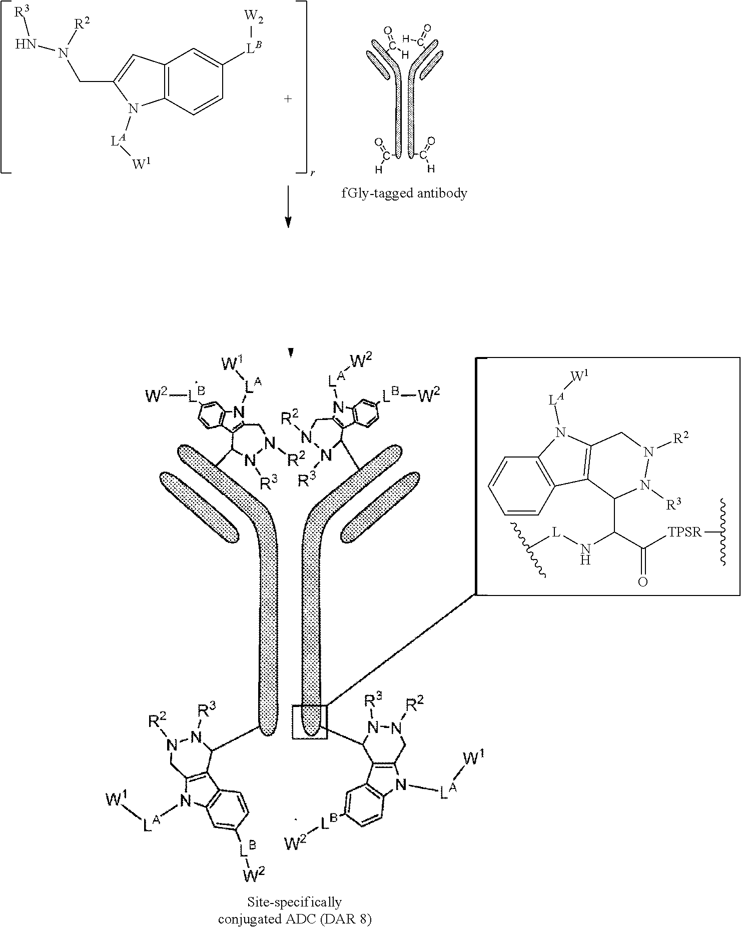

In Scheme A, the linker-drug is conjugated to a TF antibody. The TF antibody has a recognition motif, for example, a L(C/S)TPSR (SEQ ID NO:99) recognition motif in each of the CH1 constant region and c-terminus (CT) of the heavy chain (e.g., SEQ ID NO:80 or 86 as described above). The cysteine residue of a L(C/S)TPSR (SEQ ID NO:99) recognition motif can be converted to a formyl glycine (fGly) and subsequently conjugated to an azaHTPs moiety through a Pictet-Spengler reaction.

Another exemplary scheme for making a TF-ADC is shown in Scheme B:

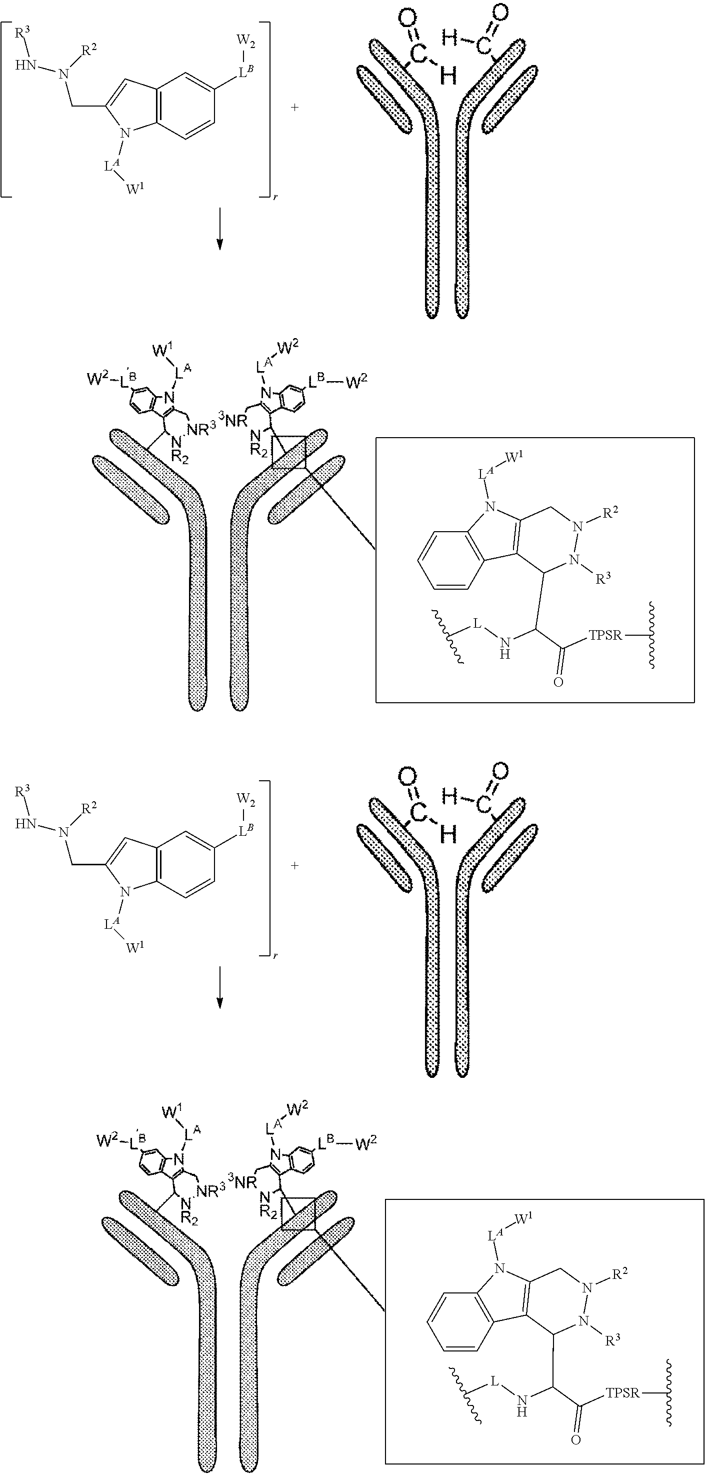

In Scheme B, the linker-drug is conjugated to a TF antibody. The TF antibody has a recognition motif, for example, a LCTPSR (SEQ ID NO:100) recognition motif in the CH1 constant region of the heavy chain (e.g., SEQ ID NO:77 or 83 as described above). The cysteine residue of a LCTPSR (SEQ ID NO:100) recognition motif can be converted to a formyl glycine (fGly) and subsequently conjugated to an azaHTPs moiety through a Pictet-Spengler reaction.

In some embodiments, the TF-ADC is of Formula (II), s is 2, Ab comprises EXMA006 as described herein, and as listed in Table 3 below, and the conjugation sites of the pyridazine-pyrrolo moiety to the antibody are in the CH1 region of each heavy chain of the antibody, as shown in Scheme B. In some embodiments, the Ab comprises a heavy chain comprising an amino acid sequence as set forth in SEQ ID NO:79 and a light chain comprising an amino acid sequence as set forth in SEQ ID NO:91. Accordingly, the corresponding ADC is also referred to herein as TF-ADC 6-4. As it would be understood by one of skill in the art, the drug-to-antibody ratio of TF-ADC 6-4 is 4. In further embodiments, a composition comprising TF-ADC 6-4 exhibits a DAR of about 3 to about 4, for example, about 3.1, about 3.2, about 3.3, about 3.4, about 3.5, about 3.6, about 3.7, about 3.8, about 3.9, or about 4.0. In some embodiments, the DAR of the composition is about 3.4.

TABLE 3

| Ab Conjug Site(s) | CH1 | CH1/CT |
|---|---|---|
| EXMA006 | TF-ADC 6-4 | TF-ADC 6-8 |
| EXMA007 | TF-ADC 7-4 | TF-ADC 7-8 |

In some embodiments, the TF-ADC is of Formula (II), s is 4, Ab comprises EXMA006 as described herein, and as listed in Table 3 above, and the conjugation sites of the pyridazine-pyrrolo moiety to the antibody are the CH1 and CT regions of each heavy chain of the antibody, as shown in Scheme A. In some embodiments, the Ab comprises a heavy chain comprising an amino acid sequence as set forth in SEQ ID NO:82 and a light chain comprising an amino acid sequence as set forth in SEQ ID NO:91. Accordingly, the corresponding ADC is also referred to herein as TF-ADC 6-8. As it would be understood by one of skill in the art, the drug-to-antibody ratio of TF-ADC 6-8 is 8. In further embodiments, a composition comprising TF-ADC 6-8 exhibits a DAR of about 6 to about 8, for example, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0. In some embodiments, the DAR of the composition is about 6.6. In some embodiments, the DAR of the composition is about 7.5.

In some embodiments, the TF-ADC is of Formula (II), s is 2, Ab comprises EXMA007 as described herein, and as listed in Table 3 above, and the conjugation sites of the pyridazine-pyrrolo moiety to the antibody are in the CH1 region of each heavy chain of the antibody, as shown in Scheme B. In some embodiments, the Ab comprises a heavy chain comprising an amino acid sequence as set forth in SEQ ID NO:85 and a light chain comprising an amino acid sequence as set forth in SEQ ID NO:92. Accordingly, the corresponding ADC is also referred to herein as TF-ADC 7-4. As it would be understood by one of skill in the art, the drug-to-antibody ratio of TF-ADC 7-4 is 4. In further embodiments, a composition comprising TF-ADC 7-4 exhibits a DAR of about 3 to about 4, for example, about 3.1, about 3.2, about 3.3, about 3.4, about 3.5, about 3.6, about 3.7, about 3.8, about 3.9, or about 4.0. In some embodiments, the DAR of the composition is about 3.1.

In some embodiments, the TF-ADC is of Formula (II), s is 4, Ab comprises EXMA007 as described herein, and as listed in Table 3 above, and the conjugation sites of the pyridazine-pyrrolo moiety to the antibody are the CH1 and CT regions of each heavy chain of the antibody, as shown in Scheme A. In some embodiments, the Ab comprises a heavy chain comprising an amino acid sequence as set forth in SEQ ID NO:88 and a light chain comprising an amino acid sequence as set forth in SEQ ID NO:92. Accordingly, the corresponding ADC is also referred to herein as TF-ADC 7-8. As it would be understood by one of skill in the art, the drug-to-antibody ratio of TF-ADC 7-8 is 8. In further embodiments, a composition comprising TF-ADC 7-8 exhibits a DAR of about 6 to about 8, for example, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0. In some embodiments, the DAR of the composition is about 6.5. In some embodiments, the DAR of the composition is about 7.1.

In some embodiments, a TF-ADC includes one or more linker-drug conjugated to each heavy chain constant region of an TF antibody via a pyridazine-pyrrolo coupling moiety and can be characterized by a stoichiometric ratio of antibody to linker-drug of about 1 to about 20, for example, a DAR of about 2 to about 8, about 1 to about 4, about 2 to about 4, about 3 to about 4, about 4, about 1 to about 8, about 2 to about 8, about 3 to about 8, about 4 to about 8, about 5 to about 8, about 6 to about 8, about 6.5 to about 8, about 6 to about 7, about 6.5 to about 7.5, about 7 to about 8, about 6.5, about 7, about 7.5, or about 8.

### Drugs for Conjugation

As noted above, a conjugate or a compound of the present disclosure can include as substituents W¹ and W² a drug or active agent. Any of a number of drugs are suitable for use, or can be modified to be rendered suitable for use, as a reactive partner to conjugate to an antibody. Examples of drugs include small molecule drugs and peptide drugs.

“Small molecule drug” as used herein refers to a compound, e.g., an organic compound, which exhibits a pharmaceutical activity of interest and which is generally of a molecular weight of 800 Da or less, or 2000 Da or less, but can encompass molecules of up to 5 kDa and can be as large as 10 kDa. A small inorganic molecule refers to a molecule containing no carbon atoms, while a small organic molecule refers to a compound containing at least one carbon atom.

For example, the drug or active agent can be a topoisomerase inhibitor (e.g., a topoisomerase I inhibitor), such as a camptothecin, or an analog or derivative thereof, or a pharmaceutically active camptothecin moiety and/or a portion thereof. A topoisomerase inhibitor (e.g., camptothecin, or analog or derivative thereof) conjugated to the TF antibody can be any of a variety of topoisomerase inhibitors, for example camptothecin or camptothecin moieties such as, but not limited to, camptothecin and analogs and derivatives thereof as described herein. Examples of drugs that find use in the conjugates and compounds described herein include, but are not limited to, a topoisomerase inhibitor, for example camptothecin or a camptothecin derivative, such as SN-38, Belotecan, Exatecan, 9-aminocamptothecin (9-AC), topotecan, des-Me-topotecan, derivatives thereof, and the like.

Additional examples of topoisomerase inhibitors that find use in the present disclosure are described in PCT/US2022/012325, the disclosure of which is incorporated herein by reference.

In other embodiments, the drug or active agent can be a maytansine. “Maytansine”, “maytansine moiety”, “maytansine active agent moiety” and “maytansinoid” refer to a maytansine and analogs and derivatives thereof, and pharmaceutically active maytansine moieties and/or portions thereof. A maytansine conjugated to the TF antibody can be any of a variety of maytansinoid moieties such as, but not limited to, maytansine and analogs and derivatives thereof as described herein (e.g., deacylmaytansine).

In other instances, the drug or active agent can be an auristatin, or an analog or derivative thereof, or a pharmaceutically active auristatin moiety and/or a portion thereof. An auristatin conjugated to the TF antibody can be any of a variety of auristatin moieties such as, but not limited to, an auristatin and analogs and derivatives thereof as described herein.

Examples of drugs that find use in the conjugates and compounds described herein include, but are not limited to an auristatin or an auristatin derivative, such as monomethyl auristatin D (MMAD), monomethyl auristatin E (MMAE), monomethyl auristatin F (MMAF), derivatives thereof, and the like.

In other cases, the drug or active agent can be a duocarmycin, or an analog or derivative thereof, or a pharmaceutically active duocarmycin moiety and/or a portion thereof.

A duocarmycin conjugated to the TF antibody can be any of a variety of duocarmycin moieties such as, but not limited to, a duocarmycin and analogs and derivatives thereof as described herein. Examples of drugs that find use in the conjugates and compounds described herein include, but are not limited to a duocarmycin or a duocarmycin derivative, such as duocarmycin A, duocarmycin B1, duocarmycin B2, duocarmycin C1, duocarmycin C2, duocarmycin D, duocarmycin SA, and CC-1065, derivatives thereof, and the like. In some embodiments, the duocarmycin is a duocarmycin analog, such as, but not limited to, adozelesin, bizelesin, or carzelesin.

In certain embodiments, the drug is selected from a cytotoxin, a kinase inhibitor, a selective estrogen receptor modulator, an immunostimulatory agent, a toll-like receptor (TLR) agonist, an oligonucleotide, an aptamer, a cytokine, a steroid, and a peptide.

For example, a cytotoxin can include any compound that leads to cell death (e.g., necrosis or apoptosis) or a decrease in cell viability.

Kinase inhibitors can include, but are not limited to, Adavosertib, Afatinib, Axitinib, Bosutinib, Cetuximab, Cobimetinib, Crizotinib, Cabozantinib, Dacomitinib, Dasatinib, Entrectinib, Erdafitinib, Erlotinib, Fostamatinib, Gefitinib, Ibrutinib, Imatinib, Lapatinib, Lenvatinib, Mubritinib, Nilotinib, Pazopanib, Pegaptanib, Ruxolitinib, Sorafenib, Sunitinib, Tucatinib, Vandetanib, Vemurafenib, and the like.

For example, selective estrogen receptor modulators include, but are not limited to, Endoxifen, Tamoxifen, Afimoxifene, Toremifene, and the like.

Immunostimulatory agents can include, but are not limited to, vaccines (e.g., bacterial or viral vaccines), colony stimulating factors, interferons, interleukins, and the like. TLR agonists include, but are not limited to, imiquimod, resiquimod, and the like.

Oligonucleotide dugs include, but are not limited to, fomivirsen, pegaptanib, mipomersen, eteplirsen, defibrotide, nusinersen, golodirsen, viltolarsen, volanesorsen, inotersen, tofersen, tominersen, and the like.

Aptamer drugs include, but are not limited to, pegaptanib, AS1411, REG1, ARC1779, NU172, ARC1905, E10030, NOX-A12, NOX-E36, and the like. Cytokines include, but are not limited to, Albinterferon Alfa-2B, Aldesleukin, ALT-801, Anakinra, Ancestim, Avotermin, Balugrastim, Bempegaldesleukin, Binetrakin, Cintredekin Besudotox, CTCE-0214, Darbepoetin alfa, Denileukin diftitox, Dulanermin, Edodekin alfa, Emfilermin, Epoetin delta, Erythropoietin, Human interleukin-2, Interferon alfa, Interferon alfa-2c, Interferon alfa-n1, Interferon alfa-n3, Interferon alfacon-1, Interferon beta-1a, Interferon beta-1b, Interferon gamma-1b, Interferon Kappa, Interleukin-1 alpha, Interleukin-10, Interleukin-7, Lenograstim, Leridistim, Lipegfilgrastim, Lorukafusp alfa, Maxy-G34, Methoxy polyethylene glycol-epoetin beta, Molgramostim, Muplestim, Nagrestipen, Oprelvekin, Pegfilgrastim, Pegilodecakin, Peginterferon alfa-2a, Peginterferon alfa-2b, Peginterferon beta-1a, Peginterferon lambda-1a, Recombinant CD40-ligand, Regramostim, Romiplostim, Sargramostim, Thrombopoietin, Tucotuzumab celmoleukin, Viral Macrophage-Inflammatory Protein, and the like.

Steroid drugs include, but are not limited to, prednisolone, betamethasone, dexamethasone, hydrocortisone, methylprednisolone, deflazacort, and the like.

“Peptide drug” as used herein refers to amino-acid containing polymeric compounds, and is meant to encompass naturally-occurring and non-naturally-occurring peptides, oligopeptides, cyclic peptides, polypeptides, and proteins, as well as peptide mimetics. The peptide drugs may be obtained by chemical synthesis or be produced from a genetically encoded source (e.g., recombinant source). Peptide drugs can range in molecular weight, and can be from 200 Da to 10 kDa or greater in molecular weight. Suitable peptides include, but are not limited to, cytotoxic peptides; angiogenic peptides; anti-angiogenic peptides; peptides that activate B cells; peptides that activate T cells; anti-viral peptides; peptides that inhibit viral fusion; peptides that increase production of one or more lymphocyte populations; anti-microbial peptides; growth factors; growth hormone-releasing factors; vasoactive peptides; anti-inflammatory peptides; peptides that regulate glucose metabolism; an anti-thrombotic peptide; an anti-nociceptive peptide; a vasodilator peptide; a platelet aggregation inhibitor; an analgesic; and the like.

Additional examples of drugs that find use in the conjugates and compounds described herein include, but are not limited to Tubulysin M, Calicheamicin, a STAT3 inhibitor, alpha-Amanitin, an aurora kinase inhibitor, belotecan, and an anthracycline.

Other examples of drugs include small molecule drugs, such as a cancer chemotherapeutic agent. For example, where a tumor cell expresses or overexpresses TF, the TF antibody can be produced as described herein to include a modified amino acid, which can be subsequently conjugated to a cancer chemotherapeutic agent. Cancer chemotherapeutic agents include non-peptidic (i.e., non-proteinaceous) compounds that reduce proliferation of cancer cells, and encompass cytotoxic agents and cytostatic agents.

Non-limiting examples of chemotherapeutic agents include alkylating agents, nitrosoureas, antimetabolites, antitumor antibiotics, plant (vinca) alkaloids, and steroid hormones. Peptidic compounds can also be used.

Suitable cancer chemotherapeutic agents include dolastatin and active analogs and derivatives thereof, and auristatin and active analogs and derivatives thereof (e.g., Monomethyl auristatin D (MMAD), monomethyl auristatin E (MMAE), monomethyl auristatin F (MMAF), and the like). See, e.g., WO 96/33212, WO 96/14856, and U.S. Pat. No. 6,323,315. For example, dolastatin 10 or auristatin PE can be included in an antibody-drug conjugate of the present disclosure. Suitable cancer chemotherapeutic agents also include maytansinoids and active analogs and derivatives thereof (see, e.g., EP 1391213; and Liu et al (1996) Proc. Natl. Acad. Sci. USA 93:8618-8623); duocarmycins and active analogs and derivatives thereof (e.g., including the synthetic analogues, KW-2189 and CB 1-TM1); and benzodiazepines and active analogs and derivatives thereof (e.g., pyrrolobenzodiazepine (PBD).

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine(methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR), 6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore compounds that bind to the estrogen receptor are used to block this activity. Corticosteroids may inhibit T cell proliferation.

Other suitable chemotherapeutic agents include metal complexes, e.g., cisplatin (cis-DDP), carboplatin, etc.; ureas, e.g., hydroxyurea; and hydrazines, e.g., N-methylhydrazine; epidophyllotoxin; a topoisomerase inhibitor; procarbazine; mitoxantrone; leucovorin; tegafur; etc. Other anti-proliferative agents of interest include immunosuppressants, e.g., mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

Taxanes are suitable for use. “Taxanes” include paclitaxel, as well as any active taxane derivative or pro-drug. “Paclitaxel” (which should be understood herein to include analogues, formulations, and derivatives such as, for example, docetaxel, TAXOL Q, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., TaxotereD docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

Biological response modifiers suitable for use include, but are not limited to, (1) inhibitors of tyrosine kinase (RTK) activity; (2) inhibitors of serine/threonine kinase activity; (3) tumor-associated antigen antagonists, such as antibodies that bind specifically to a tumor antigen; (4) apoptosis receptor agonists; (5) interleukin-2; (6) IFN-α; (7) IFN-γ; (8) colony-stimulating factors; and (9) inhibitors of angiogenesis.

Examples of drugs include small molecule drugs, such as a cancer chemotherapeutic agent. For example, where a tumor cell expresses or overexpresses TF, the TF antibody can be produced as described herein to include a modified amino acid, which can be subsequently conjugated to a cancer chemotherapeutic agent, such as a microtubule affecting agent. In certain embodiments, the drug is a microtubule affecting agent that has antiproliferative activity, such as a maytansinoid.

Embodiments of the present disclosure include conjugates where an antibody is conjugated to two or more drug moieties, such as 3 drug moieties, 4 drug moieties, 5 drug moieties, 6 drug moieties, 7 drug moieties, 8 drug moieties, 9 drug moieties, 10 drug moieties, 11 drug moieties, 12 drug moieties, 13 drug moieties, 14 drug moieties, 15 drug moieties, 16 drug moieties, 17 drug moieties, 18 drug moieties, 19 drug moieties, or 20 or more drug moieties. The drug moieties may be conjugated to the antibody at one or more sites in the antibody, as described herein. In certain embodiments, the conjugates have an average drug-to-antibody ratio (DAR) (molar ratio) in the range of from 0.1 to 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2. In certain embodiments, the conjugates have an average DAR from 1 to 10, such as 1, 2, 3, 4, 5, 6, 7, 8, 9, or 10. In certain embodiments, the conjugates have an average DAR of 1 to 10. In certain embodiments, the conjugates have an average DAR of 1 to 5 (e.g., 4). In certain embodiments, the conjugates have an average DAR of 5 to 10 (e.g., 8). By average is meant the arithmetic mean.

In certain embodiments, the two drugs or active agents attached to the branched linker are the same drug or active agent. For example, a first branch of a branched linker may be attached to a drug or an active agent and a second branch of the branched linker may be attached to the same drug or the same active agent as the first branch. In other embodiments, the two drugs or active agents attached to the branched linker are different drugs or active agents. For example, a first branch of a branched linker may be attached to a first drug or a first active agent and a second branch of the branched linker may be attached to a second drug or a second active agent different from the first drug or the first active agent attached to the first branch.

In some embodiments, where two different drugs or active agents are attached to the branched linker, the drugs or active agents may be selected from drugs and active agents that have a synergistic therapeutic effect. For example, in some instances, the use of two different drugs or active agents attached to the branched linker may provide a lower therapeutically effective concentration at which both payloads act, thereby increasing overall potency of the ADC.

In some embodiments, where two different drugs or active agents are attached to the branched linker, the drugs or active agents may be selected from drugs and active agents that provide an enhanced therapeutic benefit as compared to the use of the drugs or active agents separately, For example, the drugs or active agents may provide an increased effect on drug delivery of the ADC (e.g., some payloads, such as the iRGD peptide, can increase extravasation into tissues and augment tumor penetration).

In some embodiments, where two different drugs or active agents are attached to the branched linker, the drugs or active agents may be selected from drugs and active agents that use different mechanisms of action. In some cases, this may provide a decrease in tumor drug resistance by targeting multiple pathways. Examples of payload combinations can include, but are not limited to, cytotoxic drugs, immunomodulatory molecules to activate or inhibit immune cell populations, cytokines, hormones, chelating agents loaded with radioisotopes, and the like.

In some embodiments, where two different drugs or active agents are attached to the branched linker, the two different drugs or active agents are a topoisomerase inhibitor (e.g., belotecan) as described herein and an auristatin (e.g., MMAE) as described herein. In some embodiments, where two different drugs or active agents are attached to the branched linker, the two different drugs or active agents are a topoisomerase inhibitor (e.g., belotecan) as described herein and an iRGD peptide as described herein. In some embodiments, where two different drugs or active agents are attached to the branched linker, the two different drugs or active agents are an auristatin (e.g., MMAE) as described herein and an iRGD peptide as described herein. In some embodiments, where two different drugs or active agents are attached to the branched linker, the two different drugs or active agents are an auristatin (e.g., MMAE) as described herein and a kinase inhibitor (e.g., Sorafenib, Lapatinib, Gefitinib, and the like) as described herein. In some embodiments, where two different drugs or active agents are attached to the branched linker, the two different drugs or active agents are a topoisomerase inhibitor (e.g., belotecan) as described herein and a kinase inhibitor (e.g., Sorafenib, Lapatinib, Gefitinib, and the like) as described herein. In some embodiments, where two different drugs or active agents are attached to the branched linker, the two different drugs or active agents are an auristatin (e.g., MMAE) as described herein and a selective estrogen receptor modulator (e.g., Endoxifen) as described herein. In some embodiments, where two different drugs or active agents are attached to the branched linker, the two different drugs or active agents are a topoisomerase inhibitor (e.g., belotecan) as described herein and a selective estrogen receptor modulator (e.g., Endoxifen) as described herein.

Drugs to be conjugated to a TF antibody may be modified to incorporate a reactive partner for reaction with the TF antibody. Where the drug is a peptide drug, the reactive moiety (e.g., aminooxy or hydrazide can be positioned at an N-terminal region, the N-terminus, a C-terminal region, the C-terminus, or at a position internal to the peptide. For example, an example of a method involves synthesizing a peptide drug having an aminooxy group. In this example, the peptide is synthesized from a Boc-protected precursor. An amino group of a peptide can react with a compound comprising a carboxylic acid group and oxy-N-Boc group. As an example, the amino group of the peptide reacts with 3-(2,5-dioxopyrrolidin-1-yloxy)propanoic acid. Other variations on the compound comprising a carboxylic acid group and oxy-N-protecting group can include different number of carbons in the alkylene linker and substituents on the alkylene linker. The reaction between the amino group of the peptide and the compound comprising a carboxylic acid group and oxy-N-protecting group occurs through standard peptide coupling chemistry. Examples of peptide coupling reagents that can be used include, but not limited to, DCC (dicyclohexylcarbodiimide), DIC (diisopropylcarbodiimide), di-p-toluoylcarbodiimide, BDP (1-benzotriazole diethylphosphate-1-cyclohexyl-3-(2-morpholinylethyl)carbodiimide), EDC (1-(3-dimethylaminopropyl-3-ethyl-carbodiimide hydrochloride), cyanuric fluoride, cyanuric chloride, TFFH (tetramethyl fluoroformamidinium hexafluorophosphosphate), DPPA (diphenylphosphorazidate), BOP (benzotriazol-1-yloxytris(dimethylamino)phosphonium hexafluorophosphate), HBTU (O-benzotriazol-1-yl-N,N,N′,N′-tetramethyluronium hexafluorophosphate), TBTU (O-benzotriazol-1-yl-N,N,N′,N′-tetramethyluronium tetrafluoroborate), TSTU (O—(N-succinimidyl)-N,N,N′,N′-tetramethyluronium tetrafluoroborate), HATU (N-[(dimethylamino)-1-H-1,2,3-triazolo[4,5,6]-pyridin-1-ylmethylene]- -N-methylmethanaminium hexafluorophosphate N-oxide), BOP-Cl(bis(2-oxo-3-oxazolidinyl)phosphinic chloride), PyBOP ((1-H-1,2,3-benzotriazol-1-yloxy)-tris(pyrrolidino)phosphonium tetrafluorophopsphate), BrOP (bromotris(dimethylamino)phosphonium hexafluorophosphate), DEPBT (3-(diethoxyphosphoryloxy)-1,2,3-benzotriazin-4(3H)-one) PyBrOP (bromotris(pyrrolidino)phosphonium hexafluorophosphate). As a non-limiting example, HOBt and DIC can be used as peptide coupling reagents.

Deprotection to expose the amino-oxy functionality is performed on the peptide comprising an N-protecting group. Deprotection of the N-oxysuccinimide group, for example, occurs according to standard deprotection conditions for a cyclic amide group.

Deprotecting conditions can be found in Greene and Wuts, Protective Groups in Organic Chemistry, 3rd Ed., 1999, John Wiley & Sons, NY and Harrison et al. Certain deprotection conditions include a hydrazine reagent, amino reagent, or sodium borohydride. Deprotection of a Boc protecting group can occur with TFA. Other reagents for deprotection include, but are not limited to, hydrazine, methylhydrazine, phenylhydrazine, sodium borohydride, and methylamine. The product and intermediates can be purified by conventional means, such as HPLC purification.

The ordinarily skilled artisan will appreciate that factors such as pH and steric hindrance (i.e., the accessibility of the amino acid residue to reaction with a reactive partner of interest) are of importance, Modifying reaction conditions to provide for optimal conjugation conditions is well within the skill of the ordinary artisan, and is routine in the art. Where conjugation is conducted with a TF antibody present in or on a living cell, the conditions are selected so as to be physiologically compatible. For example, the pH can be dropped temporarily for a time sufficient to allow for the reaction to occur but within a period tolerated by the cell (e.g., from about 30 min to 1 hour). Physiological conditions for conducting modification of antibodies on a cell surface can be similar to those used in a ketone-azide reaction in modification of cells bearing cell-surface azides (see, e.g., U.S. Pat. No. 6,570,040).

Small molecule compounds containing, or modified to contain, an α-nucleophilic group that serves as a reactive partner with a compound or conjugate disclosed herein are also contemplated for use as drugs in the antibody-drug conjugates of the present disclosure. General methods are known in the art for chemical synthetic schemes and conditions useful for synthesizing a compound of interest (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

### Formulations & Compositions

TF-ADCs can be included in a pharmaceutical composition for administration, e.g., to a subject for treating a disease, disorder, or condition. A pharmaceutical composition can comprise TF-ADCs which, on average, can exhibit a drug-to-antibody ratio (“DAR”) of about 0.1 to about 20, or from 0.5 to 20, or from 1 to 20. In other words, each antibody is conjugated to one or two linker-drug conjugates. Methods to determine DAR are well known to the skilled person and include methods using Reverse Phase Chromatography, or HPLC-MS.

For example, in any embodiment, a pharmaceutical composition comprising a TF-ADC can exhibit a DAR of about 0.1 to about 20, or from 0.5 to 20, or from 1 to 20, such as from 1 to 19, or from 1 to 18, or from 1 to 17, or from 1 to 16, or from 1 to 15, or from 1 to 14, or from 1 to 13, or from 1 to 12, or from 1 to 11, or from 1 to 10, or from 1 to 9, or from 1 to 8, or from 1 to 7, or from 1 to 6, or from 1 to 5, or from 1 to 4, or from 1 to 3, or from 1 to 2, or from 2 to 20, or from 2 to 19, or from 2 to 18, or from 2 to 17, or from 2 to 16, or from 2 to 15, or from 2 to 14, or from 2 to 13, or from 2 to 12, or from 2 to 11, or from 2 to 10, or from 2 to 9, or from 2 to 8, or from 2 to 7, or from 2 to 6, or from 2 to 5, or from 2 to 4, or from 2 to 3, or from 3 to 20, or from 3 to 19, or from 3 to 18, or from 3 to 17, or from 3 to 16, or from 3 to 15, or from 3 to 14, or from 3 to 13, or from 3 to 12, or from 3 to 11, or from 3 to 10, or from 3 to 9, or from 3 to 8, or from 3 to 7, or from 3 to 6, or from 3 to 5, or from 3 to 4, or from 4 to 20, or from 4 to 19, or from 4 to 18, or from 4 to 17, or from 4 to 16, or from 4 to 15, or from 4 to 14, or from 4 to 13, or from 4 to 12, or from 4 to 11, or from 4 to 10, or from 4 to 9, or from 4 to 8, or from 4 to 7, or from 4 to 6, or from 4 to 5, or from 5 to 20, or from 5 to 19, or from 5 to 18, or from 5 to 17, or from 5 to 16, or from 5 to 15, or from 5 to 14, or from 5 to 13, or from 5 to 12, or from 5 to 11, or from 5 to 10, or from 5 to 9, or from 5 to 8, or from 5 to 7, or from 5 to 6, or from 6 to 20, or from 6 to 19, or from 6 to 18, or from 6 to 17, or from 6 to 16, or from 6 to 15, or from 6 to 14, or from 6 to 13, or from 6 to 12, or from 6 to 11, or from 6 to 10, or from 6 to 9, or from 6 to 8, or from 6 to 7, or from 7 to 20, or from 7 to 19, or from 7 to 18, or from 7 to 17, or from 7 to 16, or from 7 to 15, or from 7 to 14, or from 7 to 13, or from 7 to 12, or from 7 to 11, or from 7 to 10, or from 7 to 9, or from 7 to 8.

In certain embodiments, the composition exhibits a DAR from 1 to 10, such as 1, 2, 3, 4, 5, 6, 7, 8, 9, or 10. In certain embodiments, the composition exhibits a DAR of 1 to 10. In certain embodiments, the composition exhibits a DAR of 1 to 5 (e.g., 3 or 3.5 or 4). In certain embodiments, composition exhibits a DAR of 5 to 10 (e.g., 6, or 6.5, or 7, or 7.5 or 8). In some embodiments, a pharmaceutical composition comprises a TF-ADC comprising a TF antibody as described herein, including EXMA006 or EXMA007 (see, e.g., Tables 1-2), and a pharmaceutically acceptable excipient.

In some embodiments, a pharmaceutical composition comprises TF-ADC 6-4. In further embodiments, the composition comprising TF-ADC 6-4 exhibits a DAR of about 3 to about 4, for example, about 3.1, about 3.2, about 3.3, about 3.4, about 3.5, about 3.6, about 3.7, about 3.8, about 3.9, or about 4.0. In some embodiments, the DAR of the composition is about 3.4.

In some embodiments, a pharmaceutical composition comprises TF-ADC 7-4. In further embodiments, the composition comprising TF-ADC 7-4 exhibits a DAR of about 3 to about 4, for example, about 3.1, about 3.2, about 3.3, about 3.4, about 3.5, about 3.6, about 3.7, about 3.8, about 3.9, or about 4.0. In some embodiments, the DAR of the composition is about 3.1.

In some embodiments, a pharmaceutical composition comprises TF-ADC 6-8. In further embodiments, the composition comprising TF-ADC 6-8 exhibits a DAR of about 6 to about 8, for example, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0. In some embodiments, the DAR of the composition is about 6.6. In some embodiments, the DAR of the composition is about 7.5.

In some embodiments, a pharmaceutical composition comprises TF-ADC 7-8. In further embodiments, the composition comprising TF-ADC 7-8 exhibits a DAR of about 6 to about 8, for example, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0. In some embodiments, the DAR of the composition is about 6.5. In some embodiments, the DAR of the composition is about 7.1.

A TF-ADC can be formulated in any of a variety of different ways. A TF-ADC of the present disclosure can be provided in any suitable form, e.g., in the form of a pharmaceutically acceptable salt, and can be formulated for any suitable route of administration, e.g., oral, topical, or parenteral administration. Where a TF-ADC is provided as a liquid injectable (such as in those embodiments where they it can be administered intravenously or directly into a tissue), a TF-ADC can be provided as a ready-to-use dosage form, or as a storage-stable powder for reconstitution or liquid composed of pharmaceutically acceptable excipients and excipients.

Methods for formulating a TF-ADC can be adapted from those available in the art. For example, TF-ADCs can be provided in a pharmaceutical composition comprising an effective amount of a TF-ADC and a pharmaceutically acceptable excipient (e.g., saline). The pharmaceutical composition can optionally include other additives (e.g., buffers, stabilizers, preservatives, and the like). In some embodiments, the formulations are suitable for administration to a mammal, such as those that are suitable for administration to a human.

Also provided herein are pharmaceutical compositions that contain an effective amount of a TF-ADC described herein and a pharmaceutically acceptable excipient. In some embodiments, the TF-ADC comprises a TF antibody as described herein, including EXMA006 and/or EXMA007, as described in any one of Tables 1-2. In some embodiments, the TF-ADC is TF-ADC 6-4 as disclosed herein. In some embodiments, the TF-ADC is TF-ADC 6-8 as disclosed herein. In some embodiments, the TF-ADC is TF-ADC 7-4 as disclosed herein. In some embodiments, the TF-ADC is TF-ADC 7-8 as disclosed herein. In some embodiments, a pharmaceutical composition comprises an effective amount of a TF-ADC of Formula (I) and a pharmaceutically acceptable excipient.

The pharmaceutically acceptable excipient can be one or more compatible solid or liquid fillers, diluents, other excipients, or encapsulating substances that are suitable for administration into a human or veterinary subject (e.g., a physiologically acceptable and/or pharmacologically acceptable). The pharmaceutically acceptable excipient can be co-mingled with one or more of the active components, e.g., a hybrid molecule, and with each other, when more than one pharmaceutically acceptable excipient is present in the pharmaceutical composition, in a manner so as not to substantially impair the desired pharmaceutical efficacy. Pharmaceutically acceptable materials typically are capable of administration to a subject without the production of significant undesirable physiological effects such as nausea, dizziness, rash, or gastric upset. It is, for example, desirable for a composition comprising a pharmaceutically acceptable excipient not to be immunogenic when administered to a human subject for therapeutic purposes.

Pharmaceutical compositions of the invention can additionally contain suitable buffering agents, including, for example, acetic acid in a salt, citric acid in a salt, boric acid in a salt, and phosphoric acid in a salt. The pharmaceutical compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include the step of bringing the antibody or antigen-binding fragment of the invention into association with an excipient that constitutes one or more accessory ingredients. In general, the pharmaceutical composition is prepared by uniformly and intimately bringing the active agent into association with a liquid excipient, a finely divided solid excipient, or both, and then, if necessary, shaping the product.

A composition suitable for parenteral administration conveniently comprises a sterile aqueous preparation of the composition, which preferably is isotonic with the blood of the recipient. This aqueous preparation can be formulated of known methods using suitable dispersing or wetting agents and suspending agents. The sterile injectable preparation also can be a sterile injectable solution or suspension in a non-toxic parenterally acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed, such as synthetic mono- or di-glycerides. In addition, fatty acids such as oleic acid can be used in the preparation of injectables. Excipient formulations suitable for oral, subcutaneous, intravenous, intramuscular, and the like, administrations can be found in Remington's Pharmaceutical Sciences, Mack Publishing Co., Easton, PA.

Preparation of pharmaceutical compositions of the invention and their various routes of administration can be carried out in accordance with methods well known in the art. The delivery systems useful in the context of the invention include time-released, delayed release, and sustained release delivery systems such that the delivery of the composition occurs prior to, and with sufficient time to cause, sensitization of the site to be treated. The composition can be used in conjunction with other therapeutic agents or therapies. Such systems can avoid repeated administrations of the composition, thereby increasing convenience to the subject and the physician, and can be particularly suitable for some compositions of the invention.

Many types of release delivery systems are available and known to those of ordinary skill in the art. Suitable release delivery systems include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids such as sterols such as cholesterol, cholesterol esters, and fatty acids or neutral fats such as mono-di- and triglycerides; hydrogel release systems; sylastic systems; peptide-based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation.

Generally, a TF-ADC or pharmaceutical composition is suitably packaged, e.g., in a vial, pouch, ampoule, and/or any container appropriate for a therapeutic method. Components can be provided as concentrates (including lyophilized compositions), which can be further diluted prior to use, or they can be provided at the concentration of use. For use of a TF-ADC in vivo, single dosages can be provided in sterilized containers having the desired amount and concentration of components.

### Methods of Treatment

Also provided herein are methods of treating, preventing, or alleviating a TF-mediated disease, disorder, or condition, including one or more symptoms of the TF-mediated disease, disorder, or condition with a TF-ADC comprising a TF antibody, such as anti-human TF, and a drug conjugated directly or indirectly thereto. Also provided herein are methods of killing tumor cells with a TF-ADC comprising a TF antibody, such as human TF, and a drug conjugated directly or indirectly thereto.

The antibody that binds to TF can include any as described herein, such as EXMA006 and/or EXMA007, as described in any one of Tables 1-2. In some embodiments, a method of killing a tumor cell comprises contacting the tumor cell with a TF-ADC of Formula (I). In some embodiments, a method of killing a tumor cell comprises contacting the tumor cell with a TF-ADC comprising a camptothecin analogue (e.g., belotecan), or any derivative thereof. In some embodiments, a method of killing a tumor cell comprises contacting the tumor cell with a TF-ADC of Formula (I) or (II). In some embodiments, a method of killing a tumor cell comprises contacting the tumor cell with a TF-ADC of TF-ADC-6-4, TF-ADC-7-4, TF-ADC-6-8, or TF-ADC-7-8. In some embodiments, the contacting is in vivo or in vitro.

In any embodiment, the method of contacting the tumor cell with a TF-ADC can comprise contacting the tumor cell with a composition comprising the TF-ADC and one or more pharmaceutically acceptable excipients. In some embodiments, the TF-ADC in the composition can be characterized by a DAR of about 1 to about 20, about 1 to about 10, about 2 to about 4, about 3 to about 4, about 5 to about 8, about 6 to about 8, about 6 to about 7, or about 7 to about 8.

Also provided herein is a method of treating a cancer in a subject in need thereof comprising administering an effective amount of a TF-ADC to the subject, wherein the TF-ADC comprises a TF antibody and a drug conjugated thereto via a linker, as described herein.

In some embodiments, the TF antibody is EXMA006 or EXMA007, as described herein, such as in any one of Tables 1-2. In some embodiments, the TF-ADC is TF-ADC 6-4 as disclosed herein. In some embodiments, the TF-ADC is TF-ADC 6-8 as disclosed herein. In some embodiments, the TF-ADC is TF-ADC 7-4 as disclosed herein. In some embodiments, the TF-ADC is TF-ADC 7-8 as disclosed herein.

In some embodiments, a method of treating cancer in a subject in need thereof comprises administering to the subject an effective amount of a TF-ADC of Formula (I) or (II).

In any embodiment, the method of treating cancer in a subject in need thereof can comprising administering to the subject a pharmaceutical composition comprising the TF-ADC and a pharmaceutically acceptable excipient. In some embodiments, the TF-ADC in the pharmaceutical composition can be characterized by a DAR of about 1 to about 20, about 1 to about 10, about 2 to about 4, about 3 to about 4, about 5 to about 8, about 6 to about 8, about 6 to about 7, or about 7 to about 8.

In some embodiments, the method of treating cancer in a subject in need thereof is effective to enhance the removal of the cancer cells, for example, tumor cells, in the subject.

“Enhancing” the removal of cancer or tumor cells does not require a 100% enhancement of removal. Any enhancement in the rate of removal is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal can be enhanced by, for example, at least about 5%, at least about 10% or at least about 20% compared to levels of removal observed in the absence of the method (e.g., in a biologically matched control subject or specimen that is not exposed to the agent of the method). The effect is detected by, for example, a reduction in tumor size, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of a TF binding agent (e.g., an antibody) of the method.

The present disclosure also provides a method of modulating (e.g., inhibiting, reducing, preventing) tumor growth in a subject in need thereof, the method comprising administering to the subject a TF-ADC comprising a TF antibody and a drug linked thereto via a linker, as described herein, in an amount effective to modulate the tumor growth in the subject. In some embodiments, a method of modulating tumor growth in a subject in need thereof comprising administering an effective amount of a TF-ADC comprising a TF antibody and a drug conjugated thereto via a linker, as described herein. In some embodiments, the antibody that binds to TF is EXMA006 or EXMA007, as described in any one of Tables 1-2. In some embodiments, the TF-ADC is TF-ADC 6-4 as disclosed herein. In some embodiments, the TF-ADC is TF-ADC 6-8 as disclosed herein. In some embodiments, the TF-ADC is TF-ADC 7-4 as disclosed herein. In some embodiments, the TF-ADC is TF-ADC 7-8 as disclosed herein. In some embodiments, a method of modulating tumor growth in a subject in need thereof comprises administering to the subject an effective amount of a TF-ADC of Formula (I) or (II).

In any embodiment, the method of modulating tumor growth in a subject in need thereof can comprise administering to the subject a pharmaceutical composition comprising the TF-ADC and one or more pharmaceutically acceptable excipients. In some embodiments, the TF-ADC in the pharmaceutical composition can be characterized by a DAR of about 1 to about 20, about 1 to about 10, about 2 to about 4, about 3 to about 4, about 5 to about 8, about 6 to about 8, about 6 to about 7, or about 7 to about 8.

In some embodiments, the cancer and tumor cells described herein that may be treated and/or killed by the methods described herein express TF, for example, as on a surface of the cancer or tumor cell. In some embodiments, a tumor or cancer cell may overexpress TF. As used herein, the term “overexpress” means to transcribe and translate more genetic product than normal (such as in normal cells), the process of which is often a characteristic of cancer cells.

Examples of cancers that can be treated with a TF-ADC described herein include, but are not limited to head and neck cancer, ovarian cancer, gastric cancer, esophageal cancer, cervical cancer, prostate cancer, pancreatic cancer, estrogen receptors negative (ER-), progesterone receptors negative (PR-), and HER2 negative (HER2-) triple negative breast cancer, glioblastoma, cancer, bladder cancer, melanoma, and kidney cancer.

Additionally, a TF-ADC comprising a TF antibody can be used to alleviate or reduce side effects associated with cancer such as, for example, bone deterioration, vertebral collapse, and paralysis. In one embodiment, the subject suffers from or is at risk of suffering from bone metastases and a TF-ADC comprising a TF antibody is administered in an amount to reduce deterioration of surrounding bone. Accordingly, in some embodiments, a TF-ADC comprising a TF antibody prevents bone deterioration due to bone metastases, wherein tumor cell proliferation is or is not reduced. In some embodiments, a TF binding agent (e.g., an antibody or ADC) a TF-ADC comprising a TF antibody both prevents bone deterioration due to bone metastases and reduces tumor cell proliferation. In general, the effect on tumor cell proliferation (e.g., inhibition of proliferation or no effect on proliferation) depends on the microenvironment of a particular metastasis. For example, proliferation of metastases located in microenvironments with substantial amounts of type 1 collagen can be inhibited. In contrast, proliferation of metastases located in microenvironments lacking substantial amounts of type 1 collagen cannot be inhibited, yet bone deterioration near the metastasis is reduced or prevented.

Therefore, the present disclosure also provides a method of alleviating or reducing side effects associated with cancer comprising administering an effective amount of a TF-ADC to a subject in need thereof. In some embodiments, a method of alleviating or reducing side effects associated with cancer comprises administering an effective amount of a TF-ADC comprising a TF antibody and a drug conjugated thereto via a linker, as described herein. In some embodiments, the antibody that binds to TF is EXMA006 or EXMA007, as described in any one of Tables 1-2. In some embodiments, the TF-ADC is TF-ADC 6-4 as disclosed herein. In some embodiments, the TF-ADC is TF-ADC 6-8 as disclosed herein. In some embodiments, the TF-ADC is TF-ADC 7-4 as disclosed herein. In some embodiments, the TF-ADC is TF-ADC 7-8 as disclosed herein. In some embodiments, a method of alleviating or reducing side effects associated with cancer comprises administering to the subject an effective amount of a TF-ADC of Formula (I) or (II).

In any embodiment, the method of alleviating or reducing side effects associated with cancer in a subject in need thereof can comprising administering to the subject a pharmaceutical composition comprising a TF-ADC as disclosed herein and one or more pharmaceutically acceptable excipients. In some embodiments, the TF-ADC in the pharmaceutical composition can be characterized by a DAR of about 1 to about 20, about 1 to about 10, about 2 to about 4, about 3 to about 4, about 5 to about 8, about 6 to about 8, about 6 to about 7, or about 7 to about 8.

Additionally, a TF-ADC comprising a TF antibody can be used to treating, preventing, or alleviating a TF-mediated disease, disorder, or condition, including one or more symptoms of the TF-mediated disease, disorder, or condition with a TF-ADC comprising a TF antibody. In some embodiments, the disease or condition involves neovascularization. In some embodiments, the disease or condition involving neovascularization is age-related macular degeneration (AMD), diabetic retinopathy, or cancer. In some embodiments, the disease or condition involves vascular inflammation.

In another aspect, provided herein is a method of detecting TF in a subject having or suspected of having a disease or condition, the method comprising: (a) administering to the subject any antibody above; and (b) detecting the presence or the level of TF in the subject.

In some embodiments, the disease or condition is cancer. In some embodiments, the cancer is head and neck cancer. In some embodiments, the cancer is ovarian cancer. In some embodiments, the cancer is gastric cancer. In some embodiments, the cancer is esophageal cancer. In some embodiments, the cancer is cervical cancer. In some embodiments, the cancer is prostate cancer. In some embodiments, the cancer is pancreatic cancer. In some embodiments, the cancer is estrogen receptors negative (ER-), progesterone receptors negative (PR-), and HER2 negative (HER2-) triple negative breast cancer. In some embodiments, the cancer is glioblastoma. In some embodiments, the cancer is lung cancer. In some embodiments, the cancer is bladder cancer. In some embodiments, the cancer is melanoma. In some embodiments, the cancer is kidney cancer.

In some embodiments, the disease or condition involves neovascularization. In some embodiments, the disease or condition involving neovascularization is age-related macular degeneration (AMD), diabetic retinopathy, or cancer. In some embodiments, the disease or condition involves vascular inflammation.

It is understood that modifications that do not substantially affect the activity of the various embodiments described herein are also provided within the definition of the subject matter described herein. Accordingly, the following examples are intended to illustrate but not limit the present disclosure.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

### Example 1—Preparation of ADCs

### a. Preparation of Tagged Antibodies

Tagged antibodies were prepared according to methods known in the art, such as those described in Rabuka D. et al., “Site-specific chemical protein conjugation using genetically encoded aldehyde tags,” Nat Protoc., 2012; 7(6):1052-1067 and in U.S. Pat. No. 7,985,783 B2, each of which is incorporated in its entirety herein by reference. Briefly, an antibody containing a heterologous sulfatase motif can be contacted with a formylglycine-generating enzyme to convert a cysteine or serine in the motif to a formylglycine having an aldehyde moiety, thereby generating an “aldehyde-tagged antibody.” Any formylglycine-generating enzyme can be used, such as Mycobacterium tuberculosis or Mycobacterium tuberculosis.

### B. Synthesis of Formula (IIa) Linker-Payload

### Material and Methods

Synthetic reagents were purchased from Sigma-Aldrich, Acros, AK Scientific, or other commercial sources and used as received. Anhydrous solvents were obtained from commercial sources in sealed bottles. Compound 12 (previously reported in Chuprakov et al. Bioconjugate Chem. 2021, 32, 746-754) was obtained commercially from Shanghai Medicilon and used without purification. Belotecan 13 as well as compounds 16 and 17 were obtained from commercial sources and used as received. Column chromatography was performed using a Biotage Isolera or Biotage Selekt chromatography system. Low-resolution mass spectra (LRMS) were acquired on Agilent Technology 6120 Quadrupole LC/MS, equipped with Agilent 1260 Infinity IPLC system, G1314 variable wavelength detector, and Agilent Poroshell 120 SB C18, 4.6 mm×50 mm column at room temperature using 10-100% gradient of water and acetonitrile containing 0.1% formic acid. HPLCs were monitored at 254 or 205 nm.

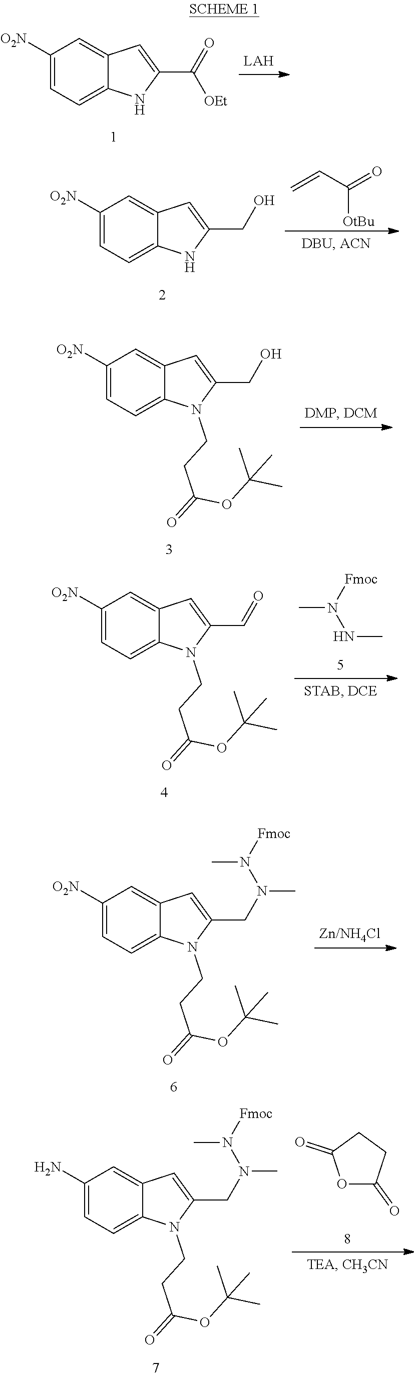

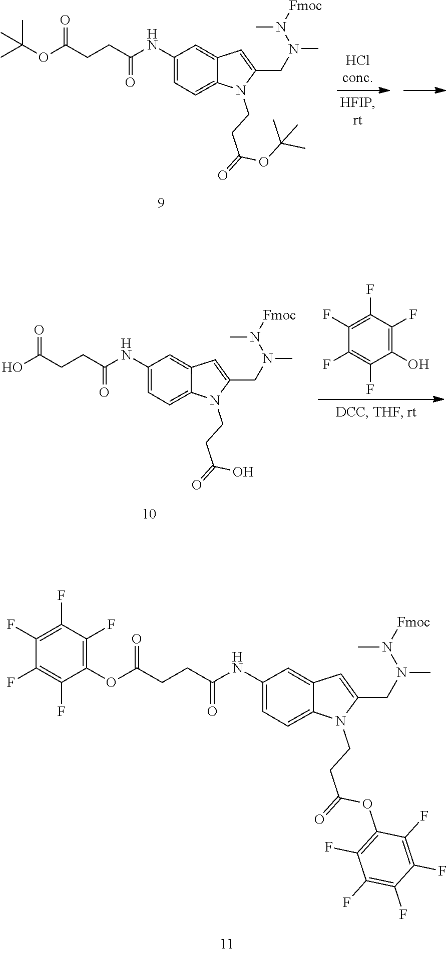

### Preparation of (5-nitro-1H-indol-2-yl)methanol (2)

To an oven-dried round-bottom flask were added ethyl 5-nitro-1H-indole-2-carboxylate (1, 25 g, 107 mmol) and 250 mL of anhydrous THIF. The resulting solution was cooled down to 0° C. and treated with lithium aluminum hydride (4.9 g, 129 mmol) in small portions over 30 minutes with vigorous stirring under nitrogen. Reaction mixture was stirred at room temperature for 2 h, poured into 100 mL of water, and extracted with ethyl acetate (400 mL). Organic layer was concentrated under vacuum and purified on silica gel (75% v/v EtOAc/hexane) to afford 14 g (73 mmol, 68% yield) of product 2 as a brown solid. LRMS (ESI): m/z 192.9 [M+H]⁺, calculated for C₉H₈N₂O₃ m/z 193.1.

### Preparation of tert-butyl 3-(2-(hydroxymethyl)-5-nitro-1H-indol-1-yl)propanoate (3)

In an oven-dried round-bottom flask were combined 5-nitro-1H-indol-2-yl)methanol (2, 14 g, 73 mmol), 140 mL of acetonitrile, and 12 g (94 mmol) of tert-butyl acrylate. The resulting solution was treated with DBU (16.6 g, 109 mmol) at room temperature over 30 minutes. Reaction mixture was stirred at 80° C. for 3 h, then poured into 100 mL of water and concentrated under reduced pressure to remove acetonitrile. Aqueous residue was then extracted with dichloromethane (300 mL). Organic layer was washed with brine, dried over sodium sulfate, concentrated under vacuum, and purified on silica gel (25% v/v EtOAc-hexane) to give 18 g (56 mmol, 77% yield) of product 3 as a yellow solid. LRMS (ESI): m z 320.9 [M+H]⁺, calculated for C₁₆H₂₀N₂O₅ m/z 321.1.

### Preparation of tert-butyl 3-(2-formyl-5-nitro-1H-indol-1-yl)propanoate (4)

To a stirred solution of compound 3 (18 g, 56 mmol) in 200 mL of anhydrous DCM were added Dess-Martin periodinane (28 g, 66 mmol) in small portions over 15 minutes at room temperature. The resulting mixture was stirred for 1 h at RT and quenched by adding 100 mL of water. Aqueous layer was extracted with DCM (200 mL), combined organic layers were washed with brined and dried over sodium sulfate. After removal of solvents, the residue was purified on silica gel (0-25% v/v EtOAc-hexanes) to give 14 g (41 mmol, 73% yield) of aldehyde 4 as a yellow solid. LRMS (ESI): m/z 340.9 [M+Na]⁺, calculated for C₁₆H₁₈N₂O₅ m/z 341.1.

### Preparation of (9H-fluoren-9-yl)methyl 1,2-dimethylhydrazine-1-carboxylate (5)

To a solution of 1,2-dimethylhydrazine dihydrochloride (20 g, 150 mmol) in DCM (200 mL) at 25° C. were added triethylamine (45.6 g, 452 mmol) slowly over 10 min. The mixture was stirred at 25° C. for 30 min and treated with (9H-fluoren-9-yl)methyl carbonochloridate (Fmoc chloride, 19.4 g, 75 mmol) at room temperature and stirred for 3 h. Reaction mixture was poured into water (300 mL) and extracted with ethyl acetate (200 mL). Combined organic phase was dried over sodium sulfate, concentrated under vacuum, and purified by silica-gel chromatography (eluting with 30% v/v EtOAc-hexanes) to give compound 5 (20 g, 71 mmol, 95% yield) as a yellow low-melting solid. LRMS (ESI): m z 283.2 [M+H]⁺, calculated for C₁₇H₁₈N₂O₂ m/z 283.1.

### Preparation of (9H-fluoren-9-yl)methyl 2-((1-(3-(tert-butoxy)-3-oxopropyl)-5-nitro-1H-indol-2-yl)methyl)-1,2-dimethylhydrazine-1-carboxylate (6)

In an oven-dried round-bottom flask were combined aldehyde 4 (14 g, 41 mmol) and hydrazine 5 (11 g, 39 mmol) in 140 mL of anhydrous DCE at ambient temperature. The mixture was stirred for 1 h and then treated with STAB (17 g, 80 mmol) in portions over 30 minutes. Stirring continued for 2 h at room temperature, then reaction mixture was quenched with 100 mL of water, aqueous layer was extracted with DCM (300 mL). Combined organic layer was washed with brine and dried over sodium sulfate. After removal of solvents, the residue was purified on silica gel (0-25% v/v EtOAc-hexane) to obtain 18 g (31 mmol, 80% yield) of compound 6 as a brown solid. LRMS (ESI): m/z 584.9 [M+H]⁺, calculated for C₃₃H₃₆N₄O₆ m/z 585.3.

### Preparation of (9H-fluoren-9-yl)methyl 2-((5-amino-1-(3-(tert-butoxy)-3-oxopropyl)-1H-indol-2-yl)methyl)-1,2-dimethylhydrazine-1-carboxylate (7)

A solution of nitro compound 6 (18 g, 31 mmol) in 180 mL of THF was combined with a solution of ammonium chloride (13 g, 243 mmol) in 180 mL of water, and 90 mL of methanol at ambient temperature. The resulting mixture was treated with zinc powder (16 g, 245 mmol) in several portions at room temperature. Reaction mixture was stirred vigorously for 2 hours, solids were filtered off, filtrate was concentrated under vacuum and partitioned between saturated aqueous ammonium chloride (100 mL) and ethyl acetate (100 mL). Aqueous layer was separated and extracted with ethyl acetate (100 mL). Combined organic layer was washed with brine, and dried over sodium sulfate. Removal of solvents under vacuum afforded 15 g of crude product 7 as a brown oil which was used further without purification. LRMS (ESI): m/z 555.3 [M+H]⁺, calculated for C₃₃H₃₈N₄O₄ m/z 555.3.

### Preparation of 4-((2-((2-(((9H-fluoren-9-yl)methoxy)carbonyl)-1,2-dimethylhydrazineyl)methyl)-1-(3-(tert-butoxy)-3-oxopropyl)-1H-indol-5-yl)amino)-4-oxobutanoic acid (9)

To a solution of compound 7 (1.0 g, 1.8 mmol) in MeCN (10 mL) were added succinic anhydride (905 mg, 5.0 equiv., 9 mmol) and triethylamine (1.2 mL, 9 mmol).

Reaction mixture was stirred for 3 hours at room temperature and purified by reversed-phase chromatography (C18 column, 0-100% v/v MeCN/H2O with 0.05% TFA). Pure fractions were combined, concentrated under reduced pressure until murky, and lyophilized to give 0.95 g of product 9 (1.5 mmol, 83% yield) as an off-white solid. LRMS (ESI): m/z 677.3 [M⁺Na]⁺, calculated for C₃₇H₄₂N₄O₇ m/z 677.3.

### Preparation of 4-((2-((2-(((9H-fluoren-9-yl)methoxy)carbonyl)-1,2-dimethylhydrazinyl)methyl)-1-(2-carboxyethyl)-1H-indol-5-yl)amino)-4-oxobutanoic acid (10)

Compound 9 (9 g, 14 mmol) was dissolved in 90 mL of hexafluoro isopropanol and treated with 10 mL of concentrated HCl at room temperature. Reaction mixture was stirred for 1 h at room temperature, then solvent was removed in vacuum, and the residue was purified by reversed-phase flash chromatography (C18, 0-100% v/v CH3CN-H₂O with 0.05% TFA). Fractions containing product were combined and lyophilized to give diacid 10 (6.5 g, 11 mmol, 79% yield) as a tan solid. LRMS (ESI): m/z 621.3 [M⁺Na]⁺, calculated for C₃₃H₃₄N₄O₇ m/z 621.2.

### Preparation of (9H-fluoren-9-yl)methyl 1,2-dimethyl-2-((1-(3-oxo-3-(perfluorophenoxy)propyl)-5-(4-oxo-4-(perfluorophenoxy)butanamido)-1H-indol-2-yl)methyl)hydrazine-1-carboxylate (11)

A solution of diacid 10 (2.0 g, 3.34 mmol) in 40 mL of anhydrous THE was combined with 6.2 g (33.4 mmol) of pentafluorophenol. The mixture was stirred and treated with DCC (2.0 g, 10 mmol) in a few small portions at room temperature. Reaction mixture was stirred for 48 hours at RT, then all solids were removed by filtration and washed with THE on filter. Combined filtrates were concentrated under vacuum and purified by silica gel chromatography (0-25-35% gradient of EtOAc-Hexanes) to give 2.4 g of bis-PFP ester 11 (2.6 mmol, 77% yield) as a white foaming solid. LRMS (ESI): m/z 931.2 [M+H]⁺, calculated for C₄₅H₃₂F₁₀N₄O₇ m/z 931.2.

### Synthesis of linker-payload (Ha)

### Preparation of (2S,3S,4S,5R,6S)-6-(2-((S)-2-((S)-2-amino-3-methylbutanamido)propanamido)-5-((((2-((S)-4-ethyl-4-hydroxy-3,14-dioxo-3, 4,12,14-tetrahydro-1H-pyrano[3′,4′:6, 7]indolizino[1,2-b]quinolin-]1-yl)ethyl)(isopropyl)carbamoyl)oxy)methyl)phenoxy)-3,4,5-trihydroxytetrahydro-2H-pyran-2-carboxylic acid (15)

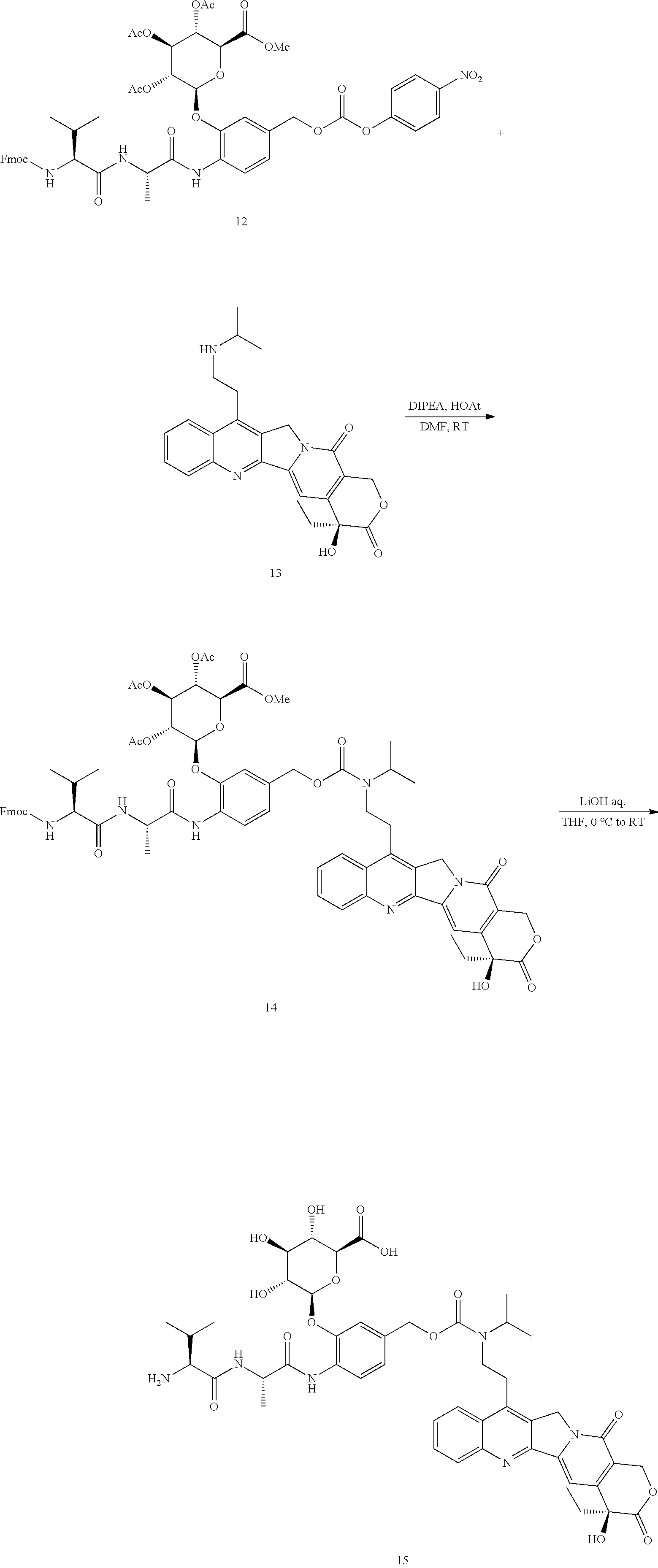

Belotecan hydrochloride 13 (2.35 g, 5.0 mmol) was suspended in a mixture of 30 mL of anhydrous DMIF and 1.75 mL of DIPEA (10 mmol). The resulting mixture was stirred and treated with HOAt (0.68 g, 5 mmol), followed by PNP-carbonate 12 (5.1 g, 5 mmol) in small portions at room temperature. Reaction mixture was stirred at RT for 8 h until starting materials were judged fully consumed based on HPLC analysis. The mixture was poured onto 300 mL of ice with vigorous stirring, the resulting yellowish precipitate was collected by filtration, washed with 30 mL of water twice, dried on air overnight to give 6.7 g of crude coupling product 14 as a light-yellow powder. LRMS (ESI): m/z 1307.5 [M+H]⁺, calculated for C₆₉H₇₄N₆O₂₀ m/z 1307.5.

A solution of crude intermediate 14 (6.7 g) in 30 mL of THF was cooled down to 0° C. in an ice bath and treated slowly with 2 M aqueous lithium hydroxide solution (10 mL). Reaction mixture was stirred at 0° C. for 1 h, then another 10 mL of 2 M LiGH solution was added and stirring continued for 15 minutes before warming the reaction mixture to room temperature and adding another 10 mL of 2 M lithium hydroxide and 5 mL of methanol. The resulting mixture was stirred for 1 h at room temperature, then quenched by adding 2 M aqueous HCl solution to pH 2-3 and let stir for 30 minutes. The mixture was transferred to a separatory funnel and washed with MTBE (2×50 mL). Aqueous layer was separated and directly purified by reversed-phase chromatography (C18 column, 0-40% CH3CN-H₂O with 0.05% TFA). Pure fractions were combined, concentrated under reduced pressure, and lyophilized to give 3.4 g of product 15 (3.6 mmol, 72% yield over 2 steps) as a bright-yellow fluffy powder. LRMS (ESI): m z 945.4 [M+H]⁺, calculated for C₄₇H₅₆N₆O₁₅ m/z 945.4.

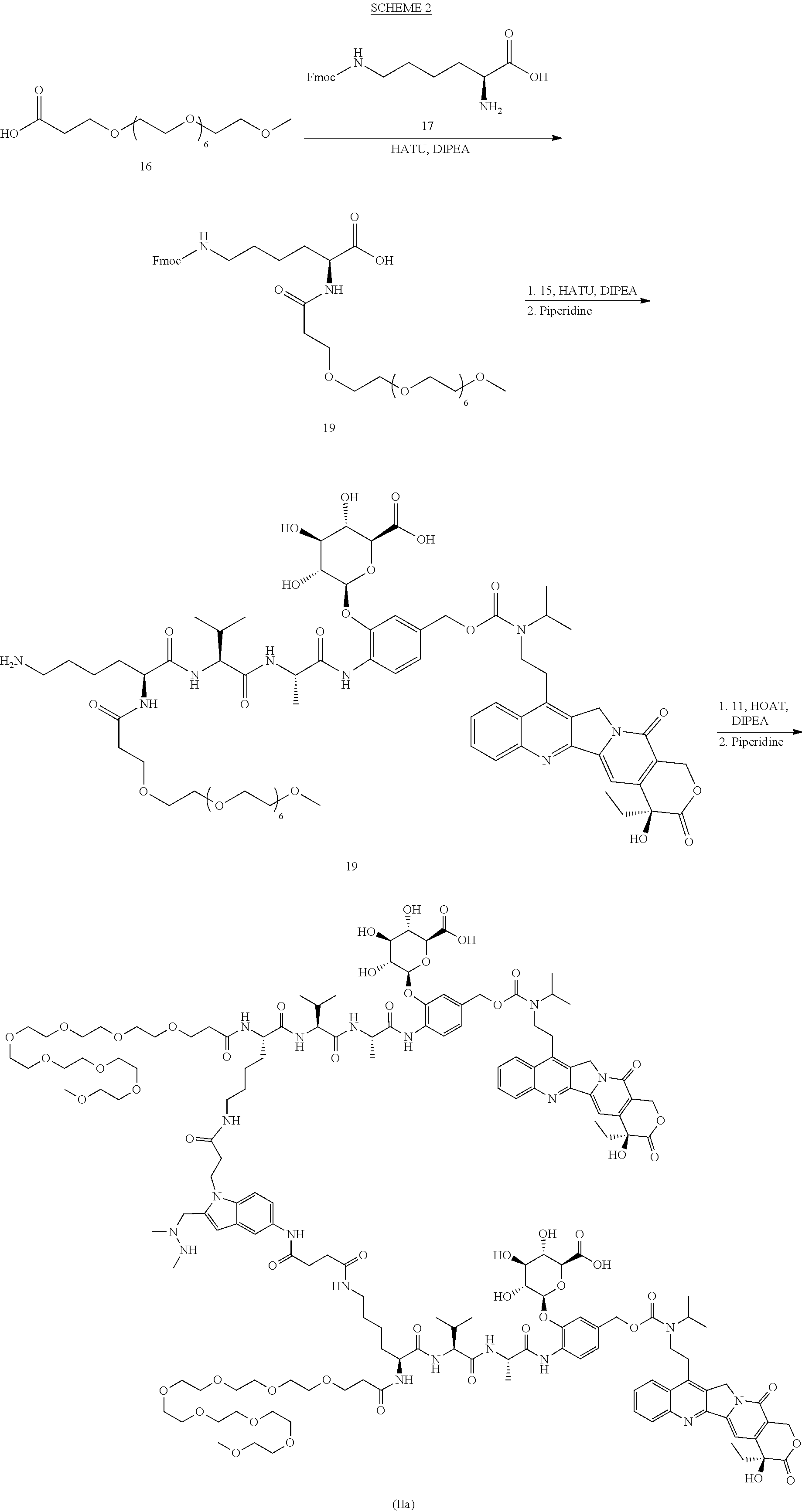

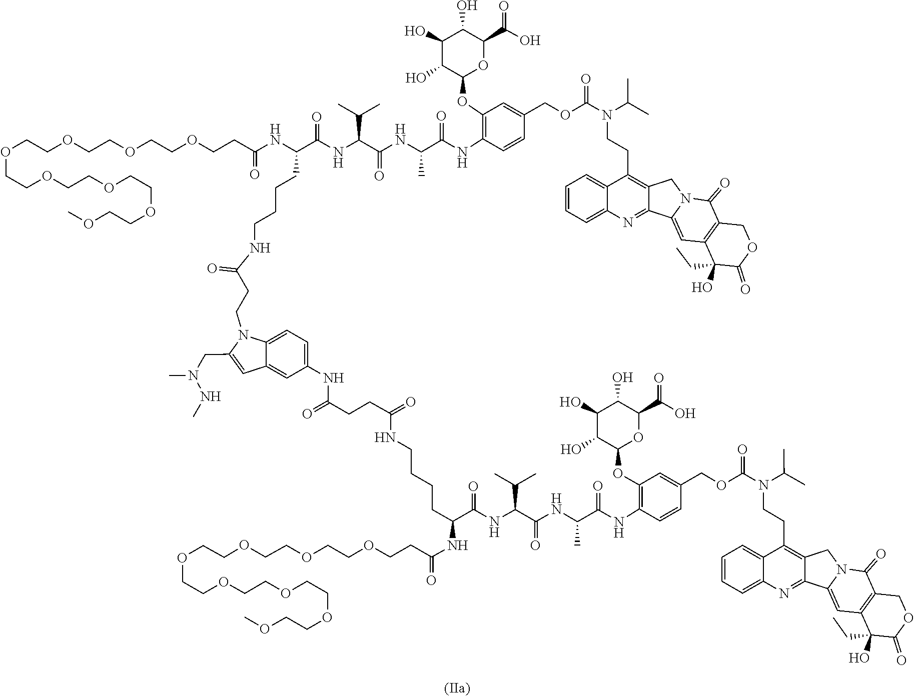 [SMILES: CC[C@@]1(O)C(=O)OCc2c1cc1n(c2=O)Cc2c-1nc1ccccc1c2CCN(C(=O)OCc1ccc(NC(=O)[C@H](C)NC(=O)[C@@H](NC(=O)[C@H](CCCCNC(=O)CCC(=O)Nc2ccc3c(c2)cc(CN(C)NC)n3CCC(=O)NCCCC[C@H](NC(=O)CCOCCOCCOCCOCCOCCOCCOCCOC)C(=O)N[C@H](C(=O)N[C@@H](C)C(=O)Nc2ccc(COC(=O)N(CCc3c4c(nc5ccccc35)-c3cc5c(c(=O)n3C4)COC(=O)[C@]5(O)CC)C(C)C)cc2O[C@@H]2O[C@H](C(=O)O)[C@@H](O)[C@H](O)[C@H]2O)C(C)C)NC(=O)CCOCCOCCOCCOCCOCCOCCOCCOC)C(C)C)c(O[C@@H]2O[C@H](C(=O)O)[C@@H](O)[C@H](O)[C@H]2O)c1)C(C)C]

### Preparation of N⁶-(((9H-fluoren-9-yl)methoxy)carbonyl)-N²-(3-(2-(2-methoxyethoxy)ethoxy)propanoyl)-L-lysine (18)

Carboxylic acid 16 (5.0 g, 12 mmol) was dissolved in anhydrous DMF (10 mL) and treated with DIPEA (2.1 mL, 12 mmol), followed by HATU (4.6 g, 12 mmol) at ambient temperature. The resulting mixture was stirred for 30 minutes and then combined with solid H-Lys(Fmoc)—OH 17 (4.5 g, 12 mmol). Reaction mixture was stirred for 1 h at RT and then directly purified by reversed-phase chromatography (C18 column, 0-70% CH₃CN—H₂O with 0.05% TFA). Fractions containing product were combined and solvents were removed in vacuum. The residue was dried under high vacuum overnight to give product 18 (5.5 g, 7.2 mmol, 60% yield) as a clear colorless oil. LRMS (ESI): m/z 763.4 [M+H]⁺, calculated for C₃₉H₅₈N₂O₁₃ m/z 763.4.

### Preparation of (2S,3S,4S,5R,6S)-6-(2-((28S,31]S,34S)-28-(4-aminobutyl)-31-isopropyl-34-methyl-26,29,32-trioxo-2, 5,8,11,14,17,20,23-octaoxa-27,30,33-triazapentatriacontan-35-amido)-5-((((2-((S)-4-ethyl-4-hydroxy-3,14-dioxo-3, 4,12,14-tetrahydro-1H-pyrano[3′,4′:6, 7]indolizino[1,2-b]quinolin-]1-yl)ethyl)(isopropyl)carbamoyl)oxy)methyl)phenoxy)-3,4,5-trihydroxytetrahydro-2H-pyran-2-carboxylic acid (19)

Carboxylic acid 18 (2.9 g, 3.8 mmol) was combined with DIPEA (1.3 mL, 7.6 mmol) and HATU (1.45 g, 3.8 mmol) in 12 mL of anhydrous DMF at room temperature. The mixture was stirred for 20 minutes before combining with a solution of compound 15 (3.3 g, 3.5 mmol) in 15 mL of DMF. The resulting mixture was stirred at room temperature for 1 hour, then treated with 13 mL of triethylamine and stirred at room temperature for 7 h until reaction was judged complete by LCMS analysis. Reaction mixture was then concentrated under reduced pressure to remove triethylamine and purified by reversed-phase chromatography (C18 column, 0-40% CH₃CN—H₂O with 0.05% TFA). Pure fractions were combined, concentrated under reduced pressure to −100 mL final volume, and lyophilized to give 3.5 g of product 19 as a bright-yellow light powder (2.4 mmol, 69% yield over 2 steps). LRMS (ESI): m/z 1467.7 [M+H]⁺, calculated for C₇₁H₁₀₂N₈O₂₅ m/z 1467.7.

### Preparation of (2S,3S,4S,5R,6S)-6-(2-((28S,31S, 34S)-28-(4-(3-(5-((S)-28-(((S)-1-(((S)-1-((2-(((2S,3R,4S,5S,6S)-6-carboxy-3,4,5-trihydroxytetrahydro-2H-pyran-2-yl)oxy)-4-((((2-((S)-4-ethyl-4-hydroxy-3,14-dioxo-3, 4,12,14-tetrahydro-1H-pyrano[3′, 4′: 6, 7]indolizino[1,2-b]quinolin-]1-yl)ethyl)(isopropyl)carbamoyl)oxy)methyl)phenyl)amino)-1-oxopropan-2-yl)amino)-3-methyl-1-oxobutan-2-yl)carbamoyl)-26,34-dioxo-2, 5,8,11,14,17,20,23-octaoxa-27,33-diazaheptatriacontan-37-amido)-2-((1,2-dimethylhydrazineyl)methyl)-1H-indol-1-yl)propanamido)butyl)-31-isopropyl-34-methyl-26,29,32-trioxo-2, 5,8,11,14,17,20,23-octaoxa-27,30,33-triazapentatriacontan-35-amido)-5-((((2-((S)-4-ethyl-4-hydroxy-3,14-dioxo-3,4,12,14-tetrahydro-1H-pyrano[3′,4′: 6, 7]indolizino[1,2-b]quinolin-]]-yl)ethyl)(isopropyl)carbamoyl)oxy)methyl)phenoxy)-3,4,5-trihydroxytetrahydro-2H-pyran-2-carboxylic acid (20)

To a stirred solution of compound 19 (3.5 g, 2.4 mmol) in 16 mL of anhydrous DMA were added DIPEA (0.84 mL, 4.8 mmol) and HOAt (0.33 g, 2.4 mmol) at room temperature. The resulting mixture was treated with a separately prepared solution of bis-PFP ester 11 ester (1.0 g, 1.1 mmol in 2 mL of DMA) in a few small portions with 10 minutes intervals between additions. After the addition was complete, reaction mixture was stirred for 15 minutes at room temperature and treated with 2.1 mL of piperidine (22 mmol). After 20 minutes, reaction mixture was directly purified by reversed-phase chromatography (C18 column, 0-40% CH₃CN—H₂O with 0.05% TFA). Pure fractions were combined, concentrated under reduced pressure at 30° C., and lyophilized to obtain 3.2 g of linker-payload (IIa) as a bright-yellow fluffy powder (0.98 mmol, 89% yield of two steps based on 11). LRMS (ESI): m z 1638.3 [M+H]²⁺, calculated for C₁₆₀H₂₂₄N₂₀O₅₃ m/z 1638.8.

### C. Bioconjugation and Purification of TF-ADCS

Aldehyde-tagged antibodies (15 mg/mL) were conjugated to synthesized linker-drugs (17.5 mol. equivalents drug:antibody for DAR4 and 22.5 mol. equivalents drug:antibody for DAR8) by reacting the linker-drug and aldehyde-tagged antibody for 72 hours at 37° C. in 67 mM sodium citrate, pH 5.5, 43 mM sodium chloride containing 3.5 or 4.5% DMA.

After conjugation, residual free drug was removed by diafiltration using a 115V Labscale Tangential Flow Filtration (TFF) system (Millipore, Cat. No. XX42LSS11) with a 30 kDa TFF cassette (Sigma-Aldrich, Cat. No. P3C030C00, filtration area 88 cm², NMWCO 30 kDa) and exchanging for 12 diavolumes (600 mL) into 20 mM sodium citrate, pH 5.5, 50 mM NaCl.

After the TFF purification, the TF-ADC 6-4 sample was measured as having about 2.5% free payload, while the TF-ADC 6-8 sample was measured as having about 18.5% free payload.

In some embodiments, residual free drug was further removed using SEC-FPLC (HiLoad 26/600 Superdex® 200) followed by concentration using AMICON® 0.5 mL 30 kD molecular weight cut off (MWCO) centrifugal filters (Millipore Sigma, Cat. No. #UFC5030BK). After the FPLC purification, the TF-ADC samples were measured as having about 1.4% free payload.

In some embodiments, ion exchange chromatography is further used to purify the TF-ADC samples.

To determine the drug-to-antibody ratio (DAR) of the final product, antibody-drug conjugates were examined by analytical hydrophobic interaction chromatography (HIC) or polymeric reverse phase chromatography (PLRP).

The HIC column (Tosoh Bioscience, Cat. No. 14947) was run with 1.5 M ammonium sulfate and 25 mM sodium phosphate (pH 7.0) as mobile phase A and 25% isopropanol and 18.75 mM sodium phosphate (pH 7.0) as mobile phase B. The PLRP column (Agilent, Cat. No. PL1912-1802) was run with 0.1% trifluoroacetic acid in H₂O as mobile phase A and 0.1% trifluoroacetic acid in CH₃CN for mobile phase B, with the column heated to 80° C.

In some embodiments, analytical Hydrophobic Interaction Chromatography (HIC) was performed as described below: Samples were analyzed on an Agilent 1100 Instrument equipped with a 4.6 mm×35 mm TSK gel butyl-NPR column from Tosoh (#14947) held at 25° C. Samples were prepared by adding 20 μL of 50 mM NaCl (pH 5.5) and 20 μL of Mobile Phase A to 20 μg of sample and were injected at room temperature. Gradient elution utilized mobile phase A (0.1% trifluoroacetic acid in H2O) and Mobile Phase B (0.1% trifluoroacetic acid in CH3CN) in the program reported in the table below at a flow rate of 1.0 mL/minute. Detection was via UV at 215 nm, 252 nm, and 280 nm.

In some embodiments, analytical Hydrophobic Interaction Chromatography (HIC)(Tosoh #14947) with UV/Vis detection was performed. The reduced form of the final product was analyzed by reverse phase liquid chromatography (RP-HPLC)(Agilent Cat. No. PL1912-1802) with UV/Vis detection to verify DAR.

TABLE E1
GRADIENT ELUTION PROGRAM FOR HIC ANALYSES

| Time (min) | Mobile Phase B (%) |
|---|---|
| 0.0 | 12 |
| 5.5 | 42 |
| 7.5 | 70 |
| 8.5 | 100 |
| 9.5 | 100 |
| 10 | 12 |
| 12 | 12 |

In some embodiments, Reverse Phase HPLC was performed as described below: Samples were analyzed on an Agilent 1100 Instrument equipped with a 50×2.1 mm PL1912-1802 (8 μm, 1000 Å) column from Agilent held at 80° C. Samples were prepared by adding 10 μL of a mixture of 0.5 mM DTT, 50 μL of 8 M guanidine HCl, 130 mM tris, and 1 mM EDTA (pH 7.6) to a 20 μg sample and adding PBS to a final volume of 100 μL. Samples were incubated at 37° C. for 30 minutes after preparation. Gradient elution utilized mobile phase A (25 mM Na₃PO₄, 1.5 M (NH₄)₂SO₄, pH 7.0) and Mobile Phase B (18.75 mM Na₃PO₄, pH 7.0, 25% isopropyl alcohol) in the program reported in the table below at a flow rate of 2.0 mL/minute. Detection was via UV at 215 nm, 252 nm, and 280 nm.

| Time (min) | Mobile Phase B (%) |
|---|---|
| 0.0 | 25 |
| 8 | 50 |
| 8.1 | 95 |
| 10 | 95 |

The table below reports the DAR for each ADC tested.

TABLE E2
REPRESENTATIVE TEST LOTS

| | Titer | Titer | | | | Reverse | Reverse |
|---|---|---|---|---|---|---|---|
| | (mg/L) | (mg/L) | | % | HIC Rₜ | Phase Rₜ | Phase Rₜ |
| ADC | round 1 | round 2 | DAR | Aggregate | (mAb)\hat{ } | (LC)\hat{ } | (HC)\hat{ } |
| TF-ADC 6-8 | 240 | 389 | 6.56 | 3.7 | 6.1 | 7.0 | 6.6 |
| TF-ADC 7-8 | 336 | 305 | 6.53 | 3.2 | 5.7 | 6.2 | 6.7 |
| TF-ADC 6-4 | 420 | 430 | 3.4 | 3.8 | | | |
| TF-ADC 7-4 | 355 | 297 | 3.14 | 4.4 | | | |

To determine aggregation, samples were analyzed using analytical size exclusion chromatography (Tosoh Bioscience, Cat. No. 08541) using a mobile phase of 300 mM NaCl, 25 mM sodium phosphate, and 500 isopropanol (pH 6.8).

Representative results are provided in the tables below. In one example run, 2.1 g TF-ADC 6-4 and 1.2 g TF-ADC 6-8 were produced, each of which had a concentration higher than 12 mg/mL.

TABLE E3
POST-TFF - REPRESENTATIVE TEST LOTS

| | | DAR | | | Free | Total | ADC |
|---|---|---|---|---|---|---|---|
| | Concentration | (RP, | Aggregation | Endotoxin | Drug | amount | Purification |
| ADC | (mg/mL) | HIC) | (%) | (EU/mg) | (%) | (g) | Yield (%) |
| TF-ADC 6-4 | 13.0 | 3.7, | 5.2 | <0.01 | 1.5, | 2.7 | 78.4 |
| | | 3.7 | | | 3.4 | | |
| TF-ADC 6-8 | 13.9 | 7.0, | 2.9 | 0.01 | 15, | 1.7 | 72.1 |
| | | 7.4 | | | 22 | | |

TABLE E4
POST-SEC-FPLC - REPRESENTATIVE TEST LOTS

| | concentra- | DAR | Aggrega- | | Free | Total |
|---|---|---|---|---|---|---|
| | tion | (RP, | tion | Endotoxin | Drug | amount |
| ADC | (mg/mL) | HIC) | (%) | (EU/mg) | (%) | (g) |
| TF-ADC | 8.96 | 7.07, | 4.0 | 0.02 | 1.4 | 1.4 |
| 6-8 | | 7.06 | | | | |

TABLE E5
DRUG-TO-ANTIBODY (DAR) RATIOS AND
% HIGH-MOLECULAR WEIGHT SPECIES (%
HMW) - REPRESENTATIVE TEST LOTS.

| | Concentration | DAR | % HMW | Endotoxin |
|---|---|---|---|---|
| Sample name | (mg/mL) | (PLRP) | (SEC) | (EU/mg) |
| FITC-ADC 8 | 2.58 | 6.50 | 2.3 | <0.19 |
| TF-ADC 6-8 | 2.56 | 6.56 | 3.7 | <0.19 |
| TF-ADC 7-8 | 2.73 | 6.53 | 3.2 | <0.19 |
| FITC-ADC 4 | 3.96 | 3.45 | 2.4 | 0.29 |
| TF-ADC 6-4 | 2.65 | 3.40 | 3.8 | <0.19 |
| TF-ADC 7-4 | 2.61 | 3.14 | 4.4 | <0.20 |

TABLE E6

| Drug/ | | Tag | | % | Concentration | EU | |
|---|---|---|---|---|---|---|---|
| Linker | Ab | Site(s) | DAR | aggregate | mg/mL | (mg) | Note |
| IIa | FITC | CH1/CT | 6.5 | 2.3 | 2.58 | <0.19 | Referred to |
| | | | | | | | herein as |
| | | | | | | | Isotype or |
| | | | | | | | Isotype 2 |
| TF-ADC-6-8 | | CH1/CT | 6.56 | 3.7 | 2.56 | <0.2 | |
| IIa | EXMA-006 | | | | | | |
| TF-ADC-7-8 | | CH1/CT | 6.53 | 3.2 | 2.73 | <0.18 | |
| IIa | EXMA-007 | | | | | | |
| IIa | FITC | CH1 | 3.45 | 2.4 | 3.96 | 0.29 | Referred to |
| | | | | | | | herein as |
| | | | | | | | Isotype or |
| | | | | | | | Isotype 1 |
| TF-ADC-6-4 | | CH1 | 3.4 | 3.8 | 2.65 | <0.19 | |
| IIa | EXMA-006 | | | | | | |
| TF-ADC-7-4 | | CH1 | 3.14 | 4.4 | 2.61 | <0.19 | |
| IIa | EXMA-007 | | | | | | |

### Example 2-TF-ADC Characterization

Samples for protein thermal shift experiments (melting temperature) were prepared using the APPLIED BIOSYSTEMS™ PROTEIN THERMAL SHIFT™ Dye Kit (ThermoFisher Scientific Cat. No. 4461146). Data were collected using a QUANTSTUDIO™ 3 instrument (ThermoFisher Scientific Cat. No. A28567) with PROTEIN THERMAL SHIFT™ Software vl.4 (ThermoFisher Scientific Cat. No. 466038). Briefly, PROTEIN THERMAL SHIFT™ dye (1000X) was diluted in water to 8X in deionized water. Samples were prepared on ice by combining 5 μL of PROTEIN THERMAL SHIFT™ Buffer, 12.5 μL of protein solution (at 1 mg/mL), and 2.5 μL of the diluted PROTEIN THERMAL SHIFT™ Dye (8X) for a total reaction solution volume of 20 μL. The reaction solution was mixed 10 times with a pipette. Tubes were then spun down at 1000 rpm for 1 minute and then incubated on ice until analysis. Samples were loaded onto the instrument plate, which were then loaded into a QUANTSTUDIO™ 3 Real-Time PCR System instrument. Data was collected using the standard Melt Curve experiment with the following conditions: Step 1 temperature: 25° C. for 2 minutes; Step 2 temperature: 99° C. for 2 minutes with continuous fast ramp mode (Step 1: 1.6° C./see; Step 2: 0.05° C./sec). Raw melting temperature data were deconvoluted within the QUANTSTUDIO™ 3 analysis software.

The table below reports the measured melting temperature of the ADCs.

TABLE E7

| Construct | CH2 Tₘ (°C) | Fab Tₘ (°C) |
|---|---|---|
| EXMA-006 | 71 | 75.6 |
| TF-ADC 6-4 (DAR 3.68) | — | 67.5* |
| TF-ADC 6-8 (DAR 7.07) | 64 | 72.5 |

*CH2 and Fab Tₘ coalesced

### Example 3—Binding of TF-ADCS to Tf

Binding assays were employed to determine the relative affinity of each TF-ADC obtained in the Examples above to a TF protein. TF proteins of four species were tested separately, including human, cynomolgus (cyno), mouse, and rat. A TF antibody free of a drug-linker construct was tested in parallel with the ADCs, serving as a control. Anti-FITC antibody conjugated with the same linker-payload construct was also used as a negative control.

The TF proteins were produced. The interaction of the TF-ADC (or the TF antibody) to the TF protein (e.g., a human, cynomolgus (cyno), mouse, or rat TF) was monitored using an Octet (Pall ForteBio) instrument. For these assays, 36 nM antibody was immobilized on anti-human Fc (AHC) sensors in 1X kinetic buffer (ForteBio). The sensors were then dipped in wells containing a 3X serial dilution of the TF protein ranging from 1 μM to 1.37 nM. The association was allowed to reach equilibrium. Sensors were then moved to wells containing only 1X kinetic buffer and allowed to dissociate to equilibrium. Association and dissociation rates were measured and from these, monovalent KDs were calculated using the onboard analysis software (ForteBio).

Exemplary data are shown in the tables below.

TABLE E8A
QUALITATIVE BINDING AFFINITY BY OCTET

| Ligand | Analyte | kₐ (1/Ms) | k_{d} (1/s) | K_{D}(M) |
|---|---|---|---|---|
| EXMA-006 - | Cynomolgus Tissue | 7.10E+5 | 4.78E−04 | 6.73E−10 |
| representative batch 1 | Factor | | | |
| EXMA-006 - | Human Tissue | 8.31E+05 | 9.38E−04 | 1.13E−09 |
| representative batch 1 | Factor | | | |
| EXMA-006 - | Mouse Tissue Factor | 7.55E+05 | 0.08101 | 1.07E−07 |
| representative batch 1 | | | | |
| EXMA-006 - | Rat Tissue Factor | NA | NA | NA |
| representative batch 1 | | | | |

TABLE E8B
QUALITATIVE BINDING AFFINITY BY OCTET

| Ligand | Analyte | kₐ (1/Ms) | k_{d} (1/s) | K_{D}(M) |
|---|---|---|---|---|
| EXMA-006 - | Cynomolgus Tissue | 9.4E+03 | 2.2E−01 | 2.4E−04 |
| representative batch 2 | Factor | | | |
| EXMA-006 - | Human Tissue Factor | 6.5E+03 | 2.3E−01 | 3.6E−05 |
| representative batch 2 | | | | |
| EXMA-006 - | Mouse Tissue Factor | 3.2E+02 | 6.3E−01 | 2.0E−03 |
| representative batch 2 | | | | |
| TF-ADC 6-4 | Cynomolgus Tissue | 1.4E+04 | 1.6E−01 | 1.1E−05 |
| | Factor | | | |
| TF-ADC 6-4 | Human Tissue Factor | 8.2E+03 | 1.3E−01 | 1.6E−05 |
| TF-ADC 6-4 | Mouse Tissue Factor | 3.3E+03 | 4.7E−01 | 1.4E−04 |
| TF-ADC 6-8 | Cynomolgus Tissue | 1.0E+04 | 1.9E−01 | 1.9E−05 |
| | Factor | | | |
| TF-ADC 6-8 | Human Tissue Factor | 1.5E+04 | 1.1E−01 | 7.1E−06 |
| TF-ADC 6-8 | Mouse Tissue Factor | 2.5E03 | 5.4E−01 | 2.1E−04 |

### Example 4—Pharmacokinetic (PK) Analysis of TF-ADCS in Rats

Male Sprague-Dawley rats (3 per group) were dosed intravenously with a single 5 mg/kg bolus of the TF-ADC produced as described in the examples above. K2EDTA-stabilized plasma was collected at 0.5 h, 6 h and 24 h, and 2, 4, 7, 10, 14 and 21 days post-dose.

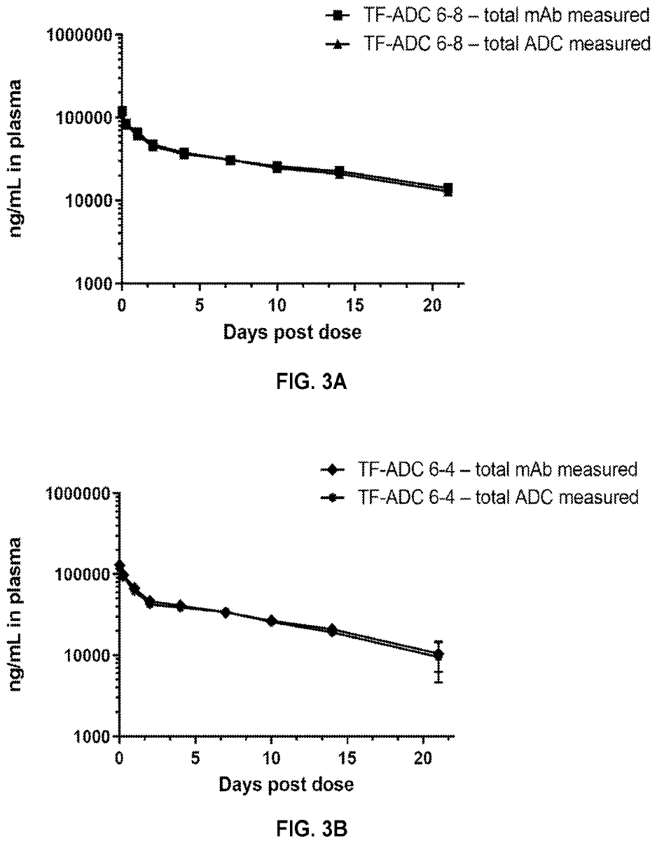

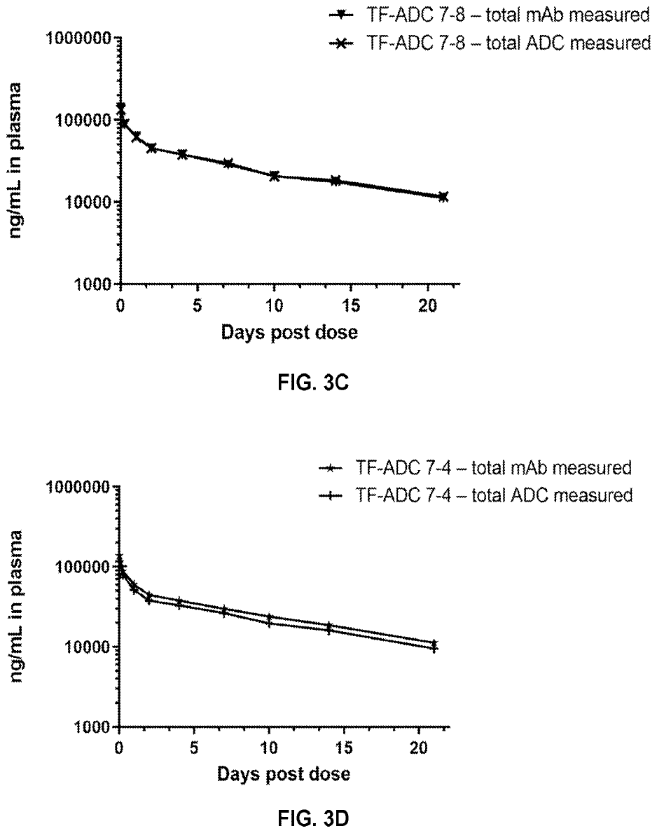

Total antibody and total ADC concentrations were quantified by ELISA. For total antibody measurements, conjugates were captured with an anti-human IgG-specific antibody and detected with an HRP-conjugated anti-human Fc-specific antibody. For total ADC measurements, conjugates were captured with an anti-human Fab-specific antibody and detected with a mouse anti-payload primary antibody, followed by an HRP-conjugated anti-mouse IgG-subclass 1-specific secondary antibody. Bound secondary antibody was detected using Ultra TMB One-Step ELISA substrate (ThermoFisher). After quenching the reaction with sulfuric acid, signals were read by taking the absorbance at 450 nm on a Molecular Devices Spectra Max M5 plate reader equipped with SoftMax Pro software. Data were analyzed using GraphPad Prism and Microsoft Excel software and plotted in FIGS. 3A-3D.

### Example 5—In Vitro Efficacy of TF-ADCS in Cell Line Derived Xenograft Models

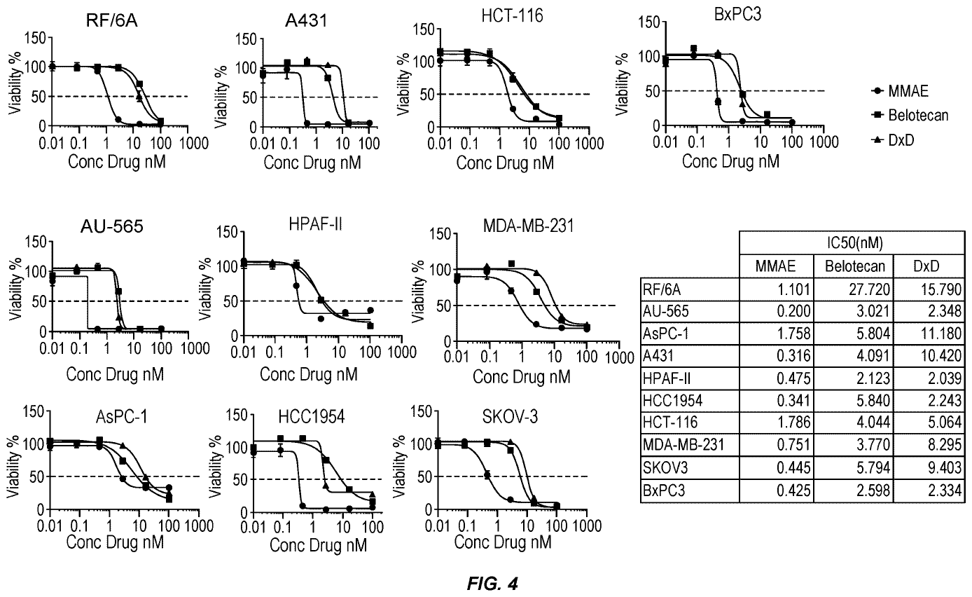

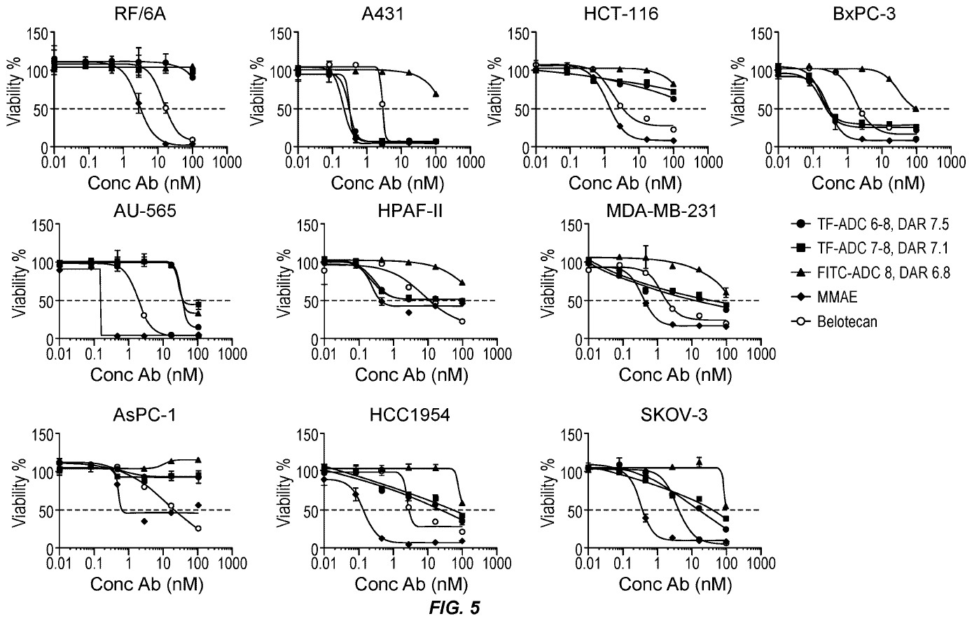

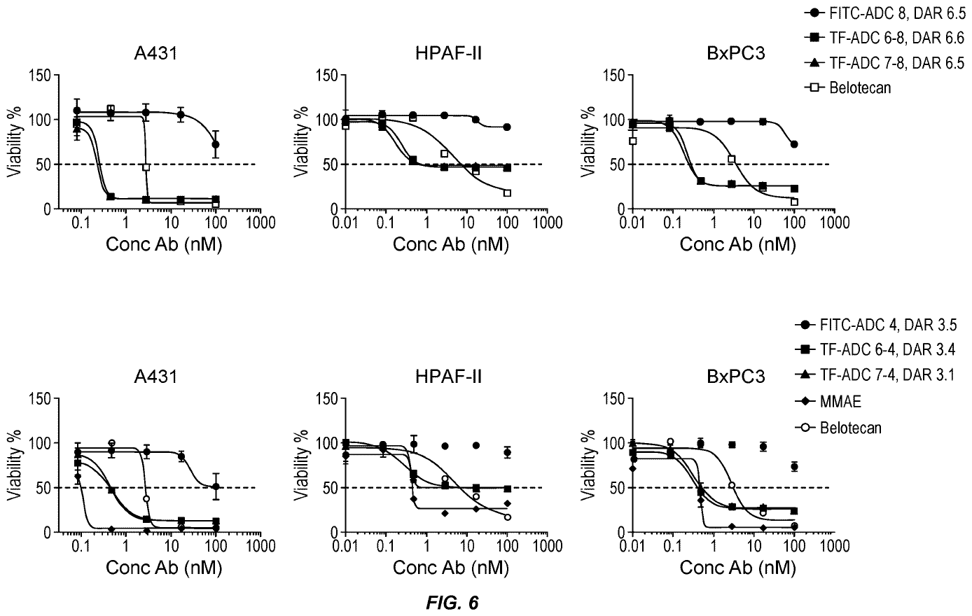

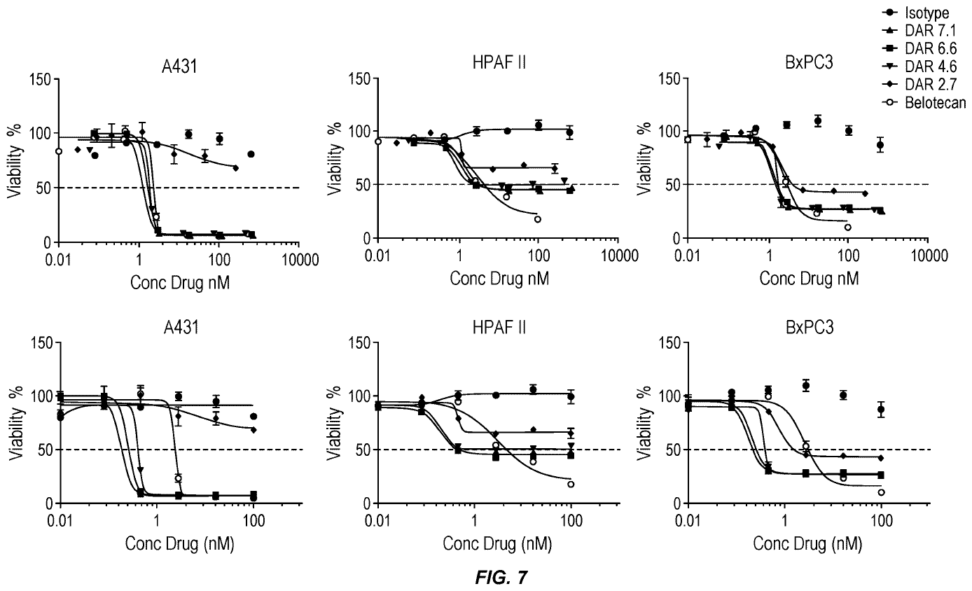

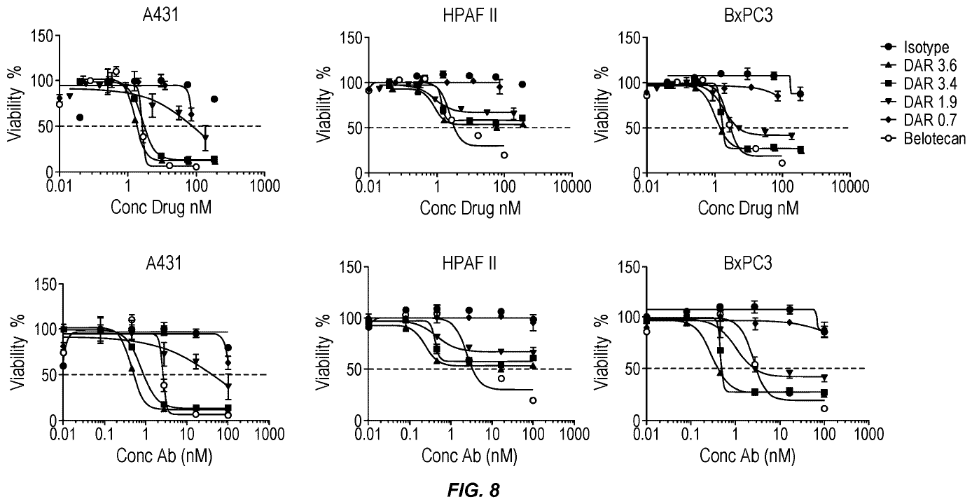

Various cancer cell lines were plated in 96-well plates (Costar 3610) at a density of 1.5×10³ cells/well in 100 μL of growth media. The next day, cells were treated with 20.L of test compounds serially-diluted in media. After incubation at 37° C. with 50 CO₂ for 5 days, viability was measured using the Promega CellTiter Glo® reagent according to the manufacturer's recommendations. GI₅₀ curves were calculated in GraphPad Prism normalized to the payload concentration. Graphs of the cytotoxicity assays (0% viability vs. drug concentration (nM)) are shown in FIGS. 4-8.

TABLE E9
BIOPHYSICAL PROPERTIES AND IC₅₀S OF TF-ADCS
FOR IN VITRO “DAR” EXPERIMENT

| | | | IC₅₀ (nM) - drug | | | IC₅₀ (nM) - mAb | | |
|---|---|---|---|---|---|---|---|---|
| | | | concentration | | | concentration | | |
| DAR | | | | HPAF- | | | HPAF- | |
| level | Sample | DAR | A431 | II | BxPC-3 | A431 | II | BxPC-3 |
| | FITC-ADC 8 | 6.36 | high | high | high | high | high | high |
| high | TF-ADC 6-8 | 7.09 | 1.635 | 1.223 | 1.361 | 0.251 | 0.189 | 0.209 |
| medium | TF-ADC 6-8 | 6.56 | 1.273 | 1.462 | 1.231 | 0.182 | 0.209 | 0.176 |
| low | TF-ADC 6-8 | 4.61 | 1.897 | 0.803 | 1.693 | 0.410 | 0.176 | 0.367 |
| very low | TF-ADC 6-8 | 2.71 | 17.910* | 1.243 | 2.076 | 6.529* | 0.454 | 0.763 |
| | belotecan | | 2.363 | 3.042 | 2.625 | 2.363 | 3.042 | 2.625 |
| | FITC-ADC 4 | 3.39 | high | high | 132.600* | 0.007* | high | 67.640* |
| high | TF-ADC 6-4 | 3.61 | 2.682 | 1.411 | 1.612 | 0.787 | 0.415 | 0.466 |
| medium | TF-ADC 6-4 | 3.4 | 1.680 | 0.859 | 1.087 | 0.463 | 0.240 | 0.302 |
| low | TF-ADC 6-4 | 1.89 | 129.100 | 0.879 | 1.955 | 67.870* | 0.457 | 1.028 |
| very low | TF-ADC 6-4 | 0.73 | 109.700 | 0.011* | 5406.0* | 187.70* | 0.012* | 7054.0* |
| | belotecan | | 2.620 | 2.448 | 2.557 | 2.620 | 2.448 | 2.557 |

*Indicates when IC₅₀s are likely not accurate due to curve shape

Bystander killing effect showing killing of adjacent non-target (i.e., non-TF) expressing cells by released payload that was taken up by the target cell was further evaluated. Briefly, a co-culture of target positive cell line MIDA-MB-231 and target negative cell line Jurkat were treated with the ADCs as disclosed herein or a vehicle control, and cytotoxicity of the treated Jurkat cells was evaluated using TrypLE™ and normalized to the vehicle control. TF-ADC 6-8 showed significant bystander cell killing activities with an IC₅₀ of about 1.203 nM, while a benchmark ADC (CAS 1418731-10-8 and INN 10148) showed an IC₅₀ of about 0.3154 nM. Cytotoxicity of the ADC in Jurkat monoculture was also evaluated in parallel. TF-ADC 6-8 showed an IC₅₀ of about 7.755 nM while the benchmark ADC showed an IC₅₀ of about 17.73 nM.

### Example 6—In Vivo Efficacy of TF-ADCS in Cell Line Derived Xenograft Models

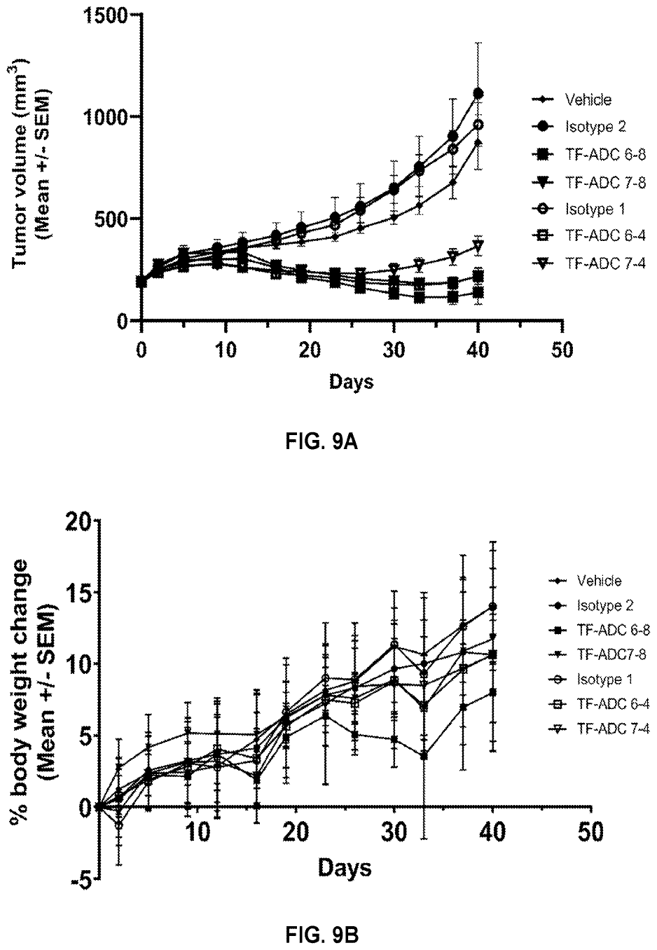

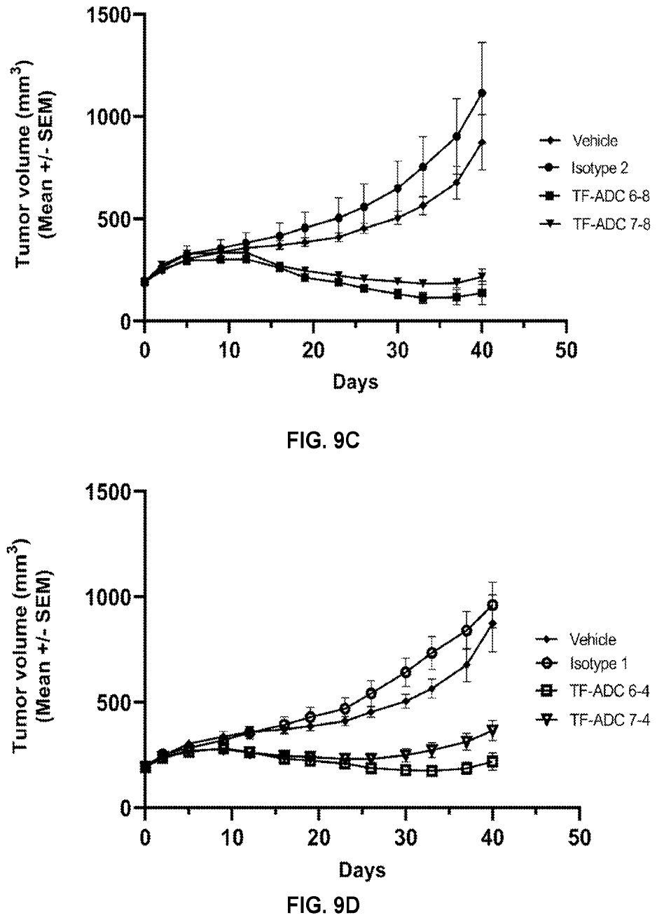

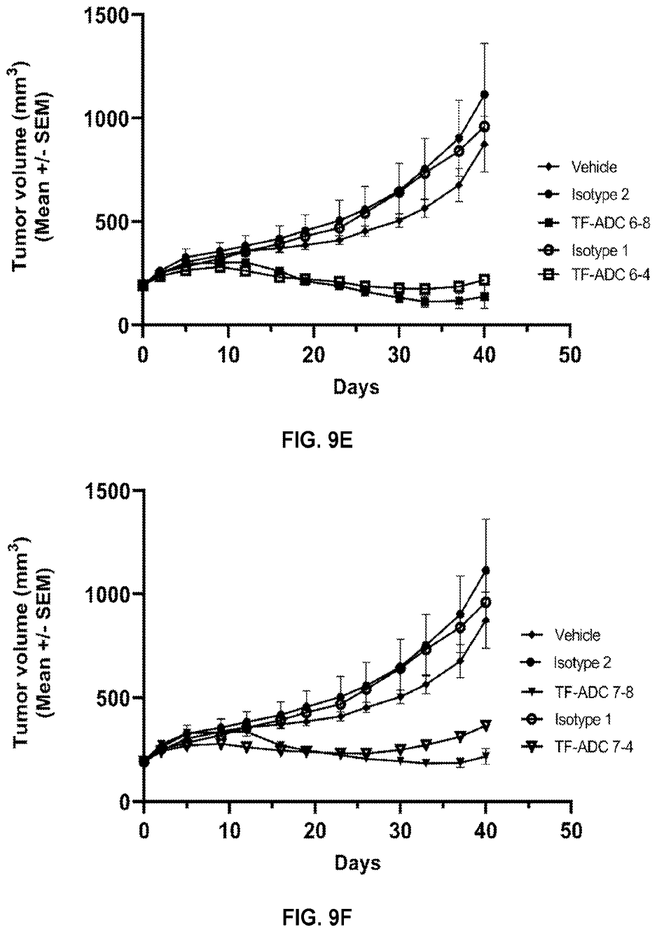

BxPC3 Pancreatic Cancer Xenograft Model: Female BalbC/nude mice (n=8 per group) were inoculated with 1×10⁷ of BxPC3 tumor cells in 1:1 MATRIGEL® matrix. When the average tumor volume reached 150-200 mm³, mice were randomized into respective treatment groups and received a single intravenous injection of vehicle, isotype control, or 10 mg/kg ADC. There were no adverse effects on body weight for any of the treatment groups through the course of the study. The effects of TF-ADC 6-4, TF-ADC 7-4, TF-ADC 6-8 and TF-ADC 7-8 on tumor regression and suppression are shown in FIGS. 9A-9F.

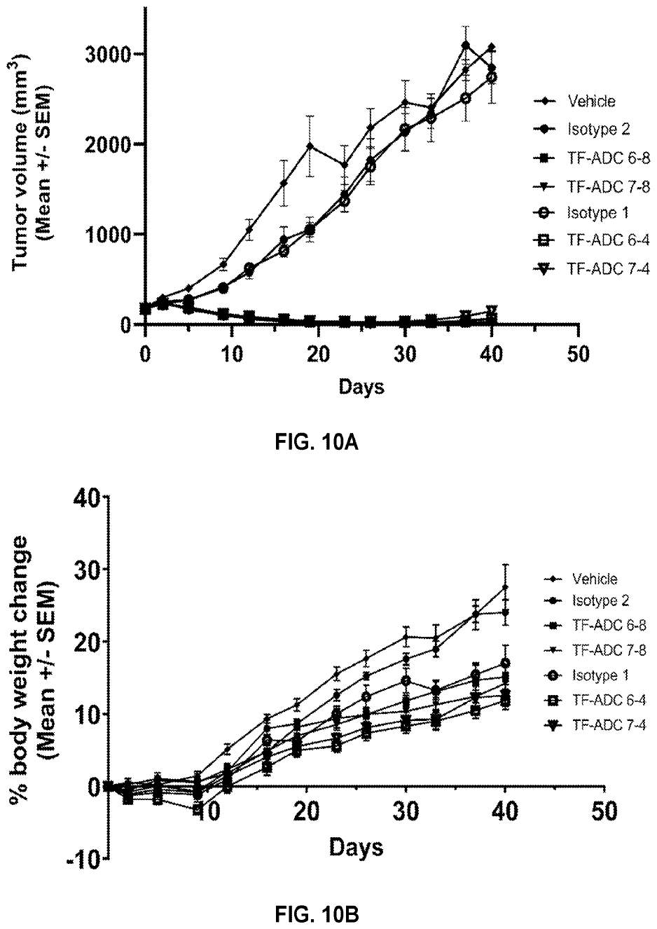

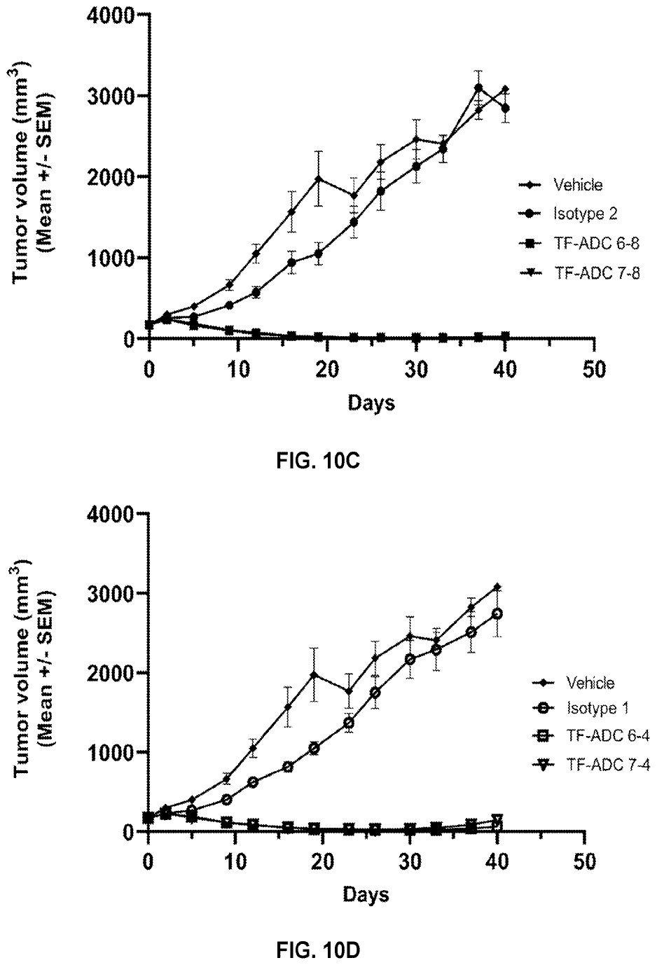

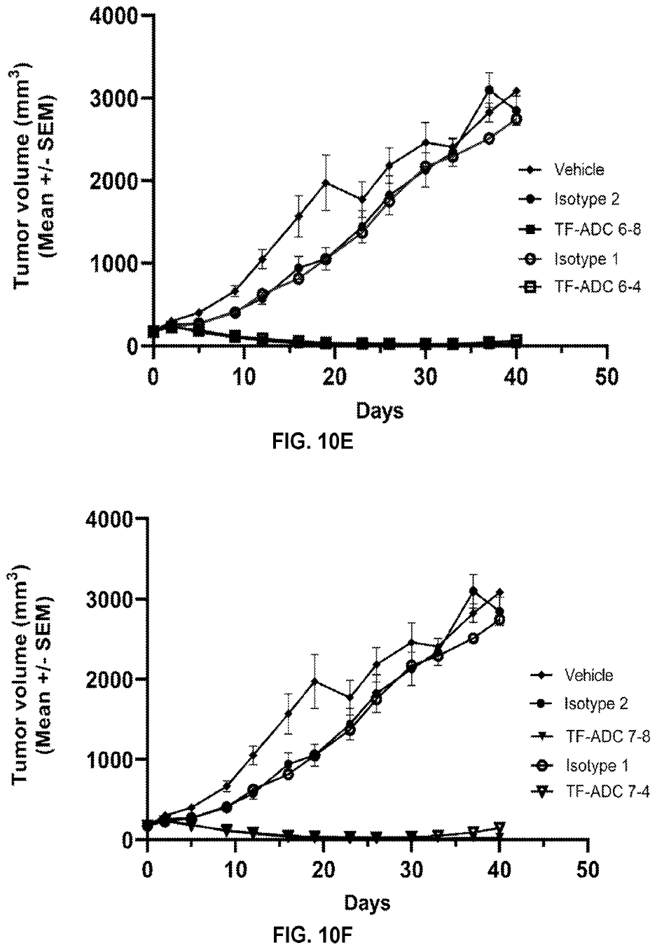

HPAF-II Pancreatic Cancer Xenograft Model: Female BalbC/nude mice (n=8 per group) were inoculated with 1×10⁷ of HPAF-II tumor cells in 1:1 MATRIGEL® matrix. When the average tumor volume reached 150-200 mm³, mice were randomized into respective treatment groups and received a single intravenous injection of vehicle, isotype control, or 10 mg/kg ADC. There were no adverse effects on body weight for any of the treatment groups through the course of the study. The effects of TF-ADC 6-4, TF-ADC 7-4, TF-ADC 6-8 and TF-ADC 7-8 on tumor regression and suppression are shown in FIGS. 10A-10F.

### Example 7—Mouse PK

TF-ADC 6-4, TF-ADC 6-8, and the corresponding TF antibody (EXMA-006) were administered to female BALB/c nude mice (8-9 weeks) at a single dosage of 3 or 10 mg/kg via IV bolus at a dose vol of 5 ml/kg (i.e., having a dosing concentration of 0.6 mg/ml or 2 mg/ml). On study day -1, animals were randomized into treatment groups based on body weight.

TABLE E10

| TA Name | Initial Concentration (mg/mL) | Amount (mg) |
|---|---|---|
| TF-ADC 6-4 | 10.58 | 5 |
| TF-ADC 6-8 | 7.58 | 5 |
| EXMA-006 | 32.1 | 5 |

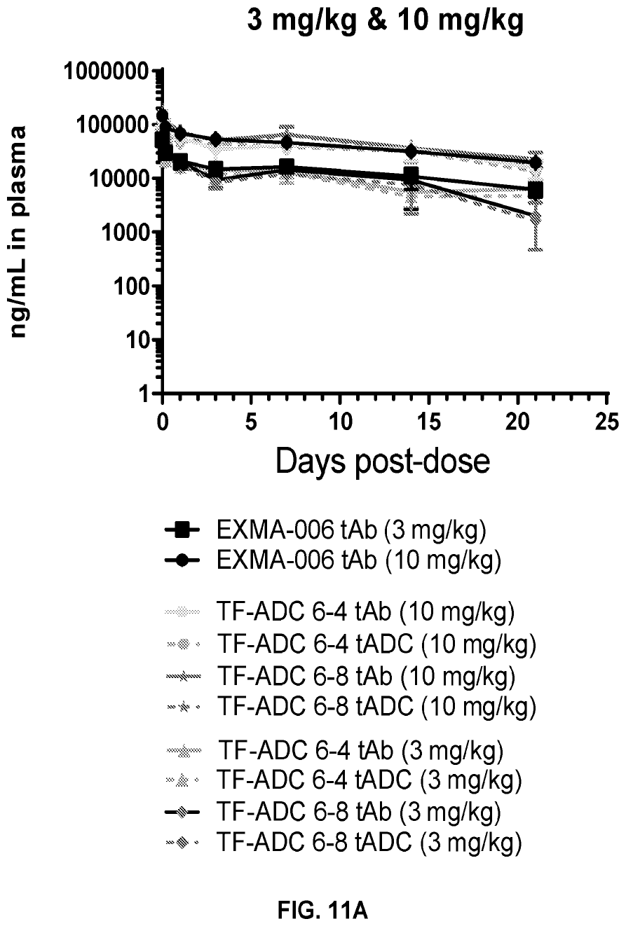

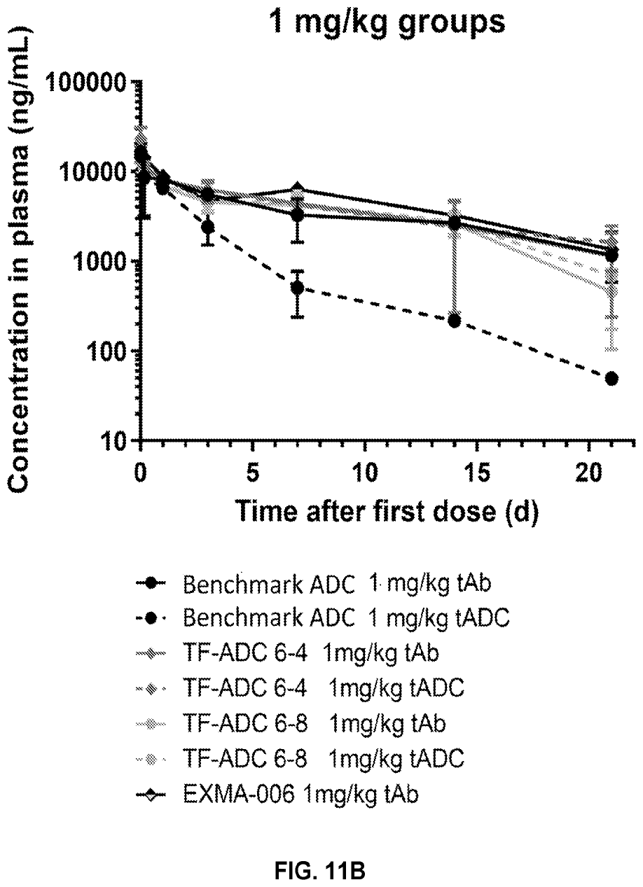

Following enrollment, treatment was initiated and continued. Body weights were recorded following enrollment (on Day 1, 7, 14, and 21). Blood samples were collected. Total antibody and total ADC concentrations were quantified by ELISA. The corresponding results are plotted in FIG. 11A.

TABLE E11

| Study | Sample | Process | Anti- | Final | Gr/ | Storage |
|---|---|---|---|---|---|---|
| Day | Type | For | Coag | Volume | An | Condition |
| See | Whole | Plasma | K2EDTA | 110 μL | All | −80 °C |
| below | Blood | | | Whole Blood | | |
| | | | | 50 μL plasma | | |

TABLE E12

| Day | Group | Animal # | Time-point(s) |
|---|---|---|---|
| 0 | All | 1-3 | 15 minutes post-dose |
| 0 | All | 4-6 | 4 hr post-dose |
| 1 | All | 7-9 | 24 hr post-dose |
| 3 | All | 1-3 | 72 hr post-dose |

On study days 7, 14 or 21, animals were anesthetized with isoflurane and bled to exsanguination followed by bilateral pneumothorax. Necropsy sample and data collection were performed.

TABLE E13

| | Process | Anti- | | | Storage |
|---|---|---|---|---|---|
| Type | For | Coag | Gr/An | Final Volume | Condition |
| Whole | Plasma | K2EDTA | All | >0.5 mL | −80 °C |
| blood, | | | | whole blood | |
| cardiac | | | | >0.25 mL | |
| | | | | plasma | |

In addition, a second study was performed to collect plasma from female BALB/c nude mice for pharmacokinetic (PK) monitoring at various time points after a single intravenous (IV) dose of TF-ADC 6-4, TF-ADC 6-8, a benchmark ADC (CAS 1418731-10-8 and INN 10148) and the corresponding TF antibody (EXMA-006).

On study day -3, the animals were randomized by body weight into treatment groups. On study day 0, the mice were dosed IV with the benchmark ADC dosed at 1, 3, or 10 mg/kg or with TF-ADC 6-4, TF-ADC 6-8, or EXMA-006 dosed at 1 mg/kg. Animal body weight measurements were taken on study days -3, 0, 7, 14, and 21. The animals were bled via retro-orbital blood draw for plasma 15 minutes post-dose (animals 1-3/group), 4 hours post-dose (animals 4-6/group), 24 hours post-dose (animals 7-9/group), and 72 hours post-dose (animals 1-3/group). See, Table E12. Whole blood (110 μL/mouse per time point) was processed to plasma (K2EDTA, 50 μL/mouse per time point). Terminal plasma was collected via cardiac blood draw on study day 7 (animals 4-6/group), day 14 (animals 7-9/group), or day 21 (animals 1-3/group). Whole blood (>0.5 mL/mouse) was processed to plasma (K2EDTA, >0.25 mL/mouse). The study was completed successfully including animal weighing, dosing, and biological sample collection. Plasma samples were stored frozen at −80° C. until testing. All animals survived to the scheduled termination points.

Total antibody and total ADC concentrations were quantified. The corresponding results are plotted in FIG. 11B.

### Example 8—Dose-Response HPAF-II Xenograft Study

The HPAF-II (CVCL_0313) tumor cells were maintained in vitro with MEM medium supplemented with 10% fetal bovine serum, 1% NEAA and sodium pyruvate at 37° C. in an atmosphere of 5% CO₂ in air. The cells in exponential growth phase were harvested and quantitated by cell counter before tumor inoculation.

Female BalbC/nude mice were inoculated subcutaneously in the right upper flank region with 1×10⁷ of HPAF-II tumor cells in 0.2 mL of PBS mixed with MATRIGEL® (1:1) for tumor development. When the average tumor volume reached 150-200 mm³, 88 mice were randomized into respective treatment groups (8 mice in each group) and received a single intravenous injection of vehicle, isotype control, or TF-ADC at the doses indicated in the table below.

TABLE E14

| Group | Test article | DAR | Dose (mg/kg) |
|---|---|---|---|
| 1 | vehicle | — | — |
| 2 | Isotype 2 | 8 | 10 |
| 3 | TF-ADC 6-8 | 8 | 1 |
| 4 | | 8 | 3 |
| 5 | | 8 | 6 |
| 6 | | 8 | 10 |
| 7 | Isotype 1 | 4 | 10 |
| 8 | TF-ADC 6-4 | 4 | 1 |
| 9 | | 4 | 3 |
| 10 | | 4 | 6 |
| 11 | | 4 | 10 |

After tumor cells inoculation, the animals were checked daily for morbidity and mortality. During routine monitoring, the animals were checked for any effects of tumor growth and treatments on behavior such as mobility, food and water consumption, body weight gain/loss (Body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume were expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet.

The body weights and tumor volumes were measured by using StudyDirector™ software (version 3.1.399.19). The study concluded when the mean tumor volume of the vehicle control group reached 2000 mm³ or 6 weeks after the final dose, whichever came first.

Bartlett's test was used to check the assumption of homogeneity of variance across all groups.

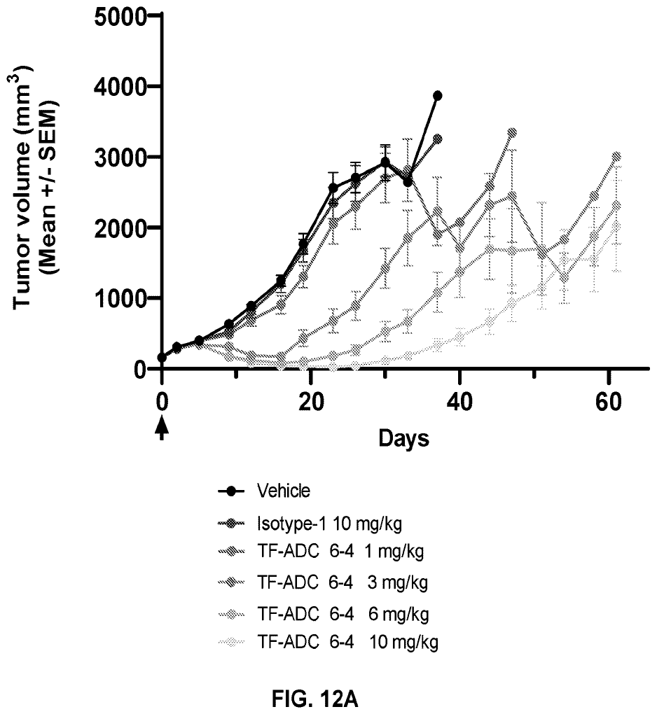

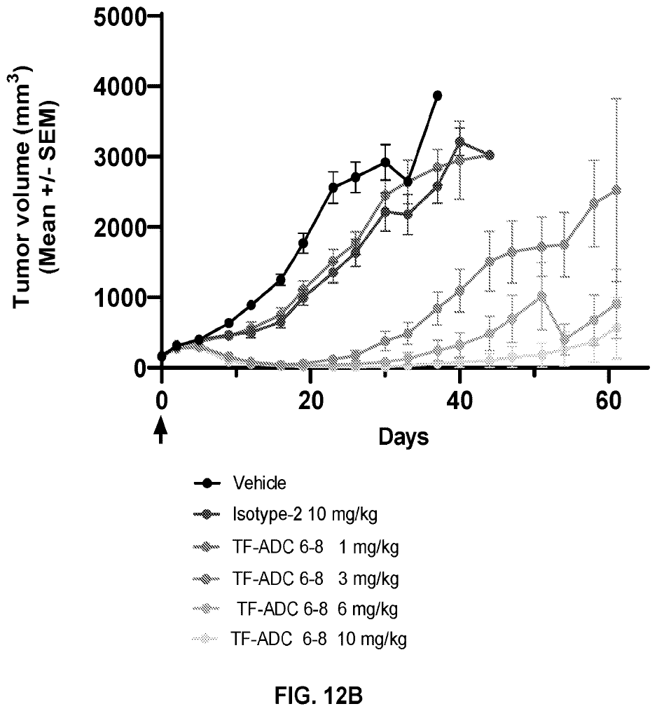

The effects of TF-ADC 6-4, and TF-ADC 6-8 on tumor regression and suppression in the HPAF-II xenograft model are shown in FIGS. 12A and 12B. Notably, These DAR4 and DAR8 ADCs demonstrated dose-related tumor suppression and comparable maximal inhibition in the HPAF-II xenograft model.

### Example 9—NHP Toxicity Study

To assess toxicity of the TF-ADCs, female cynomolgus (“cyno”) monkeys (n=2 per group) were treated via IV bolus with test articles at the doses indicated in the table below on days 1 and 22 (q3wX2). All monkeys that survived until day 43 of the study underwent euthanasia for necropsy and anatomic pathology evaluation. Clinical observations, ECG, eye exams, clinical and anatomic pathology, and TK analysis were performed.

TABLE E15

| Group No. | Test article | DAR | Animal # | Doses (mg/kg) |
|---|---|---|---|---|
| 1 | Vehicle control | — | 2 | — |
| 2 | TF-ADC 6-4 | 4 | 2 | 5 |
| 3 | | 4 | 2 | 10 |
| 4 | | 4 | 2 | 20 |
| 5 | | 4 | 2 | 40 |
| 6 | | 4 | 2 | 60 |
| 7 | TF-ADC 6-8 | 8 | 2 | 5 |
| 8 | | 8 | 2 | 10 |
| 9 | | 8 | 2 | 20 |
| 10 | | 8 | 2 | 40 |

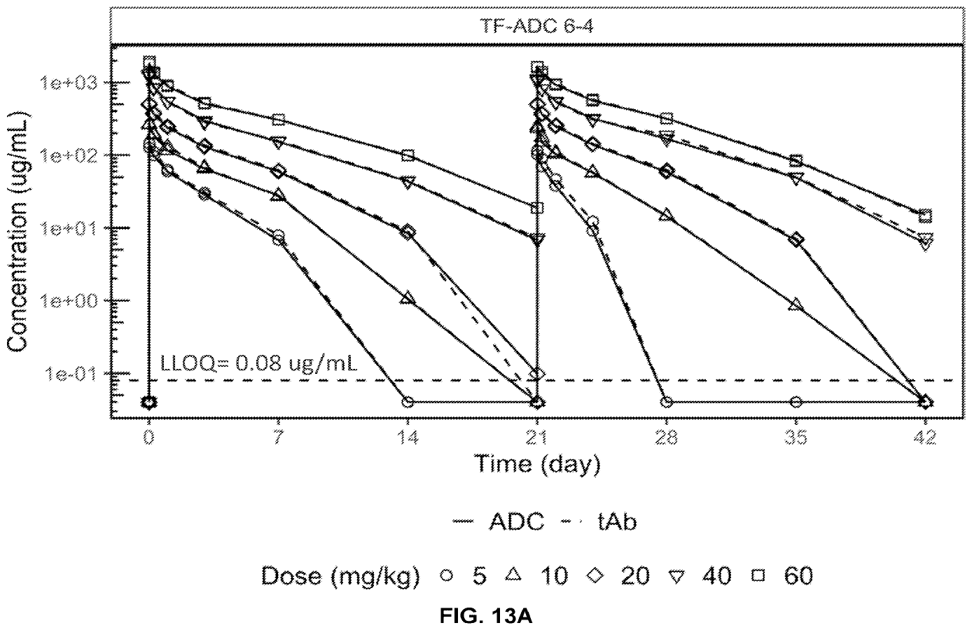

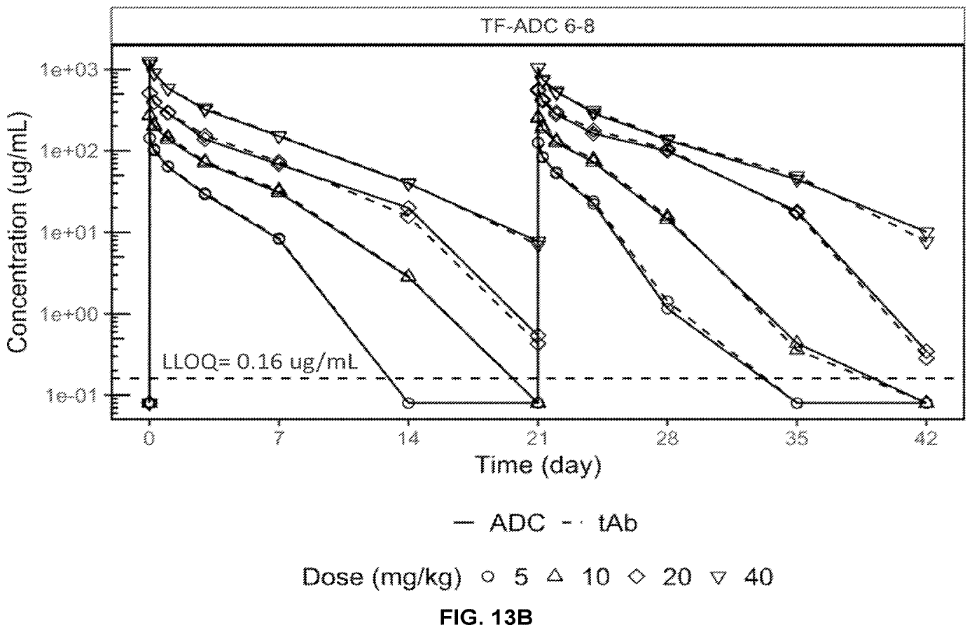

Concentrations of the antibodies and the whole ADCs of TF-ADC 6-4 and TF-ADC 6-8 ADC in cyno plasma were evaluated and the data is shown in FIGS. 13A and 13B.

This result shows that the total antibody and ADC concentration-time profiles are comparable for both tested ADCs. This result further indicates that TF-ADC 6-4 and TF-ADC 6-8 were tolerated up to the highest doses tested of 60 mg/kg and 40 mg/kg, respectively, when administered to NHP (cyno) on a q3wX2 schedule. Also, the serum concentration for ADCs and total antibody was increased more than dose-proportional. No accumulation was observed, and for low dose, the accumulation ratio was <1 suggesting a potential impact of anti-drug antibodies (ADA). The terminal half-life ranged from 1.8 to 3.77 days for TF-ADC 6-4 and 1.96 to 2.85 days for TF-ADC 6-8, similar to the TF ADC as described in WO2021003399 where half-life ranged from 2.07 to 3.47 days.

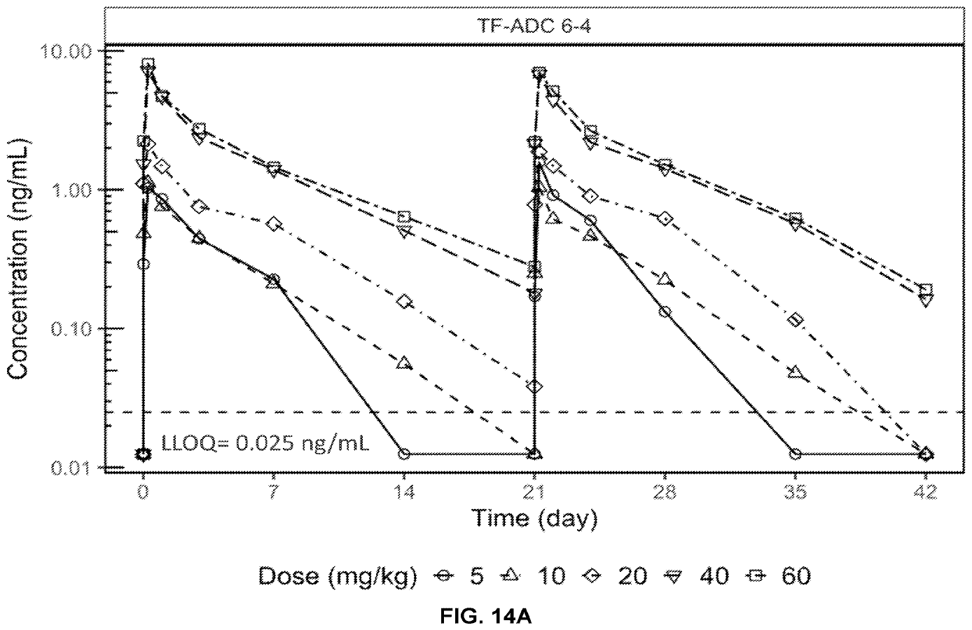

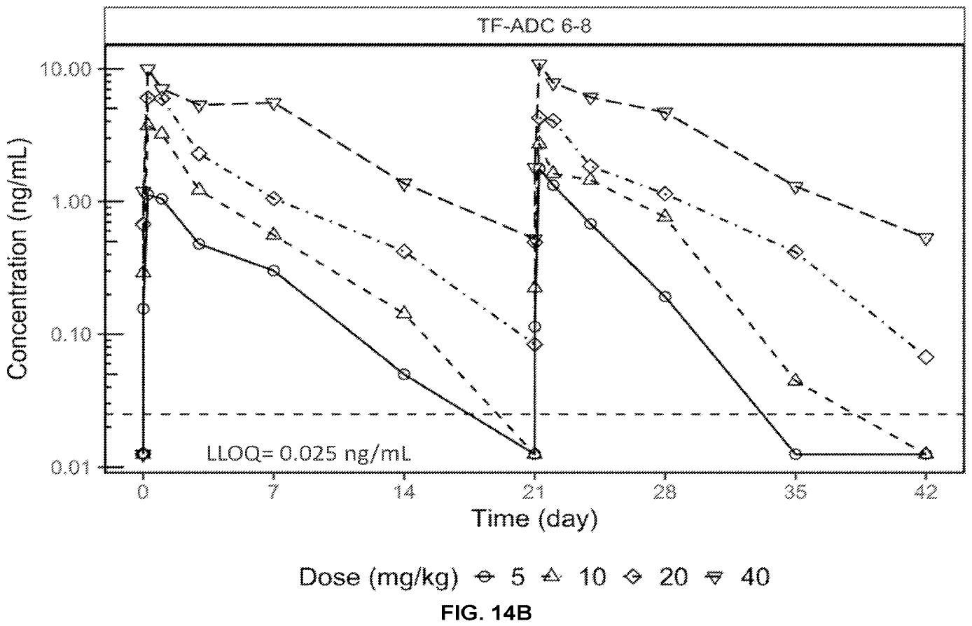

Further, payload concentrations in plasma of cyno treated with various dosages of TF-ADC 6-4 and TF-ADC 6-8 were evaluated and the data is shown in FIGS. 14A and 14B. This result indicates that low level of payload concentration for both ADCs suggested minimal deconjugation of the drug from the antibody in vivo. The plasma level of belotecan was low relative to ADCs and total antibody with a molar ratio (AUC_{0-21d}) ranging from 169 to 519 for TF-ADC 6-4 and 130 to 156 for TF-ADC 6-8. TF-ADC 6-4 had approximately 2-fold higher belotecan compared to TF-ADC 6-8 for the same dose level. Minimal accumulation of payload was observed. Belotecan's apparent half-life ranged between 3 to 6 days (Formation rate-limited).

### Example 10—Immune Activation

Treatment-induced immunogenic cell death (ICD) can stimulate anti-tumor immune responses that reinforce therapeutic effects (Fucikova et al. Cell Death Dis. 2020 Nov. 26; 11(11):1013). Accordingly, the TF-ADC 6-8 was further assessed for its effects on immune activation, e.g., ICD, immune cell activation (myeloid and T cells), and cytokine release.

Tumor cell treatment: Tumor cells (such as A431 or SKOV-3) were trypsinized by washing with PBS, then 3 mL of TrypLE express was added to each T75 flask and incubated for 3-5 minutes at 37° C. DMEM containing 10% FBS (Gibco) was added to each flask to gently wash cells. Cells were then transferred to a conical tube and centrifuged at 1000RPM for 5 minutes and resuspended in fresh medium. Cells were counted and the cell concentration was adjusted to 10,000 cells per 80 μL. Cells were plated at 80 μL per well and incubated at 37° C., 5% CO₂ for 5 hours to allow cells to adhere to the plate surface. 5× dilutions of treatment compounds were made in medium. 20 L of treatment were added to each well and mixed gently and incubated at 37° C., 5% CO₂. Belotecan, MMAE, and a benchmark ADC (also referred to herein as BM ADC, CAS 1418731-10-8 and INN 10148) were also tested in parallel and served as additional controls.

ICD molecule, such as ATP and HMGB1, readouts were performed as follows. For ATP release measurement, Promega RealTimeGlo extracellular ATP assay kit (Promega, Cat #GA5010) was used according to the manufacturer's instructions. Cells were cultured using Leibovitz L15 CO₂-less medium (Gibco). ATP release was measured beginning 24 hours after treatment with readings every 10 minutes on a Spectramax i3× with temperature control at 37° C. For HMGB1 release measurement, Lumit HMGB1 assay kit (Promega, Cat #W6110) was used according to the manufacturer's instructions. HMGB1 release was measured at 48- and 72-hours post-treatment.

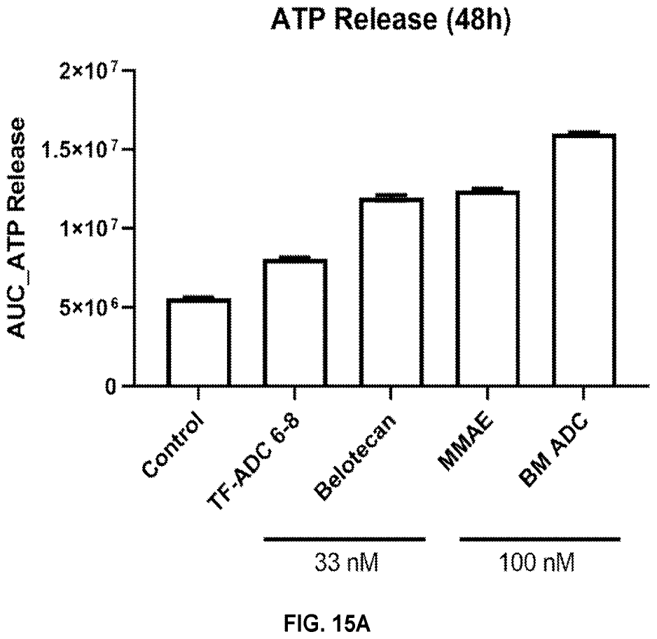

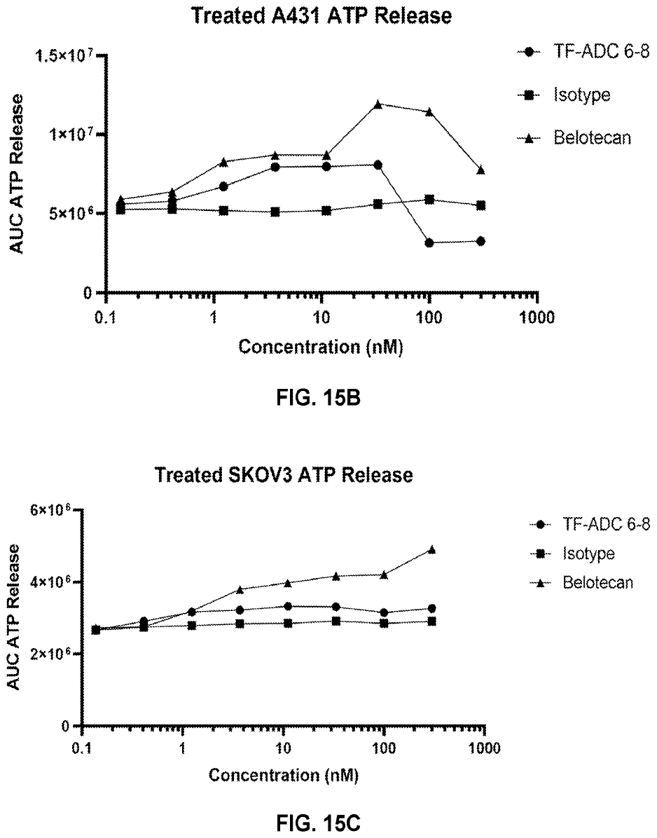

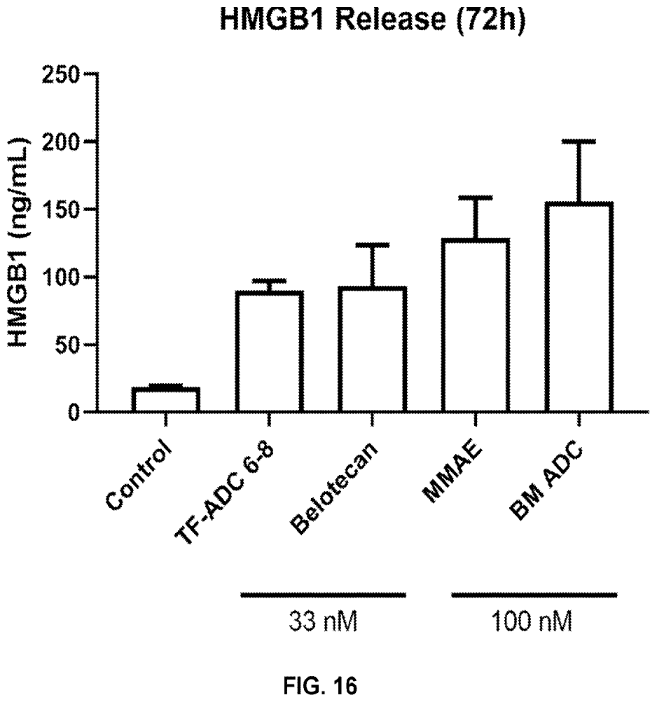

Exemplary ATP release results are provided in FIGS. 15A-15C (FIGS. 15A and 15B, A431; FIG. 15C, SKOV3), while FIG. 16 plots HMGB1 released by treated A431 tumor cells. FIGS. 15B and 15C provide area under the curve (AUC) of the ATP release results of tumor cells treated at various concentrations of TF-ADC 6-8, isotype control (an isotype IgG1 antibody conjugated to belotecan), or belotecan, while FIG. 15A and FIG. 16 plot the AUCs in the treatment groups of control (an isotype IgG1 antibody conjugated to belotecan), 33 nM TF-ADC 6-8, 33 nM belotecan, 100 nM MMAE, or 100 nM benchmark ADC.

These results showed that TF-ADC 6-8 or belotecan induced the release of immunostimulatory molecules, such as HMGB1 and ATP, in the TF+ tumor cell line, e.g., A431; and TF-ADC 6-8 treated tumor cells released higher levels of ATP over 24 hours.

Peripheral blood mononuclear cell (PBMC) co-culture: After 48 hours of tumor cell treatment, frozen human PBMCs were thawed into pre-warmed medium (RPMI with 10% FBS, HEPES, Glutamax, NEAA, and PenStrep). Cells were centrifuged at 1000RPM for 5 minutes. Cells were resuspended in PBS with Cell Trace Violet (CTV 1:6000) for 15 minutes at 37° C. An equivalent volume of medium was added and incubated for another 5 minutes to neutralize the cell trace violet stain. Cells were centrifuged and resuspended at 1×10⁶ cells/mL. 40U/mL of IL2 was added to maintain cell viability. For the free MMAE combination treatment, an anti-PD1 antibody (also referred to herein as aPD1, CAS Number 1374853-91-4) was added at 50 nM. 50 μL of PBMCs (50,000 cells) were added to each well and co-cultured for 24 to 96 hours and analyzed by flow cytometry. Supernatants from 24 to 96 hours were also collected for cytokine production analysis.

Cytokine production analysis: For immune activation cytokine production, a custom UPLEX kit was ordered from Meso Scale Discovery to measure MIP-1α, MIP-1β, IP-10, IFNγ, and TNFα. Supernatants were tested according to the manufacturer's instructions and read out on a Meso QuickPlex instrument and analyzed on Discovery Workbench software.

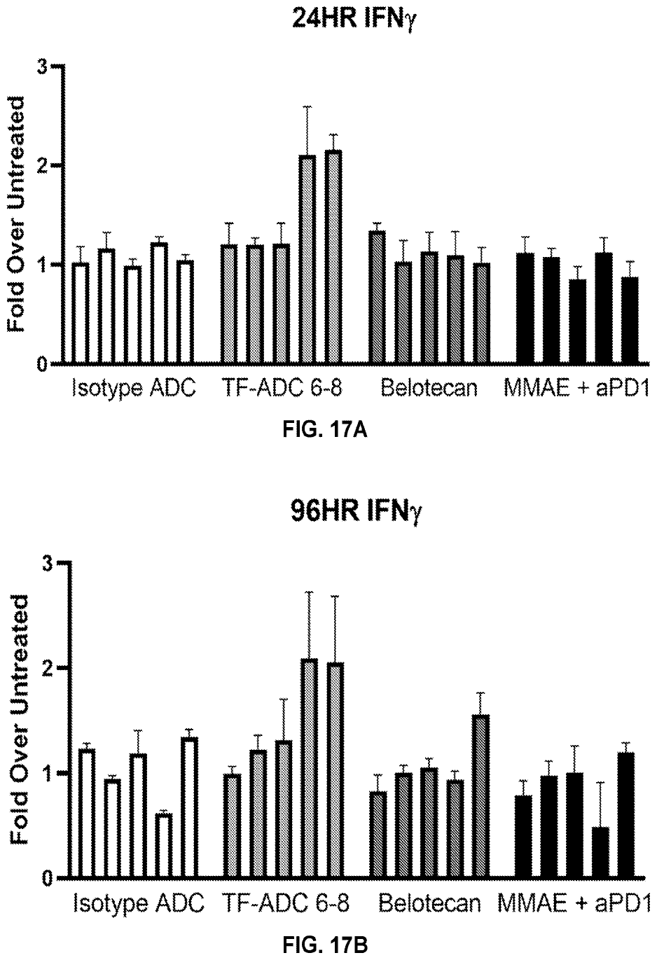

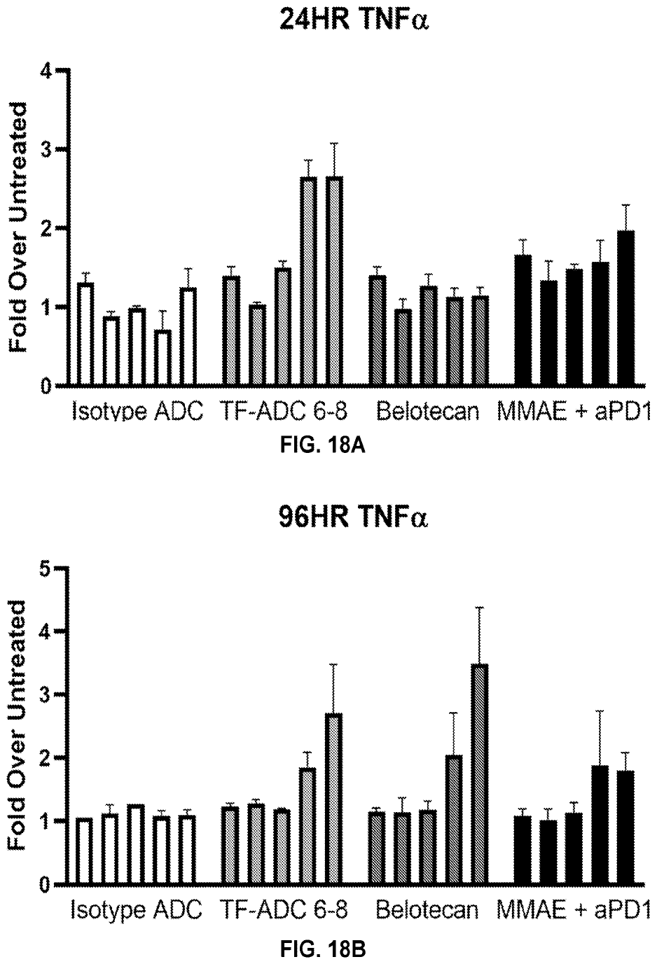

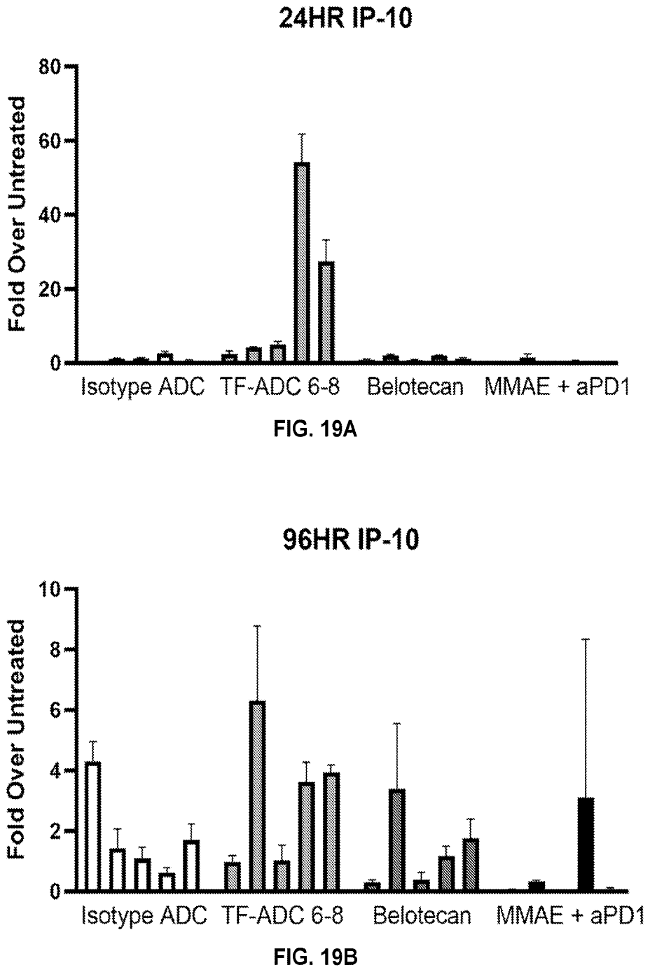

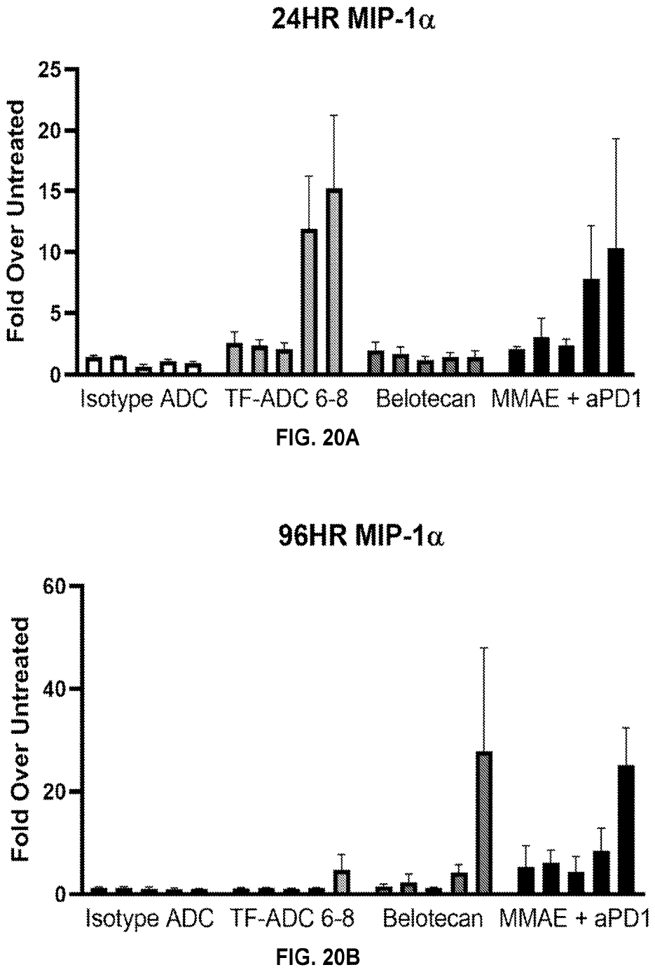

Results are provided in FIGS. 17A-20B, indicating that TF-ADC 6-8 treatment of TF-expressing tumor cells can cause immunogenic cell death (ICD) that can activate immune cells to induce production of inflammatory cytokines.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. An antibody-drug conjugate (ADC) of Formula (I) comprising:
a. an antibody that binds to tissue factor (TF); and
b. two or more drugs conjugated to a pyridazine-pyrrolo coupling moiety, each via a linker 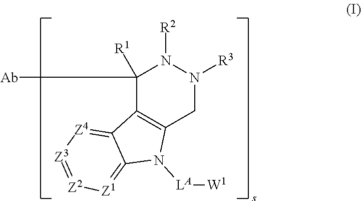
wherein:
Ab represents the antibody that binds to TF;
Z¹, Z², and Z⁴ are each independently CR⁴;
Z³ is C—L^{B}—W²;
R¹, R², R³, and R⁴ are each selected from hydrogen and alkyl;
L^{A} is a first linker comprising: 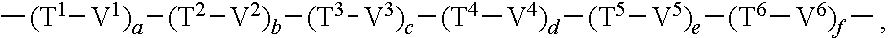
wherein:
a, b, c, d, e and f are each independently 0 or 1, provided at least one of a, b, c, d, e, and f are 1;
T¹, T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V¹, V², V³, V⁴ V⁵ and V⁶ are each independently selected from the group consisting of a covalent bond, —CO—, —NR—, —NR¹⁵(CH₂)_{q}—, —NR¹⁵(C₆H₄)—, —CONR⁵—, —NR¹⁵CO—, —C(O)O—, —OC(O)—, —O—, —S—, —S(O)—, —SO₂—, —SO₂NR⁵—, —NR¹⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
L^{B} is a second linker comprising: 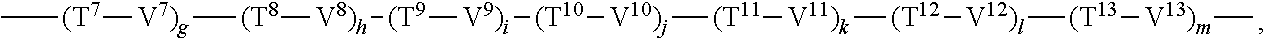
wherein:
g, h, i, j, k, l and m are each independently 0 or 1, provided that at least one of g, h, i, j, k, 1, and m is 1;
T⁷, T⁸, T⁹, T¹⁰, T¹¹, T¹² and T¹³ are each independently selected from a covalent bond, (C₁-C₁₂)alkyl, substituted (C₁-C₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), meta-amino-benzyloxy (MABO), meta-amino-benzyloxycarbonyl (MABC), para-amino-benzyloxy (PABO), para-amino-benzyloxycarbonyl (PABC), para-aminobenzyl (PAB), para-amino-benzylamino (PABA), para-amino-phenyl (PAP), para-hydroxy-phenyl (PUP), an acetal group, a hydrazine, a disulfide, and an ester, wherein EDA is an ethylene diamine moiety, PEG is a polyethylene glycol, and AA is an amino acid residue or an amino acid analog, wherein each w is an integer from 1 to 20, each n is an integer from 1 to 30, each p is an integer from 1 to 20, and each x is an integer from 1 to 12;
V⁷, V⁸, V⁹, V¹⁰, V¹¹, V¹² and V¹³ are each independently selected from the group consisting of a covalent bond, —CO—, —NR⁵—, —NR¹⁵(CH₂)_{q}—, —NR⁵(C₆H₄)—, —CONR¹⁵, —NR⁵CO—, —C(O)O—, —OC(O)—, —O—, —S—, —S(O)—, —SO₂—, —SO₂NR¹⁵—, —NR⁵SO₂— and —P(O)OH—, wherein each q is an integer from 1 to 6;
each R¹³ is independently selected from hydrogen, an alkyl, a substituted alkyl, an aryl, and a substituted aryl; and
each R¹⁵ is independently selected from hydrogen, alkyl, substituted alkyl, alkenyl, substituted alkenyl, alkynyl, substituted alkynyl, carboxyl, carboxyl ester, acyl, aryl, substituted aryl, heteroaryl, substituted heteroaryl, cycloalkyl, substituted cycloalkyl, heterocyclyl, and substituted heterocyclyl;
s is an integer from 1 to 10;
W¹ is a first drug; and
W² is a second drug.

2. The ADC of claim 1, wherein:
T¹ is selected from a (C₁-C₁₂)alkyl and a substituted (C₁-C₁₂)alkyl;
T², T³, T⁴, T⁵ and T⁶ are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V¹, V², V³, V⁴ V⁵ and V⁶ are each independently selected from the group consisting of a covalent bond, —CO—, —NR⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
(PEG)ₙ is 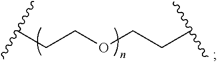
EDA is an ethylene diamine moiety having the following structure: 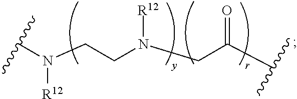
4-amino-piperidine (4AP) is 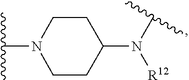
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
q is an integer from 1 to 6;
r is 0 or 1; and
y is an integer from 1 to 6.

3. The ADC of claim 1 or 2, wherein:
T¹ is (C₁-C₁₂)alkyl and V¹ is —CONH—;
T² is substituted (C₁-C₁₂)alkyl and V² is —CO—;
T³ is (AA)ₚ and V³ is absent;
T⁴ is PABC and V⁴ is absent;
p is an integer from 1 to 10; and
a, b, c, and d are each 1; and
e and f are each 0.

4. The ADC of any one of claims 1-3, wherein:
T⁷ is a covalent bond;
T⁸, T⁹, T¹⁰, T¹¹ and T¹² are each independently selected from a covalent bond, (C₁-C₁₂)alkyl, substituted (C₁-C₁₂)alkyl, aryl, substituted aryl, heteroaryl, substituted heteroaryl, cycloalkyl, substituted cycloalkyl, heterocyclyl, and substituted heterocyclyl, (EDA)_{w}, (PEG)ₙ, (AA)ₚ, —(CR¹³OH)ₓ—, 4-amino-piperidine (4AP), MABO, MABC, PABO, PABC, PAB, PABA, PAP, PUP, an acetal group, a hydrazine, and an ester; and
V⁷, V⁸, V⁹, V¹⁰ V¹¹ and V¹² are each independently selected from the group consisting of a covalent bond, —CO—, —NR¹⁵—, —NR¹⁵(CH₂)_{q}—, —NR¹⁵(C₆H₄)—, —CONR¹⁵—, —NR¹⁵CO—, —C(O)O—, —OC(O)—, —O—, —S—, —S(O)—, —SO₂—, —SO₂NR¹⁵—, —NR¹⁵SO₂—, and —P(O)OH—;
wherein:
(PEG)ₙ is 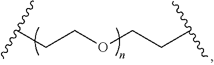
where n is an integer from 1 to 30;
EDA is an ethylene diamine moiety having the following structure: 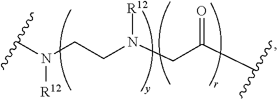
where y is an integer from 1 to 6 and r is 0 or 1;
4-amino-piperidine (4AP) is 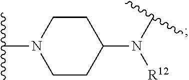
and
each R¹² is independently selected from hydrogen, an alkyl, a substituted alkyl, a polyethylene glycol moiety, an aryl and a substituted aryl, wherein any two adjacent R¹² groups may be cyclically linked to form a piperazinyl ring;
g, h, i, j, and k are each 1; and
l and m is 0.

5. The ADC of any one of claims 1-4, wherein:
T⁷ is absent and V⁷ is —NHCO—;
T⁸ is (C₁-C₁₂)alkyl and V⁸ is —CONH—;
T⁹ is substituted (C₁-C₁₂)alkyl and V⁹ is —CO—;
T¹⁰ is (AA)ₚ and V¹⁰ is absent;
T¹¹ is PABC and V¹¹ is absent;
p is an integer from 1 to 10; and
g, h, i, j, and k are each 1; and
l and m are each 0.

6. The ADC of any one of claims 1-5 wherein one or both of T² and T⁹ is (C₁-C₆)alkylene substituted with —NHCO(PEG)ₖ, wherein k is an integer from 2 to 10.

7. The ADC of any one of claims 1-6, wherein p is 2.

8. The ADC of any one of claims 1-7, wherein s is 2 or 4.

9. The ADC of any one of claims 1-8, wherein one or both of W¹ and W² is a camptothecin analog.

10. The ADC of any one of claims 1-9, wherein the camptothecin analog is belotecan.

11. The ADC of any one of claim 1-10, wherein each of W¹ and W² is belotecan.

12. An ADC represented by Formula (II): 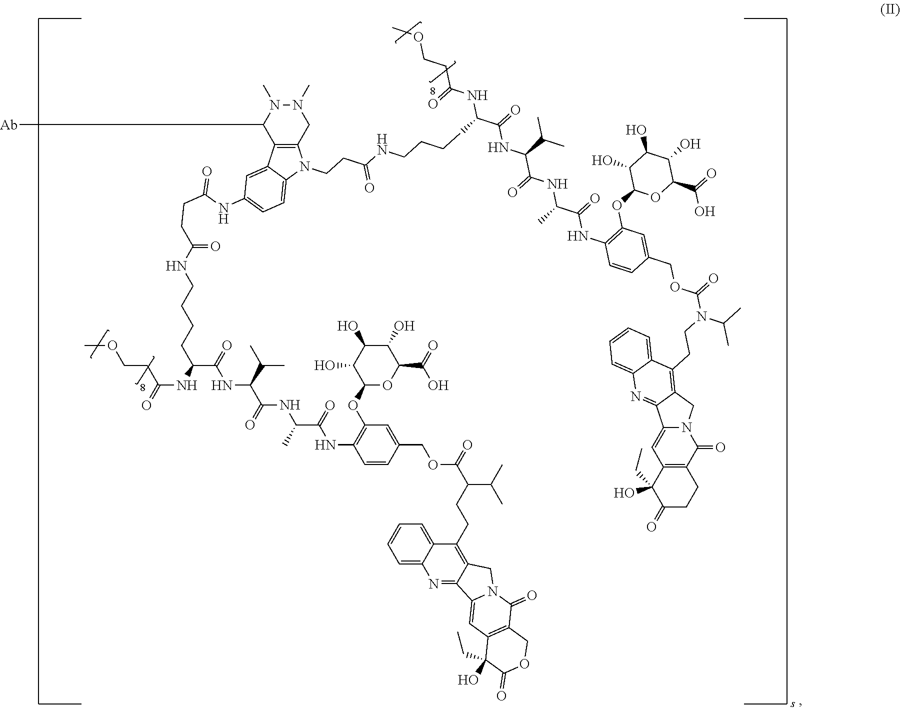
wherein:
Ab represents the antibody that binds to TF; and
s is an integer from 1 to 10.

13. The ADC of any one of claims 1-12, wherein Ab comprises: (i) a VH CDR1, a VH CDR2, and a VH CDR3 as set forth in a VH comprising the amino acid sequence of SEQ ID NO:25 and a VL CDR1, a VL CDR2, and a VL CDR3 as set forth in a VL comprising the amino acid sequence of SEQ ID NO:26; or (ii) a VH CDR1, a VH CDR2, and a VH CDR3 as set forth in a VH comprising the amino acid sequence of SEQ ID NO:41 and a VL CDR1, a VL CDR2, and a VL CDR3 as set forth in a VL comprising the amino acid sequence of SEQ ID NO:42.

14. The ADC of any one of claims 1-13, wherein Ab comprises: (i) a VH region comprises a VH CDR1 comprising the amino acid sequence of SEQ ID NOs:1, 7, 8, 15, 21, 27, 31, 32, 35, or 39, a VH CDR2 comprising the amino acid sequence of SEQ ID NOs:2, 9, 14, 16, or 22, and a VH CDR3 comprising the amino acid sequence of SEQ ID NOs:3, 10, 17, or 23; and a VL region comprising a VL CDR1 comprising the amino acid sequence of SEQ ID NOs:4, 11, 18, 24, 28, 33, 36, or 40, a VL CDR2 comprising the amino acid sequence of SEQ ID NOs:5, 12, 19, 29, or 37, and a VL CDR3 comprising the amino acid sequence of SEQ ID NOs:6, 13, 20, 30, 34, or 38.

15. The ADC of any one of claims 1-14, wherein Ab comprises one or more of a framework 1 (FR1), a framework 2 (FR2), a framework 3 (FR3) and a framework 4 (FR4) sequence as set forth in any one of SEQ ID NOs:25, 26, 41, and 42.

16. The ADC of any one of claims 1-14, wherein Ab comprises human framework sequences.

17. The ADC of any one of claims 1-15, wherein Ab comprises a VH comprising the amino acid sequence of SEQ ID NO:25 and a VL comprising the amino acid sequence of SEQ ID NO:26.

18. The ADC of any one of claims 1-17, wherein the Ab comprises a heavy chain comprising the amino acid sequence of SEQ ID NO:82.

19. The ADC of any one of claims 1-18, wherein the Ab comprises a heavy chain comprising the amino acid sequence of SEQ ID NO:82 and a light chain comprising the amino acid sequence of SEQ ID NO: 91.

20. The ADC of any one of claims 1-17, wherein the Ab comprises a heavy chain comprising the amino acid sequence of SEQ ID NO:79.

21. The ADC of any one of claims 1-17 and 20, wherein the Ab comprises a heavy chain comprising the amino acid sequence of SEQ ID NO:79 and a light chain comprising the amino acid sequence of SEQ ID NO: 91.

22. The ADC of any one of claims 1-15, wherein Ab comprises: (i) a VH comprising the amino acid sequence of SEQ ID NO:41 and a VL comprising the amino acid sequence of SEQ ID NO:42.

23. The ADC of any one of claims 1-15 and 22, wherein the Ab comprises a heavy chain comprising the amino acid sequence of SEQ ID NO:85.

24. The ADC of any one of claims 1-15 and 22-23, wherein the Ab comprises a heavy chain comprising the amino acid sequence of SEQ ID NO:85 and a light chain comprising the amino acid sequence of SEQ ID NO:92.

25. The ADC of any one of claims 1-15 and 22, wherein the Ab comprises a heavy chain comprising the amino acid sequence of SEQ ID NO:88.

26. The ADC of any one of claims 1-15, 22 and 25, wherein the Ab comprises a heavy chain comprising the amino acid sequence of SEQ ID NO:88 and a light chain comprising the amino acid sequence of SEQ ID NO:92.

27. A pharmaceutical composition comprising the ADC of any one of claims 1-26 and a pharmaceutically acceptable excipient.

28. The pharmaceutical composition of claim 27, characterized by an ADC drug-to-antibody ratio (DAR) of about 1 to about 20.

29. The pharmaceutical composition of claim 27 or 28, wherein the DAR is about 2 to about 8.

30. A method of treating a cancer or a tumor in a subject comprising administering to the subject the ADC of any one of claims 1-18 or the pharmaceutical composition of any one of claims 27-29.

31. The method of claim 30, wherein the cancer or tumor is characterized by expression of tissue factor.

32. The method of claim 30 or 31, wherein the cancer is breast cancer, colon cancer, renal cancer, lung cancer, squamous cell myeloid leukemia, hemangioma, melanoma, astrocytoma, glioblastoma, heart cancer, gastrointestinal cancer, pancreatic cancer, genitourinary cancer, liver cancer, bone cancer, skin cancer, adrenal cancer, or cancer of the nervous system.
